(19) United States
(12) Patent Application Publication
(10) Pub. No.: US 2020/0315678 A1
(43) Pub. Date: Oct. 8, 2020

(54) APPARATUS AND METHODS FOR BONE TREATMENT

(71) Applicant: Conventus Orthopaedics, Inc., Maple Grove, MN (US)

(72) Inventors: Michael Charles Mazzio, Schwenksville, PA (US); Jeffrey Lincoln Carver, Minnetonka, MN (US); Grazyna Wlodarski, Andover, MN (US); Eric Fulkerson, Winding Circle, CA (US); Frank Liporace, Englewood Cliffs, NJ (US); Michael Morwood, Salt Lake City, UT (US); Anjan Shah, Tampa, FL (US); Roy W. Sanders, Tampa, FL (US); Dylan J. Paproski, Wayne, PA (US); Christopher H. Scholl, West Chester, PA (US)

(21) Appl. No.: 16/739,107

(22) Filed: Jan. 9, 2020

Related U.S. Application Data
(60) Provisional application No. 62/790,158, filed on Jan. 9, 2019.
(60) Provisional application No. 62/790,449, filed on Jan. 9, 2019.
(60) Provisional application No. 62/823,767, filed on Mar. 26, 2019.
(60) Provisional application No. 62/927,199, filed on Oct. 29, 2019.
(60) Provisional application No. 62/927,200, filed on Oct. 29, 2019.

(51) Int. Cl. A61B 17/84; A61B 17/34

(52) U.S. Cl. CPC A61B 17/844; A61B 2017/681; A61B 17/3472; A61B 17/3421

(57) ABSTRACT

Apparatus and methods for treatment of a bone. The apparatus may include a mesh anchoring substrate extending longitudinally between a base and a hub. The base may have a base first end adjacent the mesh, a base second end opposite the base first end, and a cannula extending through the base. The apparatus may include a support. The support may include an elongated body extending between an end of the support and a flange. The end of the support may be configured to be removably coupled to a coupling mechanism in the hub. The flange may be configured for being seated outside the base against the base second end. A maximum circumference of the elongated body may be not greater than a cannula circumference.

### CROSS REFERENCE TO RELATED APPLICATIONS

This application is a nonprovisional of U.S. Provisional Application No. 62/790,158, filed on Jan. 9, 2019, and U.S. Provisional Application No. 62/790,449, filed on Jan. 9, 2019, and U.S. Provisional Application No. 62/823,767, filed on Mar. 26, 2019, and U.S. Provisional Application No. 62/927,199, filed on Oct. 29, 2019, and U.S. Provisional Application No. 62/927,200, filed on Oct. 29, 2019, all of which are hereby incorporated herein by reference in their entireties.

### BACKGROUND

Bone fracture fixation may involve using structures to counteract or partially counteract forces on a fractured bone or associated bone fragments. In general, fracture fixation may provide longitudinal (along the longitudinal axis of the bone), transverse (across the longitudinal axis of the bone), and rotational (about the longitudinal axis of the bone) stability. Fracture fixation may also preserve normal biologic and healing function.

Bone fracture fixation often involves addressing loading conditions, fracture patterns, alignment, compression force, and other factors, which may differ for different types of fractures. For example, midshaft fractures may have ample bone material on either side of the fracture in which anchors may be driven. End-bone fractures, especially near the articular surface may have thin cortical bone, soft cancellous bone, and relatively fewer possible anchoring locations.

Some fractures may require alignment and stability in a manner that generates adequate fixation in multiple directions.

Placement of the structures may be important for proper healing of the bone.

It would be desirable, therefore, to provide apparatus and methods for placement of structures.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows illustrative apparatus in accordance with principles of the invention;

FIG. 2 shows illustrative apparatus and methods in accordance with principles of the invention;

FIG. 3 shows illustrative apparatus accordance with principles of the invention;

FIG. 4 shows illustrative apparatus in accordance with principles of the invention;

FIG. 5 shows illustrative apparatus and methods in accordance with principles of the invention;

FIG. 6 shows illustrative apparatus in accordance with principles of the invention;

FIG. 7 shows illustrative apparatus and methods in accordance with principles of the invention;

FIG. 8 shows illustrative apparatus in accordance with principles of the invention;

FIG. 9 shows illustrative apparatus and methods in accordance with principles of the invention;

FIG. 10 shows illustrative apparatus and methods in accordance with principles of the invention;

FIG. 11 shows illustrative apparatus and methods in accordance with principles of the invention;

FIG. 12 shows illustrative apparatus and methods in accordance with principles of the invention;

FIG. 13 shows illustrative apparatus and methods in accordance with principles of the invention;

FIG. 14 shows illustrative apparatus and methods in accordance with principles of the invention;

FIG. 15 shows illustrative apparatus in accordance with principles of the invention;

FIG. 16 shows illustrative apparatus and methods in accordance with principles of the invention;

FIG. 17 shows illustrative apparatus and methods in accordance with principles of the invention;

FIG. 18 shows illustrative apparatus in accordance with principles of the invention;

FIG. 19 shows illustrative apparatus in accordance with principles of the invention;

FIG. 20 shows illustrative apparatus and methods in accordance with principles of the invention;

FIG. 21 shows illustrative apparatus and methods in accordance with principles of the invention;

FIG. 22 shows illustrative apparatus and methods in accordance with principles of the invention;

FIG. 23 shows illustrative apparatus in accordance with principles of the invention;

FIG. 24 shows illustrative apparatus in accordance with principles of the invention;

FIG. 25 shows illustrative apparatus in accordance with principles of the invention;

FIG. 26 shows illustrative apparatus in accordance with principles of the invention;

FIG. 27 shows illustrative apparatus in accordance with principles of the invention;

FIG. 28 shows illustrative apparatus in accordance with principles of the invention;

FIG. 29 shows illustrative apparatus and methods in accordance with principles of the invention;

FIG. 30 shows illustrative apparatus in accordance with principles of the invention;

FIG. 31 shows illustrative apparatus and methods in accordance with principles of the invention;

FIG. 32 shows illustrative apparatus in accordance with principles of the invention;

FIG. 33 shows illustrative apparatus in accordance with principles of the invention;

FIG. 34 shows illustrative apparatus in accordance with principles of the invention;

FIG. 35 shows illustrative apparatus in accordance with principles of the invention;

FIG. 36 shows illustrative apparatus in accordance with principles of the invention;

FIGS. 37A and 37B show illustrative apparatus and methods in accordance with principles of the invention;

FIG. 38 shows illustrative method steps in accordance with principles of the invention;

FIG. 39 shows illustrative apparatus in accordance with principles of the invention;

FIG. 40 shows illustrative apparatus in accordance with principles of the invention;

FIG. 41 shows illustrative apparatus and methods in accordance with principles of the invention;

FIG. 42 shows illustrative apparatus in accordance with principles of the invention;

FIGS. 43-48 show illustrative apparatus and methods in accordance with principles of the invention;

FIG. 49 shows illustrative apparatus in accordance with principles of the invention;

FIGS. 50-54 show illustrative apparatus and methods in accordance with principles of the invention;

FIG. 55 shows illustrative apparatus in accordance with principles of the invention;

FIG. 56 shows illustrative apparatus in accordance with principles of the invention;

FIGS. 57-58 show illustrative apparatus and methods in accordance with principles of the invention;

FIG. 59 shows illustrative apparatus and methods in accordance with principles of the invention;

FIG. 60 shows illustrative apparatus and methods in accordance with principles of the invention;

FIG. 61 shows illustrative apparatus and methods in accordance with principles of the invention;

FIG. 62 shows illustrative apparatus and methods in accordance with principles of the invention;

FIG. 63 shows illustrative apparatus and methods in accordance with principles of the invention;

FIG. 64 shows illustrative apparatus and methods in accordance with principles of the invention;

FIG. 65 shows illustrative apparatus and methods in accordance with principles of the invention;

FIG. 66 shows illustrative apparatus in accordance with principles of the invention;

FIG. 67 shows illustrative apparatus in accordance with principles of the invention;

FIG. 68 shows illustrative apparatus and methods in accordance with principles of the invention;

FIG. 69 shows illustrative apparatus and methods in accordance with principles of the invention;

FIG. 70 shows illustrative apparatus in accordance with principles of the invention;

FIG. 71 shows illustrative apparatus in accordance with principles of the invention;

FIG. 72 shows illustrative apparatus in accordance with principles of the invention;

FIG. 73 shows illustrative apparatus in accordance with principles of the invention;

FIG. 74 shows illustrative apparatus in accordance with principles of the invention;

FIG. 75 shows illustrative apparatus in accordance with principles of the invention;

FIGS. 76A, 76B, and 77-87 show illustrative implant shapes;

FIGS. 88A, 88B, and 89-102 show illustrative implants in bones;

FIG. 103 shows illustrative apparatus and methods in accordance with principles of the invention

FIG. 104 shows illustrative apparatus and methods in accordance with principles of the invention;

FIG. 105 shows illustrative apparatus in accordance with principles of the invention;

FIG. 106 shows a human skeleton.

FIG. 107 shows illustrative apparatus in accordance with principles of the invention;

FIG. 108 shows illustrative apparatus in accordance with principles of the invention;

FIG. 109 shows illustrative apparatus in accordance with principles of the invention;

FIG. 110 shows illustrative apparatus in accordance with principles of the invention;

FIG. 111 shows illustrative apparatus in accordance with principles of the invention;

FIG. 112 shows illustrative apparatus in accordance with principles of the invention;

FIG. 113 shows illustrative apparatus in accordance with principles of the invention;

FIG. 114 shows illustrative apparatus in accordance with principles of the invention;

FIG. 115 shows illustrative apparatus in accordance with principles of the invention;

FIG. 116 shows illustrative apparatus in accordance with principles of the invention;

FIG. 117 shows illustrative apparatus in accordance with principles of the invention;

FIG. 118 shows illustrative apparatus in accordance with principles of the invention;

FIG. 119 shows illustrative apparatus in accordance with principles of the invention;

FIG. 120 shows illustrative apparatus in accordance with principles of the invention;

FIG. 121 shows illustrative apparatus in accordance with principles of the invention;

FIG. 122 shows illustrative apparatus in accordance with principles of the invention;

FIG. 123 shows illustrative apparatus in accordance with principles of the invention;

FIG. 124 shows illustrative apparatus in accordance with principles of the invention;

FIG. 125 shows illustrative apparatus in accordance with principles of the invention;

FIG. 126 shows illustrative apparatus in accordance with principles of the invention;

FIG. 127 shows illustrative apparatus and methods in accordance with principles of the invention;

FIGS. 128 and 128A shows illustrative apparatus and methods in accordance with principles of the invention;

FIG. 129 shows illustrative apparatus and methods in accordance with principles of the invention;

FIGS. 130 and 130A shows illustrative apparatus in accordance with principles of the invention;

FIG. 131 shows illustrative apparatus in accordance with principles of the invention;

FIGS. 132A and 132B shows illustrative method steps in accordance with principles of the invention;

FIG. 133 shows illustrative apparatus and methods in accordance with principles of the invention;

FIG. 134 shows illustrative apparatus and methods in accordance with principles of the invention;

FIG. 135 shows illustrative apparatus in accordance with principles of the invention;

FIG. 136 shows illustrative apparatus and methods in accordance with principles of the invention;

FIG. 136A shows illustrative apparatus in accordance with principles of the invention;

FIG. 137 shows illustrative apparatus in accordance with principles of the invention;

FIG. 138 shows illustrative apparatus in accordance with principles of the invention;

FIG. 139 shows illustrative apparatus in accordance with principles of the invention;

FIG. 140 shows illustrative apparatus in accordance with principles of the invention;

FIG. 141 shows illustrative apparatus in accordance with principles of the invention;

FIG. 142 shows illustrative apparatus in accordance with principles of the invention;

FIG. 143 shows illustrative apparatus in accordance with principles of the invention;

FIG. 144 shows illustrative apparatus in accordance with principles of the invention;

FIG. 145 shows illustrative apparatus in accordance with principles of the invention;

FIG. 146 shows illustrative apparatus in accordance with principles of the invention;

FIG. 147 shows illustrative apparatus in accordance with principles of the invention;

FIG. 148A shows illustrative apparatus in accordance with principles of the invention;

FIG. 148B shows illustrative apparatus in accordance with principles of the invention;

FIG. 149 shows illustrative apparatus in accordance with principles of the invention;

FIG. 150 shows illustrative apparatus and methods in accordance with principles of the invention;

FIG. 151 shows illustrative apparatus and methods in accordance with principles of the invention;

FIG. 152 shows illustrative apparatus in accordance with principles of the invention;

FIG. 152A shows illustrative apparatus in accordance with principles of the invention;

FIG. 153 shows illustrative apparatus in accordance with principles of the invention;

FIG. 154 shows illustrative apparatus in accordance with principles of the invention;

FIG. 155 shows illustrative apparatus and methods in accordance with principles of the invention;

FIG. 156 shows illustrative apparatus and methods in accordance with principles of the invention;

FIG. 157 shows illustrative apparatus and methods in accordance with principles of the invention;

FIG. 158 shows illustrative apparatus in accordance with principles of the invention;

FIG. 159 shows illustrative apparatus in accordance with principles of the invention; and

FIG. 160 shows illustrative apparatus and methods in accordance with principles of the invention.

### DETAILED DESCRIPTION OF THE INVENTION

Apparatus and methods for reducing a fracture of a bone are provided.

Apparatus and methods for implanting an implant into an interior of the bone are provided.

Apparatus and methods for implanting a nail into an interior of the bone are provided.

Apparatus and methods for securing the fractured bone are provided.

The apparatus may be used with, and the methods may involve, a jig. The jig may include features that spatially register external bone anatomy to an implant target site in the bone. The jig features may be used to direct one or more wires, screws, broaching tools, plates and implants, or other suitable items, so that the implant may be delivered to the site.

The implant may include apparatus and methods described in U.S. patent application Ser. No. 12/353,855, filed on Jan. 14, 2009, now U.S. Pat. No. 8,287,538, U.S. patent application Ser. No. 13/43,190, filed on Mar. 8, 2011, now U.S. Pat. No. 8,906,22, U.S. patent application Ser. No. 13/945,137, filed on Jul. 18, 2013, and/or in U.S. patent application Ser. No. 16/27,338, filed on Jul. 4, 2018, all of which are hereby incorporated by reference herein in their entireties.

The broaching tool, cavity preparation and drilling may be performed using apparatus and methods described in U.S. patent application Ser. No. 13/009,657, filed on Jan. 19, 2011, now U.S. Pat. No. 8,961,518, U.S. patent application Ser. No. 14/568,301, filed on Dec. 12, 2014, and/or in U.S. patent application Ser. No. 16/27,338, filed on Jul. 4, 2018, all of which are hereby incorporated by reference herein in their entireties.

Reduction of a fracture and apparatus and methods for inserting an implant into a bone, may be performed using apparatus and methods described in U.S. patent application Ser. No. 13/43,330, filed on Mar. 8, 2011, U.S. patent application Ser. No. 13/414,695, filed on Mar. 7, 2012, and/or in U.S. patent application Ser. No. 16/27,338, filed on Jul. 4, 2018, all of which are hereby incorporated by reference herein in their entireties.

The implant may be formed from laser-cut tube stock. The implant may be formed from super elastic materials including nitinol, NiTiCu, titanium alloys, nickel alloys, spring steel alloys, carbon fiber composites, carbon-graphene, shape-memory polymers, polyisoprene-based polymers, calcium iron arsenide CaFe₂As₂ and similar materials.

The implant may be an implant that is not expandable. The implant may be an expandable implant. The implant may be radially expandable. The implant may be a self-expandable implant. The implant may include a shape memory alloy. The shape memory alloy may be provided with a pre-set shape. The implant may be compressed from the pre-set shape into a collapsed configuration in which the implant may be delivered through an access hole. After when released from the collapsed configuration, the implant may self-expand to the preset shape or to near the preset shape, if constrained by anatomy or instruments.

The implant may have any suitable shape in the expanded state. The shape may be defined by one or more shape factors. The shape factors may include a proximal base diameter xₐ, non-dimensional diameters D*(x) corresponding to implant landmarks along x from the proximal end of the base, and non-dimensional positions X* of the landmarks. Diameter D(x) is diameter, as a function of x, in millimeters.

One or more of the landmarks may be present in the implant. Table 1 shows illustrative landmarks.

TABLE 1
Illustrative landmarks.

| Landmark | Description |
|---|---|
| xₐ | Proximal end of base |
| x_{b} | Distal end of base |
| x_{c} | First critical value |
| x_{d} | Second critical value |
| xₑ | Third critical value |
| x_{f} | Fourth critical value |
| x_{g} | Distal end of implant |

A diameter at a critical value may be an inflection point in the profile of the implant. A diameter at a critical value may be a diameter that is not at an inflection point in the profile of the implant.

Table 2 shows illustrative value of diameter at xₐ.

```
TABLE 2
Illustrative values of diameter at xₐ.
D(xₐ) (mm)
  1
  2
  3
  4
  5
  6
  7
  8
  9
10
11
12
13
14
15
16
17
18
19
20
21
22
23
24
25
26
27
28
29
30
31
32
33
34
35
36
37
38
39
40
41
42
43
44
45
46
47
48
49
50
  50+
```

Table 3 shows illustrative non-dimensional diameters that may be associated with the different landmarks.

TABLE 3
Illustrative non-dimensional diameters that
may be associated with the different landmarks.
D*
(D* = D(xᵢ)/D(xₐ),
i = a, b, c, d, e, f, g)

| Xₐ | X_{b} | X_{c} | X_{d} | | Xₑ | | X_{f} | X_{g} |
|---|---|---|---|---|---|---|---|---|
| 1 | 1 | 1 | 1 | 1.6 | 1 | 2.4 | 1 | 1 |
| | | 1.05 | 0.4 | 1.65 | 0.4 | 2.5 | 1.05 | 1.05 |
| | | 1.1 | 0.45 | 1.7 | 0.5 | 2.6 | 1.1 | 1.1 |
| | | 1.15 | 0.5 | 1.75 | 0.6 | 2.7 | 1.15 | 1.15 |
| | | 1.2 | 0.55 | 1.8 | 0.7 | 2.8 | 1.2 | 1.2 |
| | | 1.25 | 0.6 | 1.85 | 0.8 | 2.9 | 1.25 | 1.25 |
| | | 1.3 | 0.65 | 1.9 | 0.9 | 3 | 1.3 | 1.3 |
| | | 1.35 | 0.75 | 1.95 | 1 | 3.1 | 1.35 | 1.35 |
| | | 1.4 | 0.85 | 2 | 1.1 | 3.2 | 1.4 | 1.4 |
| | | 1.45 | 0.95 | 2.05 | 1.2 | 3.3 | 1.45 | 1.45 |
| | | 1.5 | 1 | 2.1 | 1.3 | 3.4 | 1.5 | 1.5 |
| | | >1.5 | 1.05 | 2.15 | 1.4 | 3.5 | >1.5 | >1.5 |
| | | | 1.1 | 2.2 | 1.5 | 3.6 | | |
| | | | 1.15 | 2.25 | 1.6 | 3.7 | | |
| | | | 1.2 | 2.3 | 1.7 | 3.8 | | |
| | | | 1.25 | 2.35 | 1.8 | 3.9 | | |
| | | | 1.3 | 2.4 | 1.9 | 4 | | |
| | | | 1.35 | 2.45 | 2 | 4.1 | | |
| | | | 1.4 | 2.5 | 2.1 | 4.2 | | |
| | | | 1.45 | >2.5 | 2.2 | 4.3 | | |
| | | | 1.5 | | 2.3 | 4.4 | | |
| | | | 1.55 | | >2.3 | 4.5 | | |
| | | | >1.55 | | | 4.6 | | |
| | | | | | | 4.7 | | |
| | | | | | | 4.8 | | |
| | | | | | | 4.9 | | |
| | | | | | | 5 | | |
| | | | | | | >5 | | |

Table 4 shows illustrative diameters D(x), showing contours that may be present over different intervals of x.

TABLE 4
Illustrative diameters D(x), showing contours that
may be present over different intervals of x.
D(x) over the interval xᵢ to xᵢ₊₁

Constant
Linearly increasing
Linearly decreasing
Quadratically increasing
Quadratically decreasing
Any other suitable form

Table 5 shows illustrative values of X* for each landmark.

TABLE 5
Illustrative values of X* for each landmark.
X*
(X* = xᵢ/x_{g},
i = a, b, c, d, e, f g)

| xₐ | x_{b} | x_{c} | x_{d} | xₑ | x_{f} | x_{g} |
|---|---|---|---|---|---|---|
| 0 | 1 | 1 | 1 | 1 | 1 | 1 |
| | 0.01 | 0.01 | 0.01 | 0.01 | 0.01 | |
| | 0.05 | 0.05 | 0.05 | 0.05 | 0.05 | |
| | 0.1 | 0.1 | 0.1 | 0.1 | 0.1 | |
| | 0.15 | 0.15 | 0.15 | 0.15 | 0.15 | |
| | 0.2 | 0.2 | 0.2 | 0.2 | 0.2 | |
| | 0.25 | 0.25 | 0.25 | 0.25 | 0.25 | |
| | 0.3 | 0.3 | 0.3 | 0.3 | 0.3 | |
| | 0.35 | 0.35 | 0.35 | 0.35 | 0.35 | |
| | 0.4 | 0.4 | 0.4 | 0.4 | 0.4 | |
| | 0.45 | 0.45 | 0.45 | 0.45 | 0.45 | |
| | 0.5 | 0.5 | 0.5 | 0.5 | 0.5 | |
| | | 0.55 | 0.55 | 0.55 | 0.55 | |
| | | 0.6 | 0.6 | 0.6 | 0.6 | |
| | | 0.65 | 0.65 | 0.65 | 0.65 | |
| | | 0.7 | 0.7 | 0.7 | 0.7 | |
| | | 0.75 | 0.75 | 0.75 | 0.75 | |
| | | 0.8 | 0.8 | 0.8 | 0.8 | |
| | | 0.85 | 0.85 | 0.85 | 0.85 | |
| | | 0.9 | 0.9 | 0.9 | 0.9 | |
| | | 0.95 | 0.95 | 0.95 | 0.95 | |
| | | >0.95 | >0.95 | >0.95 | 0.99 | |
| | | | | | >0.99 | |

Table 6 Illustrative examples of selected shape factor combinations.

TABLE 6
Illustrative examples of selected shape factor combinations
(wherein “—” indicates unspecified).

| | | | |
|---|---|---|---|
| | x | D(xᵢ) | D(x) |
| Landmark | (mm) | (mm) | (mm) |

Example A.

| | | | |
|---|---|---|---|
| xₐ | — | — | |
| xₐ to x_{b} | — | — | Constant |
| x_{b} | — | — | |
| x_{b} to x_{c} | — | — | Increasing at quadratic or higher- |
| | | | order, convex-in. |
| x_{c} | — | 30 | |
| x_{c} to x_{d} | — | — | Constant |
| x_{d} | — | 30 | |
| x_{d} to xₑ | — | — | Constant, continuation of D(x) for |
| | | | x_{c} to x_{d}. |
| xₑ | — | 30 | |
| xₑ to x_{f} | — | — | Spherical, convex outward |
| x_{f} | — | At spherical | |
| | | surface of | |
| | | D(x) for xₑ | |
| | | to x_{f} | |
| x_{f} to x_{g} | — | — | Spherical, convex outward, continu- |
| | | | ation of D(x) for xₑ to x_{f} |
| x_{g} | 90 | — | Non-zero |

Example B.

| | | | |
|---|---|---|---|
| xₐ | — | — | |
| xₐ to x_{b} | — | — | Constant |
| x_{b} | — | — | |
| x_{b} to x_{c} | — | — | Increasing nonlinearly |
| x_{c} | — | — | |
| x_{c} to x_{d} | — | — | Linearly increasing at first slope |
| x_{d} | — | — | |
| x_{d} to xₑ | — | — | Linearly increasing at first slope, |
| | | | continuation of D(x) for x_{c} to x_{d}. |
| xₑ | — | 38 | |
| xₑ to x_{f} | — | — | Linearly decreasing at second slope |
| | | | having magnitude greater than |
| | | | magnitude of second slope |
| x_{f} | — | At surface | |
| | | of D(x) for | |
| | | xₑ to x_{f} | |
| x_{f} to x_{g} | — | — | Linearly decreasing, continuation of |
| | | | D(x) for xₑ to x_{f} |
| x_{g} | 90 | — | Non-zero |

Example C.

| | | | |
|---|---|---|---|
| xₐ | — | — | |
| xₐ to x_{b} | — | — | Constant |
| x_{b} | — | — | |
| x_{b} to x_{c} | — | — | Increasing nonlinearly, quadratic or |
| | | | higher-order, convex-in |
| x_{c} | — | 21 | |
| x_{c} to x_{d} | — | — | Linearly increasing at first slope |
| x_{d} | — | — | |
| x_{d} to xₑ | — | — | Linearly increasing at first slope, |
| | | | continuation of D(x) for x_{c} to x_{d}. |
| xₑ | — | 38 | |
| xₑ to x_{f} | — | — | Linearly decreasing at second slope |
| | | | having magnitude greater than |
| | | | magnitude of first slope |
| x_{f} | — | At surface | |
| | | of D(x) for | |
| | | xₑ to x_{f} | |
| x_{f} to x_{g} | — | — | Linearly decreasing, continuation of |
| | | | D(x) for xₑ to x_{f} |
| x_{g} | 90 | — | Non-zero |

Example D.

| | | | |
|---|---|---|---|
| xₐ | — | — | |
| xₐ to x_{b} | — | — | Constant |
| x_{b} | — | — | |
| x_{b} to x_{c} | — | — | Nonlinearly increasing, convex-in |
| x_{c} | — | — | |
| x_{c} to x_{d} | — | — | Convex-out, slope gradually flattening |
| x_{d} | — | — | |
| x_{d} to xₑ | — | — | Convex-out, slope gradually flattening |
| | | | until x = xₑ, continuation of D(x) |
| | | | for x_{c} to x_{d}. |
| xₑ | — | 36 | |
| xₑ to x_{f} | — | — | Spherical, convex outward |
| x_{f} | — | At spherical | |
| | | surface of | |
| | | D(x) for xₑ | |
| | | to x_{f} | |
| x_{f} to x_{g} | — | — | Spherical, convex outward, continuation |
| | | | of D(x) for xₑ to x_{f} |
| x_{g} | 90 | — | Non-zero |

Example E.

| | | | |
|---|---|---|---|
| xₐ | — | — | |
| xₐ to x_{b} | — | — | Constant |
| x_{b} | — | — | |
| x_{b} to x_{c} | — | — | Nonlinearly increasing |
| x_{c} | — | 30 | |
| x_{c} to x_{d} | — | — | Linearly decreasing |
| x_{d} | — | <30 | |
| x_{d} to xₑ | — | — | Linearly increasing. |
| xₑ | — | 38 | |
| xₑ to x_{f} | — | — | Linearly decreasing. |
| x_{f} | — | At surface | |
| | | of D(x) for | |
| | | xₑ to x_{f} | |
| x_{f} to x_{g} | — | — | Linearly decreasing, continuation of |
| | | | D(x) for xₑ to x_{f} |
| x_{g} | 90 | — | Non-zero |

Example F.

| | | | |
|---|---|---|---|
| xₐ | — | — | |
| xₐ to x_{b} | — | — | Constant |
| x_{b} | — | — | |
| x_{b} to x_{c} | — | — | Nonlinearly increasing |
| x_{c} | — | — | |
| x_{c} to x_{d} | — | — | Linearly increasing at first slope |
| x_{d} | — | — | |
| x_{d} to xₑ | — | — | Linearly increasing at first slope, |
| | | | continuation of D(x) for x_{c} to x_{d}. |
| xₑ | — | 37 | |
| xₑ to x_{f} | — | — | Linearly decreasing at second slope |
| | | | having magnitude greater than |
| | | | magnitude of first slope |
| x_{f} | — | At surface | |
| | | of D(x) for | |
| | | xₑ to x_{f} | |
| x_{f} to x_{g} | — | — | Linearly decreasing, continuation of |
| | | | D(x) for xₑ to x_{f} |
| x_{g} | 89 | — | Non-zero |

Example G.

| | | | |
|---|---|---|---|
| xₐ | — | — | |
| xₐ to x_{b} | — | — | Constant |
| x_{b} | — | — | |
| x_{b} to x_{c} | — | — | Nonlinearly increasing, convex-in |
| x_{c} | — | — | |
| x_{c} to x_{d} | — | — | Convex-out, slope gradually flat- |
| | | | tening |
| x_{d} | — | — | |
| x_{d} to xₑ | — | — | Convex-out, slope gradually flat- |
| | | | tening until x = xₑ, continuation |
| | | | of D(x) for x_{c} to x_{d}. |
| xₑ | — | 35 | |
| xₑ to x_{f} | — | — | Spherical, convex outward |
| x_{f} | — | At spherical | |
| | | surface of | |
| | | D(x) for xₑ | |
| | | to x_{f} | |
| x_{f} to x_{g} | — | — | Spherical, convex outward, continu- |
| | | | ation of D(x) for xₑ to x_{f} |
| x_{g} | 89 | — | Non-zero |

Example H.

| | | | |
|---|---|---|---|
| xₐ | — | — | |
| xₐ to x_{b} | — | — | Constant |
| x_{b} | — | — | |
| x_{b} to x_{c} | — | — | Nonlinearly increasing, convex-in |
| x_{c} | — | — | |
| x_{c} to x_{d} | — | — | Convex-out, slope gradually flat- |
| | | | tening with increasing x |
| x_{d} | — | — | |
| x_{d} to xₑ | — | — | Convex-out, slope gradually flat- |
| | | | tening until x = xₑ, continuation |
| | | | of D(x) for x_{c} to x_{d}. |
| xₑ | — | 36 | |
| xₑ to x_{f} | — | — | Linearly decreasing |
| x_{f} | — | At spherical | |
| | | surface of | |
| | | D(x) for xₑ | |
| | | to x_{f} | |
| x_{f} to x_{g} | — | — | Linearly decreasing, continuation of |
| | | | D(x) for xₑ to x_{f} |
| x_{g} | 89 | — | Non-zero |

The foregoing examples are illustrative. Implants with numerous different combinations of the shape factors described herein, or other shape factors, are contemplated.

The implanted position of the implant may be defined by one or more fit factors. The fit factors that vary with x may be based on one or more selected values of x. Illustrative fit factors are listed in Table 7.

TABLE 7
Illustrative fit factors.

| Fit factor | Description, parameters |
|---|---|
| Q | Vertical angle relative to body horizontal plane |
| | (distal implant end above proximal implant end or |
| | proximal implant end above distal implant end) |
| D**(x) | Ratio of implant diameter D(x) to bone diameter |
| | inside cortical material D_{bone}(x) |
| T₁*(x), T₂*(x) | Transverse margins T₁ (x) and T₂ (x) between D(x) |
| | and D_{bone}(x) in a first direction that is orthogonal |
| | to x, normalized to (1/2)D_{bone}(x) |
| T₃*(x), T*₄(x) | Transverse margins T₃ (x) and T₄ (x) between D(x) and |
| | D_{bone}(x) in a second direction that is orthogonal to |
| | both first direction and x, normalized to (1/2)D_{bone}(x) |
| L* | Ratio of installed implant length Lᵢₙₛ to length L_{bottom}, |
| | the length between outer cortical bone at access hole and |
| | inner cortical bone opposite distal end of implant |
| | Other suitable illustrative fit factors |

Table 8 lists illustrative angles Q.

TABLE 8
Illustrative angles (″Q″).
Q (degrees of arc)

| | | | | | | | |
|---|---|---|---|---|---|---|---|
| 0 | | | | | | | |
| 1 | 11 | 21 | 31 | 41 | 51 | 61 | 71 |
| 2 | 12 | 22 | 32 | 42 | 52 | 62 | 72 |
| 3 | 13 | 23 | 33 | 43 | 53 | 63 | 73 |
| 4 | 14 | 24 | 34 | 44 | 54 | 64 | 74 |
| 5 | 15 | 25 | 35 | 45 | 55 | 65 | 75 |
| 6 | 16 | 26 | 36 | 46 | 56 | 66 | 76 |
| 7 | 17 | 27 | 37 | 47 | 57 | 67 | 77 |
| 8 | 18 | 28 | 38 | 48 | 58 | 68 | 78 |
| 9 | 19 | 29 | 39 | 49 | 59 | 69 | 79 |
| 10 | 20 | 30 | 40 | 50 | 60 | 70 | 80 |
| | | | | | | | 80+ |

Table 9 lists illustrative values of D**(x).

TABLE 9
Illustrative values of D**(x).
Illustrative values of D**(x)

<0.1
0.1
0.15
0.2
0.25
0.3
0.35
0.4
0.45
0.5
0.55
0.6
0.65
0.7
0.75
0.8
0.85
0.9
0.95
.99
>0.99

Table 10 lists illustrative values of Tᵢ*(x) for i=1, 2, 3, 4.

TABLE 10
Illustrative values of Tᵢ*(x).
Tᵢ*(x)

| i = 1 | | i = 2 | | i = 3 | | i = 4 | |
|---|---|---|---|---|---|---|---|
| <0.1 | 0.5 | <0.1 | 0.5 | <0.1 | 0.5 | <0.1 | 0.5 |
| 0.1 | 0.55 | 0.1 | 0.55 | 0.1 | 0.55 | 0.1 | 0.55 |
| 0.15 | 0.6 | 0.15 | 0.6 | 0.15 | 0.6 | 0.15 | 0.6 |
| 0.2 | 0.65 | 0.2 | 0.65 | 0.2 | 0.65 | 0.2 | 0.65 |
| 0.25 | 0.7 | 0.25 | 0.7 | 0.25 | 0.7 | 0.25 | 0.7 |
| 0.3 | 0.75 | 0.3 | 0.75 | 0.3 | 0.75 | 0.3 | 0.75 |
| 0.35 | 0.8 | 0.35 | 0.8 | 0.35 | 0.8 | 0.35 | 0.8 |
| 0.4 | 0.85 | 0.4 | 0.85 | 0.4 | 0.85 | 0.4 | 0.85 |
| 0.45 | 0.9 | 0.45 | 0.9 | 0.45 | 0.9 | 0.45 | 0.9 |
| | 0.95 | | 0.95 | | 0.95 | | 0.95 |
| | >0.95 | | >0.95 | | >0.95 | | >0.95 |

Table 11 lists illustrative values of L*.

TABLE 11
Illustrative values of L*.
Illustrative values of L*

<0.25
0.25
0.3
0.35
0.4
0.45
0.5
0.55
0.6
0.65
0.7
0.75
0.8
0.85
0.9
0.95
0.99
1

Implants with different combinations of the shape factors and the fit factors described herein, or other shape factors or fit factors, are contemplated.

An illustrative combination includes x_{g}=65 mm, Q greater than the angle from H to the central axis of the locking socket, and locking screw length of 70 mm. Another illustrative combination includes x_{g}=80 mm, Q equal to the angle from H to the central axis of the locking socket, and locking screw length of 80 mm. Another illustrative combination includes x_{g}=80 mm, Q equal to the angle from H to the central axis of the locking socket, and locking screw length of 90 mm.

The numerical values, whether disclosed as consecutive or nonconsecutive, disclosed herein will be understood to define ranges between consecutive or nonconsecutive values.

Any ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g. 1 to 6.1), and ending with a maximum value of 10 or less (e.g. 2.3 to 9.4, 3 to 8, 4 to 7), and finally to each number 1, 2, 3, 4, 5, 6, 7, 8, 9, and 10 contained within the range.

The implant may self-expand when deployed and/or when rotated. The implant may be deployed using an inserter. The implant may self-expand when a sleeve is removed from the implant. The implant may be expanded from an unexpanded state to an expanded state. The implant may be expanded using one or more actuating mechanisms. The unexpanded state may be a collapsed state.

The implant may be advanced into the bone, when the implant is in a collapsed state, in a sleeve. The implant may be advanced into the bone, when the implant head is in a collapsed state, not in a sleeve. The implant may be advanced into the bone using an inserter described herein.

The implant may include a mesh anchoring substrate (referred to alternately herein as a “mesh” or a “mesh cage”). A thickness of the mesh may be in the range of 0.10 in. to 0.40 in. or any other suitable range. The mesh, when expanded, may form a mesh cage. The mesh, when expanded, may take on any suitable shape. The mesh may be configured to self-expand. The mesh may be expanded using an actuator.

The mesh cage may form a lattice-like structure defining a plurality of openings. The mesh cage may include interconnected cells. The mesh cage may define a central axis. The mesh cage may be expandable about the central axis. The mesh may expand radially away from the central axis. The implant may be expanded when the mesh is expanded. The implant may be collapsed when the mesh is collapsed. The implant may be unexpanded when the mesh is in an unexpanded state.

The mesh may extend longitudinally between an implant hub (referred to alternately herein as a “hub”) and an implant base (referred to alternately herein as a “base”). The base may be cannulated. The base may have a tubular shape. A portion of the base may have a tubular shape. The base may be fixedly attached to the mesh, removably attached to the mesh, or of unitary or monolithic structure with the mesh. The base may include one, two three or more windows. The windows may be used to couple the implant to an inserter disclosed herein.

The base may include an implant tail (referred to alternately herein as a “tail”). The base may be fixed to the implant tail. The implant tail may be positioned in the implant base. The implant tail may have a tubular shape, atraumatic shape, or any other suitable shape.

The hub may have a smooth, rounded outer face to assist in advancing the implant through tissue and into a bone. The hub may have a flat outer face, an outer face including a protrusion, a tissue-engaging member, or any other suitable shape or feature.

The implant may include an implant head. The implant head may include the mesh, the hub and the base. The implant head may be expanded when the mesh anchoring substrate is expanded. The implant head may be collapsed when the mesh anchoring substrate is collapsed. The implant head may be self-expanding. The implant head may be expanded using an inserter. The implant head may be configured to be coupled to the inserter.

The inserter may be used for insertion of the implant. The inserter may be used for removal of the implant. The inserter may be used for both insertion and removal of the implant.

The mesh may be formed from laser-cut tube stock. The mesh may be formed from super elastic materials including nitinol, NiTiCu, titanium alloys, nickel alloys, spring steel alloys, carbon fiber composites, carbon-graphene, shape-memory polymers, polyisoprene-based polymers, calcium iron arsenide CaFe₂As₂ and similar materials.

The base may be formed from the same material as the mesh. The base may be formed from laser-cut tube stock. The base may be formed from super elastic materials including nitinol, NiTiCu, titanium alloys, nickel alloys, spring steel alloys, carbon fiber composites, carbon-graphene, shape-memory polymers, polyisoprene-based polymers, calcium iron arsenide CaFe₂As₂ and similar materials.

The hub may be formed from titanium or any other suitable material.

The support, the extension member and the fixation member may be formed from a titanium alloy, such as an alpha alloy, a near-alpha alloy, an alpha and beta alloy, a beta alloy, or a near beta alloy, or any other suitable material. Exemplary titanium alloys may include the Ti-6AI-4V alloy or the Ti-6AI-7Nb alloy.

“Implant” may refer, collectively, to apparatus such as the implant head and components coupled to the implant head. When the implant head is not coupled to components, such as a support, extension member, or fixation member, “implant” may refer to the implant head.

The implant may be inserted in the bone such that the hub is inserted first into the bone and advanced through an access hole and along a prepared passageway in the bone toward a target site. The hub may be positioned at the target site.

The implant may be inserted in the bone such that the base or tail of the implant is inserted first into the bone and advanced through an access hole and along a prepared passageway in the bone toward a target site. The base or tail of the implant may be positioned at the target site.

When the implant is configured for being inserted into the bone hub-first, an end of the implant opposite the hub may be shaped so that, after the implant is implanted in the bone, the end conforms to a contour of the bone surface abutting the end. The end may sit flush with the bone surface. The end may sit adjacent the bone surface. After the implant is implanted in the bone, the end may sit below the bone surface, such as 1 mm, 2 mm, 3 mm, 4 mm, 5 mm, or any other distance below the bone surface. After the implant is implanted in the bone, the end may protrude above the bone surface. The end may be an end of the implant base. The end may be an end of the implant tail. The end may be an end of a support. The end may be an end of an extension member.

When the implant is configured for being inserted into the bone base-first or tail-first, the portion of the implant inserted first into the bone may form an atraumatic surface.

The implant may occupy a first volume when it is positioned at the target site prior to expansion. The implant may occupy a second volume when it is positioned at the target site and after it has been expanded. The implant may occupy the second volume when the implant is positioned at the target site and expanded to form a mesh cage. The second volume may define a three-dimensional area in the interior. The second volume may occupy a portion of the interior.

The bone may define a longitudinal axis. The bone may include a surface contour. The surface contour may extend along a surface of the bone. The surface contour may be a two-dimensional or three-dimensional shape. The surface contour may encompass two, three, four, or more different points or demarcation points on the surface of the bone. The points or lines may be at different elevations from the longitudinal axis. The points or lines may be at different angular displacements about the bone longitudinal axis. Thus, the surface contour may encompass a region of the bone surface having “topography” that varies longitudinally along the bone. Thus, the surface contour may encompass a region of the bone surface having “topography” that varies circumferentially about the bone. The bone may include two or more surface contours. Each surface contour may extend along a portion of the bone surface.

The implant may be implanted in any suitable bone in the human body.

The bone may be a bone with a metaphyseal and a diaphyseal region, a bone having an articular surface, or any other suitable bone, such as any bone Sᵢ shown in FIG. 106. The implant may be implanted in any suitable bone in an animal. For example, the bone may be a lateral condylar fracture in a horse. The lateral condylar fracture may be a fracture of the third metacarpal or cannon bone on the distal or lower end of the horse.

The nail may be an intramedullary nail. The intramedullary nail may be referred to alternately hereinafter as “nail.” The nail may have a cylindrically shaped portion, two or more cylindrically shaped portions, a square portion, an oval-shaped portion, or any other suitable shape having a non-circular cross-section.

The apparatus may be cannulated. The implant may be cannulated. The broaching tool may be cannulated. Apparatus for coupling to the implant head may be cannulated. This may enable apparatus to be advanced over a k-wire to a target site in the interior of a bone. Using a k-wire to position apparatus may allow insertion of apparatus in the bone without having to use a plate and/or targeting features. A surgeon may confirm placement at the target site using 2 c-arms. A first plate may be placed adjacent the implant to lock the implant to the bone. A second plate may be placed spaced apart from the first plate. Fixation members, such as screws, may be extend through the second plate and into the implant.

The broaching tool may include one, two, three or more broaching members. The broaching tool may be a cavity preparation device. The broaching tool may move the broaching member(s) in the bone interior to displace, disaggregate, cut, disintegrate, dislocate, excavate, abrade, cut or otherwise broach the matter bone material.

The apparatus may include apparatus for, and the methods may involve, apparatus for bone repair. The apparatus may define a longitudinal axis. The apparatus may include the mesh anchoring substrate. The mesh anchoring substrate may be referred to alternately hereinafter as a “mesh.” The mesh may be expandable away from a central axis of the mesh. The mesh may be configured to self-expand away from a central axis of the mesh. The mesh may extend longitudinally between a base and a hub. The apparatus may include a support. The support may be a central member.

The base may include a base first end adjacent the mesh. The base may include a base second end opposite the base first end. The base second end may be defined by a thickness of the base. The thickness may extend between the cannula and an outer face of the base.

The base may define a cannula that is defined between the base first end and the base second end. The cannula may extend between the base first end and the base second end. The cannula may be cylindrical. The cannular may define a diameter. the cannula may define two, three, or more diameters. The cannula may define a diameter that various continuously along some or all of the base. The cannula may extend along the central axis of the base.

A diameter of the base may be constant along a length of the base. A diameter of the base may vary along a length of the base. The base may be cylindrically shaped, non-cylindrically shaped such that a cross-section of the base is oval, square, triangular, rhombic, or any other suitable shape.

The support may include a flange. The support may include an elongated body. The elongated body may be cylindrical. The elongated body may have a uniform circumference. The circumference may be sized such that, when the elongated body is advanced through, and positioned in, the cannula, the elongated body abuts the cannula. The elongated body may include two or more lengths, each length having a different diameter. A maximum diameter of the elongated body may be no greater than a diameter of the cannula.

The elongated body may extend away from the flange to an opposite end of the support. The opposite end may be configured to be removably coupled to a coupling mechanism of the hub. The flange may be configured for being seated against the base second end.

The opposite end of the support may be a first end of the support. The flange may be positioned at a second end of the support.

A diameter of the flange may be equal to an outer diameter of the base second end. A diameter of the flange may be substantially equal to an outer diameter of the base second end. For the purposes of the application, the term “substantially” may be understood to refer to +/−5% of a value. A diameter of the flange may be greater than a diameter of the cannula. A circumference of the flange may be greater than a circumference of the cannula.

A maximum diameter of the elongated body may not be greater than a diameter of the cannula. A maximum circumference of the elongated body may not be greater than a circumference of the cannula.

A difference in diameter between the diameter of the elongated body and the diameter of the base cannula may be 1.5 thousandths of an inch, at least 2 thousandths of an inch, greater than 2 thousandths of an inch, or any other suitable value that configures the elongated body to both abut the cannula and slide through the cannula.

A gap between the elongated body and the cannula may be 1.5 thousandths of an inch, at least 2 thousandths of an inch, greater than 2 thousandths of an inch, any other suitable value that configures the elongated body to both abut the cannula and slide through the cannula.

The difference in diameter, and the gap between, the elongated body and the cannula may apply to any other apparatus or portion of an apparatus that is configured to be seated in the cannula and abut the cannula, or that is illustrated in the drawings as being seated in the cannula and abutting the cannula.

When the mesh is in an expanded state, coupling of the opposite end to the hub such that the flange is seated against the base may set an upper limit of a separation between the hub and the base along the longitudinal axis.

Setting an upper limit of separation between the hub and the base may be referred to alternately herein as “locking” the mesh. The mesh may be locked in fully deployed state (i.e. maximum expansion of the mesh) or in a partially deployed state. A length of the support may determine whether the mesh is locked in a fully-deployed or partially-deployed state. A length of the threaded surface of the support may determine whether the mesh is locked in fully-deployed or partially-deployed state.

When the support is coupled to the hub and the flange is seated on the base, the base may not be fixed to the support other than the abutment of the base with the support. As such, the base may be slidable along an outer face of the support toward the hub to decrease a distance between the base and the hub and expand the mesh.

When the support is coupled to the hub and the extension member, the mesh cage may be unable to collapse without deforming. The mesh cage may be unable to collapse because the base abuts the flange which is longitudinally fixed to the hub (via the support). The base, in abutment with the stationary support flange, is therefore fixed at a maximum distance from the hub and cannot move longitudinally away from the hub, the movement needed to collapse the mesh.

When the mesh is in an expanded state, coupling of the opposite end to the hub such that the flange is seated against the base may set an upper limit, and not a lower limit, of a separation between the hub and the base along the longitudinal axis.

When the mesh is in an expanded state, coupling the opposite end of the support to the hub and seating the flange against the base may not prevent sliding movement of the base, over the elongated body, toward the hub.

The cannula may not define a protrusion for being seated in an indentation in the elongated body. The elongated body may not define a protrusion for being seated in an indentation extending through an inner face of the base, the inner face defining the cannula.

The cannula may have a constant diameter along a length of the base. The cannula may not be threaded. The cannula may define a smooth face.

The cannula may have a diameter that varies along a length of the base. The cannula may have a threaded surface.

The flange may be cylindrical and have a first circumference. The base may be cylindrical and have a second circumference. The first circumference may be substantially equal to the second circumference.

The cannula may define one or more protrusions and indentations for coupling to one or more indentations and protrusions in the support. The support may thread into the cannula. The cannula and the support may be coupled together in a manner other than the abutment of the base with the support.

The hub may have a thickness. The thickness may extend between a hub first end and a hub second end. The hub may define a bore extending through the thickness. The hub may define a bore extending through some of the thickness. The bore may be threaded. The bore may extend along a central axis of the hub. The bore may be transverse to a central axis of the hub. The bore may have a first length.

The coupling mechanism may include the threaded bore. The opposite end of the support may include a threaded surface. The opposite end of the support may include the threaded surface and a tip. The tip may be threaded. The tip may not be threaded. The threaded surface may be threaded to mate with the threaded bore. The threaded surface and the tip, together, may have a second length. The second length may be equal to the first length. The second length may be substantially equal to the first length. The second length may be greater than the first length. The second length may be less than the first length.

When the second length is greater than the first length, and, in operation, the mesh is expanded, threaded engagement of the threaded surface with the threaded bore may position the tip outside the hub.

The hub may include a hub first end. The hub may include, opposite the hub first end, a hub second end. The hub second end may be disposed more proximate the base than is the hub first end. The hub may include a thickness. The thickness may extend between the hub first end and the hub second end. The coupling mechanism may extend through the hub second end and into the thickness. The coupling mechanism may extend through the thickness, between the hub first end and the hub second end.

The coupling mechanism may include a bore extending through the thickness. The bore may be threaded. The bore may not be threaded.

A recess may extend through the hub second end and through some, but not all of, the thickness. The recess may extend circumferentially about the longitudinal axis. The coupling mechanism may be positioned in the recess.

The coupling mechanism may include a twist lock, where the support has one or more transverse teeth that slide along corresponding longitudinal slots in the inner wall of the hub to an annular slot in the proximal end of the wall. The support compresses a spring in the distal end of the hub. The support is rotated. The tooth or teeth move circumferentially until they reach a corresponding dead-end slot, and are pushed into the dead-end by the spring. Decoupling follows the steps in reverse.

The coupling mechanism may include a split ring. The ring may be captured in an annular slot in the inner wall of the hub. The ring diameter may be greater than the depth of the slot, so the ring at equilibrium is in positive relief with respect to the wall. The support may have a counterpart ring. When the support is inserted into the hub, the ring expands to accommodate the support, and then snaps into the counterpart ring. The ring interferes with removal of the support. To remove the support, the ring is expanded mechanically, for example, by a wedge actuator in the wall of the hub. The ring may be expanded thermally, for example, by forming the ring from a shape memory allow, and then applying heat to the up to expand the ring.

The support may include a transverse spring-loaded pin transverse to the support. The circumferential wall of the recess may include a receptacle. When the support is sufficiently inserted into the hub, the pin may spring out of a perforation in the support and into the receptacle. The pin may hold the support in place. To remove the support, the pin is retracted mechanically, for example, by an actuator that may be inserted into the support.

The recess may be the bore. The recess may be the threaded bore. The threaded bore may be threaded for threadedly engaging the opposite end of the support. The opposite end of the support may be threaded to mate with the threaded bore. In operation, the mesh may expand, the opposite end of the support may be in threaded engagement with the coupling mechanism, and the opposite end of the support may be seated in the threaded bore and not traverse the hub first end.

The threaded bore may have a first length. The opposite end of the support, including the threaded surface, may have a second length that is not greater than the first length.

The threaded bore may not traverse the hub first end. The threaded bore may have a length. The methods may include threading the threaded surface into the threaded bore such that the threaded surface is engaged with the length. The methods may include threading the threaded surface into the threaded bore such that the threaded surface is engaged with some, but not all, of the length.

The hub may include a cylindrical section. The cylindrical section may define a constant circumference. The cylindrical section may extend away from the hub second end. The hub may include a tapered section. The tapered section may extend away from the cylindrical section toward the hub first end. A diameter of the tapered section may decrease along a central axis of the hub toward the hub first end.

The hub may include one, two, three or more protrusions extending away from the hub second end. The hub may include one, two, three or more protrusions extending away from an outer face of hub extending between the hub first end and the hub second end. The protrusions may be shaped for engaging bone.

The hub first end may define a first annular opening. The hub second end may define a second annular opening. The coupling mechanism may include a threaded bore that is defined between the hub first end and the hub second end.

The hub second end may define an annular opening. The hub first end may include a flat, circular surface. The coupling mechanism may include a threaded bore that is contiguous with the annular opening and terminates in the thickness without traversing the flat, circular surface.

When the opposite end of the support is a support first end, the apparatus may include a support second end, opposite, along a central axis of the support, the support first end.

The support second end may include a bore. A first length of the bore may not be threaded and a second length of the bore may be threaded. The first length of the bore may be more proximate to the support second end than is the second length of the bore. The first length of the bore may have a first diameter. The second length of the bore may have a second diameter that is smaller than the first diameter.

The fixation member may include a head, a threaded surface configured for threaded engagement with the second length of the bore, and a body disposed between the head and the threaded surface. In operation, when the support is coupled to the hub and seated against the base, the head of the fixation member may be seated in an opening defined in a plate, the threaded surface of the fixation member may be engaged with the second length of the bore, and the body may be seated in the first length of the bore.

The fixation member may be a bolt. The fixation member may be a screw. The fixation member may be a fastening member. The fixation member may be any other suitable member.

The fixation member may couple the support to a plate. The fixation member may couple the implant to a plate. When the fixation member is secured to the plate and the fixation member is coupled to the support, the fixation member may secure the implant to the plate. When the support is secured to the plate (e.g., via the fixation member), and a screw supported by the plate engages a head of the implant, a triangular support structure may be formed. The triangular support structure may support loads on the bone. The triangular support structure may support loads on the bone that promote healing of a fracture in the bone.

The support second end may include the bore. The bore may extend along a central axis of the support. The bore may be threaded. The apparatus may include the fixation member. The fixation member may include a head and a threaded surface for threaded engagement with the bore extending through the support.

The flange may include a flange first face and a flange second face, opposite, along a central axis of the support, the flange first face. The flange first face may be configured to be seated against the base second end. The flange second face may be the support second end. The bore may traverse the flange second face and extend through the flange.

The flange second face may include two or more slots extending axially along the central axis of the support.

The apparatus may include a nut. The nut may be a hexagonal nut. The flange first face may be configured to be seated against the base second end and the nut may extend away from the flange second face. Apparatus described herein including a nut may be driven in rotational motion using, for example, a socket wrench. Apparatus described herein including slots may be driven in rotational motion using, for example, a cruciform screwdriver. Apparatus described herein including a nut may be delivered into, and/or removed from, the implant using, for example, a socket wrench. Apparatus described herein including slots may be delivered into, and/or removed from, the implant using, for example, a cruciform screwdriver.

The support may include a protrusion. A threaded bore may extend through the protrusion. The nut may be disposed between the flange and the protrusion. A face of the protrusion extending circumferentially about a central axis of the support may be configured to be seated in an opening defined in a plate. In operation, when the protrusion is seated in the opening defined in the plate, a fixation member may be engaged in with the threaded bore, thereby fixing the support to the plate. A face of the protrusion may have a shape that conforms to a surface contour of a bone

The apparatus may include the plate. An opening defined in the plate may be configured to receive the protrusion.

The support may include an atraumatic member. The atraumatic member may include at least one opening to receive a fixation member. The atraumatic member may be sized to be placed on a lateral surface of a bone.

The atraumatic member may be fixed to the nut. The atraumatic member may have a thickness that increases along a length of the atraumatic member toward the nut. The atraumatic member may have a thickness that decreases along a length of the atraumatic member toward the nut. The atraumatic member may define at least one opening for receiving a fixation member. The nut may be positioned longitudinally between the flange and the atraumatic member.

The nut may define a threaded bore. The atraumatic member may include a threaded surface for threadedly engaging the threaded bore.

The elongated body may include a first length and a second length. The first length may have a first circumference. The second length may have a second circumference. The first circumference may be greater than the second circumference. The first length may be more proximate the base second end than is the second length.

The elongated body may comprise a third length disposed between the first and second lengths. The third length may have a third circumference. The third circumference may decrease from the second circumference to the first circumference along a central axis of the support toward the first length.

The cannula may be a cylindrical cannula extending along a central axis of the base. In operation, the support may be coupled to the hub and the first length may be positioned in the cannula and abut the cannula.

The support may define a transverse opening that, in operation, is disposed between the base and the hub. The opening may be sized for clearance of a screw. The opening may be circular. The opening may be a non-circular shape. The opening may be one of a plurality of openings. The screw may be configured to penetrate and engage the mesh. The opening may be sized for clearance of a screw that is configured to penetrate and engage of the mesh. The opening may have a diameter that is 0.0005″-0.001″ smaller than a thread diameter of a screw that is configured to penetrate and engage of the mesh. The thread diameter may be a largest thread diameter on the screw. A face of the support surrounding the opening may include threads for threadedly engaging a screw that is configured to penetrate and engage of the substrate.

The apparatus may include apparatus for, and the methods may involve, apparatus for bone repair. The apparatus may define a longitudinal axis. The apparatus may include the mesh anchoring substrate, the base and the hub. The mesh anchoring substrate may be expandable away from a central axis of the mesh, and extend between the base and the hub. The hub may include a first coupling mechanism. The first coupling mechanism may be the coupling mechanism.

The base may include the base first end adjacent the mesh and the base second end opposite the base first end. The base may define the cannula extending through the base between the base first end and the base second end. The cannula may have a cannula circumference.

The support may include the support first end, the support second end, and may be configured to be removably coupled to the first coupling mechanism. The support second end may be opposite the support first end and may include a second coupling mechanism. The second coupling mechanism may have features in common with the coupling mechanism. A maximum circumference of the support may not be greater than the cannula circumference.

The apparatus may include a fixation member. The fixation member may include a body extending between a head and an elongated member. The elongated member may be configured to be coupled to the second coupling mechanism. The head may be configured to be seated against the base second end.

When the mesh is in an expanded state, coupling the support to the hub, and coupling the fixation member to the support such that the head of the fixation member is seated against the base may set an upper limit of a separation between the hub and the base along the longitudinal axis.

A distance between the hub and the base second end defines, when the mesh is expanded, a first length. The support may have a second length. The second length may be smaller than the first length. When the mesh is in an expanded state, coupling the support first end to the hub may position the support second end in the cannula.

The head of the fixation member may be configured to be seated against the base second end. The body of the fixation member may be configured to be seated in, and in abutment with, the cannula.

The first coupling mechanism may include a threaded bore. The first end of the support may include a threaded surface for threadedly engaging the threaded bore.

The threaded bore may be a first threaded bore. The second coupling mechanism may be a second threaded bore. The elongated member may include threads configured to threadedly engage the second threaded bore.

The support second end may include a nut. The second coupling mechanism may extend through the nut.

The support second end may include longitudinal slots extending axially along a central axis of the support.

The apparatus may include a plate. A face of the fixation member extending circumferentially about the head of the fixation member may be configured to be seated in an opening defined in the plate. In operation, the face may be seated in the opening and a bottom face of the head is seated against the base second end.

The second coupling mechanism may be for coupling the implant to a plate. The nut may be positioned longitudinally between the flange and the second coupling mechanism. The apparatus may include the plate. The plate may be configured to be removably coupled to the second coupling mechanism.

The apparatus may include apparatus for, and the methods may involve, apparatus for implanting in a bone. The apparatus defining a longitudinal axis and include the mesh anchoring substrate that is expandable away from a central axis of the mesh extends longitudinally between the base and the hub.

The apparatus may include the support. The support may be configured to slide within a cannula extending through the base. The support may include the first end configured to be removably coupled to the hub and the second end.

The apparatus may include an extension member. The extension member may be configured to be removably coupled to the second end. Coupling the first end to the hub and the extension member to the second end may set an upper limit of a separation between the hub and the base along the longitudinal axis.

The apparatus may include an extension member. The extension member may be configured to be removably coupled to the second end. Coupling the first end to the hub and the extension member to the second end so that a flange of the extension member is seated on the base may set an upper limit of a separation between the hub and the base along the longitudinal axis.

The mesh may be locked in fully deployed state (i.e. maximum expansion of the mesh) or in a partially deployed state. A combined length of the support and the extension member may determine whether the mesh is locked in fully-deployed or partially-deployed state. A length of the threaded surface of the extension member may determine whether the mesh is locked in a fully-deployed or partially-deployed state.

When the support is coupled to the hub, the extension member is coupled to the support and the flange of the extension member is seated on the base, the base may not be fixed to either the support or the extension member other than the abutment of the base with the support and the extension member. As such, the base may be slidable along an outer face of the support and extension member toward the hub to decrease a distance between the base and the hub and expand the implant head.

When the support is coupled to the hub, the extension member is coupled to the support and the flange of the extension member is seated on the base, the mesh cage may be unable to collapse without deforming. The mesh cage may be unable to collapse because the base abuts the flange which is longitudinally fixed to hub (via the support and the extension member). The base, in abutment with the stationary extension member flange, is therefore fixed at a maximum distance from the hub and cannot move longitudinally away from the hub, the movement needed to collapse the mesh.

When the mesh is in an expanded state, coupling of the opposite end to the hub such that the flange is seated against the base may set an upper limit, and not a lower limit, of a separation between the hub and the base along the longitudinal axis.

When the mesh is in an expanded state, coupling the first end to the hub and the extension member to the second end may not prevent sliding movement of the base, over the elongated body, toward the hub.

The second end of the support may terminate inside the base when the first end is coupled to the hub.

The extension member may include a flange. Coupling the extension member to the second end may place the flange in abutment with the base.

Fixing the upper limit of separation may prevent the mesh from collapsing.

The apparatus may include a fixation member. The fixation member may be a locking cap. The fixation member may be configured to be removably coupled to the extension member. A head of the fixation member may be configured to mate with a plate positioned on an outer surface of the bone.

Positioning the fixation member in an opening defined by the plate and coupling the fixation member to the extension member may lock the assembled implant to the plate. Depending on how deep the implant is positioned in the interior of the bone, some or all of threads extending around a threaded surface of the fixation member may be screwed into threads in the plate extending around the opening. A gap may extend between a bottom face of a head of the fixation member and the extension member. A bottom face of the head of the fixation member may be seated on the extension member.

An outer face of the extension member may be threaded. The fixation member may include a tubular member extending away from the head of the fixation member. The tubular member may define an inner threaded surface that may be configured to engage the outer face of the extension member.

The first end of the support may include a first support engagement feature that may be configured to engage with the hub. The second end of the support may include a second support engagement feature. A first end of the extension member may include a first extension member engagement feature. A second end of the extension member may include a second extension member engagement feature. The second end of the support may include a coupling mechanism for coupling the second end of the support to the first extension member engagement feature.

The base may have a base first end coupled to the mesh and a base second end, opposite the base first end, defining an opening. The extension member may include a flange configured to be seated the base second end. The flange may be may be disposed between the first extension member engagement feature and the second extension member engagement feature.

The base second end may have an outer diameter. The flange may have the outer diameter.

The first support engagement feature may include a threaded surface. The second support engagement feature may include a hexagonal nut. The first extension member engagement feature may include a threaded surface. The second extension member engagement feature may include a hexagonal nut.

The second end of the support may define a threaded bore extending through the hexagonal nut. The threaded bore may be sized to receive the first extension member engagement feature.

The fixation member may include a body disposed between a head and a threaded surface. The head may be configured to mate with a plate positioned on an outer surface of the bone.

The extension member may include an extension member first end configured to be removably coupled to the second end of the support and an extension member second end, opposite the extension member first end, along a central axis of the extension member. The second end of the extension member may define a threaded bore sized to receive the threaded surface of the fixation member.

The support may include slots at the second end. The slots may be configured to be engaged by a tool for rotating the support.

The first support engagement feature may be a threaded surface. The second support engagement feature may be two or more slots. The first extension member engagement feature may be a threaded surface. The second extension member engagement feature may be two or more slots.

The second end of the support may define a threaded bore extending through the second end of the support. The threaded bore may be sized to receive the first extension member engagement feature.

A circumference of the support may be constant along a length of the support between the second end of the support and the threaded surface.

The apparatus may include a fixation member. The fixation member may be a locking cap. The fixation member may be configured to be removably coupled to the extension member. The fixation member may include a body disposed between a head and a threaded surface. The head may be configured to mate with a plate positioned on an outer surface of the bone.

The extension member may include an extension member first end configured to be removably coupled to the second end of the support and an extension member second end, opposite the extension member first end, along a central axis of the extension member. The second end of the extension member may define a threaded bore sized to receive the threaded surface of the fixation member.

The methods, which may involve the apparatus shown and described herein, may include a method for implanting the implant in an interior of a bone. The methods may include advancing, in a tensioned, collapsed state, the self-expanding mesh anchoring substrate into the interior. The mesh may extend between the hub and the cannulated base. The base may have the base first end adjacent the mesh and the base second end opposite the base first end. The methods may include releasing the mesh from the tensioned, collapsed state, to allow the mesh to self-expand in the interior.

The methods may include selecting a support from two or more supports for coupling to the hub. Each of the supports may have a first end for coupling to the hub and a second end including a flange having a length. The first end may include the threaded surface. The selecting may include identifying a flange with a length sufficient to span between a surface of the bone and the base second end. The selecting may include identifying a flange with a length sufficient to span between plate positioned on a surface of the bone and the base second end.

A kit may be provided including apparatus described herein. The kit may include two or more extension members having varying lengths. The extension members may have varying lengths of one or more of the threaded surface of the extension member, the nut and the flange. A longer threaded surface may expand the implant head more than a shorter threaded surface. A longer flange and/or hexagonal nut may allow a practitioner to position the implant deeper into an interior of a bone and still couple the implant to a plate positioned on a surface of the bone. The methods may include selecting an extension member from the two or more extension members so that, when the extension member is coupled to the support, the extension member second end is positioned on, or proximal to, an outer surface of the bone.

The methods may include setting an upper limit of a separation between the hub and the base along a longitudinal axis of the mesh by coupling the first end of the support to the hub and placing the flange in abutment with the base second end. Coupling the first end of the support to the hub may include threading a threaded surface of the support into a threaded bore extending through the hub.

The methods may include further expanding the mesh after the flange is in abutment with the base second end by threading the threaded surface an additional turn into the threaded bore. The methods may include threading the threaded surface a first number of turns into the threaded bore to place the flange in abutment with the base second end. The methods may include threading the threaded surface an addition turn, or additional turns, to further expand the mesh. Further expanding the mesh may elevate a fracture.

The methods may include placing a plate on a surface of the bone. The methods may include coupling the plate to the support. The coupling may include advancing a fixation member through an opening on the plate. The coupling may include seating a head of the fixation member in the opening. The coupling may include engaging a threaded surface of the threaded surface with a threaded bore extending through the flange.

The methods, which may involve the apparatus shown and described herein, may include a method for implanting the implant in an interior of a bone. The methods may include advancing, in a tensioned, collapsed state, the self-expanding mesh anchoring substrate into the interior. The mesh may extend between the hub and the cannulated base. The base may have the base first end, adjacent the mesh, the base second end opposite the base first end.

The methods may include releasing the mesh from the tensioned, collapsed state, to allow the mesh to self-expand. The methods may include advancing the support through the base. The methods may include coupling a first end of a support to the hub.

The methods may include selecting a support from two or more supports for coupling to the hub. Each of the supports may have a different length.

The methods may include selecting an extension member from two or more extension members for coupling to the support. Each of the extension members may have an end for coupling to the support and a flange configured for seating on the base and having a length. The selecting may include identifying a flange with a length sufficient to span between a surface of the bone and the base second end. The selecting may include identifying a flange with a length sufficient to span between a plate positioned on a surface of the bone and the base second end. The methods may include setting an upper limit of a separation between the hub and the base along a longitudinal axis of the mesh by coupling an end of the support to the hub.

The methods may include coupling a first end of the extension member to a second end of the support. The methods may include seating the flange against the base second end.

Coupling the first end of the support to the hub may include threading a threaded surface of the support into a threaded bore extending through the hub.

Coupling the first end of the extension member to the second end of the support may include threading a threaded surface of the extension member into a threaded bore in the support.

The methods may include further expanding the mesh after the flange is in abutment with the base second end. The further expanding may include threading the threaded surface of the extension member a first number of turns into the threaded bore in the support. The first number of turns may seat the flange on the base. After threading the threaded surface the first number of turns, the methods may include drawing the support toward the base. The drawing may be effected by threading the threaded surface of the extension member an additional turn into the threaded bore in the support.

The methods may include placing a plate on a surface of the bone. The methods may include coupling the plate to the extension member. The coupling may include advancing a fixation member through an opening on the plate and into a bore extending through the second end of the extension member. The coupling may include seating a head of the fixation member in the opening. The coupling may include engaging the threaded surface of the fixation member with the threaded bore extending through the extending through the extension member.

The apparatus may include apparatus for, and the methods may involve, apparatus for bone repair. The apparatus may define a longitudinal axis. The apparatus may include an assembly.

The assembly may include the mesh anchoring substrate. The mesh may be expandable away from a central axis of the substrate. The mesh anchoring substrate may extend longitudinally between the base and the hub. The assembly may include the base. The base may define a cannula extending through a thickness of the base. The assembly may include the hub. The hub may define an opening extending through a thickness of the hub. The assembly may include a tail positioned in the cannula. The tail may be coupled to the base. The tail may define a coupling mechanism.

The cannula may extend through the thickness of the base along the longitudinal axis. The cannula may extend through the thickness of the base along an axis transverse to the longitudinal axis.

The hub may be fixedly attached to the tail. The hub may be removably coupled to the tail. The hub and the base may be, together, of monolithic construction.

The tail may include a tail first end and a tail second end. The coupling mechanism may traverse the tail first end. The tail second end may be positioned outside the base. The portion of the tail positioned outside of the base may have an atraumatic surface. An outside circumference of the atraumatic surface may decrease toward the tail second end.

The apparatus may include an elongated support. The elongated support may be configured to be removably coupled to an assembly. The elongated support may include an end cap. The end cap may be positioned at a first end of the support. The end cap may have an outer diameter greater than an outer diameter of the opening defined in the hub. The end cap may include a bottom face configured to be seated on an outer face of the hub. The end cap may include a hexagonal nut. The end cap may include one, two or more protrusions or indentations for coupling to an insertion or removal member.

The end cap may be configured to be coupled to the hub by friction fit.

The opening defined in the hub may be threaded. The support may define threads adjacent the end cap for mating with the opening defined in the hub.

The elongated support may include may include a member positioned at a second end of the support. The member may be configured to mate with the coupling mechanism. The elongated support may include an elongated body extending longitudinally between the end cap and the threaded surface.

In operation, the mesh may be expanded, the support may be coupled to the assembly, the member may be coupled to the coupling mechanism and the bottom face of the end cap may be seated on the outer face of the hub.

The coupling mechanism may be a threaded bore extending through the tail. The member may be a threaded surface. The threaded surface may be threaded to mate with the threaded bore. In operation, the mesh may be expanded, the support may be coupled to the assembly, the threaded surface may be in threaded engagement with the threaded bore and the bottom face of the end cap may be seated on the outer face of the hub.

The threaded bore may extend through the tail along the longitudinal axis. The threaded bore may extend through the tail along an axis transverse to the longitudinal axis.

The tail may include a tail first end and a tail second end. The threaded bore may traverse the tail first end. The tail second end may be positioned outside the base. The portion of the tail positioned outside of the base may have an atraumatic surface. An outside circumference of the atraumatic surface may decrease toward the tail second end.

The support may be a first support. The end cap may be a first end cap. The member may be a first member. The elongated body may be a first elongated body. The apparatus may include a second support.

The second support may include a second end cap. The second end cap may be positioned at a first end of the second support. The second support may include a second member positioned at a second end of the second support. The second member may be configured to mate with the coupling mechanism. The second support may include a second elongated body extending longitudinally between the second end cap and the second member.

The second end cap may include one, two or more protrusions or indentations for coupling to an insertion or removal member.

A length of the second member may be greater than a length of the first member.

The mesh may be a self-expanding mesh. The coupling of the second elongated body to the coupling mechanism may retain the mesh in a collapsed position.

The support may be a first support. The end cap may be a first end cap. The threaded surface may be a first threaded surface. The elongated body may be a first elongated body. The apparatus may include a second support. The second support may include a second end cap positioned at a first end of the second support. The second support may include a second threaded surface positioned at a second end of the second support. The second threaded surface may be shaped for threaded engagement with the threaded bore. The second support may include a second elongated body extending longitudinally between the second end cap and the second threaded surface.

The second end cap may include one, two or more protrusions or indentations for coupling to an insertion or removal member.

A length of the second member may be greater than a length of the first member.

The mesh may be a self-expanding mesh. The coupling of the second elongated body to the coupling mechanism may retain the mesh in a collapsed position.

The methods, which may involve the apparatus shown and described herein, may include a method for removing the implant from an interior of a bone. The methods may include disengaging the support from the implant hub. The support may be referred to alternately herein as an “implant support.” The methods may include withdrawing the implant support through the implant base. The methods may include, after the withdrawing, inserting, through the implant base, the broaching tool. The methods may include, prior to the inserting of the broaching tool, removing screws from engagement with the head of the implant. The methods may include expanding the broaching tool. The methods may include broaching, with the broaching tool, matter in a volume circumscribed by an expanded implant head.

The disengaging may include unthreading a threaded surface of the implant support from a threaded surface of the implant hub.

The methods may include after the broaching, collapsing the head and removing the implant from the bone. The head may be collapsed using the inserter. The collapsing may include coupling a removal mechanism to an implant base. The removal mechanism may be the inserter.

The collapsing may include coupling a removal mechanism to an implant hub. The collapsing may include coupling a removal mechanism to the implant base and the implant hub. The collapsing may include advancing the implant into a sleeve, the sleeve having an outer diameter that is smaller than an outer diameter of the expanded implant head

The collapsing may include inserting a removal mechanism through the implant base. The collapsing may include threading an end of the removal mechanism into the threaded surface of the implant hub. The collapsing may include coupling the removal mechanism to the implant base. The collapsing may include increasing a longitudinal distance between the implant base and the implant hub.

The collapsing may include inserting a removal mechanism through the implant base. The collapsing may include advancing an end of the removal mechanism through an opening defining by the implant hub. The collapsing may include coupling an angled member of the removal mechanism to the implant hub. The collapsing may include coupling the removal mechanism to the implant base. The collapsing may include increasing a longitudinal distance between the implant base and the implant hub.

The collapsing may include re-inserting the implant support through the implant base. The collapsing may include threadedly engaging a threaded surface of the implant support with a threaded surface of the implant hub. The collapsing may include drawing the implant base longitudinally away from the implant hub.

The methods, which may involve the apparatus shown and described herein, may include a method for removing from a bone the implant implanted an interior of the bone. The implant may include the implant head extending between the hub and the base. The implant may include the support removably coupled to the implant.

The methods may include decoupling the support from the implant. The methods may include removing the support from the implant. The methods may include positioning the broaching tool inside a volume circumscribed by the implant head. The positioning may be performed after the removing. The methods may include broaching, with the broaching tool, matter in the volume. The broaching tool may be moved in the bone interior to displace, disaggregate, cut, disintegrate, dislocate, excavate, abrade, cut or otherwise broach the matter bone material.

The matter may be organic matter accumulated in the volume after implantation of implant. The matter may be organic matter, such as bone material, bone ingrowth, tissue, or any other matter that may be inside an implanted implant. The matter may be non-organic matter placed in the volume by a practitioner during implantation of the implant. The matter may be organic matter and non-organic matter.

The methods may include removing screws from engagement with the implant head. The screws may be removed prior to the positioning of the broaching tool.

The positioning may include advancing the broaching tool along a cavity inside the implant. The cavity may be formed in the matter by removal of the support from the implant. The cavity may be enlarged by a drill before the positioning of the broaching tool.

The methods may include expanding the broaching tool in the interior during the broaching. The methods may include scraping an inner face of the implant head. The methods may include drawing a broaching member of the broaching tool along cells defined by the implant head. The broaching member may be a blade.

The methods may include collapsing the implant head after the broaching. The implant head may be collapsed using the inserter. The collapsing may include increasing a distance between the hub and the base. The methods may include removing the implant from the bone.

The methods may include collapsing the implant head after the broaching. The collapsing may include advancing the implant into a sleeve. The sleeve may have an outer diameter that is smaller than an outer diameter of the implant head in an expanded state.

The decoupling may include decoupling the support from the hub. The decoupling may include unthreading a threaded surface of the support from threaded engagement with a threaded bore in the hub.

The methods may include, prior to the removing of the support from the implant, unthreading a fixation member from threaded engagement with the support. The methods may include, prior to the removing of the support from the implant, withdrawing the fixation member through an opening in a plate.

The methods may include, prior to the removing of the support from the implant, unthreading a fixation member from threaded engagement with an extension screw. The methods may include, prior to the removing of the support from the implant, withdrawing the fixation member through an opening in a plate. The methods may include, prior to the removing of the support from the implant. removing the plate from a surface of the bone. The methods may include, prior to the removing of the support from the implant, unthreading the extension screw from threaded engagement with the support.

The removing may include withdrawing the support through a cannula extending through the base. The positioning may include advancing the broaching tool through the cannula and into the volume.

The methods may include removing matter, such as bone ingrowth, accumulated on or adjacent to a nut positioned on the support and/or the extension member. The methods may include using a burr or a pick to remove the matter.

After the broaching, the methods may include collapsing the implant. The implant may be collapsed using the inserter. The collapsing may include inserting a removal mechanism through the base. The collapsing may include coupling the removal mechanism to the base and the hub. The collapsing may include increasing a longitudinal distance between the base and the hub.

The coupling the removal mechanism to the hub may include threading an end of the removal mechanism into a threaded bore in the hub. The coupling the removal mechanism to the hub may include advancing an end of the removal mechanism through an opening defined by the hub and coupling the end of the removal mechanism to the hub.

The methods may include, after the broaching, collapsing the implant. The implant may be collapsed using the inserter.

The collapsing may include coupling the support to the hub. The coupling may include advancing the support through inner cannula and into the volume. The collapsing may include drawing the base longitudinally away from the hub.

The decoupling may include decoupling the support from the base and not decoupling the support from the hub.

The positioning may include advancing the broaching tool along a longitudinal axis of the implant. The advancing may include advancing the broaching tool along a cavity inside the implant. The cavity may be defined by a space where the support was positioned, in the implant, prior to the removing of the support from the implant.

The advancing may include advancing the broaching tool through matter, in the volume, extending along the longitudinal axis. The matter may be positioned in the volume in front of a location, in the volume, where an end of the support was positioned. The end of the support may have been positioned apart from the hub.

The methods may include using a distal tip of the broaching tool to cut matter extending along the longitudinal axis.

The base may include the implant tail. The implant tail may be positioned within the base. The support may be removably coupled to the implant tail. The decoupling may include decoupling the support from the implant tail. The decoupling may include unthreading a threaded surface of the support from threaded engagement with a threaded bore in the implant tail.

The support may include the end cap positioned adjacent to the hub. The removing the support may include drawing the end cap away from the hub. The end cap may be positioned on the hub. The removing the support may include advancing the support through an opening in the hub. The positioning may include advancing the broaching tool through the opening in the hub.

The broaching may include broaching matter adjacent the implant tail. The matter adjacent the implant tail may be broached with a portion of the broaching tool most distal to a handle of the broaching tool.

The collapsing may include inserting a removal mechanism through an opening in the hub. The collapsing may include coupling the removal mechanism to the implant tail and the hub. The collapsing may include increasing a longitudinal distance between the implant tail and the hub.

The collapsing may include inserting a removal mechanism through an opening in the hub. The collapsing may include threading an end of the removal mechanism into the threaded bore. The collapsing may include coupling the removal mechanism to the hub. The collapsing may include increasing a longitudinal distance between the base and the hub. Increasing the longitudinal distance between the base and the hub may collapse the implant head.

The methods, which may involve the apparatus shown and described herein, may include a method for removing the implant from an interior of a bone. The methods may include disengaging a first support from an implant tail. The methods may include withdrawing the first support from the implant. The withdrawing may include including advancing the first support through an opening in an implant hub.

The methods may include, after the withdrawing, inserting, through the implant hub, the broaching tool. The methods may include expanding the broaching tool within the interior. The methods may include broaching, with the broaching tool, matter in a volume circumscribed by an expanded implant head. The methods may include, prior to the inserting of the broaching tool, removing screws from engagement with the implant head.

The methods may include, after the broaching, collapsing the head and removing the implant from the bone. The inserter may be used to perform the collapsing.

The collapsing may include coupling a removal mechanism to an implant head. The removal mechanism may be the inserter.

The collapsing may include coupling a removal mechanism to an implant tail. The collapsing may include coupling a removal mechanism to the implant tail and the implant hub. The collapsing may include increasing a longitudinal distance between the implant tail and the implant hub. The collapsing may include advancing the implant into a sleeve. The sleeve may have an outer diameter that is smaller than an outer diameter of the expanded implant head.

The methods, which may involve the apparatus shown and described herein, may include a method for treating a bone having a surface and an interior. The method may include positioning the implant in the interior. The implant may be positioned in the interior when a self-expanding mesh substrate of the implant is in a collapsed state.

The methods may include expanding the mesh substrate by disengaging an end of a first support from an implant tail. The methods may include withdrawing the first support from the implant through an opening in an implant hub. The methods may include, after the withdrawing, inserting a second support through the opening in the implant hub. The methods may include coupling the second support to the implant tail.

The positioning may include advancing the implant through an access hole on the surface and into the interior. The positioning may include positioning the implant tail distal the access hole relative to the implant hub.

The disengaging may include unthreading a threaded surface of the first support from an implant threaded bore extending through the implant tail.

The coupling may include threading a threaded surface of the second support into the implant threaded bore. The threading of the threaded surface of the second support into the implant threaded bore may expand the mesh.

When the end of the first support is a support first end and the support includes a support second end, the positioning may include positioning the support second end outside the bone. When the end of the first support is a support first end and the support includes a support second end, the disengaging may include turning the support second end.

The methods, which may involve the apparatus shown and described herein, may include a method for treating a bone having a surface and an interior.

The methods may include advancing an intramedullary nail through a first access hole and into the interior. The methods may include preparing a passageway in the interior through the first access hole. The nail may be advanced along the passageway in the interior. The passageway may have an inner circumference greater than an outer circumference of the nail.

The nail may define a bore extending through the nail at an angle transverse to a central axis of the nail. The methods may include preparing an elongated channel through a second access hole. The elongated channel may be performed through a lateral side of the bone. The elongated channel may be performed through a medial side of the bone. The channel may extend through the bore. A longitudinal axis of the channel may extend through the bore along the angle transverse to the central axis of the nail. The channel may have a first length between the bore and an end of the channel in the interior. The channel may have a second length between the second access hole and the bore. The second length may be opposite the first length across the central axis. The second length may not be opposite the first length across the central axis.

The channel may be prepared in a proximal tibia, distal tibia, distal femur, proximal femur, calcaneus, or in any other suitable location in a bone in the body.

The method may include enlarging the channel along the first length to create a first cavity. The method may include enlarging the channel along the second length to create a second cavity. The method may include expanding a first implant in the first cavity. The method may include expanding a second implant in the second cavity.

The enlarging of the channel along the first length and the enlarging of the channel along the second length may be performed simultaneously. The enlarging of the channel along the first length and along the second length may be performed with a cavity preparation member having a first blade and a second blade spaced apart from the first blade.

The enlarging of the channel along the second length may be performed after the enlarging of the channel along the first length.

The methods may include positioning the first implant in the interior. The first implant may be the implant. The positioning may include positioning a first implant head in the first cavity and positioning a first implant tail in the bore. The methods may include positioning the second implant in the interior. The second implant may be the implant. The second implant may be positioned in the interior after the positioning of the first implant in the interior. The positioning may include positioning a second implant head in the second cavity and positioning a second implant tail in the bore.

The first implant may be a first expandable implant. The second implant may be a second expandable implant.

The positioning of the first expandable implant may be performed before the positioning of the second expandable implant. The positioning of the second expandable implant may include coupling the first implant to the second implant in the interior. The positioning of the second implant may include coupling the second implant tail to the first implant tail.

The first implant may be self-expanding. The second implant may be self-expanding. The expanding of the first implant may include withdrawing the first implant from a first sleeve. The expanding of the second implant may include withdrawing the second implant from a second sleeve.

The methods may include coupling the first implant to the second implant outside the bone. When the first implant is coupled to the second implant outside the bone, the first implant and the second implant may be advanced, in a coupled state, into the interior along the elongated channel. The expanding the first implant may include withdrawing the first implant from a sleeve. The expanding the second implant may include withdrawing the second implant from the sleeve.

The methods may include coupling the first implant to the second implant inside the bone.

The coupling of the first implant to the second implant may include threadedly engaging threads on an outer face the first implant with threads on an inner face of the second implant. The coupling may include threadedly engaging threads defined on an outer face of the second implant with threads defined on an inner face of the first implant. The coupling may include advancing one or more protrusions extending away from the first implant into one or more recesses extending into the second implant. The coupling may include advancing one or more protrusions extending away from the second implant into one or more recesses extending into the first implant.

The coupling may include a friction fit. The coupling may include advancing an inner face of the second implant along an outer face of the first implant. The coupling may include advancing an outer face of the second implant along an inner face of the first implant.

The methods may include, after the expanding of the first and second implant, anchoring the first implant to the bone and anchoring the second implant to the bone. The anchoring may include driving screws through a lateral side of the bone and into the first and/or second implant. The anchoring may include driving screws through a medial side of the bone and into the first and/or second implant. The anchoring may include driving screws axially along a length of the bone and into the first and/or second implant.

The methods may include anchoring the nail to the bone.

The methods may include withdrawing the second implant from the interior and withdrawing the first implant from the interior. The withdrawing may include coupling a removal device to the first implant and coupling the removal device to the second implant. The withdrawing may include increasing a longitudinal distance between the first implant and the second implant. The coupling may include compressing the second implant and the first implant by drawing the second implant and the first implant into a sleeve. Prior to the withdrawing, the methods may include advancing a broaching tool along the channel and expanding and rotating the device in the first and second cavities to broach material accumulated in the cavities after the implantation of the first and second implants.

The methods may include withdrawing the second implant from the interior and withdrawing the first implant from the interior. The withdrawing may include de-coupling the first implant from the second implant. The withdrawing may include coupling a removal device to second implant, collapsing the second implant, and removing the second implant from the bone. The withdrawing may include, after removing the second implant, removing the first implant. The withdrawing may include coupling a removal device to the first implant, collapsing the first implant, and removing the first implant from the bone.

Prior to removing the first and second implant from the bone, the methods may include advancing a cavity preparation device into volumes circumscribed by each of the first and second implants and cutting, with the broaching tool, matter.

The methods, which may involve the apparatus shown and described herein, may include a method for treating a bone having a surface and an interior. The bone may have an elongated portion, a head including an articular surface, and a neck disposed between the elongated portion and the head. The bone may be any other suitable bone in the human body.

The methods may include preparing a channel through the surface and into the interior. The methods may include placing a plate on the surface such that a barrel extending away from the plate is positioned in the channel.

The methods may include advancing the expandable implant through a bore defined by the barrel. The methods may include positioning the implant in the bone such that an expandable implant head is positioned in the head of the bone, outside of the bore, and an implant base is positioned in the bore. The implant may include the implant head.

The methods may include expanding the implant head to form the mesh cage. The implant head may be an expandable mesh substrate. The methods may include advancing an elongated member including a threaded surface through a hole defined by the plate and into the head such that the elongated member does not abut expanded implant head. The advancing of the elongated member into the head may secure the implant against rotation in the interior.

The methods may include not locking the implant to the barrel so that the implant base is in sliding engagement with the bore.

The methods may include anchoring the implant head to the bone using one or more screws. After the anchoring, the methods may include compressing the bone by advancing the implant base through the bore toward the surface of the bone and, in some embodiments, away from the plate.

The methods may include positioning a flanged end of the implant on the plate to couple the implant to the plate.

The methods may include driving a screw through an opening defined by the flanged end and through a hole defined by plate.

The apparatus may include apparatus for, and the methods may involve, apparatus for placement in an interior of the bone.

The apparatus may include the intramedullary nail. The apparatus may include the implant. The implant may include the expandable mesh anchoring substrate. The mesh may extend longitudinally between a first end and a second end. The first end may define a first opening. The second end may define a second opening.

The first end may include the hub. The second end may include the base. The base may define an annular opening. The base may be cannulated. The base may define the first opening. The hub may define the second opening.

The first end may include the base. The second end may include the hub. The hub may define an annular opening. The hub may be cannulated. The base may define the first opening. The hub may define the second opening.

The mesh may be configured to be coupled to the nail such that a length of the nail extends between the first opening and the second opening. In operation, the mesh substrate may be coupled to the nail and the first end and the second end may be coaxially mounted on an outer surface of the nail and spaced apart along a central axis of the nail.

The mesh may be configured to be coupled to the nail such that a length of the nail extends between the first opening and along some, but not all, of an elongated opening extending through an interior of the mesh. In operation, the substrate may be coupled to the nail and the first end, but not the second end, may be coaxially mounted on an outer surface of the nail.

The mesh may be mounted on the outer surface of the nail such that the first end is in physical contact with the nail. The mesh may be mounted on the outer surface of the nail such that the first and the second end are in physical contact with the nail.

The mesh may be mounted on the nail such that the nail extends through the first and second openings but does not come into physical contact with the mesh.

The nail may be positioned in any suitable bone in the human body, such as any bone Sᵢ illustrated in FIG. 103. The mesh be positioned in same bone as the nail. The mesh may be positioned in the proximal humerus, proximal tibia, distal femur, calcaneus, or adjacent any other suitable articular surface. The mesh may be positioned in any suitable bone and not adjacent an articular surface.

The mesh may have any suitable shape that defines an elongated opening for receiving the nail. For example, the mesh may be cone-shaped, doughnut shaped, ellipsoid shaped, circular, or any other suitable shape. When the mesh is adjacent an articular surface, the mesh may conform, at least in part, to a shape of the articular surface.

The first opening may be a first annular opening. The second opening may be a second annular opening. The nail may have a constant outside circumference along a length of the nail.

The first opening and/or the second opening may have a shape that is oval, square, defines one or more protrusions or indentations, or any other suitable shape. The shape of the first and/or second opening may confirm to an outer surface of the nail to enable the mesh to be advanced along a length of the nail and mounted on the nail such that the first and second ends are mounted on the nail.

The mesh may be self-expanding. The mesh may be manually expanded.

The first end may have a first inner face extending about the first opening. The first end may include protrusions extending away from the first inner face and into the first opening. In operation, the mesh may be mounted on the outer surface of the nail and the protrusions may be in physical contact with the nail and provide tension to the nail.

The protrusions may be spaced circumferentially about the first inner face. The mesh may include, one, two, three, four, five, six, seven, eight, nine, ten, twenty, fifty, one hundred, or any other suitable number of protrusions.

The protrusions of the first end may be first protrusion. The second end may have a second inner face extending about the second opening. The second end may include second protrusions extending away from the second inner face and into the second opening. In operation, the mesh may be mounted on the outer surface of the nail and the first protrusions and the second protrusions may be in physical contact with the nail and provide tension to the nail.

The protrusions may be projections, tabs, barbs, spring fingers, or any other protrusions that center the nail in the interior of the bone. The protrusions may restrict longitudinal movement of the nail in the interior. The protrusions may keep the nail from wiggling in the interior. The protrusions may apply tension, such as spring tension, to the nail.

A diameter of the first opening may be equal to a diameter of the second opening. A diameter of the first opening may be different from a diameter of the second opening.

The first opening may have a first circumference. The second opening may have the first circumference. An outer face of the nail may have a second circumference equal to the first circumference.

The first opening may have a first circumference. The second opening may have the first circumference. An outer face of the nail may have a second circumference different from the first circumference.

The first opening may have a first circumference. The second opening may have a second circumference. An outer face of the nail may have a third circumference different from the first and second circumferences.

The nail may have an outer circumference. The first and second openings may have a circumference that is 2%-35% greater than the outer circumference of the nail.

The nail may have an outer circumference of 10-12.5 mm. The first opening may have a first circumference that is 0.5-3 mm greater than the outer circumference of the nail. The second opening may have a second circumference that is 0.5-3 mm greater than the outer circumference of the nail. The aforementioned outer circumference of the nail, and the inner measurements of the implant, may be used when the nail and the implant are shaped for implanting in a distal femur.

Measurements of the nail, including the outer circumference of the nail, and measurements of the implant, including the inner circumferences defined by the implant, may be anatomy specific and sized based on the bone in which the nail and the implant are to be implanted.

An inner face of the first end may be threaded. An outer face of the nail may include a threaded face configured to threadedly engage the threads of the first end. The threaded face may be a first threaded face. An inner face of the second end may be threaded. An outer face of the nail may include a second threaded face configured to threadedly engage the threads of the second end.

The nail may include a first outer circumference along a first length of the nail and a second outer circumference along a second length of the nail. The first circumference may be smaller than the second circumference. The first opening may be smaller than the second opening. The first length may include threads for engaging the first end and the second length may include threads for engaging the second end.

The first end may include a first hole. The nail may include a second hole. In operation, the mesh may be coupled to the nail and the first hole may be coaxial with the second hole for receiving an anchor.

The second end may include a third hole. The nail may include a fourth hole. In operation, the mesh may be coupled to the nail and the third hole may be coaxial with the fourth hole for receiving an anchor.

The mesh anchoring substrate may be a first mesh anchoring substrate. The first mesh may be the mesh. The apparatus may include a second expandable mesh anchoring substrate. The second mesh may be the mesh. The second mesh may extend longitudinally between a third end defining a third opening and a fourth end defining a fourth opening. The second mesh may be configured to be coupled to the nail such that a length of the nail extends between the third opening and the fourth opening. In operation, the second mesh may be coupled to the nail such that the third end and the fourth end are coaxially mounted on the outer surface of the nail and spaced apart along the central axis of the nail.

The second mesh may be configured to be coupled to the nail using apparatus and methods described herein in connection with the mesh.

The second substrate may be configured to be coupled to the first substrate using any suitable coupling mechanism.

The first end may include a recess for receiving the third end.

The first end may include a first threaded surface extending along a thickness of the first end. The third end may include a second threaded surface extending along a thickness of the third end. The second threaded surface may be threaded to mate with the first threaded surface.

The mesh may include a first bulbous section including the first end and a second bulbous section including the second end. The mesh may include a neck that is defined between the first and second bulbous sections. The neck may define a third opening.

The first bulbous section, the second bulbous section and the neck may be, together, of monolithic construction.

The first bulbous section and the second bulbous section may be fixedly or removably coupled together. The first bulbous section may include the neck. The second bulbous section may include the neck. The first bulbous section may include a first portion of the neck and the second bulbous section may include a second portion of the neck, the first and second portions being configured to be coupled together.

The first end may include a first inner face extending about the first opening. The first inner face may include first protrusions extending away from the first inner face and into the first opening. The second end may include a second inner face extending about the second opening. The second inner face may include second protrusions extending away from the second inner face and into the second opening. The neck may include a neck inner face extending about the third opening. The neck inner face may include third protrusions extending away from the neck inner face and into the third opening. In operation, the mesh may be mounted on the outer surface of the nail and the first, second and third protrusions may be in physical contact with the nail and provide tension to the nail.

The first opening may have a first diameter. The second opening may have the first diameter. The third opening may have the first diameter.

The nail may have an outer circumference. The first and second openings may have a circumference that is 2%-35% greater than the outer circumference of the nail.

The nail may have an outer circumference of 10-12.5 mm. The first opening may have a first circumference that is 0.5-3 mm greater than the outer circumference of the nail. The second opening may have a second circumference that is 0.5-3 mm greater than the outer circumference of the nail. The third opening may have a third circumference that is 0.5-3 mm greater than the outer circumference of the nail. The aforementioned outer circumference of the nail, and the inner measurements of the implant, may be used when the nail and the implant are shaped for implanting in a distal femur.

Measurements of the nail, including the outer circumference of the nail, and measurements of the implant, including the inner circumferences defined by the implant, may be anatomy specific and sized based on the bone in which the nail and the implant are to be implanted.

The methods, which may involve the apparatus shown and described herein, may include a method for treating a bone. The methods may include inserting the implant in an interior of a bone. The implant may include the implant head. The methods may include inserting the expandable mesh substrate included in the implant in an interior of the bone.

The methods may include expanding the implant, in the interior, to form a mesh structure. The methods may include expanding the mesh, in the interior, to form the mesh structure. The mesh structure may be the mesh cage. The implant, including the mesh, may be delivered into the interior in a retracted state within a sleeve. Expanding the mesh may include withdrawing the sleeve from the implant. Expanding the mesh may include withdrawing the sleeve from the mesh.

The methods may include, after the inserting and the expanding, sliding a nail through the mesh substrate.

The methods may include, prior to the inserting, preparing an elongated channel in the interior, the channel shaped for receiving the nail. The methods may include, prior to the inserting, enlarging a length of the channel to form a cavity shaped for receiving the mesh.

The expanding may include withdrawing the mesh from a sleeve, the mesh being a self-expanding mesh that is in a collapsed state within the sleeve.

The mesh may extend longitudinally between a first end defining a first opening and a second end defining a second opening. The sliding of the nail through the substrate may include advancing the nail through the first opening and through the second opening.

The first end may include the hub. The second end may include the base. The base may define an annular opening. The base may be cannulated. The base may define the first opening. The hub may define the second opening.

The first end may include the base. The second end may include the hub. The hub may define an annular opening. The hub may be cannulated. The base may define the first opening. The hub may define the second opening.

The first end may have a first face extending around the first opening that includes first protrusions. The second end may have a second face extending around the second opening that includes second protrusions. The sliding may include engaging the nail with the first protrusions and the second protrusions. The engaging of the nail with the first and second protrusions may stabilize the nail in the interior.

The methods may include anchoring the mesh to the bone.

The methods may include, when the implant is a first implant and the mesh is a first mesh, inserting a second implant in the bone. The second implant may include a second expandable mesh substrate in the interior. The first and second expandable substrates may each, or both, be the mesh described herein.

The methods may include, prior to the inserting, preparing an elongated channel in the interior, the channel shaped for receiving the nail. The methods may include, prior to the inserting, enlarging a first length of the channel to form a first cavity shaped for receiving the first mesh. The methods may include, prior to the inserting, enlarging a second length of the channel to form a second cavity shaped for receiving the second mesh.

The methods may include, after the enlarging of the channel along the first length and the second length, inserting the first mesh in the first cavity. The methods may include, after the inserting of the first mesh in the first cavity, inserting the second mesh in the second cavity.

The methods may include coupling the first mesh to the second mesh in the interior.

The methods may include coupling the first mesh to the second mesh outside the bone. The methods may include, after the coupling and after the enlarging of the channel along the first length and the second length, advancing the coupled first and second mesh into the bone.

The methods may include inserting the first mesh in the first cavity after the enlarging of the channel along the first length and before the enlarging of the channel along the second length. The methods may include inserting the second mesh in the second cavity after the enlarging of the channel along the second length.

The first mesh may be more proximate an access hole than is the second mesh. The access hole may be a hole on the surface of the bone through which the first and second mesh cages were inserted. The first mesh may be distal the second mesh relative to the access hole.

The methods may include coupling the first mesh to the second mesh in the interior.

When the apparatus includes a third mesh, the method may include coupling the second mesh to the third mesh in the interior. The third mesh may be the mesh.

The methods may include preparing an access hole on the surface of the bone. The channel may be prepared through the access hole. The first cavity may be more proximate the access hole than is the second cavity.

The methods may include preparing an access hole on the surface of the bone. The channel may be prepared through the access hole. The first cavity may be distal the access hole relative to the second cavity.

The mesh may include a first bulbous shape, a second bulbous shape and a neck. The methods may include preparing an elongated channel in the interior, the channel shaped for receiving the nail. The methods may include enlarging the channel along a first length of the channel to form a first cavity shaped for receiving the first bulbous shape. The methods may include enlarging the channel along a second length of the channel to form a second cavity shaped for receiving the second bulbous shape.

The mesh may extend between a first end having a first opening and a second end having a second opening. The first end may have first protrusions extending away from the first end and into the first opening. The second end may have second protrusions extending away from the second end and into the second opening. The neck may have third protrusions extending away from a neck opening and into a neck opening. The sliding may include engaging the nail with the first, second and third protrusions. The engaging of the nail with the first, second and third protrusions may stabilize the nail in the interior.

The bone may be a humerus bone and the mesh may be positioned in a proximal portion of the humerus.

The bone may be a femur and the mesh may be positioned in a distal portion of the femur.

The bone may be a tibia and the mesh may be positioned in a proximal portion of the tibia.

The bone may be a calcaneus bone and the mesh may be positioned adjacent a surface of the calcaneus.

The bone may be any other suitable bone in the human body, such as any bone Skin FIG. 106. The mesh may be positioned adjacent an articular surface of the bone. The method may be positioned adjacent a surface that is not an articular surface.

When the bone is a first bone and the mesh is a first mesh, the methods may include placing a second mesh in a second bone. The second bone may be opposite the first bone across an articular surface.

The methods may include removing the nail from the bone. The methods may include broaching matter in an interior of the mesh. The methods may include collapsing the mesh. The methods may include removing the mesh from the bone. The mesh may be collapsed using the inserter.

The collapsing may include coupling a removal device to a first end of the mesh. The removal device may be the inserter.

The collapsing may include coupling the removal device to a second end of the mesh. The collapsing may include increasing a distance between the first end and the second end. The collapsing may include advancing the mesh into a tubular sleeve to compress the mesh.

The methods may include removing the nail from the bone. The methods may include broaching matter in an interior of the first mesh. The methods may include broaching matter in an interior of the second mesh. The methods may include collapsing the first mesh and collapsing the second mesh. The methods may include removing the first mesh and the second mesh from the bone.

The methods may include removing the nail from the bone. The methods may include broaching matter in an interior of the first bulbous shape. The methods may include broaching matter in an interior of the second bulbous shape. The methods may include collapsing the mesh. The methods may include removing the mesh from the bone.

The methods, which may involve the apparatus shown and described herein, may include a method for treating a bone. The methods may include mounting the implant head in a collapsed state, on an outer surface of the nail. The mounting of the implant head may include mounting the mesh substrate, in a collapsed state, on an outer surface of a nail.

The methods may include preparing an elongated channel in an interior of the bone for receiving the nail. The methods may include enlarging the channel along a length of the channel to form a cavity for receiving the mesh in an expanded state.

The mesh substrate may be mounted on the outer surface of the nail outside the bone. The methods may include inserting the nail and the mesh, mounted to the nail, in the elongated channel. The methods may include expanding the mesh, in the cavity, to form a mesh structure. The mounting of the mesh substrate may include sliding a sleeve over the outer surface of the nail, the mesh substrate being in a collapsed state within the sleeve.

The mesh substrate may be mounted on the outer surface of the nail inside the bone. The nail may slide through the mesh substrate, in a collapsed state within a sleeve, when the mesh substrate is positioned inside the bone. The methods may include inserting mesh in the collapsed state in the elongated channel and, after the inserting of the mesh, sliding the nail through the collapsed mesh. The methods may include expanding the mesh, in the cavity, to form a mesh structure.

The expanding of the mesh may include withdrawing the sleeve from the mesh.

The mesh may include a first bulbous portion and a second bulbous portion. The cavity may be a first cavity and the length of the channel may be a first length of the channel. The first cavity may be shaped to receive the first bulbous portion. The methods may include enlarging the channel along a second length of the channel to form a second cavity for receiving the second bulbous portion. The methods may include positioning the nail in the channel so that, when the sleeve is withdrawn from the mesh, the first bulbous portion expands into the first cavity and the second bulbous portion expands into the second cavity.

The methods, which may involve the apparatus shown and described herein, may include a method for treating a bone. The methods may include placing the expandable implant in an interior of a bone. The methods may include expanding a head of the implant to form a mesh cage. The methods may include positioning a plate on a surface of the bone. A tail of the implant may be seated in an opening defined by the plate.

The methods may include placing a nail in the interior of the bone such that nail is spaced apart from the implant.

The methods may include placing the nail in the interior of the bone such that the nail does not abut the implant.

The methods may include placing the nail in the interior of the bone such that the nail abuts, but is not coupled to, the implant.

The nail may be positioned medial in the bone relative to the implant. The nail may be positioned distal in the bone relative to the implant.

The apparatus may include apparatus for, and the methods may involve, collapsing the expandable implant head. The apparatus may include a shaft. The apparatus may include a guide tube for guiding movement of the shaft. The apparatus may include a lock configured to fix the shaft in a position relative to the guide tube.

The apparatus may include retaining fingers fixed relative to the guide tube and configured to engage windows in an implant base. The apparatus may include an actuator configured to move the shaft relative to the guide tube. The actuator may include a handle and a trigger. The trigger may be is moveable relative to the handle. The actuator may include a turn-able knob.

The retaining fingers may engage the implant base. When the retaining fingers are engaged with the implant base, movement of the shaft may push a hub of the implant apart from the base and collapse the expandable implant head.

The expandable implant may be inserted into a bone. The expandable implant may be inserted into a bone in a collapsed state. The apparatus may include a pusher configured to release the retaining fingers from the windows. The pusher may be used to release the implant from the retaining finger after the implant is positioned in the bone.

The apparatus may include the shaft. The apparatus may include a guide tube. The guide tube may guide movement of the shaft. The apparatus may include a handle. The handle may be configured to move the shaft relative to the guide tube.

The apparatus may include a retaining finger. The retaining finger may extend from a distal end of the guide tube. The retaining finger may be configured to engage a window in a base of the expandable implant. The retaining finger may be engaged with the window.

The expandable implant may include a head biased to self-expand. The self-expansion properties of the implant may bias a hub of the expandable implant to be positioned a default distance from the base. The distal end of the shaft may push the hub, against self-expansion bias, further apart from the base than the default distance. Separating the hub and the base by more than the default distance may collapse the implant.

The apparatus may include a locking mechanism. The locking mechanism may be configured to lock the shaft relative to the guide tube. The shaft may be locked, relative to the guide tube, such that a distal end of the shaft protrudes beyond a distal end of the retaining finger. In a locked position, the shaft may be configured to space the hub further apart from the base than the default distance.

When the shaft is locked relative to the guide tube, the shaft may be configured to space a hub of the implant a predetermined distance apart from the base. When the shaft is locked relative to the guide tube, the expandable implant may be locked in a collapsed state. When the retaining finger is engaged with the base, the locking mechanism may be configured to resist force applied to the shaft by an implant head that biases a hub to move towards the base.

The actuator may include a handle affixed to the shaft. The actuator may include a flange affixed to the guide tube. The handle may be a T-shaped handle. The handle and the flange may be configured such that the handle and the flange may both be held simultaneously by one human hand. The handle and the flange may be configured such that the handle and the flange may be held simultaneously by one human hand. The flange may be configured to be held by at least one human finger and the handle may be configured to be supported by a human palm, thumb or any other part of the same hand.

The locking mechanism may include a dowel protruding from the shaft. The locking mechanism may include a locking channel in the guide tube. Wherein the channel may include a guide segment that guides the dowel along a longitudinal axis of the guide tube. The channel may include a locking channel that may be configured to hold the dowel at a position along the longitudinal axis of the guide tube.

The locking channel may be one of a plurality of locking channels. Each locking channel may correspond to a different length expandable implant. A length of the expandable implant in a collapsed state may be longer than a length of the implant in the expanded state. A length of the expandable implant in an expanded state may be an equilibrium spacing of a hub of the implant apart from the base when the implant is free to fully self-expand.

The plurality of locking channels may include a first locking channel and a second locking channel. The first locking channel may be configured to hold the dowel at a first position along the longitudinal axis such that the distal end of the shaft protrudes a first distance beyond the retaining finger. The second locking channel may be configured to hold the dowel at a second position along the longitudinal axis such that the distal end of the shaft protrudes a second distance past the retaining finger. The first distance may be greater than the second distance. The second distance may be greater than the first distance.

The plurality of locking channels may include a third locking channel. The third locking channel may be configured to hold the dowel at a third position along the longitudinal axis such that the distal end of the shaft protrudes a third distance past the retaining finger. The third distance may be greater than the second distance and less than the first distance. The third distance may be greater than the first distance and less than the second distance.

Each locking channel may be configured to resist a compressive force applied to the distal end by a hub of the expandable implant. The hub may be biased to move towards the base. When the base of the implant is coupled to the retaining finger, the shaft may need to overcome the bias to collapse the implant by increasing a distance between the implant hub and implant base.

The shaft may be configured to rotate relative to the guide tube to position the dowel in the locking channel. The apparatus may include a handle affixed to the shaft and configured to rotate the shaft relative to the guide tube. The apparatus may include a handle that is affixed to the shaft. The apparatus may include a handle that is configured to move the shaft axially along a longitudinal axis of the guide tube and relative to the guide tube.

When the shaft is locked relative to the guide tube, the shaft may lock the retaining finger in the window of the base. The retaining finger may be one of a plurality of retaining fingers. The plurality of retaining fingers may be distributed about a central longitudinal axis of the guide tube. The plurality of retaining fingers may be configured to be compressed toward the central longitudinal axis of the guide tube.

When the shaft is in a locked position, the shaft may prevent the retaining fingers from compressing toward the longitudinal axis of the guide tube. When the shaft is in a retracted position, the plurality of retaining fingers may be configured to be compressed about the longitudinal axis of the guide tube.

The apparatus may include apparatus for, and the methods may involve, inserting the self-expanding implant into a bone. The apparatus may include a guide tube. The apparatus may include a shaft. The shaft may be configured to slide within the guide tube. The apparatus may include a locking mechanism. The locking mechanism may be configured to lock a position of the shaft relative to the guide tube.

The locking mechanism may be configured to resist compressive force applied to the shaft by the self-expanding implant when the implant is coupled to the guide tube. The compressive force may have a magnitude at least as great as force needed to space a distal end of the expandable implant apart from a proximal end such that the self-expanding implant is in a collapsed state.

The guide tube may include a collar that includes a plurality of retaining fingers. The collar may be affixed to a distal end of the guide tube. The retaining fingers may be configured to flex radially relative to a longitudinal axis of the collar and/or guide tube. Each retaining finger may be configured to releasably engage a base of the self-expanding implant.

Each retaining finger may include a protrusion configured to engage a window in a base of the self-expanding implant. Each retaining finger may be biased to position the protrusion at a first radial distance from the longitudinal axis. Each retaining finger may be configured to flex and reposition the protrusion at a second radial distance from the longitudinal axis.

The first radial distance may be greater than the second radial distance. A difference between the first radial distance and the second radial distance may be greater than or equal to a height of the protrusion. The second radial distance may be less than an inner radius of a base of the self-expanding implant. The second radial distance may be closer to a central longitudinal axis of the collar and/or guide tube than the first radial distance.

The shaft may be configured to be positioned inside the collar such that the retaining fingers cannot move from the first radial distance to the second radial distance. The shaft may be retractable, proximally, inside the collar such that the retaining fingers are moveable from the first distance to the second radial distance.

The shaft may be positioned inside the collar such that the retaining fingers cannot move from the first radial distance to the second radial distance. The shaft may be locked in a position such that the retaining fingers cannot move from the first radial distance to the second radial distance. In a locked state, the shaft may be configured to protrude a targeted distance beyond a distal end of the collar.

The collar may include a guide segment between adjacent retaining fingers. Each guide segment may be spaced apart from each retaining finger. The shaft may define a shoulder. Each guide segment may define a catch configured to be seated against the shoulder of the shaft and prevent distal movement of the shaft relative to the collar. Each retaining finger may include a catch configured to be seated against the shoulder of the shaft and prevent distal movement of the shaft relative to the collar.

The collar may have a proximal end defining an inner diameter. The guide tube may include a distal end having an outer diameter. The outer diameter may be configured to be received by the proximal end of the collar. The proximal end of the collar and the distal end of the shaft may be configured to form a press fit. The press fit may be configured to withstand a biasing force urging a distal end of the expandable implant apart from a proximal end such that the self-expanding implant, when coupled to the collar, may be held in a collapsed state.

The collar may define a first shoulder. The shaft may define a second shoulder. The second shoulder may be configured to be seated against the first shoulder and stop the shaft from extending more than a maximum distance beyond the distal end of the collar.

The apparatus may include a key defined by the guide tube. The apparatus may include a slot defined by the collar. The key may be configured to fit into the slot and prevent the collar from rotating about a longitudinal axis of the guide tube. The apparatus may include a slot defined by the guide tube. The apparatus may include a key defined by the collar. The key may be configured to fit into the slot and prevent the collar from rotating about longitudinal axis of guide tube.

The apparatus may include apparatus for, and the methods may involve, a delivery system for the expandable implant. The system may include a containment tube. The containment tube may be configured to hold an expandable implant in a collapsed state. The system may include a guide tube. The guide tube may be configured to be releasably coupled to the expandable implant. The system may include a shaft. The shaft may be moveable relative to the guide tube. The shaft may be configured to position a hub of the expandable implant relative to a base of the expandable implant such that when the expandable implant is removed from the containment tube, the expandable implant is held in a collapsed state.

The system may include an actuator for moving the guide tube relative to the shaft. The shaft may be threadedly coupled to the guide tube. The shaft may be threaded through the guide tube. The actuator may include a threaded bushing. The shaft may be threaded. The shaft may be configured to threadedly engage the threaded bushing. A knob may be affixed to the shaft. Turning the knob may threadedly engage the shaft and the threaded bushing.

The actuator may include a grip plate. The actuator may include a trigger. The trigger may be configured to rotate the grip plate and move the shaft distally relative to the guide tube.

The system may include a T-shaped handle affixed to the shaft. The system may include a flange affixed to the guide tube. The guide tube may define a cylindrical surface. The shaft may be positioned within the cylindrical surface.

The system may include a collar. The collar may be affixed to the guide tube. The collar may include a plurality of retaining fingers. Each finger may be configured to engage a window in the base.

The collar may be fixed axially and rotationally relative to the guide tube. The collar may be rotationally fixed relative to the guide tube by a slot and key arrangement. The collar may be axially fixed relative to the guide tube by a press fit with the guide tube. The press fit may be configured to resist tension applied to the collar when the collar holds the expandable implant in a collapsed state. The collar may be axially fixed relative to the guide tube by threaded engagement with the guide tube.

The system may include a shaft lock. The shaft lock may be configured to fix the shaft axially relative to the guide tube. The shaft lock may include a spring-loaded pawl configured to engage a groove in the shaft. The shaft lock may include a pawl configured to engage a groove in the shaft.

The shaft lock may include a dowel mounted on the shaft. The shaft lock may include a channel in the guide tube. The shaft may be rotatable relative to the guide tube. Rotating the shaft may position the dowel within the channel. The channel may be one of a plurality of channels. Each channel may be spaced apart from each other along a longitudinal axis of the guide tube.

The methods, which may involve the apparatus shown and described herein, may include methods for inserting the expandable implant into a bone. Methods may include coupling the implant to the guide tube. The methods may include positioning a distal end of the shaft in contact with a hub the implant.

The methods may include locking a position of the distal end inside the implant. The methods may include inserting the implant into a bone. The methods may include retracting the distal end of the shaft into the guide tube. The methods may include disengaging the implant from the guide tube.

The methods may include rotating the implant after inserting the implant into the bone and before disengaging the implant from the guide tube. The methods may include coupling the implant to the guide tube by positioning a protrusion into a window of a base of the implant. The methods may include locking the distal end of the shaft inside the implant such that a hub of the implant is spaced apart from the base by a distance that maintains the implant in a collapsed state.

The methods may include inserting the implant into the bone in the collapsed state. The methods may include inserting the implant into the bone in the collapsed state and allowing the implant to self-expand inside the bone while the implant is coupled to the guide tube.

The coupling may include compressing a retaining finger extending from the guide tube about a longitudinal axis of the guide tube. The coupling may include positioning a protrusion extending from the retaining finger in a window defined by a base of the implant.

The disengaging may include compressing a retaining finger that extends from the guide tube about a longitudinal axis of the guide tube. The disengaging may include compressing the retaining finger such that a protrusion extending from the retaining finger disengages from a window defined by a base of the implant. The disengaging may include, after the implant self-expands inside the bone, pulling the guide tube in a proximal direction. Pulling the guide tube in the proximal direction to compress the retaining finger such that a protrusion extending from the retaining finger disengages from a window defined by a base of the implant.

The locking may include twisting the shaft. The locking may include moving a dowel extending from the shaft into a locking channel defined by the guide tube. The methods may include, before the retracting, twisting the shaft. The methods may include, before the retracting, twisting the shaft to move a dowel extending from the shaft out of a locking channel defined by the guide tube.

The retracting may include moving the dowel along a guide channel defined by the guide tube. The positioning of the distal end may include grasping, with fingers of a hand, a handle affixed to the guide tube. The positioning of the distal end may include pushing, using a palm of the hand, the shaft out of the guide tube.

The methods may include, after the coupling, pulling the implant out of a containment tube. The methods may include pulling the implant out of the containment tube in a collapsed state. The methods may include, after the disengaging, repositioning a head of the implant inside the bone. The methods may include repositioning the head of the implant by inserting a support into the implant.

The methods, which may involve the apparatus shown and described herein, may include a method for removing an implant from a bone. The methods may include coupling the guide tube to a proximal base of the implant positioned inside a bone. The methods may include moving a distal end of a shaft into the proximal base. The methods may include collapsing the implant inside the bone by pushing the distal end of the shaft against a distal hub of the implant. The methods may include pulling the collapsed implant out of the bone.

The methods may include locking the distal end of the shaft inside the implant by twisting the shaft. The methods may include locking the distal end of the shaft inside the implant by shifting a dowel extending from the shaft into a locking channel defined by the guide tube.

The coupling may include compressing a retaining finger extending from the guide tube about a longitudinal axis of the guide tube. The coupling may include positioning a protrusion extending from a retaining finger in a window defined by the base of the implant.

The methods may include locking the protrusion in the window by shifting the shaft distally relative to the guide tube.

The methods may include broaching bone ingrowth inside a head of the implant. The methods may include, after the coupling, twisting the implant to separate a head of the implant from the bone. The methods may include twisting the implant to break up bone ingrowth that penetrated a head of the implant. The methods may include grasping a handle affixed to the guide tube with fingers of a hand. The methods may include moving the distal end of the shaft using the hand.

The apparatus may include apparatus for, and the methods may involve, apparatus for repairing a fractured bone. The apparatus may define a longitudinal axis. The apparatus may include the expandable implant head. The apparatus may include the extension member. the extension member may be configured to be removably coupled to the implant head. The apparatus may include a locking cap. The locking cap may be the fixation member. The locking cap may be configured to be removably coupled to the extension member.

The locking cap may be configured to mate with a plate positioned outside the fractured bone. The locking cap may be configured to fix a position of the extension member inside the bone.

The apparatus may include a locking socket. The locking socket may be configured to be removably coupled to the extension member. The locking socket may include locking fingers. The locking socket may include a tooth protruding from each locking finger.

The apparatus may include the plate. The plate may define a longitudinal plate axis L_{P}. The plate may be shaped to fit over anatomical features of the bone. The plate may conform to a surface contour of the bone. The plate may define an opening shaped to receive an implant inserted into an interior of the bone. The plate may define a plurality of ridges around the opening. The tooth may be configured to be seated between a first of the ridges and a second of the ridges. The first and second ridges may define a groove. The tooth may be seated in the groove.

The locking cap may be configured to engage the locking socket and push the locking fingers away from a central longitudinal axis of the locking socket.

The locking socket may be configured to be positioned in the opening. Threaded engagement of the locking cap with the locking socket may be configured to fix a longitudinal axis of the locking socket with respect to a longitudinal axis of the plate.

The implant head may comprise a plurality of cells. Each cell, when expanded, may be configured to engage a screw. The implant head, be an expandable mesh cage. The implant head may be configured to self-expand from a collapsed state to an expanded state.

The extension member may include a dimpled head. The plate may comprise a dimpled surface. A dimpled surface may include an uneven surface that includes dips and rises. A first dimpled surface may be engaged with second dimpled surface. For example, a first dimpled surface may be pressed against a second dimpled surface. Pressing the first dimpled surface against the second dimpled surface may position rises of one surface in dips of the other surface.

Engagement of the first and second dimpled surfaces may prevent the first and second dimpled surfaces from moving or sliding relative to each other. The locking cap may be configured to threadedly engage the plate and press one dimpled surface against another dimpled surface. One dimpled surface may include a dimpled head of an elongated member. The locking cap may press the two dimpled surfaces together, locking a position of the elongated member.

The apparatus may include a locking screw. The extension member may include a pitted head. A pitted head may include a polarity of discrete indentations in the head of the elongated members. Each indentation may be spaced apart from a neighboring indentation by a land. The land between the indentations may be contiguous and smooth with non-pitted portions of the head. The locking screw may threadedly engage the locking cap. A tip of the locking screw may mate with a pit in the pitted head to fix the position of the extension member inside the bone.

The plate may include a high friction surface. The extension member may include a high friction surface. The high friction surface of the extension member may be configured to be seated on the high friction surface of the plate to form an interface having a first coefficient of friction. The extension member may include a low friction surface.

The locking cap may include a low friction surface that may be configured to form with the low friction surface of the extension member an interface having a second coefficient of friction. The low friction surface of the locking cap may articulate against the low friction surface of the extension member.

The locking cap may be configured to threadedly engage the plate. The locking cap may be configured to press the high friction surface of the extension member against the high friction surface of the plate to fix the position of the extension member inside the bone.

The apparatus may include apparatus for, and the methods may involve, apparatus for repairing a fractured bone. The apparatus may include a plate that defines an opening. The plate may include a first threaded surface. The plate may include a supporting articular surface;

The apparatus may include elongated member. The elongated member may include an inner articular surface. The elongated member may include an outer articular surface. The outer articular surface may be configured to be supported by the supporting articular surface.

The apparatus may include a locking cap. The locking cap may be the fixation member. The locking cap may include a second threaded surface. The second threaded surface may be configured to threadedly engage the first threaded surface. The locking cap may include a locking articular surface configured to engage the inner articular surface.

The inner articular surface may be configured to receive the locking articular surface and press the outer articular surface against the supporting articular surface.

When the elongated member is positioned in the opening, threaded engagement of the first and second threaded surface may lock a position of the elongated member with respect to the plate.

The plate may define a longitudinal plate axis. The elongated member may define an longitudinal implant axis. Engagement of the locking articular surface and the inner articular surface may fixes a position of the longitudinal plate axis with respect to the longitudinal implant axis.

Threaded engagement of the first and second threaded surfaces may be configured to press the locking surface against the inner articular surface and the outer articular surface against the supporting articular surface.

The supporting articular surface may define a circumference that varies along a thickness of the plate.

The circumference of the supporting articular surface may be larger than a maximum circumference of the outer articular surface at a first depth along a thickness of the plate. The circumference of the supporting articular surface may be smaller than a maximum circumference of the outer articular at a second depth along a thickness of the plate. The first depth may be positioned between the first threaded surface and the second depth.

The plate may define a relief surface. The relief surface may be configured to provide the elongated member an angular range of motion relative to a longitudinal plate axis. The relief surface may extend, along a thickness of the plate, between a distal surface of the plate and the supporting articular surface. The angular range of motion may be substantially 15°. The distal surface of the plate may be configured to be seated on a bone.

A first coefficient of friction between the supporting articular surface and the outer articular surface may be greater than a second coefficient of friction between the locking articular surface and the inner articular surface. A normal force applied by the locking surface to the supporting articular surface and outer articular surfaces may be greater than a normal force applied by the locking cap to the inner articular surface. When the locking cap is threaded into an opening of the plate, a force of friction applied by the outer articular surface to the supporting articular surface may be greater than a force of friction applied by the locking articular surface to the inner articular surface.

The elongated member may include a cylindrical segment. The outer articular surface may extend from the cylindrical segment.

The cylindrical segment may define a first diameter. The outer articular surface may have a second diameter that is greater than the first diameter.

The supporting articular surface may define a third diameter. The third diameter may be less than the second diameter and greater than the first diameter.

The elongated member may include a threaded end. The outer articular surface may be at a proximal end of the elongated member. The threaded end may be at a distal end of the elongated member. The threaded end may be configured to be threaded into a hub of an implant. The threaded end may be configured to be threaded into the support of the implant.

The locking cap may define a drive that provides access to the elongated member after the locking cap is secured to the plate.

The elongated member may define a cannula configured to receive a tool inserted through the drive of the locking cap.

The methods, which may involve the apparatus shown and described herein, may include a method for securing an implant inside a bone. The methods may include affixing a plate to an outer surface of the bone.

The methods may include inserting an elongated member into the bone through an opening in the plate. The methods may include seating an outer surface of the elongated member on an articular surface that extends into the opening. The methods may include locking, in a position with respect to the plate, the elongated member by pressing the outer surface of the elongated member against the articular surface.

The methods may include locking the elongated member in the position by driving a locking cap into the opening.

The methods may include driving the locking cap into the opening such that the locking cap presses the outer surface against the articular surface. The methods may include driving the locking cap into the opening increases slip-resistance between the outer surface and the articular surface. The methods may include driving the locking cap into the opening to generate a first slip-resistance between the outer surface and the articular surface.

The methods may include adjusting a position of the elongated member with respect to the plate. The methods may include driving the locking cap further into the opening to generate a second slip-resistance between the outer surface and the articular surface. The second slip-resistance may be greater than the first slip-resistance. Slip-resistance may be a function of a coefficient of friction of each surface. Slip-resistance may be a function of a normal force applied by one surface to another.

The methods may include affixing the elongated member to a hub of the implant. The methods may include affixing the elongated member to a support of the implant.

The apparatus may include apparatus for, and the methods may involve, apparatus for repairing a fractured bone. The apparatus may include a plate. The plate may define a supporting articular surface. The apparatus may include an elongated member. The elongated member may include an outer surface. The outer surface may be configured to be seated on the supporting surface of the plate.

The apparatus may include a locking cap. The locking cap may be configured to be removably coupled to the plate. The locking cap may be configured to press the outer surface of the elongated member against the supporting surface of the plate. The locking cap may be threadedly couple to the plate. Threaded engagement of the locking cap and the plate may press the outer surface of the elongated member against the supporting surface of the plate.

The locking cap may be configured to press the outer surface of the elongated member against the supporting surface of the plate. The locking cap may press the outer surface of the elongated member against the supporting surface of the plate such that friction between the outer surface and the supporting surface fixes a position of the implant with respect to the plate.

The elongated member may include an inner surface. The locking cap may include a locking surface. The locking surface may be configured to articulate against the inner surface. The plate may include an opening that defines threads. The locking cap may be configured to threadedly engage the threads. The locking surface may articulate against the inner surface as the locking cap threadedly engages the plate. The locking cap may be configured to press the locking surface against the inner surface. The locking cap may be configured to press the outer surface against the supporting surface.

Apparatus may include an implant affixed to the elongated member. Friction between the outer surface and the supporting surface may fix a position of the implant with respect to the plate. The elongated member may include a cylindrical body. The elongated member may include a flared end. The flared end may include the outer surface.

The plate may have a thickness. A first segment of the thickness, a second segment of the thickness, a third segment of the thickness may together define an opening in the plate that penetrates the thickness. The first segment may include threads for engaging the locking cap. The second segment may include the supporting surface. The third segment may include a relief surface.

The relief surface may be configured to provide the elongated member an angular range of motion relative to the plate. The elongated member may include a threaded end configured to engage a hub of an implant. The elongated member may include a threaded end that may be configured to engage a support of an implant.

The steps of illustrative methods may be performed in an order other than the order shown and/or described herein. Some embodiments may omit steps shown and/or described in connection with the illustrative methods. Some embodiments may include steps that are neither shown nor described in connection with the illustrative methods. Illustrative method steps may be combined. For example, one illustrative method may include steps shown in connection with another illustrative method.

Some embodiments may omit features shown and/or described in connection with the illustrative apparatus. Some embodiments may include features that are neither shown nor described in connection with the illustrative apparatus. Features of illustrative apparatus may be combined. For example, one illustrative embodiment may include features shown in connection with another illustrative embodiment.

Embodiments may involve some or all of the features of the illustrative apparatus and/or some or all of the steps of the illustrative methods.

The illustrative apparatus and therapeutic scenarios will now be described with reference to the accompanying drawings in the Figures, which form a part hereof. It is to be understood that other embodiments may be utilized and that structural, functional and procedural modifications may be made without departing from the scope and spirit of the present disclosure.

For the sake of clarity, figures may illustrate therapeutic treatment of bones without showing fracture lines.

Apparatus and methods illustrated in the figures are shown in reference to a bone ‘B’. The apparatus and methods described in respect to bone ‘B’ may be utilized in any bone Sᵢ included in Table 11 below, or any other bone in the human body.

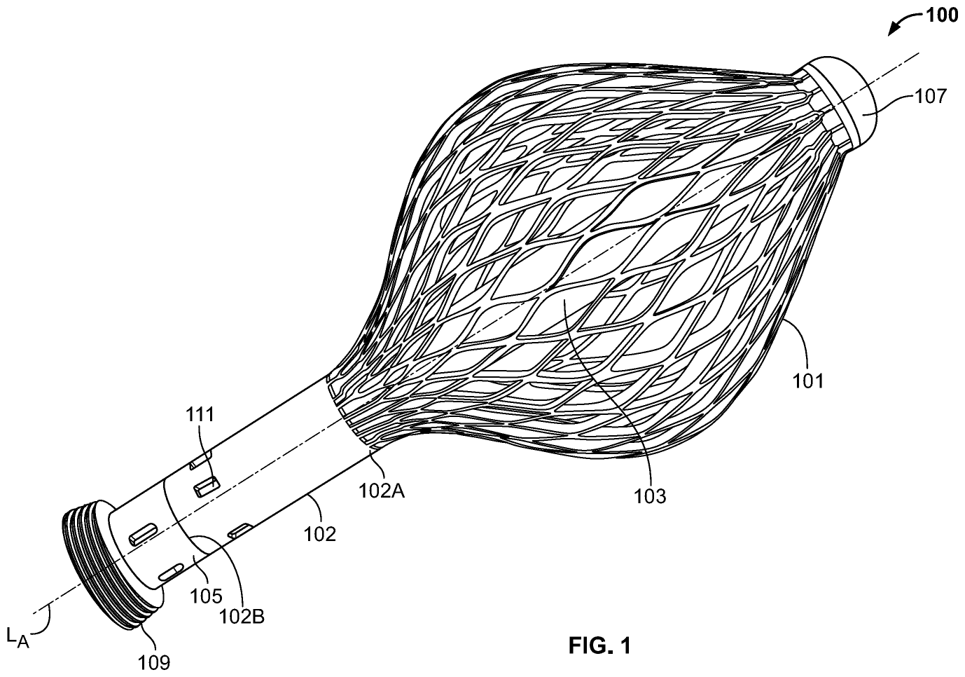

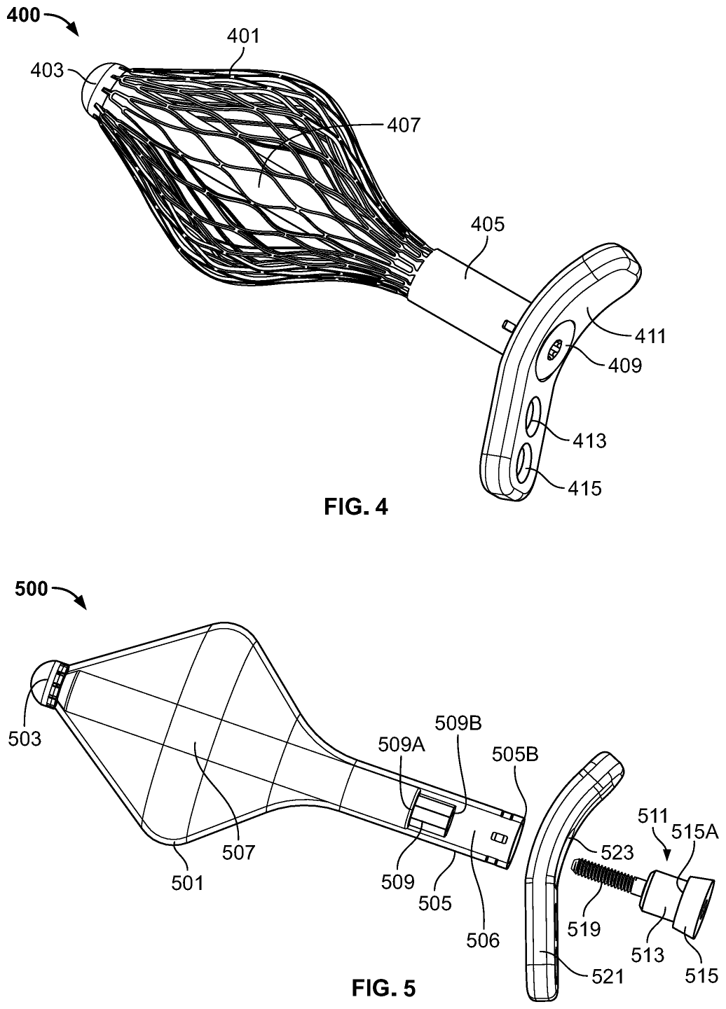

In some figures, the mesh anchoring substrate included in the implant head, when expanded, is illustrated as a bulbous shape without showing features of the mesh structure (such as features of the mesh structure illustrated in FIGS. 1 and 4, for example). It is to be understood that each of the expanded implant heads illustrated in the figures may form a mesh structure including a plurality of interconnected cells coupled to the hub and the base as illustrated in FIGS. 1, 4, and the remaining figures showing the mesh.

Figures below describe illustrative implant heads, implants, supports, fixation members, extension members, mesh anchoring substrates, bases, hubs, implant tails, nails, and plates. Each of the illustrative implants may have features in common with any of the other illustrative implants disclosed herein. Each illustrative implant head may have features shown in connection with any of the other illustrative implants disclosed herein. Each of the illustrative supports may have features in common with any of the other illustrative supports disclosed herein. Each of the illustrative fixation members may have features in common with any of the other illustrative fixation members disclosed herein. Each of the illustrative extension members may have features in common with any of the other illustrative extension members disclosed herein. Each of the illustrative mesh anchoring substrates may have features in common with any of the other illustrative mesh anchoring substrates disclosed herein. Each of the illustrative bases may have features in common with any of the other illustrative bases disclosed herein. Each of the illustrative hubs may have features in common with the hub. Each of the illustrative implant tails may have features in common with any of the other illustrative implant tails disclosed herein. Each of the illustrative nails may have features in common with any of the other illustrative nails disclosed herein. Each of the illustrative plates may have features in common with any of the other illustrative plates disclosed herein.

Each of the illustrative implants may have features in common with the implant. Each of the illustrative implant heads may have features in common with the implant. Each of the illustrative supports may have features in common with the support. Each of the illustrative fixation members may have features in common with the fixation member. Each of the illustrative extension members may have features in common with the extension member. Each of the illustrative mesh anchoring substrates may have features in common with the mesh anchoring substrate. Each of the illustrative bases may have features in common with the base. Each of the illustrative hubs may have features in common with the support. Each of the illustrative implant tails may have features in common with the implant tail. Each of the illustrative nails may have features in common with the nail. Each of the illustrative plates may have features in common with the plate.

FIG. 1 shows illustrative apparatus 100. Apparatus 100 may define longitudinal axis LA. Apparatus 100 may include mesh anchoring substrate 101 extending between hub 107 and base 102. Support 103 may have one or more features in common with support 208 (described below). Fixation member 109 may have one or more features in common with fixation member 215 (described below). Hub 107 may have one or more features in common with hub 203 (described below).

Base 102 may be cannulated. Support 103 may extend through the cannula defined by base 102. Support 103 may extend through a volume defined by mesh 101. A first end of support 103 may be coupled to a coupling mechanism in hub 107. The coupling mechanism may have features of hub coupling mechanisms described herein. Flange 105 may be positioned at a second end of support 103.

Base first end 102A may be adjacent mesh 101. Base second end 102B may be opposite first end 102A. Flange 105 may be seated on base second end 102B. Base 102 may include windows 111. Windows 111, and windows disclosed herein, may be coupled to protrusions on a retaining figure included on an inserter. The inserter may include features of apparatus 10700. The inserter may include features of 12900. The inserter may include features of apparatus 13300. The inserter may include features of apparatus 14100.

A diameter defined by flange 105 may be equal to, or substantially equal to, a diameter defined by base 102. A circumference defined by flange 105 may be equal to, or substantially equal to, a diameter defined by base 102. Similar outer shapes of flange 105 and base 102 may enable base 102 and flange 105 to be implanted in a bore having an outer circumference that was drilled by a drill having a single drill-bit.

Fixation member 109 may be coupled to flange 105. Head of fixation member 109 may be threaded for threadedly engaging a threaded opening in a plate. Fixation member 109 may be used to fix the remainder of apparatus 100 to a plate in which the head of fixation member 109 is seated.

When apparatus 100 is assembled as shown in FIG. 1, longitudinal axis LA may be coaxial with a central axis of hub 107, a central axis of base 102, a central axis of flange 105 and a central axis of fixation member 109.

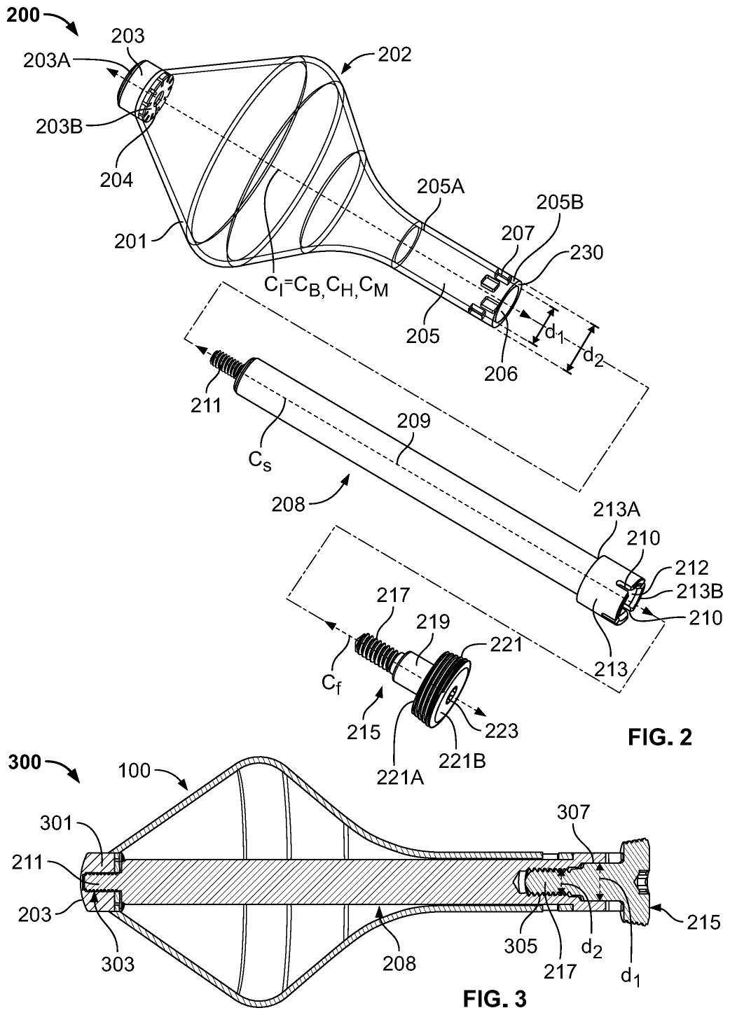

FIG. 2 shows an exploded view of apparatus 200. Exemplary apparatus may include implant 202, support 208 and fixation member 215. The exploded view illustrated in FIG. 1 includes modular parts that may be used to assemble an implant, such as the implant illustrated in FIG. 1.

Implant 202 may include mesh anchoring substrate 201. Mesh anchoring substrate 201 may extend between hub 203 and base 205. Hub 203 may include hub first end 203A and hub second end 203B. Second end 203B may define opening 204. A threaded bore (illustrated in FIG. 3) may extend through opening 204 and into a thickness of hub 203. The threaded bore may be sized for threaded engagement with threaded surface 211.

Base 205 may be cannulated. Cannula 206 may extend through base 205. Cannula 206 may extend axially along central axis C_{B} of base 205. Base 205 may have thickness 230. thickness 230 may define base second end 203B. Thickness 230 may extend between diameter d₁ of cannula 206 and diameter d₂ of base 205. Base 205 may define one, two, three, four, five, or more windows 207. Windows 207 may be may be coupled to protrusions on a retaining figure included on an inserter.

Implant 202 may define central axis C₁. Central axis C₁ may be coaxial with a central axis C_{H} of hub 203, central axis C_{B} of base 205, and central axis C_{M} of mesh 201.

Support 208 may include elongated body 209 that is defined between threaded surface 211 and flange 213. Support 208 may define central axis C_{S}.

Flange 213 may have flange first face 213A and flange second face 213B. Flange first end 213A may be shaped configured to be seated on base second end 205B. Flange second end 213B may define opening 212 for receiving threaded surface 217 and body 219. Slots 210 may extend through flange second face 213B axially along central axis C_{S}. Slots 210, and slots described herein positioned at an end of a flange, may be for coupling to a screwdriver, such as a cruciform shape screwdriver.

Fixation member 215 may define central axis CF. Fixation member 215 may include body 219 that is defined between threaded surface 217 and head 221. In operation, bottom face 221A of head 221 may be seated on flange second face 213B. Circumferential threads extending about head 221 may be shaped for threadedly engaging a threaded opening in a plate. Fixation member 215 may include indentation 223 for receiving a tool. Indentation 223 may be used by the tool to drive fixation member 215 in rotation about central axis C_{S}.

Support 208 may include a bore having a first, smooth portion for seating body 219 and a second, threaded portion for threadedly engaging threaded surface 217 inside support 208 (illustrated in FIG. 3, below).

The methods may include selecting one or more of an implant, a support, a plate and a fixation member for treating a bone from a kit including one or more implants, supports, plates and/or fixation members. One or more of the plates may define different bottom surface contours and/or angles for resting on the bone.

The methods may include selecting a support from two or more supports. Each of the flanges of the supports may have a different length. After implanting the implant in the bone, the methods may include selecting a support from the two or more supports having a flange whose length extends between the base second end and a surface of the bone such that the flange second face is positioned adjacent the surface of the bone, such as within 5 mm of the bone, 2 mm, 3 mm, 4 mm, or any other suitable distance.

FIG. 3 shows cross-sectional view 300 of apparatus 200, assembled. Cross-sectional view 300 shows threaded bore 303 extending through thickness 301 of hub 203. Cross-sectional view 300 also shows a bore extending through support 208. The bore may include first length 307 shaped for seating body 219 of fixation member 215. The bore may include second length 305. Second length 305 may be threaded for threadedly engaging threaded surface 217 of fixation member 215. First length 307 may have diameter d₁. Second length 305 may have diameter d₂. Diameter d₁ may be greater than diameter d₂.

FIG. 4 shows illustrative apparatus 400. Apparatus 400 may include mesh 401 extending between hub 403 and base 405. Support 407 is shown extending between hub 403 and base 405. Support 407 may be removably coupled to hub 403 by any of the coupling mechanisms described herein. Apparatus 400 may include fixation member 409. A head of fixation member 409 may be seated in plate 411. Fixation member 409 may include a threaded surface which may be coupled to support 407. The threaded surface may have one or more features in common with threaded surface 519. Plate 411 may define hole 413 and hole 415 for receiving fixation members.

Apparatus 400 may have one or more features in common with apparatus 500. Support 407 may have one or more features in common with support 507. Fixation member 409 may have one or more features in common with fixation member 511.

FIG. 5 shows illustrative apparatus 500. Apparatus 500 may include mesh 501 extending between hub 503 and base 505. Cannula 506 may extend through base 505. A first end of support 507 may be removably coupled to hub 503 as described herein. A second end of support 507 may include nut 509. Nut 509 may be a hexagonal nut. Nut 509 may be shaped for coupling to a tool such as a screw driver having a bit, such as a hex bit, for driving support 507 in rotation.

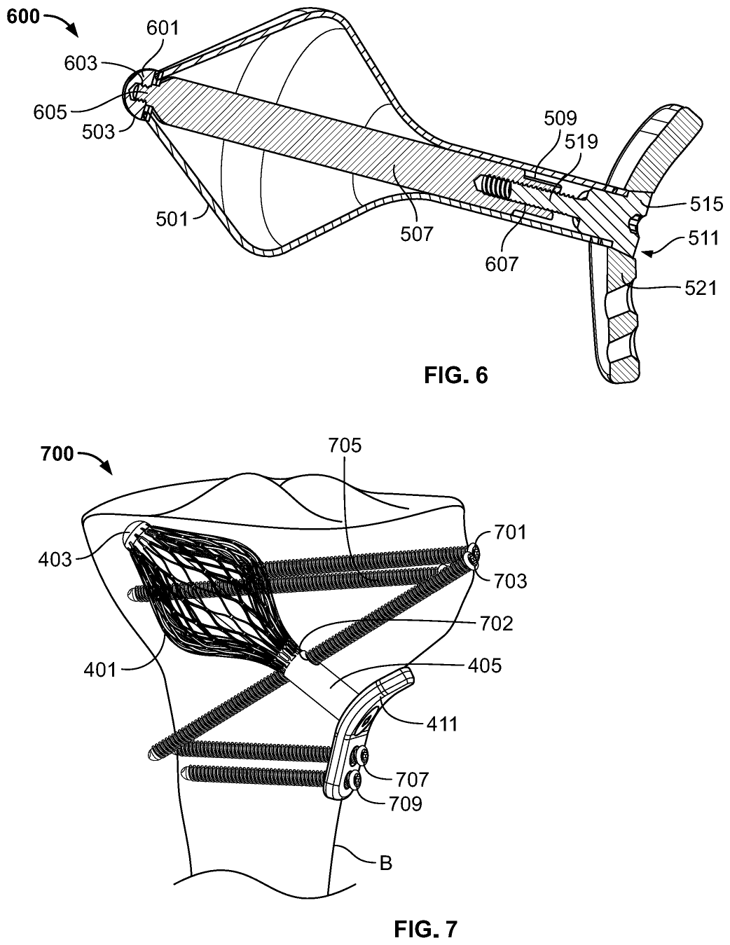

Nut 509 may have first end 509A and second end 509B opposite first end 509A. A threaded bore may extend through second end 509B of nut 509 (illustrated in FIG. 6, below). The threaded bore may be sized for threadedly engaging threaded surface 519.

In some embodiments, support 507 may have a constant circumference between a first end of support 507 and a second end of support 507. The first end of the support may be configured for being removably coupled to the hub as described herein. The second end of the support may include axial slots, such as slots 210. Slots 210 may be used by a tool, such as a cruciform shape screwdriver, to drive support 507 in rotation. In some of these embodiments, a threaded bore may extend through the second end of the support for threadedly engaging threaded surface 519.

Apparatus 500 may include fixation member 511. Fixation member 511 may include body 513 that is defined between threaded surface 519 and head 515. Head 515 may be shaped for seating in opening 523 of 521. Bottom face 515A of threaded surface may be sized configured to be seated on base second end 505B. Body 513 may be sized for being seated inside cannula 506.

FIG. 6 shows cross-sectional view 600 of apparatus 500, assembled. FIG. 6 illustrates threaded member 605 positioned at a first end of support 507. Threaded member 605 is shown to be in threaded engagement with threaded bore 603. Threaded bore 603 may extend through thickness 601 of hub 503. FIG. 6 also illustrates threaded bore 607 extending through nut 509. Threaded bore 607 is shown to be in threaded engagement with threaded surface 519 when apparatus 500 is assembled.

FIG. 7 shows apparatus 400 implanted in bone B. In FIG. 7, base 405 may define opening 702. In FIG. 7, a bottom face of plate 411 is positioned on a surface of the bone. Illustrative screw 701 and screw 705 may engage mesh 401. Illustrative screw 701 may engage opening 702. Illustrative screw 707 and 709 may engage hole 413 and hole 415, respectively.

Methods for inserting the implant in a bone B may include reducing bone B. Methods may include reducing bone fragments of bone B using fixation elements such as k-wires. Methods may include driving a target wire, such as a k-wire, into bone B and advancing the wire to a target site. Methods may include drilling over the targeting wire to the target site to create a passageway. The passageway may have a circumference greater than a circumference of the implant when collapsed. The methods may include advancing a broaching tool along the passageway.

The methods may include expanding and rotating the broaching tool in the bone to broach bone and create a cavity for receiving the expanded implant head.

The methods may include advancing the implant along the passageway. The implant may be advanced along the passageway using an inserter. The methods may include inserting the implant into the cavity using an inserter such as apparatus 10700, apparatus 12900, apparatus 13300 and apparatus 14100. The inserter may hold the implant in a tensioned state to keep the implant head collapsed. The implant may be a self-expanding implant.

In other embodiments, the implant may be retained in a collapsed state by keeping a support coupled to the implant in an advanced, tensioned state. The support may be removably coupled to one or both of an implant hub and an implant tail. Expanding the implant may include releasing the support from the tensioned state to allow the implant to self-expand. Expanding the implant may include de-coupling the support from the implant, allowing the implant to self-expand, and coupling the same or a different support to the implant after the expansion.

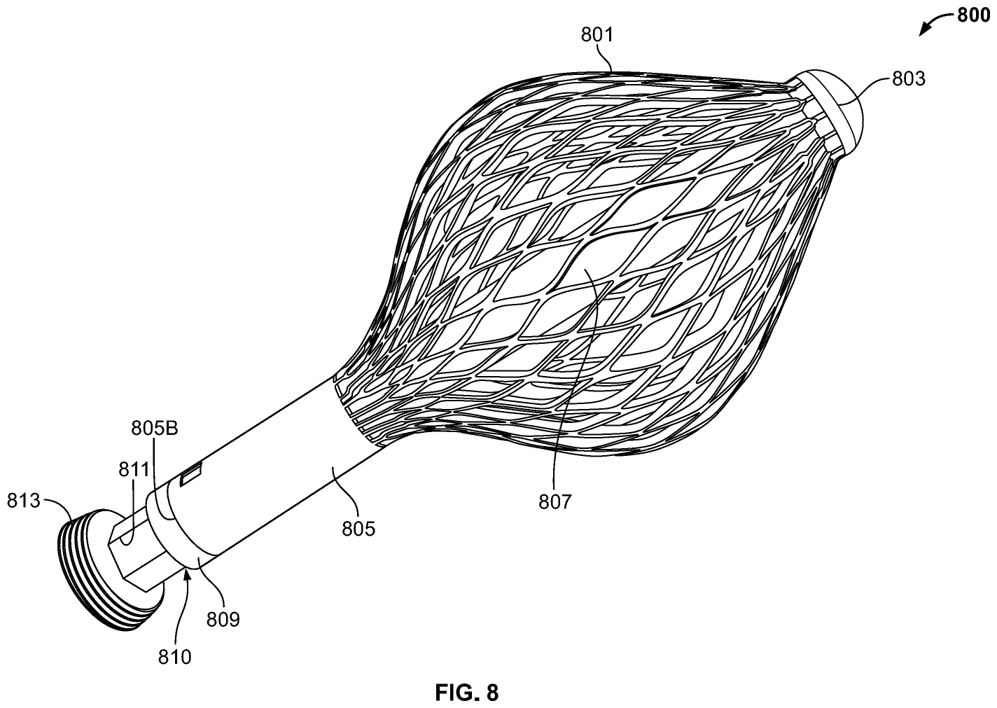

FIG. 8 shows illustrative apparatus 800. Apparatus 800 may include mesh 801 extending between hub 803 and base 805. Support 807 may be removably coupled to hub 803 as described herein.

Extension member 810 may be coupled to support 807 as disclosed herein. A threaded surface (not shown) of extension member 810 may be in threaded engagement with a threaded bore (not shown) extending through support 807. Extension member 810 may include flange 809 and nut 811. Flange 809 may be seated on base second end 805B. Fixation member 813 is shown seated on nut 811. A threaded surface (not shown) of fixation member 813 may be in threaded engagement with a threaded bore (not shown) extending through nut 811.

Support 807 may have one or more features in common with support 909. Extension member 810 may have one or more features in common with extension member 917. Fixation member 813 may have one or more features in common with fixation member 927.

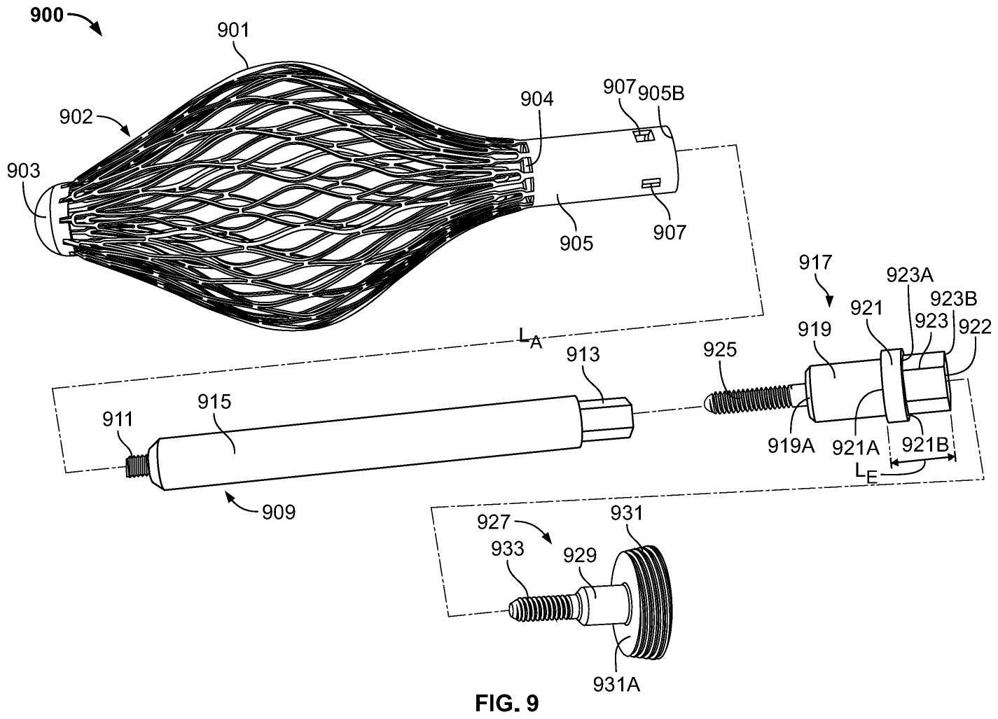

FIG. 9 shows illustrative apparatus 900. Apparatus 900 may include implant 902, support 909, extension member 917 and fixation member 927.

Implant 902 may include mesh 901 extending between hub 903 and base 905. Base 905 may include windows 907 for coupling to protrusions on a retaining figure included on an inserter. Cannula 904 may extend through base 905.

Support 909 may include elongated body 915 that is defined between threaded surface 911 and nut 913. Elongated body 915 may be configured to be seated in cannula 904 such that, when elongated body 915 is positioned in cannula 904, elongated body 915 abuts cannula 904 and can slide along cannula 904. Elongated body 915 may define a diameter smaller than a diameter of cannula 904 by 1.5 thousandths of an inch, at least 2 thousandths of an inch, greater than 2 thousandths of an inch, any other suitable value that configures elongated body 915 to both abut the cannula 904 and slide through the cannula 904. Threaded surface 911 may be shaped for threadedly engaging a threaded bore extending through hub 903 (not shown). Nut 913 may be shaped for coupling to an insertion or removal tool.

Extension member 917 may include body 919 configured for being seated in cannula 904. Body 919 may be configured to be seated in cannula 904 such that, when body 919 is positioned in cannula 904, body 919 abuts cannula 904 and can slide along cannula 904 until flange 921 abuts base second end 905B. Body 919 may define a diameter smaller than a diameter of cannula 904 by 1.5 thousandths of an inch, at least 2 thousandths of an inch, greater than 2 thousandths of an inch, any other suitable value that configures body 919 to both abut cannula 904 and slide through cannula 904.

Threaded member 925 may be shaped for threadedly engaging a threaded bore extending through nut 913 and elongated body 915 (not shown). First end 919A of body 919 may be shaped configured to be seated on nut 913. Nut 923 may be shaped for coupling to an insertion or removal tool. First end 923A of nut 923 may be adjacent flange 921. Second end 923B of nut 923 may be at an end of extension member 917.

Flange 921 may have a diameter equal to, or substantially equal to the same as, a diameter of base 905. When threaded surface 911 is coupled to hub 903 and flange 921 abuts base second end 905B, threading threaded surface 911 further into the threaded bore (not shown) extending through support 909 may draw hub 903 toward base 905 and further expand mesh 901. When hub 903 is shallow and/or does define a lot of thread travel, additional compression of the mesh cage may be desired. Extension member 917 may include sufficient threads in threaded surface 911 to provide the desired additional compression to mesh 901 to further expand mesh 901.

First end 921A of flange 921 may be shaped configured to be seated on base second end 905B. Second end 921B of flange 921 may be fixed to nut 923. Bore 922 may extend through second end 923B of nut 923. Bore 922 may be shaped for seating body 929 and for threadedly engaging threaded surface 933.

Fixation member 927 may include body 929, threaded surface 933 and head 931. Head 931 may be shaped for threaded engagement with a plate. Body 929 may be configured for being seated in a first length of a bore defined by extension member 917. Threaded surface 933 may be shaped for threaded engagement for a second length of the bore defined by extension member 917. The second length of the bore may be threaded.

When implant 902 is implanted in an interior of a bone, base second end 905B may be positioned away from a surface of the bone. Methods may include a practitioner selecting an extension member having length Lₑ from two or more extension members, each having a different length Lₑ, such that the selected extension member define an Lₑ that spans most or all of the distance between base second end 905B and the surface of the bone. Methods may include selecting an extension member having length Lₑ such that, when support 909 is coupled to implant 902 and extension member is coupled to support 909, second end 923B is proximal to a surface of the bone. Lₑ may be selected so that, coupling extension member 917 to support 909, second end 923B is flush with the surface of the bone, within 5 mm of the surface of the bone, 3 mm, 1 mm, or any other desirable position relative to the surface of the bone.

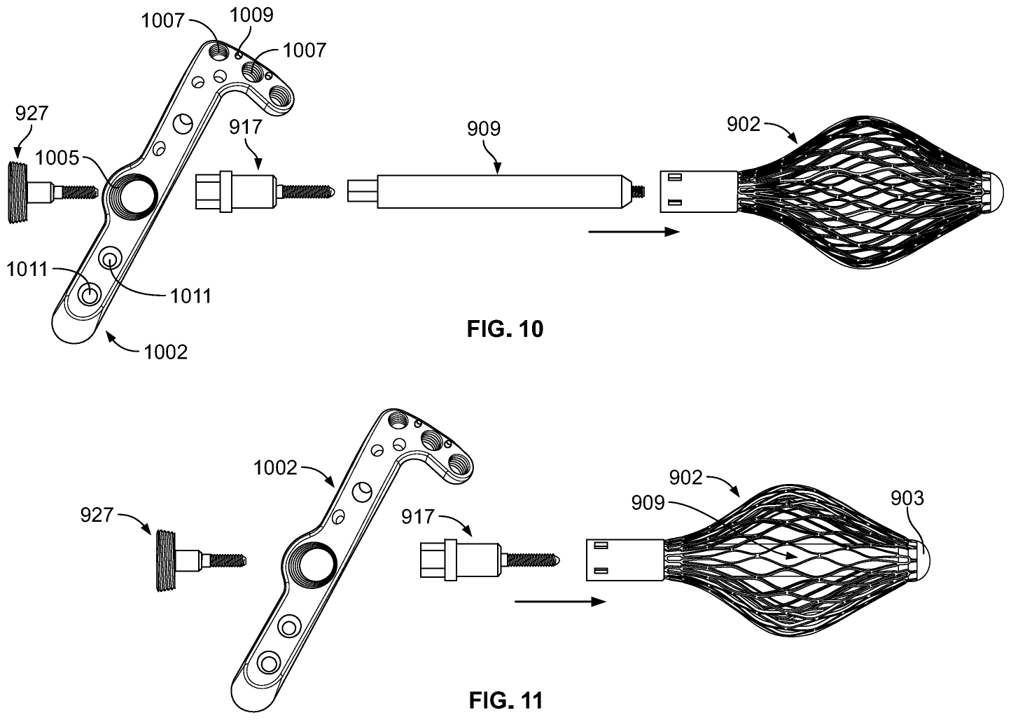

FIGS. 10-13 shows illustrative steps for assembling apparatus 900.

FIG. 10 shows apparatus 900 in addition to plate 1002. Plate 1002 may include threaded opening 1005 for threadedly engaging head 931 of fixation member 927. Plate 1002 may define holes 1001 and 1007 for receiving screws. Plate 1002 may define openings 1009 for receiving reduction apparatus, such as a k-wire.

In some embodiments, a kit may be provided including the implant, the support, the fixation member, the plate, and the extension member. In some embodiments, the kit may include two or more extension members. In the kit, the implant may be coupled to the support. In the kit, the implant may not be coupled to the support and may be compressed within a tube.

Each extension member in the kit provided may have a different length, such as a different length Lₑ. Methods may include selecting an extension member from two or more extension members, the selected extension member having a length Lₑ needed to couple the implant to a plate on the surface of the bone. The length needed may be determined based on an anatomical placement of the implant in a bone and a distance, along an access path, between the second end of the base and a surface of the bone. The length may be length described above in connection with FIG. 9.

FIG. 11 shows support 909 in implant 902. Support 909 may be removably coupled to hub 903. Threaded surface 911 of support 909 may be in threaded engagement with a threaded bore in hub 903 (not shown).

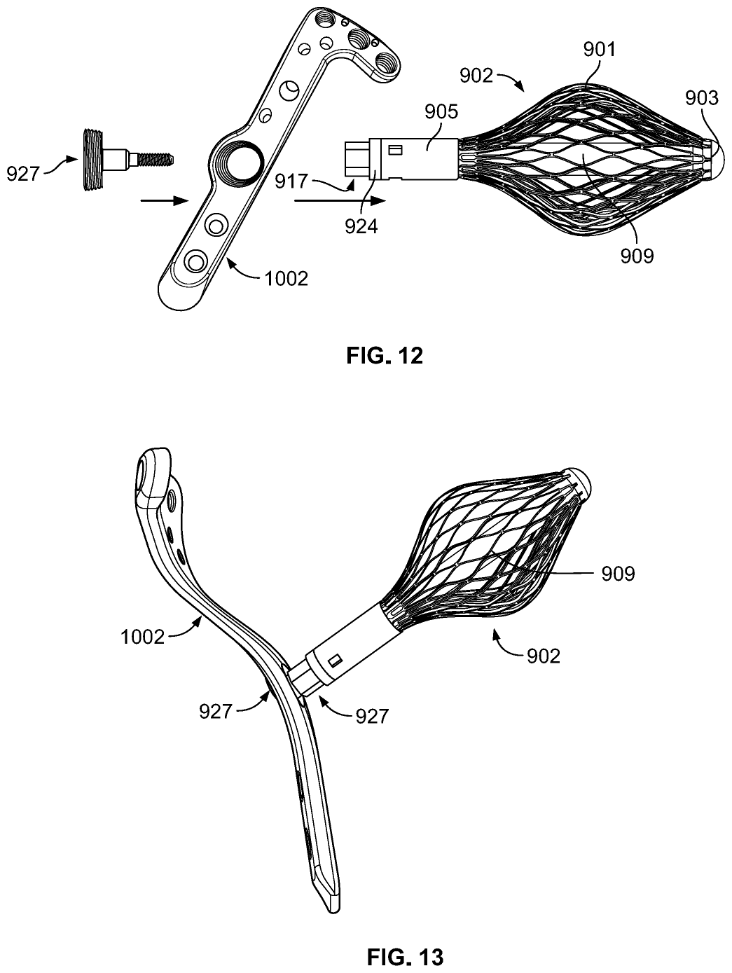

FIG. 12 shows extension member 917 coupled to support 909, and flange 921 of extension member 917 seated on base 905. Seating flange 921 on base 905 may fix a maximum distance between base 905 and hub 903. This may prevent mesh 901 from collapsing at least because, for implant 902 to collapse (without deforming), a distance base 905 and hub 903 may need to be increased.

FIG. 13 shows plate 1002 fixed to apparatus 900. Fixation member 927 may fix plate 1002 to fixation member 927 when fixation member 927 is coupled to plate 1002 and to extension member 917. Fixation member 927 may fix plate 1002 to fixation member 927 when head 931 of fixation member 927 is seated in opening 1005 and threaded surface 933 is threadedly engaged with a bore extending through extension member 917.

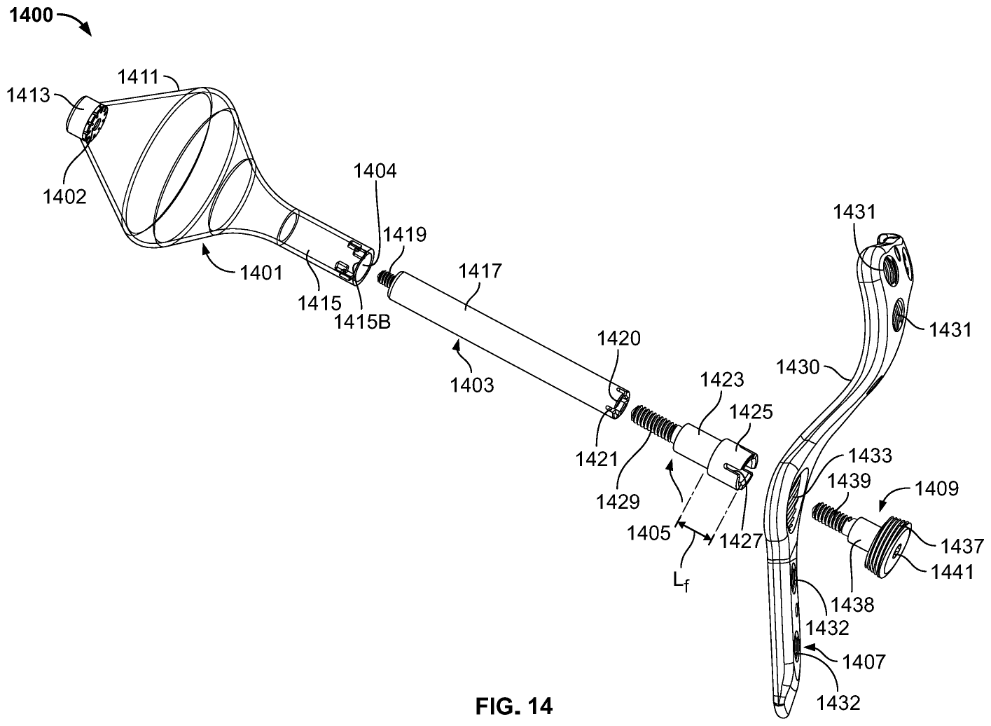

FIG. 14 shows illustrative apparatus 1400. Apparatus 1400 may include implant 1401, support 1403, extension member 1405, fixation member 1409 and plate 1407. Implant 1401 may include mesh 1411 extending between hub 1414 and base 1415. Hub may define coupling mechanism 1402. Coupling mechanism 1402 may be a threaded bore for threadedly engaging threaded surface 1419.

Support 1403 may include elongated body 1417. Elongated body 1417 may be configured to be seated in cannula 1404 such that, when elongated body 1417 is positioned in cannula 1404, elongated body 1417 abuts cannula 1404 and can slide along cannula 1404. Elongated body 1417 may define a diameter smaller than a diameter of cannula 1404. A difference in diameter between elongated body 1417 and cannula 1404 may include values described above in connection with elongated body 915 and cannula 904.

Threaded surface 1419 may be positioned at a first end of support 1403. Elongated body 1417 may extend between the first end of the support and second end 1420 of the support. Second end 1420 may include slots 1421 for engaging a tool. An advantage of slots 1421 for engaging a tool in place of a nut for engaging a tool, such as a nut 913, includes providing second end 1420 of the support with a larger circumference. A large circumference may enable machining of a threaded bore through the support having a larger diameter than otherwise would be possible if second end 1420 included a nut.

A bore may extend through second end 1420 having a first length for seating body 1423 and a second, threaded length for threadedly engaging threaded surface 1429.

Extension member 1405 may include body 1423 that is defined between threaded surface 1429 and flange 1425. Body 1423 may be configured to be seated in cannula 1415 as described above in connection with elongated body 1417.

Flange 1425 may include slots 1427. Slots 1427, instead of a nut such as nut 923, may include advantages described above in connection with slots 1421. Flange 1425 may be seized configured to be seated on second end 1420 of support 1403. Flange 1425 may have a diameter equal to, or substantially equal to the same as, a diameter of base 1415. When threaded surface 1419 is coupled to hub 1413 and flange 1425 is seated on base 1415, threading threaded surface 1429 further into the threaded bore extending through support 1403 (not shown) may draw hub 1413 toward base 1415 and further expand mesh 1411.

A bore may extend through flange 1425. The bore may be shaped for seating body 1438 and for threadedly engaging threaded surface 1439.

Fixation member 1409 (referred to alternately herein as a ‘fixation member’) may include body 1438 that is defined between threaded surface 1439 and head 1437. Head 1437 may be shaped for threaded engagement with threaded opening 1433 of plate 1407. Body 1438 may be configured for being seated in a first length of a bore defined by extension member 1405. Threaded surface 1439 may be shaped for threaded engagement for a second length of the bore defined by extension member 1405. The second length of the bore may be threaded. Threaded surface 1439 may include indentation 1441 for coupling to a tool.

When implant 1401 is implanted in an interior of a bone, second end 1415B of base 1415 may be spaced apart from a surface of the bone. Methods may include a practitioner selecting an extension member having length Lₑ from two or more extension members, each having a different length Lₑ, such that the selected extension member define an Lₑ that spans most or all of the distance between second end 1415B and the surface of the bone. Methods may include selecting an extension member having length Lₑ such that, when support 1403 is coupled to implant 1401 and extension member is coupled to support 1403, flange 1425 is proximal to a surface of the bone. Lₑ may be selected so that, after coupling extension member 1405 to support 1403, flange 1425 is flush with the surface of the bone, within 5 mm of the surface of the bone, 3 mm, 1 mm, or any other desirable position relative to the surface of the bone.

Plate 1407 may define threaded opening 1433 for threadedly engaging head 1437. Plate 1407 may include holes 1431 and 1432 for receiving screws.

FIG. 15 shows apparatus 1400 in an assembled state. In the assembled state, support 1403 may be coupled to hub 1413, extension member 1405 may be coupled to support 1403, and fixation member 1409 may be coupled to extension member 1405.

In the assembled state, threaded surface 1419 may seated in coupling mechanism 1402, threaded surface 1429 may be seated in a threaded bore extending through support second end 1420, and threaded surface 1439 may be coupled to a threaded length of a bore extending through extension member 1405.

In the assembled state, a portion of support 1403 and body 1423 may be seated in cannula 1404. In the assembled state, flange 1425 may be seated on base second end 1415B. In the assembled state, head 1437 may be seated on flange 1425.

FIG. 16 shows illustrative positioning of apparatus 1500 in bone B. FIG. 16 also shows plate 1407 coupled to apparatus 1500 and positioned on bone B. Screws 1603 are shown extending through holes 1431 and into mesh 1411. Screws 1603 may anchor plate 1407 to mesh 1411. Screws 1603 may anchor plate 1407 to bone B. Screws 1605 are shown extending through holes 1432 and into bone B. Screws 1605 may anchor plate 1407 to bone B.

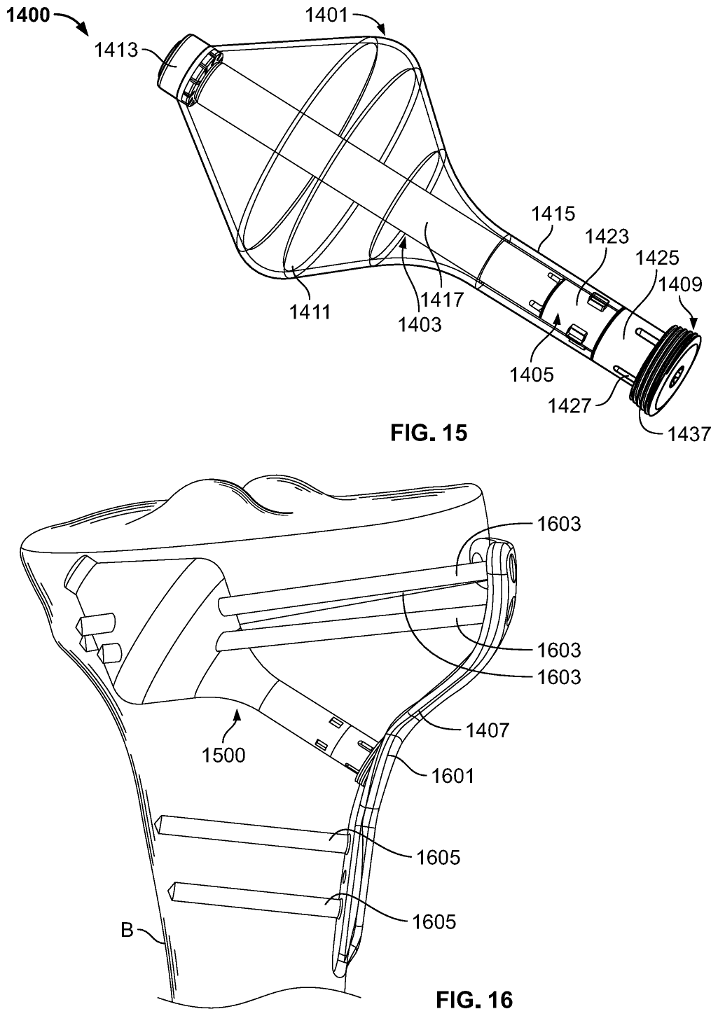

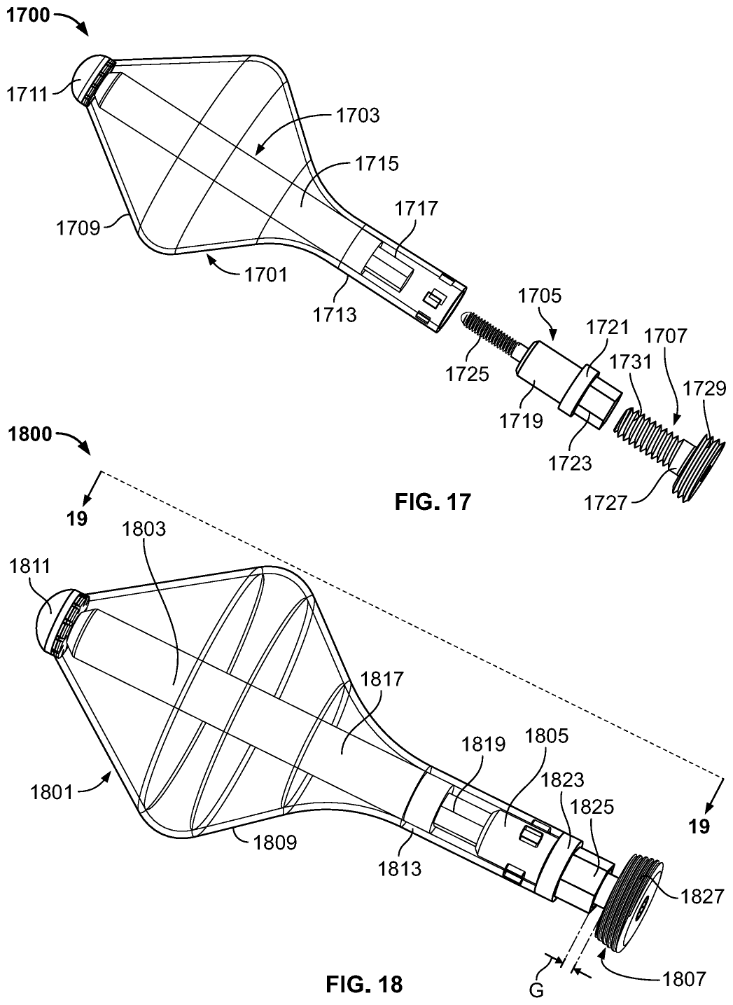

FIG. 17 shows illustrative apparatus 1700. Apparatus 1700 may include implant 1701, support 1703, extension member 1705 and fixation member 1707. Implant 1701 may include mesh 1709 extending between hub 1711 and base 1713. In FIG. 17, support 1403 is illustrated as being coupled to hub 1711. A first end of support 1403 may be removably coupled to hub 1411 as described herein. A second end of support 1403 may include nut 1717.

Fixation member may include body 1719 that is defined between threaded surface 1725 and flange 1721. Nut 1723 may be seated on flange 1721. Fixation member 1707 may include threaded head 1729 for threadedly engaging a threaded opening in a plate. Fixation member 1707 may include threaded surface 1731 for engaging a threaded bore extending through extension member 1705.

Implant 1701, support 1703, extension member 1705 and fixation member 1707 may have features of implants, supports, extension members and fixation members described herein.

FIG. 18 shows apparatus 1800. Apparatus 1800 may include implant 1801. Implant 1801 may include mesh 1809 extending between hub 1811 and base 1813. Apparatus 1800 may include support 1803 removably coupled to hub 1811. Nut 1819 may be positioned at an end of support 1803. Flange 1823 of extension member 1805 may be seated on base 1813. Nut 1825 may be positioned on flange 1823.

In FIG. 18, fixation member 1807 is shown coupled to extension member 1805. When fixation member 1807 is coupled to extension member 1805, gap G may extend between extension member 1805 and head 1827 of fixation member 1807.

Implant 1801, support 1803, extension member 1805 and fixation member 1807 may have features of implants, supports, extension members and fixation members described herein.

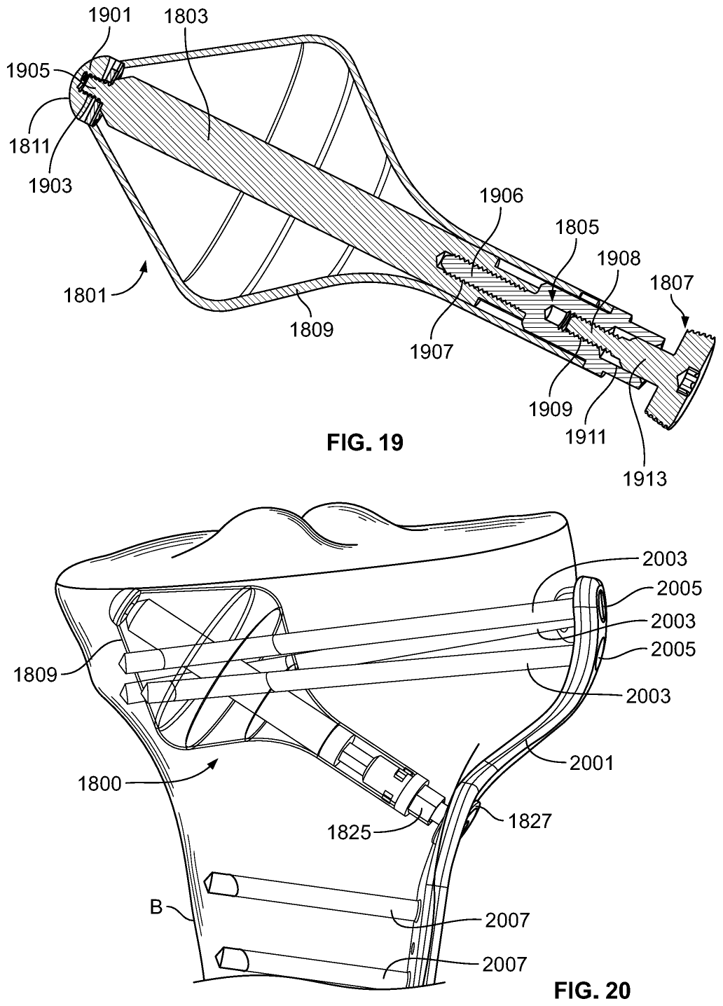

FIG. 19 shows a cross-sectional view of the apparatus 1800 taken along the lines 19-19. Hub 1811 may define threaded bore 1903 extending through a thickness of hub 1901. Support 1803 may include threaded surface 1905 in threaded engagement with threaded bore 1903.

Threaded bore 1907 may extend through support 1803. Extension member 1805 may include threaded surface 1906 for engaging threaded bore 1907.

Extension member may define a bore having first length 1911 and second length 1909. First length 1911 may be shaped for seating body 1913 of fixation member 1807. Second length 1909 may be threaded for threadedly engaging threaded surface 1908 of fixation member 1807. Threaded surface 1908 is shown threadedly engaging some, but not all, of second length 1909. The partial threading of threaded surface 1908 into second length 1909 may define gap G. Gap G may be closed when threaded surface 1908 is fully threaded into extension member 1805.

When implant 1801 is implanted in an interior of a bone and support 1803 and extension member 1805 are coupled to implant 1801, a practitioner may select a length of gap so that it spans a distance between a plate surface of a bone, in which a head of fixation member 1807 is seated, and nut 1825 of extension member 1805. Thus, a portion of body 1913 of fixation member 1807 may span a distance between a plate positioned on the surface of the bone and extension member 1805. When implant 1801 is implanted in the interior such that nut 1825 is positioned adjacent the bone surface, head 1827 may be seated on nut 1825 and gap G may be reduced to zero.

FIG. 20 shows illustrative positioning of apparatus 1800 in bone B. FIG. 20 shows plate 2001 coupled to apparatus 1800 and positioned on an outer surface of bone B. Screws 2003 are shown extending through holes 2005 and into mesh 1809. Screws 2003 may anchor plate 2001 to mesh 1809. Screws 2003 may anchor plate 2001 to bone B. Screws 2007 are shown extending through plate 2001 and into bone B. Screws 2007 may anchor plate 2001 to bone B.

FIG. 20 shows head 1827 seated in an opening defined in plate 2001 and a portion of body 1913 defining a gap between head 1827 and nut 1825. The gap may be resultant from threading threaded surface 1908 into some, but not all, of the threaded bore defined by second length 1909.

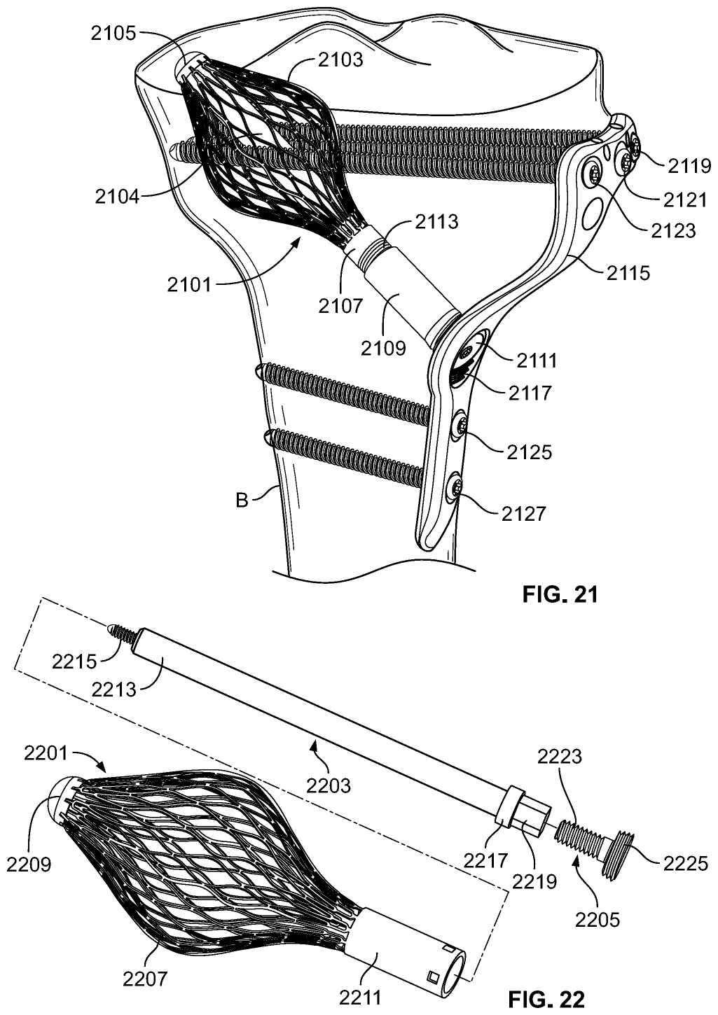

FIG. 21 shows illustrative apparatus implanted in bone B. Illustrative apparatus may include implant 2101, support 2104, extension member 2113 and fixation member 2109. Implant 2101 may include mesh 2103 extending between hub 2105 and base 2107.

Support 2104 may include a first end for coupling to hub 2105. Support 2104 may include a second end for coupling to extension member 2113.

Extension member 2113 may be a first fastening member. Extension member may be configured to be coupled to support 2104 such that, when extension member is coupled to support 2104, the externally threaded flange of extension member 2113 is seated on base 2107.

Fixation member 2109 may be a second fastening member. Fixation member 2109 may define an internally threaded cannula threaded to mate with the externally threaded extension member 2113. Fixation member 2109 may include flanged end 2111 shaped for seating in opening 2117 of plate 2115.

If a practitioner expands mesh 2103 in a smaller cavity than desired, mesh 2103 may not self-expand to a desired volume. Using apparatus illustrated in FIG. 21, the practitioner may drive fixation member 2109 to engage threads on extension member 2113 sufficient turns to advance hub 2105 toward base 2107 and, as result, further expand the mesh 2103.

FIG. 22 shows illustrative apparatus, including implant 2201, support 2203 and fixation member 2205. Implant 2201 may include mesh 2207 extending between hub 2209 and base 2211. Support 2203 may include elongated body 2213 that is defined between threaded surface 2215 and flange 2217. Nut 2219 may be positioned on flange 2217. Fixation member 2205 may have threaded head 2225 and threaded surface 2223.

Threaded surface 2215 may threadedly engage a bore extending through hub 2209. Flange 2217 may be sized configured to be seated on base 2211. Support 2203 may define a threaded bore extending through nut 2219 to threadedly engage threaded surface 2223. Threaded head 2225 may be sized for threaded engagement with a threaded opening in a plate.

Support 2203, in operation, may extend through implant 2201 such that threaded surface 2215 of support 2203 is engaged with implant hub 2209 and flange 2217 of support 2203 is seated on implant base 2211. In operation, fixation member 2205 may be threadedly engaged with a cannula extending through at least some of support 2203.

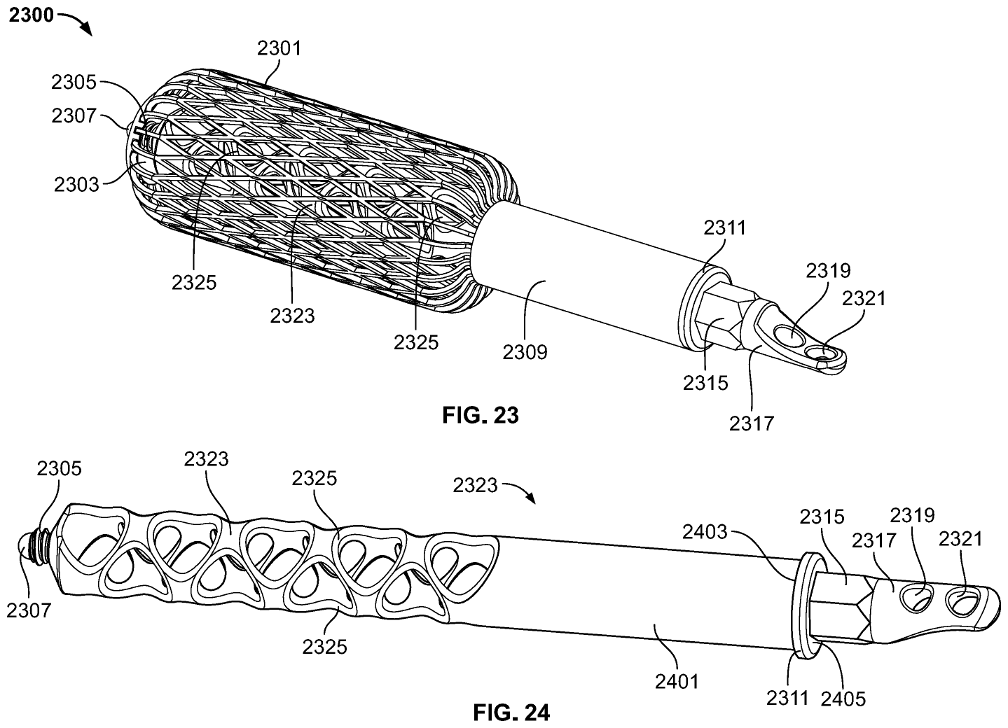

FIG. 23 shows illustrative apparatus 2300. Apparatus 2300 may include mesh 2301 extending between hub 2303 and base 2309. Apparatus 2300 may include support 2323 defining a plurality of openings 2325 transecting support 2323. Openings 2325 may be sized for receiving a screw. Support 2323 may include threaded surface 2305. When threaded surface 2305 is in threaded engagement with hub 2303, tip 2307 of support 2323 may extend through a thickness of hub 2303 and be positioned outside of hub 2303.

Support 2323 may include flange 2311. Flange 2311 may be sized configured to be seated on base 2309. Support 2323 may include nut 2315, atraumatic member 2317 and flange 2311. Nut 2315 may be disposed between atraumatic member 2317 and flange 2311. Atraumatic member 2317 may define opening 2319 and opening 2321.

FIG. 24 shows support 2323. Elongated body 2401 may extend between threaded surface 2305 and flange 2311. A length of elongated body 2401 may define openings 2325. Elongated body 2401 may have a maximum outer circumference no greater than a few thousandth of an inch less circumference of a bore extending through base 2309 (not shown). A difference in diameter between elongated body 2401 and the bore extending through base 2309 may include values described above in connection with elongated body 915 and cannula 904

Flange 2311 may have flange first face 2403 and flange second face 2405. Flange first end 2403 may be shaped configured to be seated on base 2309. Nut 2315 may extend away from flange second face 2405.

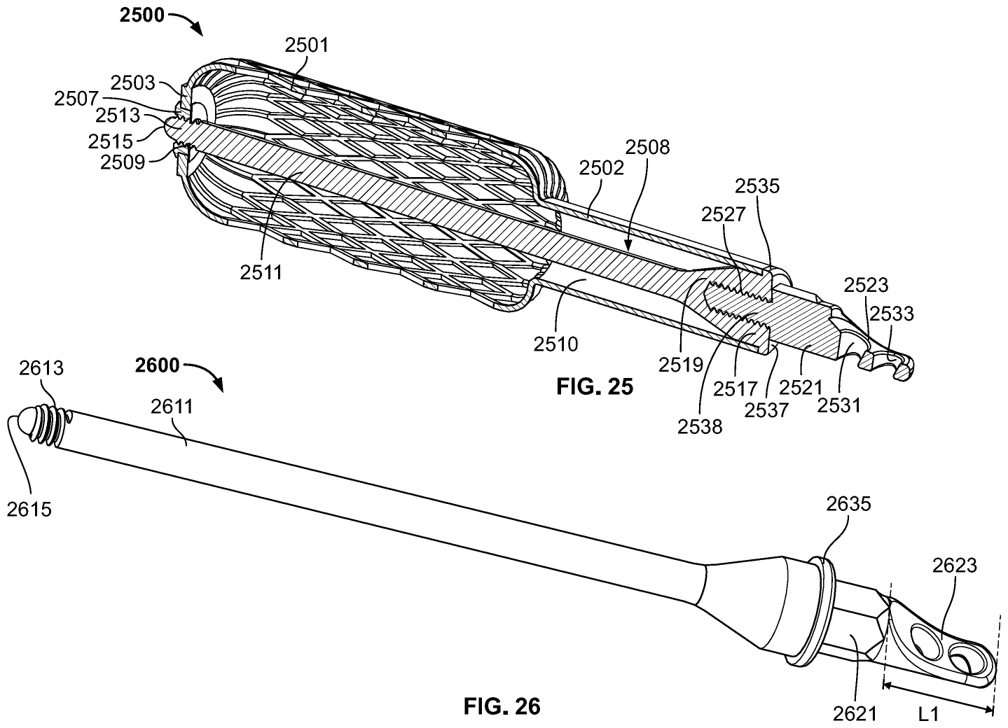

FIG. 25 shows a cross-sectional view of illustrative apparatus 2500. Apparatus 2500 may include mesh 2501 extending between hub 2503 and base 2502. Hub 2503 and hub 2507, together, may define a unitary body. Hub 2503 may be fixed to hub 2507. Hub 2507 may define threaded bore 2509 for threadedly engaging threaded surface 2513 of support 2508. In FIG. 25, threaded surface 2513 is shown engaged with threaded bore 2509 and tip 2515 is shown positioned outside of hub 2503.

In FIG. 25, flange 2535 of support 2508 is seated on base 2502. Support 2508 may include first length 2511 having a first circumference, second length 2517 having a second circumference, and third length 2519 having a third circumference decreasing from the second circumference to the first circumference along a central axis of the support toward the first length. First length 2511, second length 2517 and third length 2519 may together form the elongated body. A portion of first length 2511, second length 2517 and third length 2519 may extend through cannula 2510 define by base 2502.

Nut 2521 may be positioned on face 2537 of flange 2535. Atraumatic member 2523 may extend away from nut 2521 and define opening 2531 and 2533.

Support 2508 may define threaded bore 2527 extending through flange 2535 and into support 2508. Threaded surface 2538 may extend away from nut 2521 and be sized for threadedly engaging threaded surface 2538. Nut 2521, atraumatic member 2523 and threaded surface 2538 may, together, be of unitary construction and comprise a fixation member.

When mesh 2501 is implanted in an interior of a bone and support 2508 is coupled to hub 2507, a practitioner may select a fixation member from two or more fixation members, each of the fixation members having a nut defining a different length. A practitioner may select a fixation member with a nut having a length that spans a distance between base 2502 and a surface of a bone in which mesh 2501 has been implanted. Atraumatic member 2523 may extend along a lateral side the bone. The distance between base 2502 and the surface of the bone may have features similar to the distance Lₑ described above in connection with FIG. 14.

Mesh 2501 may be expanded in a cavity prepared in an interior of a bone. After expansion of mesh 2501, the selected fixation member may be coupled to support 2508. Support 2508 may then be coupled to hub 2507 such that flange 2535 sits on base 2502.

Nut 2521 may include flange 2535. Flange 2535 may seat nut 2531 on base second end 2501. Threading threaded surface 2538 into threaded bore 2527 by turning nut 2521 may seat flange 2535 on base second end 2501.

FIG. 26 shows exemplary support 2600 that may be used as a support for coupling to any implant disclosed herein. Support 2600 may be coupled to hub 2507 and base 2502. Support 2600 may include threaded surface 2613, tip 2615, elongated body 2611, flange 2635, nut 2621 and atraumatic member 2623. Support 2600 may have features in common with support 2508. Support 2600 be a unitary body.

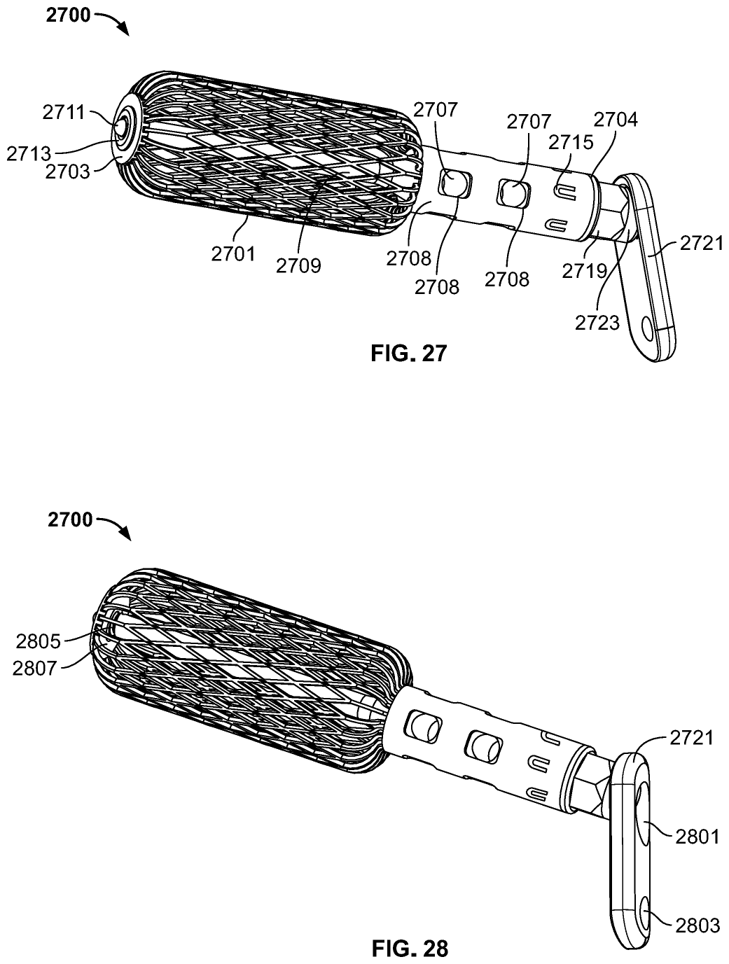

FIG. 27 shows illustrative apparatus 2700. Apparatus 2700 may include mesh 2701 extending between hub 2703 and base 2705. Tip 2711 of support 2709 is shown extending through hub 2713. Support 2709 may have one or more features in common with support 2903. Hub 2713 may define a threaded bore extending through hub 2713. A threaded surface of support 2709 may be in threaded engagement with hub 2713. Hub 2703 and hub 2713 may, together, be a unitary body. Hub 2703 may be fixed to hub 2713.

Base 2705 may define windows 2715. Windows may be used to couple base 2705 to an inserter. Base 2705 may define openings 2707. Support 2709 may define openings 2708. Openings 2708 may be positioned on support 2709 such that, when mesh 2701 is expanded, and support 2709 is coupled to hub 2703 and/or hub 2713, each opening 2708 is positioned inside an opening 2707.

One or more of openings 2707 may be circular, square, or any other suitable shape. One or more of openings 2708 may be circular, square, or any other suitable shape.

Support 2709 may include flange 2704 shaped configured to be seated on base 2705. Nut 2719 may extend between flange 2704 and protrusion 2723. Protrusion 2723 may be configured for being seated in an opening defined in plate 2721. A threaded bore (not shown) may extend through protrusion 2723. A fixation member may be coupled to the threaded bore to lock protrusion 2723 to plate 2721.

FIG. 28 shows a different view of the apparatus 2700 than the view of apparatus 2700 shown in FIG. 27. In FIG. 28, threaded surface 2805 of support 2709 is shown engaging hub bottom surface 2807. In FIG. 28, openings 2801 and 2803 of plate 2721 are illustrated. Opening 2801 may be sized to seat protrusion 2723. Opening 2803 may be sized to receive a screw.

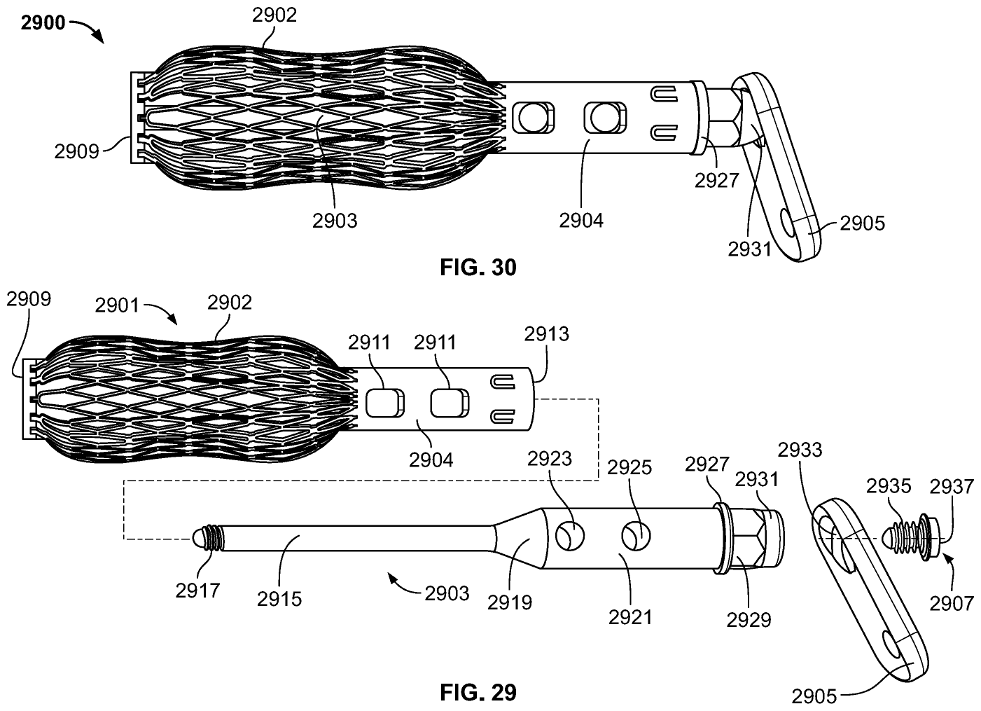

FIG. 29 shows illustrative implant 2901, support 2903, plate 2905 and fixation member 2907. Implant 2901 may include mesh 2902 extending between hub 2909 and base 2904. Base 2904 may define openings 2911 and include windows 2906. Base 2904 may be cannulated. One or more of openings 2911 may be square, circular, or any other suitable shape.

Support 2903 may include threaded surface 2917. A tip of support 2903 may not be threaded. A tip of support 2903 may be threaded (not shown). Support 2903 may include first length 2915 having a first circumference, a second length 2921 having a second circumference, and a third length 2919 having a third circumference that decreases from the second circumference to the first circumference along a central axis of the support toward the first length. The third length may define a conical shape.

Second length 2921 may be sized for being seated in the cannula extending through base 2904. A difference in diameter between second length 2921 and a cannula extending through base 2904 may include values described above in connection with elongated body 915 and cannula 904

Second length 2921 may define openings 2923. One or more of openings 2923 may be square, circular, or any other suitable shape. Openings 2923 may be positioned on support 2903 such that, when mesh 2902 is expanded, and support 2903 is coupled to hub 2909, each opening 2923 is positioned inside an opening 2911.

Support 2903 may include flange 2927. Flange 2927 may be shaped configured to be seated on base second end 2913. Nut 2929 may extend between flange 2927 and protrusion 2931. Protrusion 2931 may define a circumference. Protrusion 2931 may define a circumference that varies along a central axis of support 2903 (not shown). Protrusion 2931 may be configured for being seated in opening 2933 of plate 2905. Fixation member 2907 may include threaded surface for threading into a threaded bore extending through protrusion 2931 (not shown). Head 2937 of fixation member 2907 may be sized for being seated in opening 2933 to fix support 2903 to plate 2905.

The methods may include selecting one or more of an implant, a support, a plate and a fixation member for treating a bone from a kit including one or more implants, supports, plates and/or fixation members. One or more of the plates may define different bottom surface contours and/or angles for resting on the bone. One or more of the supports may include nuts defining different lengths, for spanning different gaps between the implant and a surface of a bone after implanting the implant in an interior of the bone.

FIG. 30 shows apparatus illustrated in FIG. 29 in an assembled state. In FIG. 30, support 2903 is coupled to hub 2909 and flange 2927 is seated on base 2904. Protrusion 2931 is seated in opening 2933 of plate 2905. Fixation member 2907 may extend through opening 2533 and fixedly couple plate 2905 to protrusion 2931.

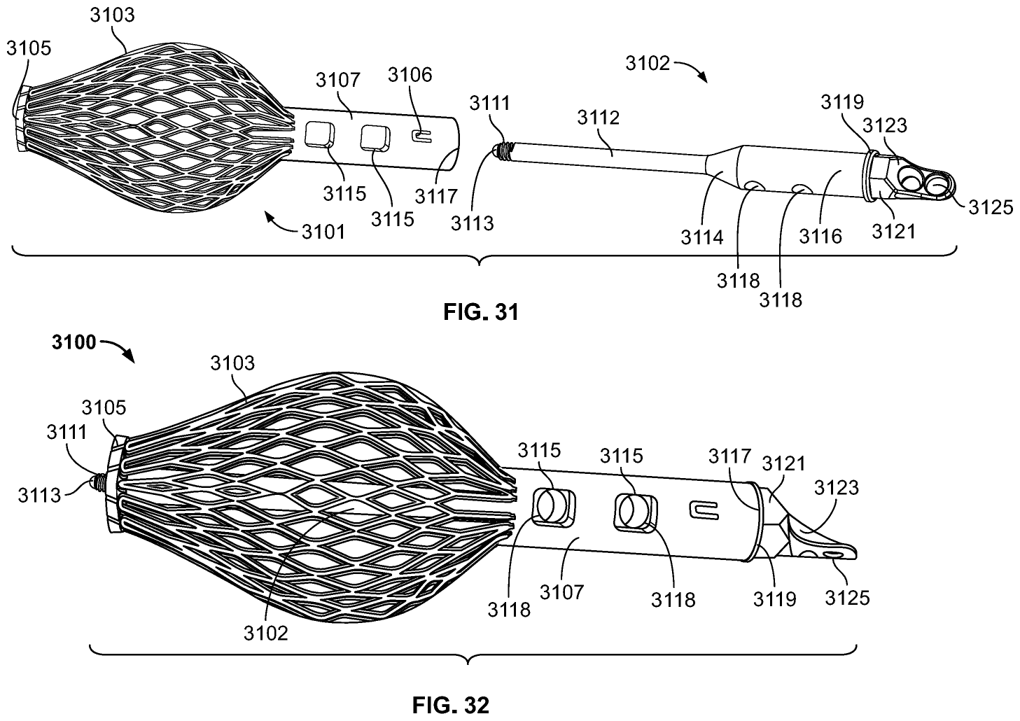

FIG. 31 shows illustrative implant 3101 and support 3102. Implant 3101 may include mesh 3103 extending between hub 3105 and base 3107. Base 3107 may be cannulated. Base 3107 may include windows 3106 for coupling to an inserter. Base may include openings 3115. Openings 3115 may be square, circular, or any other suitable shape.

Support 3102 may include threaded surface 3111 for threadedly engaging a threaded bore extending through hub 3105. Tip 3113 of support 3102 may not be threaded. Tip 3113 may be threaded. Support 3102 may include first length 31112 having a first circumference, second length 3116 having a second circumference, and third length 3114 having a variable circumference that varies from the second circumference to the first circumference along a central axis of the support toward the first length. Third length 3114 may have a conical shape.

Second length 3116 may be sized for being seated in the cannula extending through base 3107. A difference in diameter between second length 3116 and the cannula extending through base 3107 may include values described above in connection with elongated body 915 and cannula 904.

Second length 3116 may define openings 3118. One or more of openings 3118 may be square, circular, or any other suitable shape. Openings 3118 may be positioned on support 3102 such that, when mesh 3103 is expanded, and support 3102 is coupled to hub 3105, each opening 3118 is positioned inside an opening 3115.

Support 3102 may include flange 3119. Flange 3119 may be shaped configured to be seated on base second end 3117. Nut 3121 may extend between flange 3119 and atraumatic member 3123. Atraumatic member 3123 may have a thickness that increase along a length of the member toward nut 3121. Atraumatic member 3123 may define one or more openings 3125 to receive screws.

FIG. 32 shows apparatus illustrated in FIG. 31 in an assembled state. In FIG. 32, threaded surface 3111 is threadedly engaged with a threaded bore extending through hub 3105. Tip 3113, and a portion of threaded surface 3131, is positioned outside of hub 3105. Flange 3119 is seated on base second end 3117.

As threaded surface 3111 is threaded into hub 3105, flange 3119 may be advanced toward base second end 3117. When flange 3119 is seated on base second end 3117, further threading of threaded surface 3111 into hub 3105 may draw hub 3105 toward base 3107 and further expand mesh 3103.

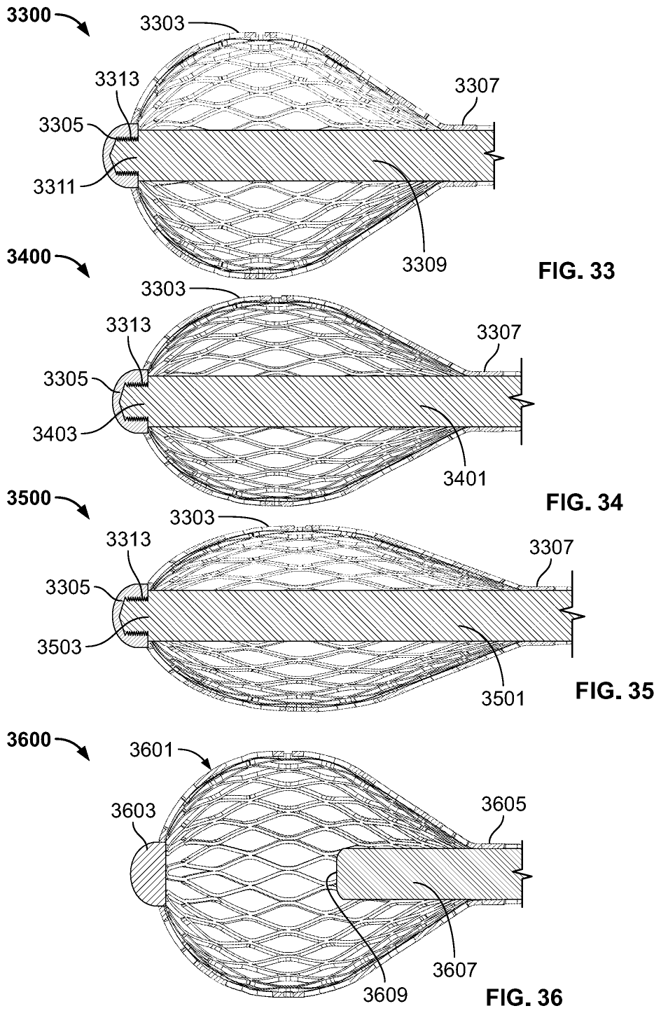

FIG. 33 shows illustrative apparatus 3300. Apparatus 3300 may include mesh 3303 extending between hub 3305 and base 3307. Hub 3305 may define threaded bore 3313.

In FIG. 33, threaded surface 3311 of support 3309 is shown coupled to hub 3305. A flange (not shown) of support 3309 may be seated on base 3307. A distance between a flange of support 3309 and threaded surface 3311, along a central axis of support 3309, may be a first length. Support 3309 may have features in common with support 208 or any other support disclosed herein.

FIG. 34 shows illustrative apparatus 3400. Apparatus 3400 may include mesh 3303 extending between hub 3305 and base 3307. Hub 3305 may define threaded bore 3313.

In FIG. 34, threaded surface 3403 of support 3401 is shown coupled to hub 3305. A flange (not shown) of support 3401 may be seated on base 3307. A distance between a flange of support 3401 and threaded surface 3403, along a central axis of support 3401, may be a second length. The second length may be greater than the first length. Support 3401 may have features in common with support 208 or any other support disclosed herein.

FIG. 35 shows illustrative apparatus 3500. Apparatus 3400 may include mesh 3303 extending between hub 3305 and base 3307. Hub 3305 may define threaded bore 3313.

In FIG. 35, threaded surface 3503 of support 3501 is shown coupled to hub 3305. A flange (not shown) of support 3501 may be seated on base 3307. A distance between a flange of support 3501 and a threaded surface 3503, along a central axis of support 3501, may be a third length. The third length may be greater than the first length and greater than the second length. Support 3501 may have features in common with support 208 or any other support disclosed herein.

An implant including mesh 3303, illustrated in FIGS. 33, 34 and 35, is illustrated in each of the figures in a different state of expansion. The different length supports coupled to mesh 3303 in FIGS. 33, 34 and 35 may each lock mesh 3303 in a different state of expansion.

Support 3309 may have a ‘small’ length and lock the mesh cage in a ‘large’ deployment state. The small length may be the first length. The large deployment state may have a relatively large radial displacement of mesh 3303 from a central axis of support 3309. The radial displacement of mesh 3303 from the central axis of support 3309 may be larger than the radial displacements in each of FIGS. 34 and 35.

Support 3401 may have a ‘medium’ length and lock the mesh cage in a ‘medium’ deployment state. The medium length may be the second length. The medium deployment state may have a relatively medium radial displacement of mesh 3303 from a central axis of support 3401. The radial displacement of mesh 3303 from the central axis of support 3401 may be larger than the radial displacement in FIG. 33 and smaller than the radial displacement in FIG. 35.

Support 3501 may have a ‘large’ length and lock the mesh cage in a ‘small’ deployment state. The large length may be the third length. The small deployment state may be a relatively small radial displacement of mesh 3303 from the central axis of support 3501. The radial displacement of mesh 3303 from the central axis of support 3501 may be smaller than the radial displacements in each of FIGS. 34 and 33.

Various lengths of supports may be used to deploy the mesh at various degrees of deployment. A support having a larger length (between support flange and support threaded surface) may lock a cage in a smaller deployment state in comparison to a support having a smaller length (between support flange and support threaded surface).

The methods may include releasing the mesh in a cavity to self-expand. The methods may include and engaging an end of the support with the implant hub without further expanding or collapsing the implant. The methods may include releasing the mesh in the cavity to self-expand. The methods may include engaging an end of the support with the implant hub, the engaging including and further collapsing or expanding the implant based on the length of the support selected.

The methods may include selecting a support from two or more supports, each support having a different length between a flange of the support and a threaded surface of the support (or other coupling mechanism positioned at the first end of the support). The methods may include selecting a support having a length between the flange and threaded surface for locking the mesh so that the radial expansion of the mesh affected by the support is the radial expansion needed for providing therapy to a bone.

FIG. 36 shows illustrative apparatus. The illustrative apparatus includes an implant including mesh 3601 extending between hub 3603 and base 3605. Support 3607 is shown positioned in a volume circumscribed by mesh 3601.

First end 3609 of support 3607 terminates in the volume. First end 3609 is not coupled to hub 3603. First end 3609 may be spaced apart from, and not abut, hub 3603. Support 3607 not engaging hub may enable mesh 3601 to flex in an interior of a bone in one, two or more directions. Flex may allow micromotion of a fracture. Flexing may aid callous formation and healing of bone.

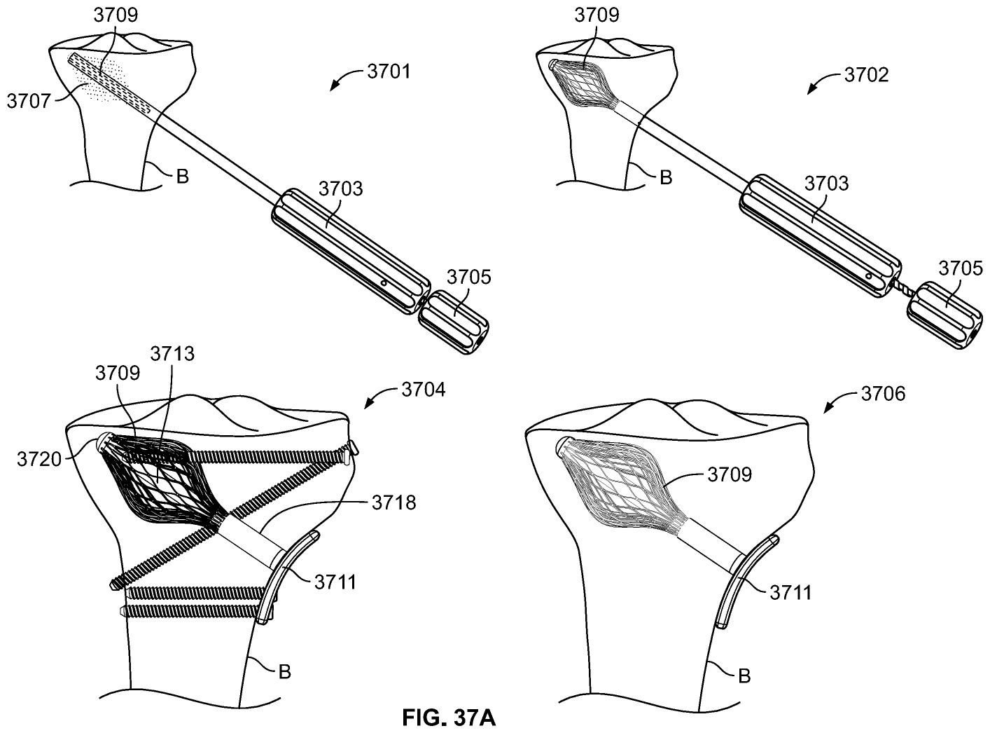

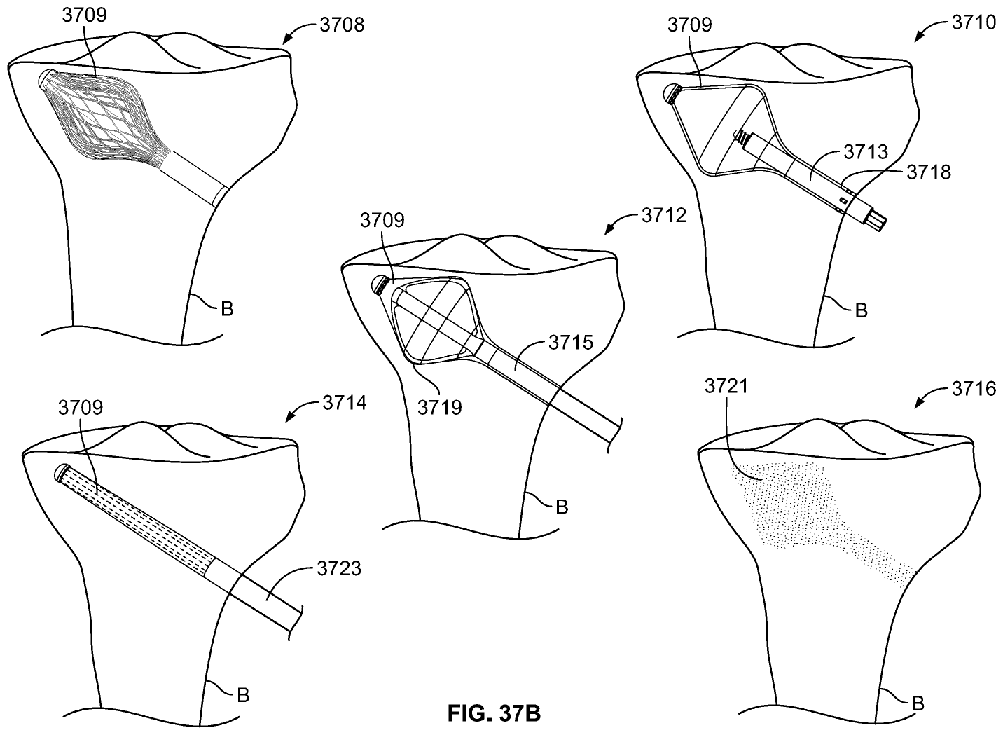

FIGS. 37A-B show illustrative methods for implanting implant 3709 in bone B and subsequently removing implant 3709. The illustrative methods in FIGS. 37A-B may be performed in the order described. The illustrative methods in FIGS. 37A-B may be performed in an order different from the order described. Steps not included in FIGS. 37A-B but disclosed herein may be included in the methods.

Implant 3709 may be the implant. At step 3701, an inserter, including first member 3703 and second member 3705 may position implant 709, in a collapsed state, in cavity 3707.

Apparatus 10700 may be used to insert implant 3709 into bone B. Apparatus 12900 may be used to insert implant 3709 into bone B. Apparatus 13300 may be used to insert implant 3709 into bone B. Apparatus 14100 may be used to insert implant 3709 into bone B.

At step 3702, implant 3709 may be released to self-expand in cavity 3707. Implant 3709 is shown to have expanded to fill cavity 3707. At step 3702, implant 3709 may be manually expanded.

At step 3704, support 3713 may be coupled to hub 3720 of implant 3709. Support may be advanced through base 3718 and coupled to hub 3720. At step 3704, implant 3709 may be anchored to bone B by screws. At step 3704, plate 3711 may be placed on bone B. Plate 3711 may conform to a surface contour of bone B. Plate 3711 may be locked to bone B using one or more screws passing through plate 3711 and into bone B. Screws may be passed through plate 3711 and into implant 3709.

Plate 3711 may be on bone B in steps 3701 and 3702. The methods may include placing plate 3711 on bone B. The methods may include preparing cavity 3707 by advancing tools through an opening on plate 3711. The methods may include coupling implant 3709 to inserter. The methods may include advancing implant 3709, in a collapsed state, through plate 3711 and into cavity 3707.

Steps for removing implant 3709 from bone B may begin at step 3706. At step 3706, screws may be removed from engagement with implant 3709 and from engagement with plate 3711. At step 3708, plate 3711 may be removed from bone B. At step 3710, support 3713 may be removed from implant 3709 through the base of implant 3709.

At step 3712, broaching tool 3715 may be advanced through base 3718 and positioned in a volume circumscribed by, or “surrounded” by, the mesh of implant 3709. Broaching tool 3715 may be advanced through base 3718 when broaching members 3719 are collapsed (“collapsed state”). At step 3712, broaching members 3719 have been expanded. At step 3712, broaching tool 3715 may be rotated. When broaching tool 3715 is rotated, broaching members 3719 may broach matter inside the volume. The matter may be organic matter that accumulated in the volume after implantation of implant 3709. The matter may be matter, including organic matter and/or non-organic matter, placed in the volume by a practitioner during the implantation of implant 3709. After broaching matter in the volume, broaching members 3719 may be collapsed, and broaching tool 3715 may be removed from the bone in a collapsed state.

At step 3714, implant 3709 may be collapsed using inserter 3723. Inserter 3723 may be any inserter disclosed herein. At step 3716, implant 3709 may be removed from bone B. Cavity 3721 is shown remaining after removal of implant 3709 from bone B.

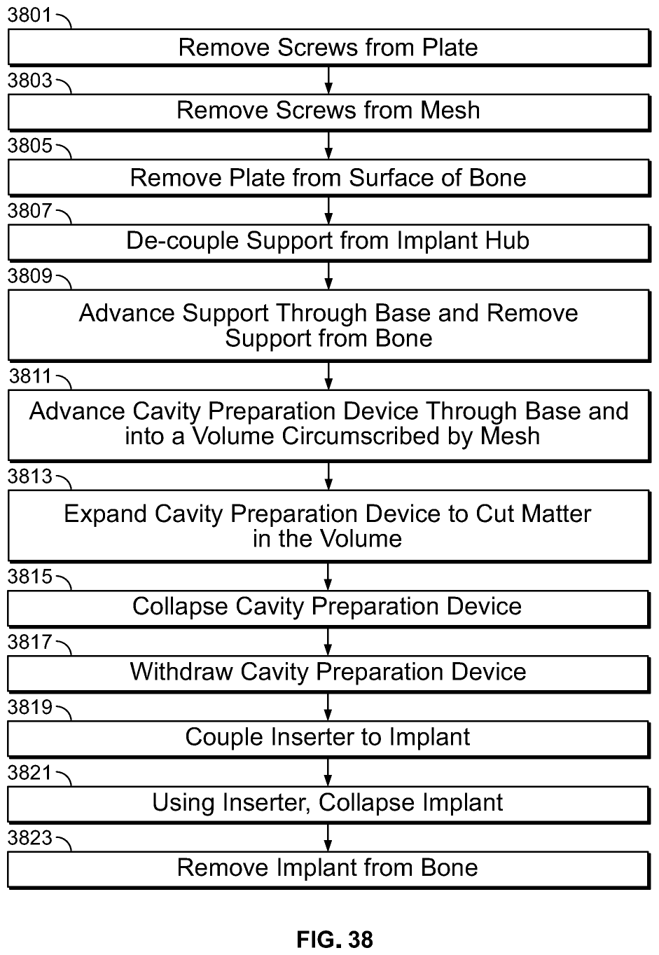

FIG. 38 shows an illustrative method including method steps 3801. Method steps 3801 may be performed in the order described. Method steps may be performed in an order different from the order described. Steps not included in FIG. 38 but disclosed herein may be included in the methods.

At step 3801, the methods may include removing screws from the plate. At step 3803, the methods may include removing screws from the mesh. At step 3805, the methods may include removing the plate from a surface of the bone. At step 3807, the methods may include de-coupling the support from the implant hub. At step 3809, the methods may include advancing the support through the base and withdrawing the support from the bone. At step 3811, the methods may include advancing the broaching tool through the base and into the volume circumscribed by the mesh. The volume circumscribed by the mesh may include a volume circumscribed by the implant head.

At step 3813, the methods may include expanding the broaching tool to cut matter in the volume. The expanding may be expanding of the broaching members. At step 3815, the methods may include collapsing the broaching tool. The collapsing may be collapsing the broaching members. At step 3817, the methods may include withdrawing the broaching tool from the bone. At step 3819, the methods may include coupling the inserter to the implant. At step 3821, the methods may include using the inserter to collapse the implant. At step 3823, the methods may include removing the implant from the bone.

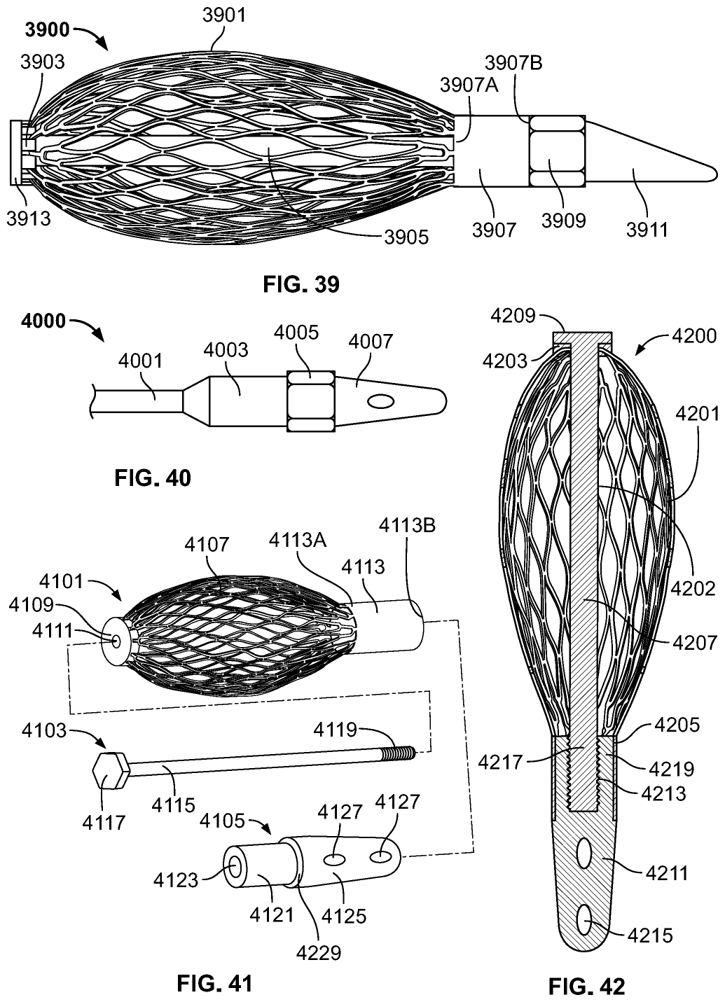

FIG. 39 shows illustrative apparatus 3900. Apparatus 3900 may include mesh 3901 extending between hub 3903 and base 3907. Base first end 3907A may be adjacent mesh 3901. Base second end 3907B may be adjacent nut 3909. Nut 3909 may be disposed between base 3907 and atraumatic member 3911. Atraumatic member may be shaped to ease implantation of apparatus 3900 in an interior of a bone.

The methods may include advancing atraumatic member 3911 through an access hole or an opening and interior. The methods may include continuing to advance atraumatic member 3911 until end cap 3913 is in the interior. The methods may include positioning end cap 3913 proximal to the access hole or opening.

Support 3905 may extend through mesh 3901. Support may have one or more features in common with support 4202. Support 3905 may include end cap 3913 and a threaded surface, such as threaded surface 4217. When threaded surface 4217 is engaged fully or partially with a threaded bore extending through base 3907, such as threaded bore 4213, end cap 3913 may be seated on hub 3903. When end cap 3913 is seated on hub 3903, further rotation of end cap 3913 to drive the threaded surface further into the threaded bore may radially expand mesh 3901 further away from a central axis of support 3905.

FIG. 40 shows illustrative apparatus 4000. Apparatus 4000 shows illustrative implant tail including elongated body 4003 for being seated in the base. The implant tail may also include nut 4005 and atraumatic member 4007. Apparatus 4000 also shows a portion of a support 4001. Support 4001 may be removably coupled to a threaded bore, such as threaded bore 4213, extending through implant elongated body 4003.

FIG. 41 shows illustrative implant 4101, support 4103 and tail 4105. Implant 4101 may include mesh 4107 extending between hub 4109 and base 4113. First end 4113A of base 4113 may be adjacent mesh 4107. Second end 4113B of base 4113 may be opposite first end 4113A along a central axis of base 4113.

Hub 4109 may define opening 4111. Opening 4111 may be sized to allow passage of elongated body 4115 of support 4103.

Support 4103 may include elongated body 4115 extending longitudinally between end cap 4117 and threaded surface 4119.

Implant tail 4105 may include atraumatic member 4125. Atraumatic member may define openings 4127. Implant tail 4105 may define body 4121. Body 4121 may be sized for being seated in, and fixedly coupled, a cannula extending through base 4113. Bore 4131 may extend through implant tail 4105. Bore 4131 may be threaded for threadedly engaging threaded surface 4119.

FIG. 42 shows a cross-sectional view of illustrative apparatus 4200. Apparatus 4200 may include mesh 4201 extending between hub 4203 and base 4205. Base 4205 is fixedly coupled to implant tail 4211. Implant tail may define openings 4215. Support 4202 may include elongated body 4207 that is defined between end cap 4209 and threaded surface 4217. In FIG. 42, end cap 4209 is seated on hub 4203 and threaded surface 4217 is in threaded engagement with threaded bore 4213. Threaded bore 4213 is shown extending through body 4219 of implant tail 4211. Threaded bore 4213 may extend through body 4219 of implant tail and into an atraumatic member included in tail 4211.

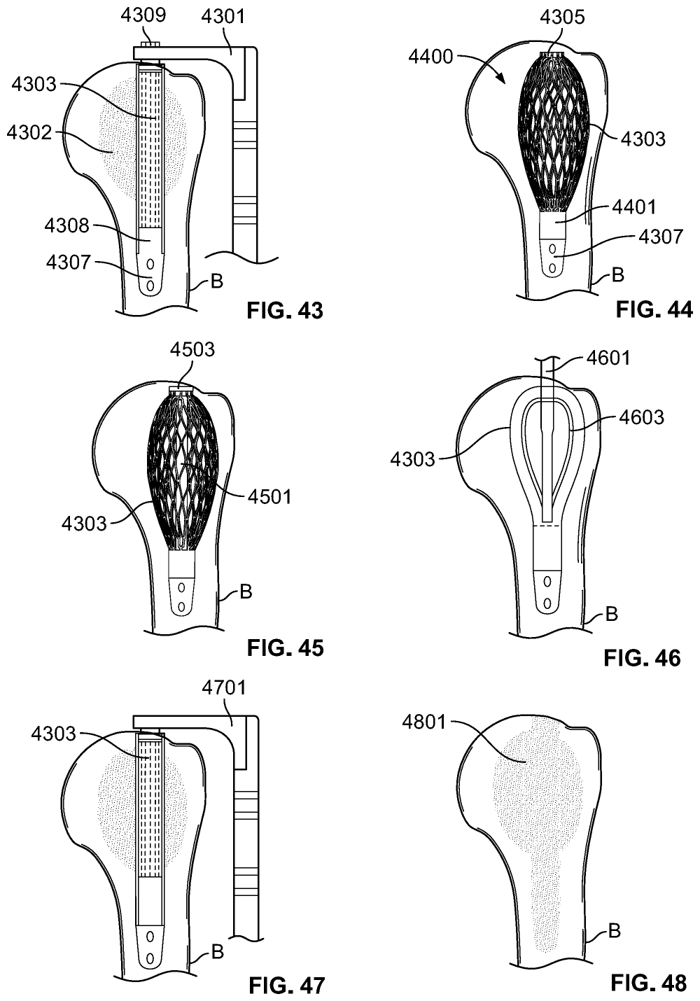

FIGS. 43-48 show illustrative methods for implanting implant 4400 in bone B and subsequently removing implant 4400. Implant 4400 may include mesh 4303 extending between hub 4305 and base 4401 and an implant tail including body 4308 and atraumatic member 4307. Support 4501 may be removably coupled to implant 4400. Hub 4305 may define an opening. Implant tail may define a threaded bore.

The illustrative methods in FIGS. 43-48 may be performed in the order described. The illustrative methods in FIGS. 43-48 may be performed in an order different from the order described. Steps not included in FIGS. 43-48 but disclosed herein may be included in the methods.

FIG. 43 shows implant 4400 including mesh 4303 positioned in cavity 4302 prepared in bone B. In FIG. 43, jig 4301 is shown coupled to implant 4400 and holding mesh 4303 in a collapsed position. End cap 4309 is positioned on jig 4301. End cap 4309 may be part of a first support having a member engaged with the implant tail. The first support may include end cap 4309. End cap 4309 may retain mesh 4303 in a collapsed position.

The methods may include using an inserter described herein to collapse mesh 4303 and insert implant into bone B in the collapsed position.

The methods may include removing the first support from implant 4400. Implant 4400 may be a self-expanding implant. Removing the first support may expand the implant. The first support may be removed by turning end cap 4309 of the first support seated on a top face of jig 4301. The first support may have a threaded surface in threaded engagement with the implant tail. Turning end cap 4309 may unscrew the threaded surface from the implant tail and release the force on the implant, allowing it to self-expand.

FIG. 44 shows implant 4400 positioned in bone B. In FIG. 44, jig 4301 or the inserter has de-coupled from implant 4400, allowing mesh 4303 to self-expand. In FIG. 44, a support is not coupled to implant 4400.

FIG. 45 shows support 4501 coupled to implant 4400, with end cap 4503 seated on hub 4305. Support 4501 may be the second support. A member of support 4501 positioned opposite the end cap 4503 may be coupled to body 4308 of the implant tail. The member may be a threaded surface such as threaded surface 4217. Turning end cap 4503 may thread the threaded surface into a threaded bore extending through body 4308. As end cap 4503 presses down on mesh 4303, mesh 4303 may further expand in bone B.

FIG. 46 show preparations being done for removing implant 4400 from bone B. In FIG. 46, support 4501 has been de-coupled from the implant tail and removed from bone B through the opening in hub 4305. In FIG. 46, broaching tool 4601 has been advanced through the opening in hub 4305. Broaching members 4603 have been expanded. Broaching tool 4601 may be rotate to broach matter circumscribed by mesh 4303. The matter may be organic matter that accumulated in the volume after implantation of implant 4400. The matter may be matter, including organic matter and/or non-organic matter, placed in the volume by a practitioner during the implantation of implant 4400.

After broaching matter in the volume, broaching members 4603 may be collapsed, and broaching tool 4601 may be removed from the bone in the collapsed state.

FIG. 47 shows mesh 4303 collapsed. Jig 4701 may collapse implant 4400. Collapsing implant 4400 may include collapsing mesh 4303. Jig 4701 may collapse mesh 4303 using a first support as described above in connection with jig 4301. An inserter described herein may be used to collapse implant 4400.

FIG. 48 shows implant 4400 removed from bone B. Cavity 4801 is shown remaining in bone B after removal of implant 4400.

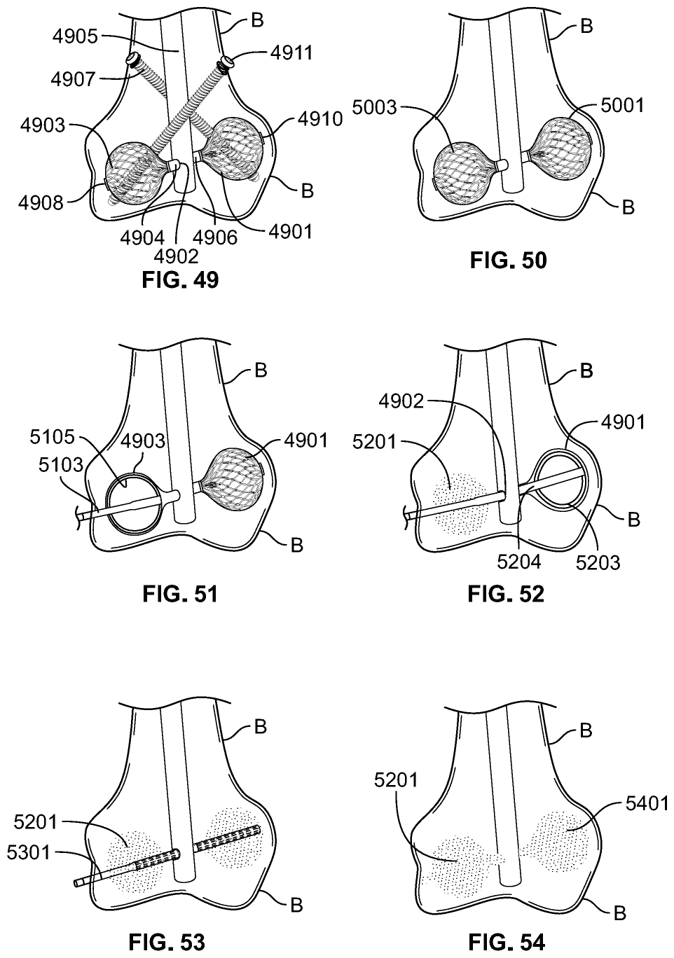

FIG. 49 shows illustrative apparatus implanted in bone B. Apparatus may include first implant 4901, second implant 4903 and intramedullary nail 4905 implanted in bone B. Bore 4902 may extend through nail 4905. Bore 4902 may transect intramedullary nail 4905 at an angle perpendicular to a central axis of intramedullary nail 4905. Bore 4902 may transect intramedullary nail 4905 at an angle transverse to a central axis of intramedullary nail 4905.

First member 4904 may extend away from first implant 4901 and into nail 4905. Second member 4906 may extend away from second implant 4903 and into nail 4905.

First member 4904 may be a first base. First member 4904 may be a first implant tail (“tail”). Second member 4906 may be a second base. Second member 4906 may be a second tail. First base may be cannulated. First implant tail may be cannulated. Second base may be cannulated. Second implant tail may be cannulated.

The first base and the second base may be coupled together inside nail 4905.

The first tail and the second tail may be coupled together inside nail 4905.

The first tail and the second base may be coupled together inside nail 4905.

The first base and the second tail may be coupled together inside nail 4905.

Screw 4911 may extend through bone B and into second implant 4903. Screw 4907 may extend through bone B and into first implant 4901.

Hub 4910 of first implant 4901 may define an opening. A broaching tool, such as broaching tool 5103, may be advanced through the opening and into a volume circumscribed by first implant 4901 to broach matter accumulated in first implant 4901. A tool for coupling first implant 4901 to second implant 4903 may be advanced through the opening to couple together first implant 4901 and second implant 4903.

Hub 4908 of second implant 4903 may define an opening. A broaching tool, such as broaching tool 5103, may be advanced through the opening and into a volume circumscribed by second implant 4903 to broach matter accumulated in second implant 4903. A tool for coupling first implant 4901 to second implant 4903 may be advanced through the opening to couple together first implant 4901 and second implant 4903.

First implant 4901 may be removed during a first procedure. Second implant 4903 may be removed during a second procedure. First implant 4901 and second implant 4903 may be removed during the same procedure.

FIGS. 50-54 show illustrative methods for removing from bone B first implant 4901 and second implant 4903, illustrated in FIG. 49. The illustrative methods in FIGS. 50-54 may be performed in the order described. The illustrative methods in FIGS. 50-54 may be performed in an order different from the order described. Steps not included in FIGS. 50-54 but disclosed herein may be included in the methods.

FIG. 50 shows nails removed from engagement with implant cage 5001 and second implant 5003.

FIG. 51 shows broaching tool 5103 extending through bone B and into a volume circumscribed by second implant 5003. Broaching tool 5103 may be advanced through an opening in hub 4910 and into a volume circumscribed by second implant 4903. Broaching tool 5103 may be expanded, and broaching members 5105 may broach matter inside second implant 4903. The matter may be organic matter or non-organic matter, as described above in connection with FIG. 46.

A drill may be used to drill a pathway through bone B, through an opening extending through hub 4908, and into a volume circumscribed by second implant 4903. Broaching tool 5103 may be advanced through the pathway and into second implant 4903 to broach matter.

After broaching matter inside second implant 4903, second implant 4903 may be collapsed and removed from bone B. Any inserter described herein may be used to collapse and remove second implant 4903 from bone B. An outer circumference of hub 4908 may include windows for coupling to the inserter. An inner circumference of hub 4908, surrounding the opening, may include engagement feature for engaging the inserter.

FIG. 52 shows cavity 5201 remaining in the bone where second implant 4903 was previously positioned.

FIG. 52 shows broaching tool 5204 extending through bore 4902 and into a volume circumscribed by first implant 4901. Broaching tool 5204 may be inserted in a collapsed state, expanded inside first implant 4901, and rotated to broach matter with broaching members 5203. The matter may be organic matter or non-organic matter, as described above in connection with FIG. 46.

A drill may be used to drill a pathway through bone B, through bore 4902, and into the volume circumscribed by first implant 4901. Broaching tool 5204 may be advanced through the pathway and into first implant 4901 to broach matter.

After broaching matter inside first implant 4901, first implant 4901 may be collapsed and removed from bone B. Any inserter described herein may be used to collapse and remove first implant 4901 from bone B. An outer circumference of hub 4910 may include windows for coupling to the inserter. An inner face of hub 4910 may include features for coupling to the inserter.

FIG. 53 shows first implant 4901 collapsed and coupled to inserter 5301.

FIG. 54 shows first implant 4901 and second implant 4903 removed from bone B. Cavity 5201 and cavity 5401 are shown remaining in bone B after removal of first implant 4901 and second implant 4903.

After removal of first implant 4901 and second implant 4903, intramedullary nail 4905 may be removed from bone B.

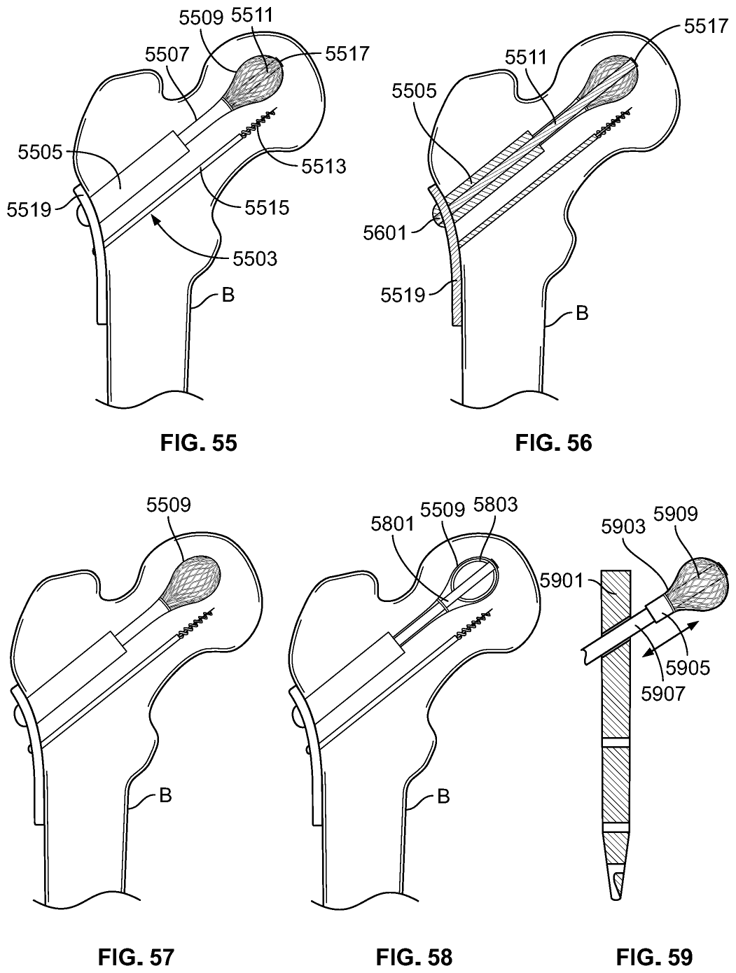

FIG. 55 shows illustrative apparatus implanted in bone B. The apparatus may include plate 5519 and elongated member 5503 extending through plate 5519. Elongated member 5503 may include body 5515 and threaded surface 5513. Elongated member 55003 may be an anti-rotation screw.

The apparatus may include barrel 5505 seated in an opening defined by plate 5519. Barrel 5505 and plate 5519, together, may form a unitary structure. Barrel 5505 may extend away from plate 5519. Implant 5509 may extend through barrel and into bone B. Bone B may be a femur. Implant 5509 may be positioned in a head of the femur. Central member 5511 may extend through implant 5509. Central member 5511 may extend between hub 5517 of implant and through barrel 5505.

Central member 5511 may be in sliding engagement with barrel 5505 (as illustrated in FIG. 59, below). Central member 5511 may be the support.

FIG. 56 shows a cross-sectional view of apparatus illustrated in FIG. 55. FIG. 56 shows central member 5511 extending along implant 5509, through barrel 5505 and through plate 5519. End 5601 of central member 5511 may extend away from plate 55519. End 5601 may be a flanged end. End 5601 may couple implant 5509 to plate 5519.

Methods may include anchoring implant 5509 to bone B by pulling end 5601 away from bone B to reduce bone B. Methods may include not pulling end 5601 and allowing bone B to naturally collapse and settle.

Methods may include anchoring implant 5509 to bone B using one or more screws and pulling end 5601 away from plate 5519 to compress the femoral head.

FIGS. 57-58 show illustrative steps for preparing implant 5509 for removal from bone B. FIG. 57 shows central member 55011 removed from implant 5509.

FIG. 58 shows broaching tool 5801 inside implant 5509. Broaching members 5803 are shown broaching matter circumscribed by a head of implant 5509.

FIG. 59 shows illustrative apparatus for implanting in a bone. Apparatus illustrated in FIG. 59 may include implant 5903 coupled to nail 5901. Implant 5903 may include base 5905. Tail 5907 of implant 5903 may be slidingly engaged with a bore extending through nail 5901. The sliding engagement may enable tail 5907 of implant 5903 to slide along the opening.

Tail 5907 may be coupled to support 5909. Tail 5907 may be the support.

Methods may include anchoring implant 5903 to a bone and pulling an end of tail 5907 protruding away from nail 5901 to reduce a fracture. Methods may include not pulling the end of tail 5907 to reduce the fracture and instead to allow the bone to naturally settle.

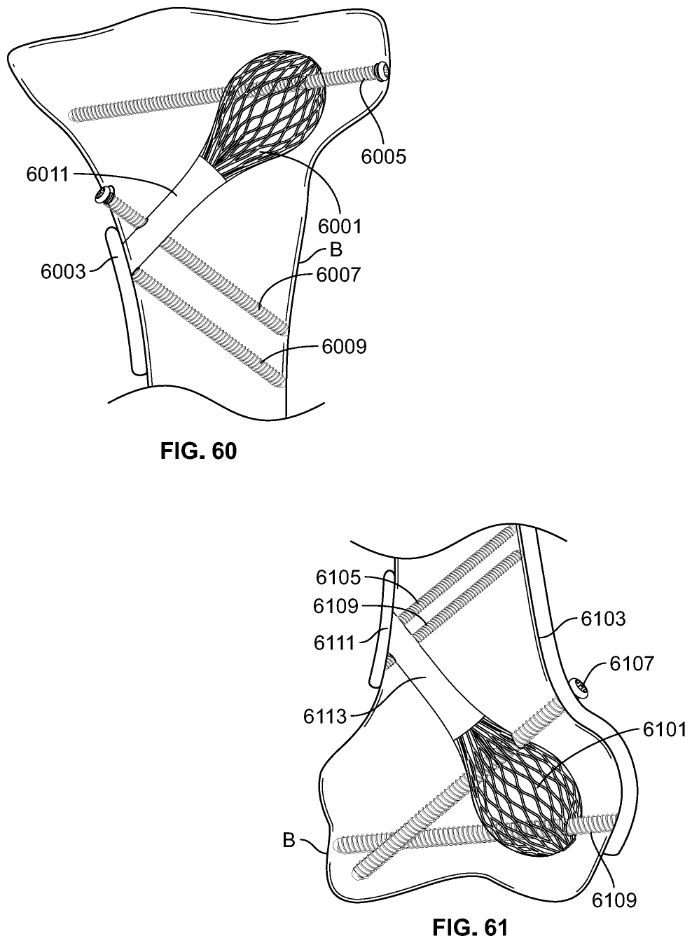

FIG. 60 shows illustrative apparatus in bone B. The apparatus may include implant 6001. Implant 6001 may include base 6011 and an expandable mesh head. Base 6011 may be coupled to plate 6003. Screw 6005 may anchor the mesh head to bone B. Screws 6007 and 6007 may extend through base 6011 and into bone B. Bone B may be a proximal tibia.

FIG. 61 shows illustrative apparatus in bone B. The apparatus may include implant 6101. Implant 6001 may include base 6113 and an expandable mesh head. Base 6113 may be coupled to plate 6111. Screws 6107 and 6109 may extend through plate 6103 and into the mesh head. Screws 6105 and 6109 may extend through base 6113 and into bone B. Bone B may be a distal femur.

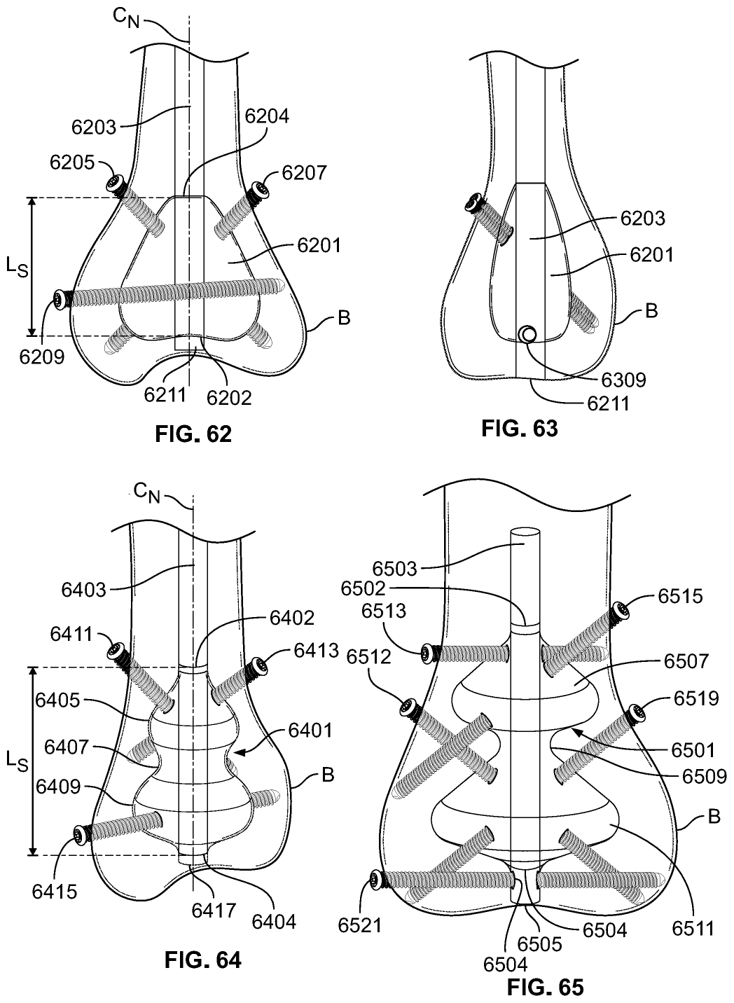

FIG. 62 shows illustrative apparatus in bone B. The apparatus may include implant 6201 coupled to nail 6203 in bone B. Implant 6201 and nail 6203 may include features of the implant and the nail described herein. Implant 6201 and nail 6203 may be coupled together and placed inside the bone using one or more of the method steps included in the methods described herein.

First end 6202 of implant 6201 may define a first annular opening. Second end 6204 of implant 6201 may define a second annular opening. Nail 6203 may define central axis C_{N}. When implant 6201 is positioned on nail 6203, a length Ls of the nail may extend between first end 6202 and second end 6204.

The methods may include mounting first end 6202 and second end 6204 on nail 6203. The methods may include sliding first end 6202 and second end 6204 of implant 6201 along nail 6203 and, after desired placement, anchoring implant 6201 in bone B.

The methods may include implanting implant 6201 in bone B using methods described herein for implanting the implant. The methods may include sliding a nail through the first end and the second end.

Screw 6205 and screw 6207 anchor implant 6201 to bone B. End 6211 of nail 6203 may be proximal to a surface of bone B.

FIG. 63 shows an illustrative side view of implant 6201 and nail 6203 illustrated in FIG. 62. In FIG. 63, bore 6301 extending through nail 6203 is visible. A screw may pass through implant 6201 and through bore 6303 to lock implant 6201 to nail 6203. The screw may prevent sliding of implant 6201 along nail 6203.

FIG. 64 shows illustrative apparatus in bone B. The apparatus may include implant 6401 coupled to nail 6403 in bone B. Implant 6401 and nail 6403 may include features of the implant and the nail described herein. Methods for coupling implant 6401 and nail 6403 may be methods described above at FIG. 62 and any other methods disclosed herein.

Implant 6401 may include first bulbous section 6405, second bulbous section 6409, and neck 6407 disposed between first bulbous section 6405 and second bulbous section 6409. First bulbous section 6405 may have features in common with the implant. Second bulbous section 6409 may have features in common with the implant.

First end 6402 of implant 6401 may define a first annular opening. Second end 6404 of implant 6401 may define a second annular opening. Nail 6403 may define central axis C_{N}. When implant 6401 is positioned on nail 6403, a length Ls of the nail may extend between first end 6402 and second end 6404.

Screw 6411, screw 6411 and screw 6415 anchor bone B to implant 6401. End 6211 of nail 6417 may be proximal to a surface of bone B.

FIG. 65 shows illustrative apparatus in bone B. The apparatus may include implant 6501 coupled to nail 6503 in bone B. Implant 6501 and nail 6503 may include features of the implant and the nail described herein. Methods for coupling implant 6501 and nail 6503 may be methods described above at FIG. 62 and any other methods disclosed herein.

Implant 6501 may include first bulbous section 6507, second bulbous section 6511, and neck 6509 disposed between first bulbous section 6507 and second bulbous section 6511. First bulbous section 6507 may have features in common with the implant. Second bulbous section 6511 may have features in common with the implant.

Screw 6515 and 6513 may anchor first bulbous section 6507 to bone B. Screw 6519 and 6512 may anchor second bulbous section 6511 to bone B. Screw 6521 may extend through bore 6504 in nail 6503 to anchor nail 6503 to bone B. End 6505 of nail 6503 may be proximal to a surface of bone B.

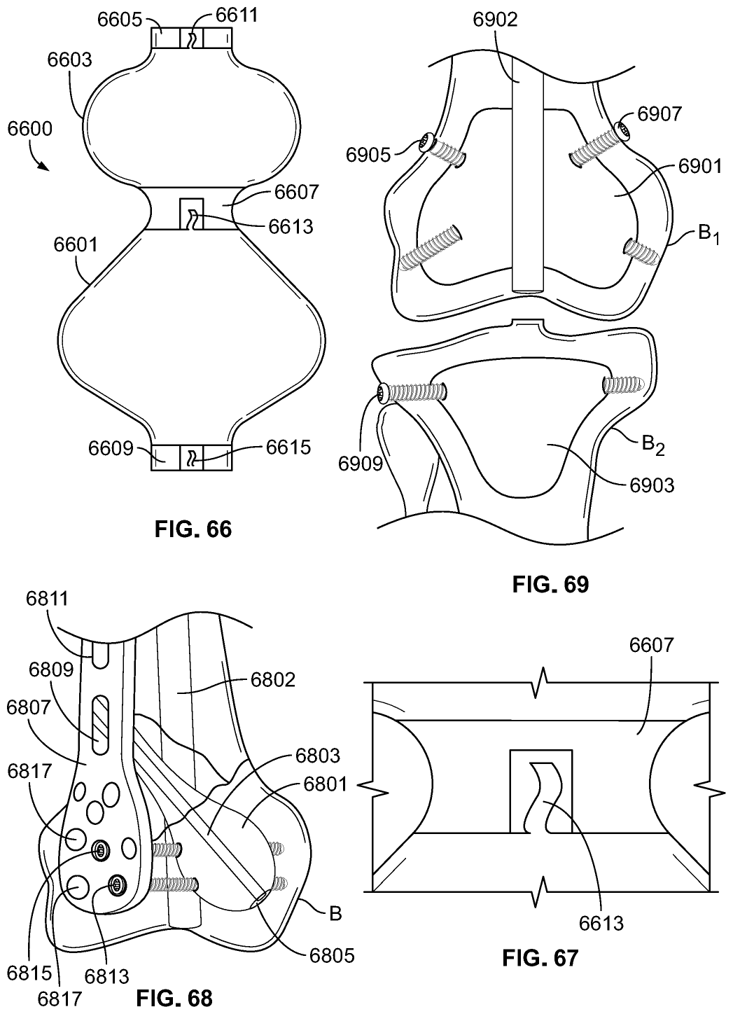

FIG. 66 shows illustrative implant 6600. Implant 6600 may include first bulbous section 6601, second bulbous section 6603, and neck 6607 disposed between first bulbous section 6601 and second bulbous section 6603. First bulbous section 6601 may have features in common with the implant. Second bulbous section 6603 may have features in common with the implant.

Neck 6607 may be a portion of mesh extending between first bulbous section 6601 and second bulbous section 6603.

Neck 6607 may be a hub having openings at a first end and a second end. Openings at a first end may receive mesh features, such as ‘tabs’, of first bulbous section 6601. Openings at a second end may receive mesh features, such as ‘tabs’, of second bulbous section 6603.

Hub 6609 may be positioned adjacent a first end of first bulbous section 6601. Neck 6607 may extend between second end of first bulbous section 6601 and first end of second bulbous section 6603. Hub 6605 may be positioned at a second end of second bulbous section 6603. Hub 6605 may be opposite hub 6605 along a central axis of implant 6600. Each of hub 6609 and hub 6605 may have one or more features in common with the hub. Hub 66011 may include projection 6611. Neck 6607 may include projection 6613. Hub 6609 may include projection 6615.

One or more of hub 6609, hub 6605 and neck 6607 may include two, three, four, five, ten, twenty, thirty, or more projections 6615. Each of hub 6609, hub 6605 and neck 6607 may define an annular opening. The annular openings may be coaxial. The annular openings may define the same diameter. Two or more of the annular openings may define different diameters.

Projections 6615 may be biased toward a central axis of implant 6600. Projections 6615 may retain implant 6600 on a nail. Projections 6615 may provide a force to couple implant 6600 to a nail so that implant 6606 does not slide down the nail when force is not being applied to implant 6606.

FIG. 67 shows an enlarged view of neck 6607 and tab 66013 illustrated in FIG. 66.

FIG. 68 shows illustrative apparatus in bone B. The apparatus includes implant 6808 in bone B. Support 6803 may be coupled to hub 6805 of implant 6808. Plate 6807 may be positioned on a surface of bone B. Plate 6807 may define elongated slot 6809 and elongated slot 6811. Plate 6807 may define openings 6817. Screw 6813 and screw 6815 may extend through plate 6807 and into implant 68901. A tail of implant 6801 may be coupled to plate 6807.

The apparatus may include nail 6802. Nail 6802 may extend along a length of bone B. Nail 6802 may be positioned adjacent implant 6801. Implant 6801 may abut nail 6802. Implant 6801 may not abut nail 6802.

Implant 6801 may include features of the implant. Nail 6802 may include features of the nail. Plate 6807 may include features of plates described herein.

FIG. 69 shows first implant 6901 positioned in bone B₁. Screws 6907 and 6905 may anchor first implant 6901 to bone B₁. Nail 6902 may extend along bone B₁. Nail 6902 may or may not abut first implant 6901. First implant 6901 may be mounted on nail 6902 as described above in connection with FIGS. 62-65. First implant 6092 and nail 6902 may have features in common with the implant and the nail.

FIG. 69 also shows second implant 6903 positioned in bone B₂. Screw 6909 may anchor second implant 6903 to bone B. Second implant 6903 may have features in common with the implant.

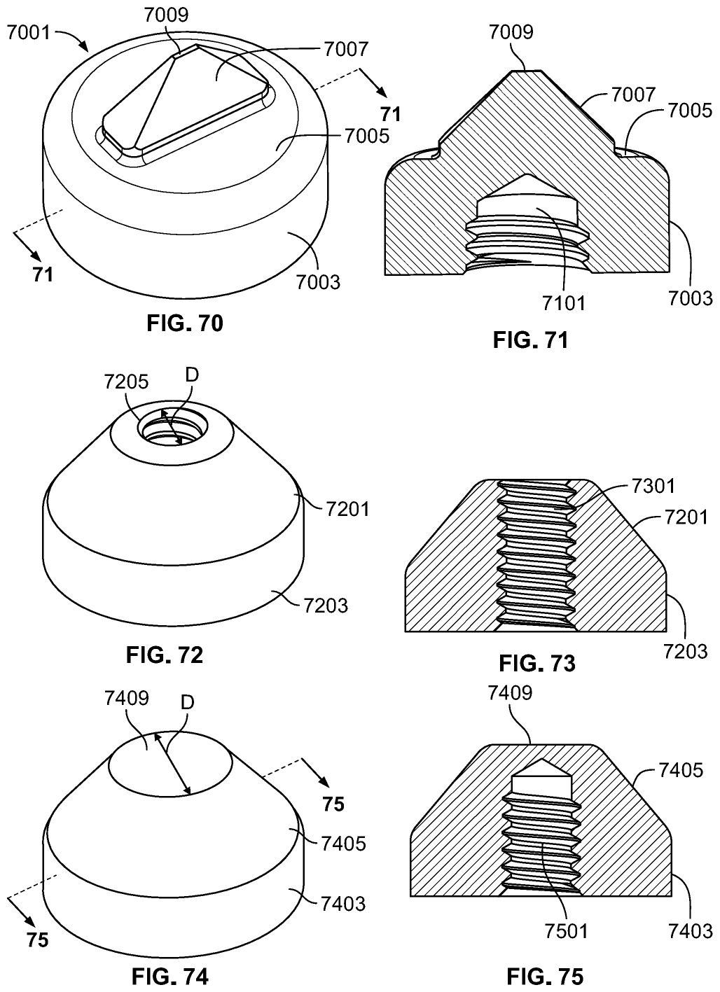

FIG. 70 shows illustrative hub 7001 that may be used with any of the implants disclosed herein. The hub may include protrusion 700. Protrusion 7009 may be a tissue-engaging member. Tissue engaging member may assist in anchoring the implant to a bone. Bottom section of the hub (not shown) may include radially-spaced cutouts which retain the terminal ends of the mesh cells of the implant head while permitting expansion and contraction of the implant head. Hub 7001 may include cylindrical surface 7103. Hub 7002 may include hub first face 7105. Protrusion 7009 may extend away from hub first face 7105.

FIG. 71 shows a cross-sectional view of hub 7001. In FIG. 71, threaded bore 7101 is shown extending through some, but not all, of hub 7001.

FIG. 72 shows an illustrative hub that may be used with any of the implants disclosed herein. The hub illustrated in FIG. 72 may include cylindrical portion 7203. The hub may include tapered portion 7201. Tapered portion may define a conical surface. Tapered portion may have a circumference that decreases between cylindrical portion 7203 and hub second end 7205 along a central axis of the hub. Hub second end 7205 may define an opening. The opening may be an annular opening.

FIG. 73 shows a cross-sectional view of hub 7200. In FIG. 73, threaded bore 7301 is shown extending through the hub and through the opening defined by hub second end 7205.

FIG. 74 shows an illustrative hub that may be used with any of the implants disclosed herein. The hub illustrated in FIG. 74 may include cylindrical portion 7403. The hub may include tapered portion 7405. Tapered portion 7405 may define a conical surface. Tapered portion 7405 may have a circumference that decreases between cylindrical portion 7403 and hub second end 7409 along a central axis of the hub. Hub second end 7409 may define a round, flat surface. Hub second end 7409 may define a diameter D.

FIG. 75 shows a cross-sectional view of the hub illustrated in FIG. 74. In FIG. 75, threaded bore 7501 is shown extending through some, but not all, of the hub.

In an interior of the bone, the cylindrical side surfaces of the hub may be placed such that it extends past a cavity created in the bone for the implant. The cylindrical side may be placed in a portion of a previously-drilled hole that extends pass the cavity. This may keep the alignment of the cage assembly regardless of the symmetry of the cage cavity and any resulting side forces on the cage due to the cage being deployed in a asymmetric cavity. The cylindrical side surface may act as a bearing surface for both ends of implant assembly—i.e. the hub and the base—that will both be positioned inside the predrilled hole and not inside the cavity. This may retain axial alignment of the implant along a central axis of the hole even if side loads are put on a middle portion of the implant due to asymmetric cavity creation.

Methods may include boring a hole through an access site on a surface of the bone. Methods may include advancing a broaching tool through the hole and placing an end of the cavity prep device spaced apart from an end of the hole in the bone. The methods may include preparing the cavity. The methods may include advancing an implant into the interior of the bone along through the access site. The methods may include positioning a hub of the implant at the end of the hole and allowing the implant to expand in the cavity. The methods may include providing axial alignment of the implant in the interior of the bone by placing the hub in a first length of the hole, and not in the cavity, and placing the base in a second length of the hole, and not in the cavity, when the cavity extends between the first length of the hole and the second length of the hole. The second length may be more proximate the access hole than is the first length.

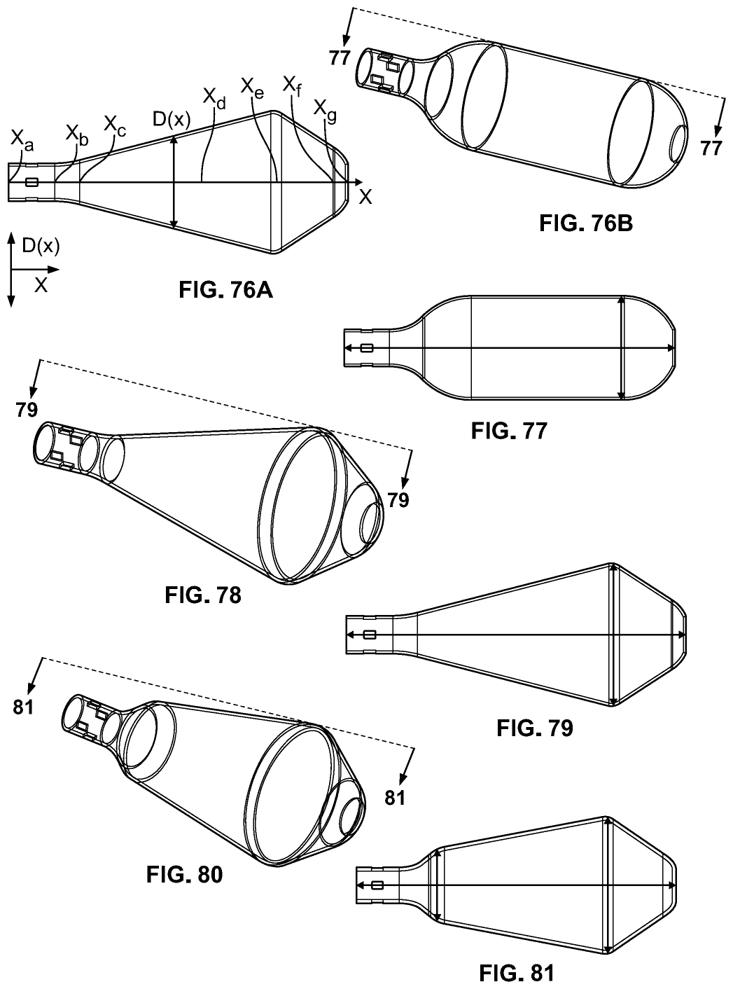

FIG. 76A shows schematically illustrative implant shape factors D(x) for implant landmarks xₐ, x_{b}, x_{c}, x_{d}, xₑ, x_{f} and x_{g}.

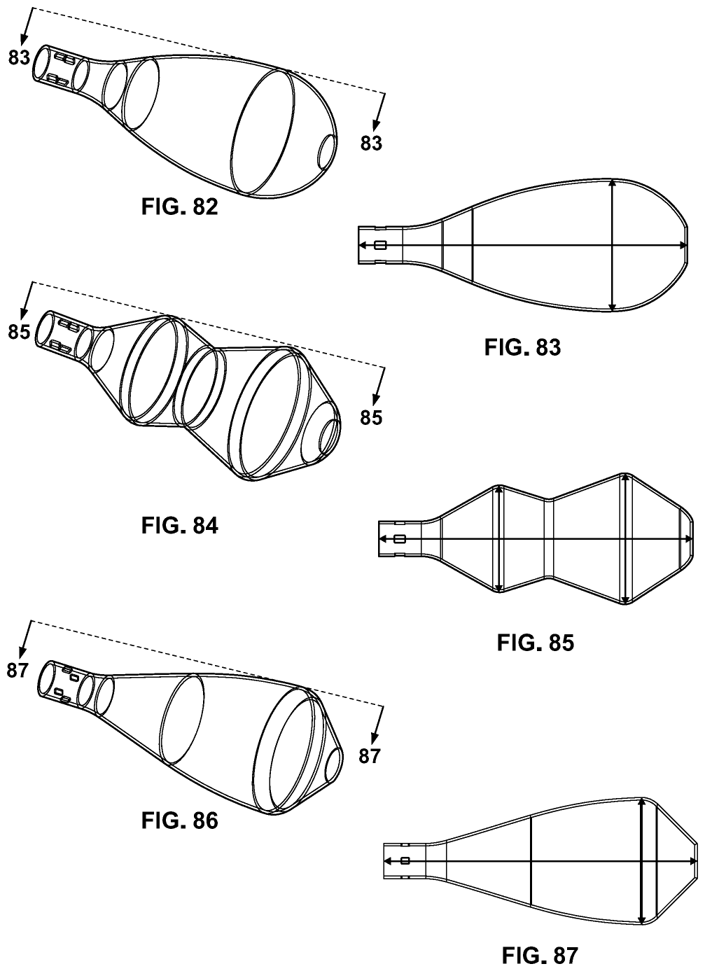

FIGS. 76B to 87 show implants with different sets of shape factors.

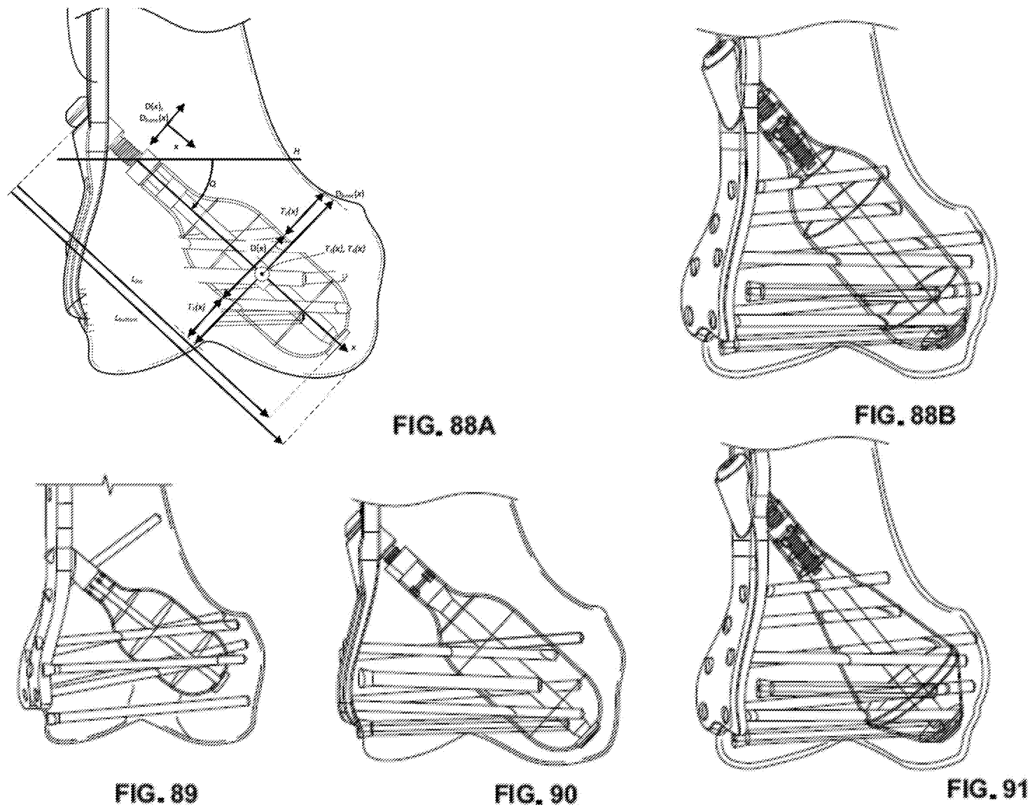

FIG. 88A shows schematically illustrative implant fit factor parameters Q, H, D_{bone}(x), T₁(x), T₂(x), T₃(x), T₄(x), Lᵢₙₛ, and L_{bottom}.

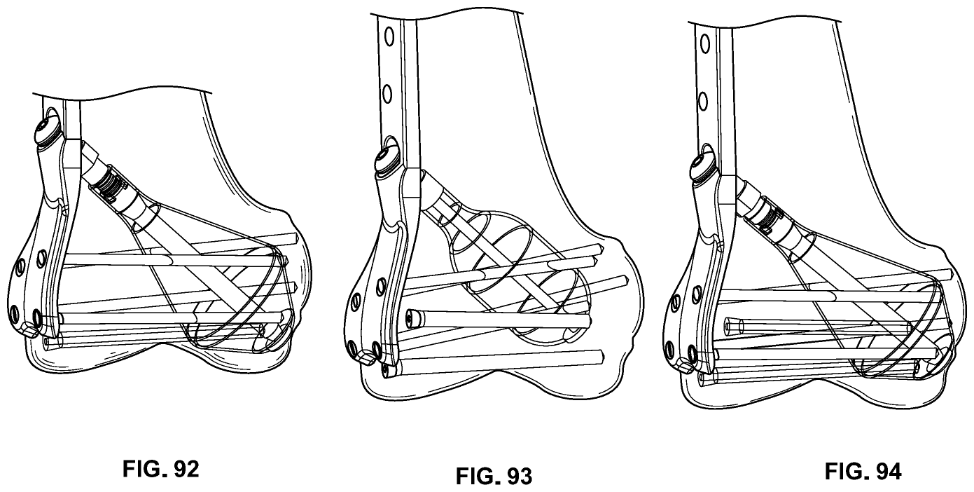

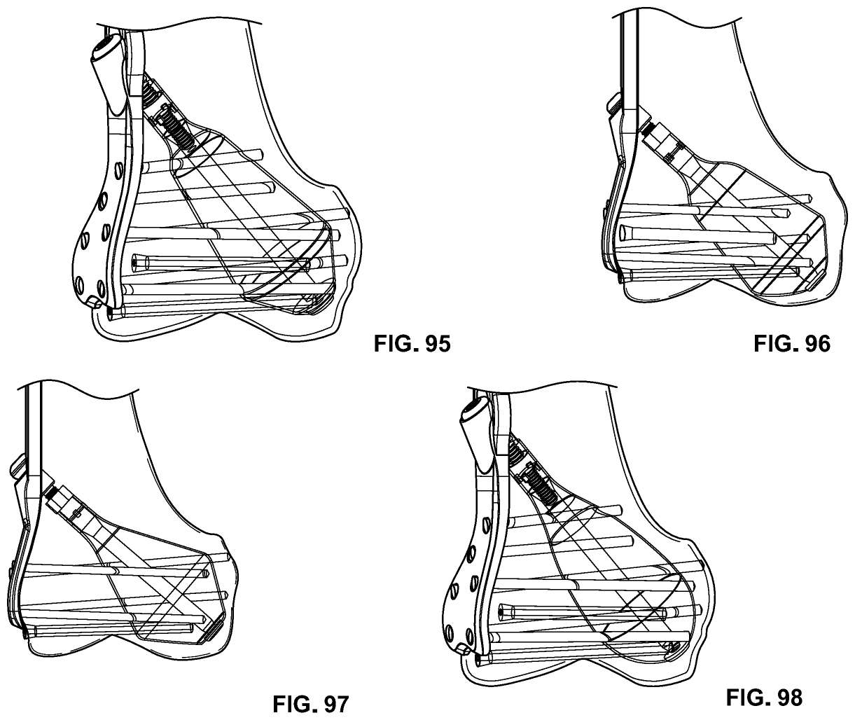

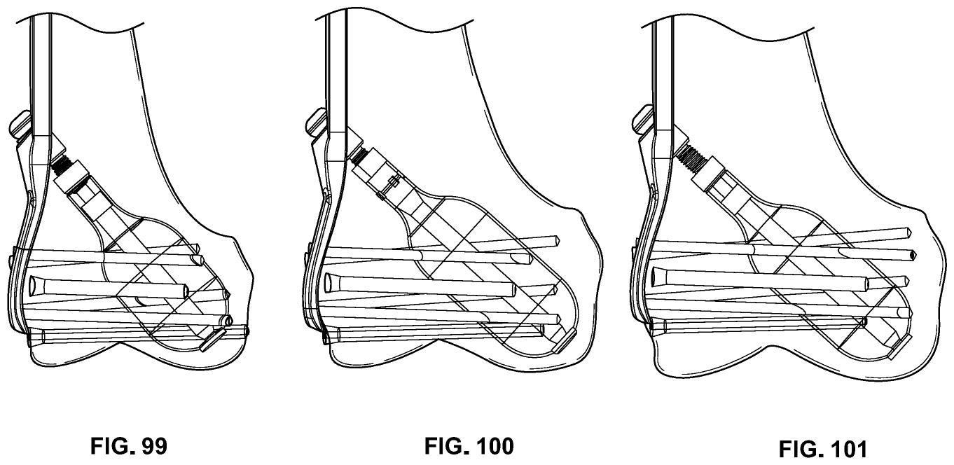

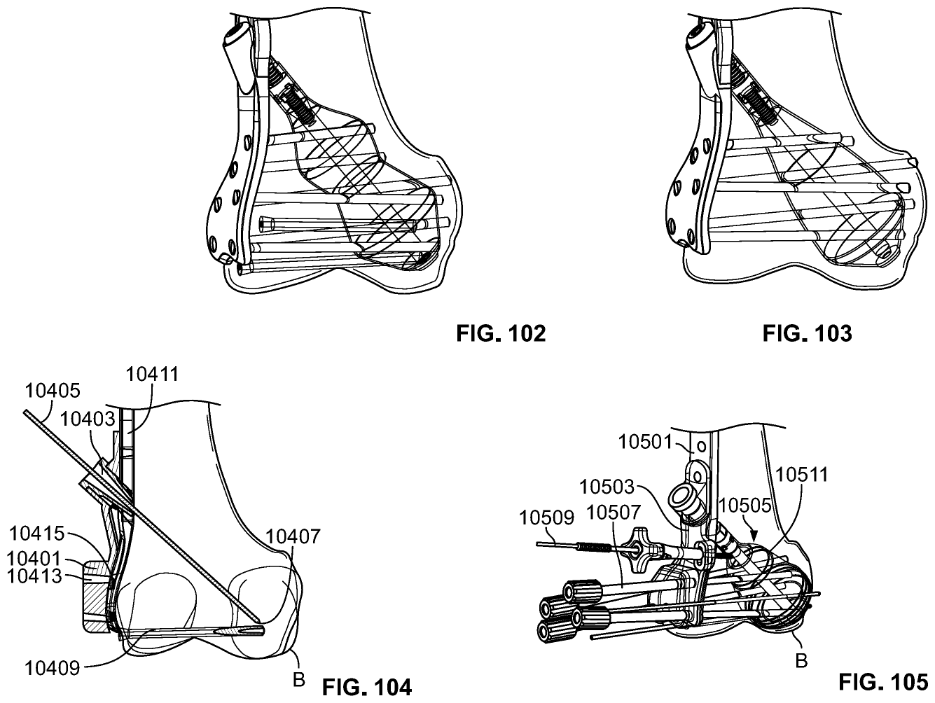

FIGS. 88B to 103 show implants with different sets of shape factors.

The methods may include selecting a shape of an implant for implanting in a bone B. Exemplary shapes may be shapes illustrated in FIGS. 76B-87, or any other suitable shape. A shape of an implant may be selected to conform to anatomical features of bone B. Selecting the shape of the implant may include analyzing a fracture pattern in bone B.

The methods may include selecting a size of the implant. Selecting a size of the implant may include analyzing a size of bone B and a size of a cavity prepared in bone B.

Placement of a hub of the implant proximal to a surface of bone B may be desired. Anatomical features of bone B, such as when bone B defines a surface with steep curvature, may make selection of an implant defining a tapered section adjacent the hub desirable.

A hub of the implant may not be desired to be placed proximal to a surface of bone B.

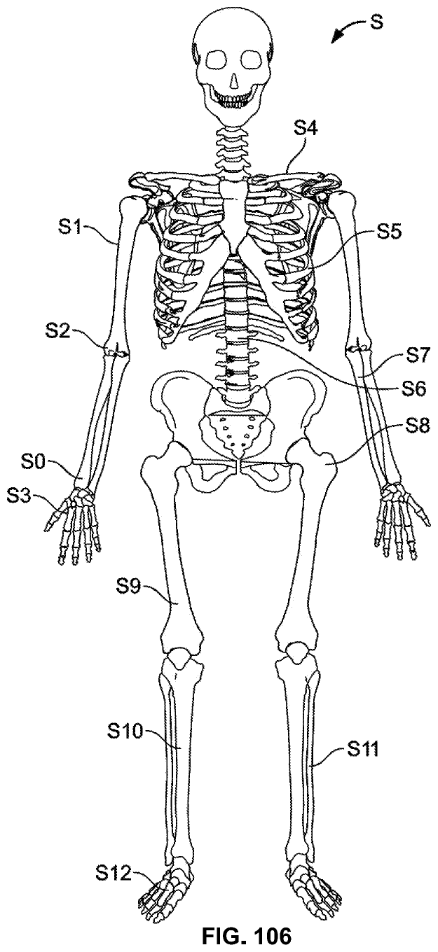

FIGS. 104-105 show illustrative instrumentation, instrumentation layout and guide block architecture for advancing the implant into a bone such as bone B.

FIG. 104 shows illustrative usage of plate 10411 for coupling to jig 10401. Jig 10401 may define openings 10413 for guiding fixation members, such as screws, into a bone. Openings 10415 may be positioned below openings 10413. In FIG. 104, screw 10490 extends into bone B. Jig 10401 may include guide 104-3. Guide 10403 may be used to guide a k-wire, drill, and/or the implant into bone B. In FIG. 104, k-wire 10405 is illustrated extending through guide 10403 and into bone B. Tip 10407 is shown positioned in bone B. Tip 10407 may be positioned at a target site. The target site may be a location in bone B at which a hub of the implant is to be positioned.

Methods may include placing a plate on bone. Methods may include using temporary fixation methods to fix the plate to the bone. Methods may include provisional reduction of one or more fractures or anomalies of the bone. Provisional reduction may include fixing bone to the plate using k-wires passing through the plate and into the bone. Methods may include placing apparatus into the bone through the guide sleeve on the plate.

FIG. 105 shows illustrative jig 10503. Jig 10503 may include one or more features of jig 10411. Jig 10503 may be jig 10401.

Jig 10503 may be seated on plate 10501. Bushings 10507 are shown positioned in openings on jib 10503. Bushings 10507 may be cannulated. Apparatus such as k-wires, drills, screws, or any other suitable apparatus may pass through one or more of bushings 10507 and into bone B. Illustrative k-wire 10509 is shown extending through a slotted opening in jig 10503. A cannulated screw may be slid over k-wire 10509, through the slotted opening and into implant 10505. In FIG. 105, implant head 10511 is in an expanded state.

FIG. 106 shows illustrative anatomy in connection with which the apparatus and methods may be used. FIG. 106 shows illustrative skeleton S. Skeleton S may include illustrative bones Sᵢ in which apparatus and methods in accordance with the principles of the invention may be used.

The apparatus and methods may be used in connection with “hollow” bones. The hollow bones may include cortical tissue. The hollow bones may include cancellous tissue. Cortical tissue may be referred to as “tissue.” Cancellous tissue may be referred to as “tissue.” Other matter in the interior of a bone may be considered “tissue.” The bone may be considered “tissue.”

The apparatus and methods may be used to create a space inside a bone. The bone may be any bone Sᵢ included in Table 11 below. The space may be a cavity. The tissue may be inside the bone. The space may be created by breaking up the tissue. The space may be created by removing the tissue from the bone. The space may be created as part of a therapeutic procedure. The apparatus and methods may displace tissue by imparting mechanical energy to the tissue, for example, through one or more of expanding motion, rotational motion, axial motion, compressive motion, cutting motion, broaching motion, and any other suitable motions of a device such as a broaching tool.

The apparatus and methods may be used to deploy an implant in the space created inside the bone. The apparatus and methods may be used to anchor the implant to the bone. The implant may be any implant disclosed herein. The implant may be used together with any plate and/or nail disclosed herein. The apparatus and methods disclosed herein may be used in any bone in the body.

The implant may be positioned in a metaphyseal area of a bone or in any other suitable area of the bone. Illustrative bones that may be repaired using the apparatus and methods disclosed herein may include the proximal humerus, proximal tibia, distal femur, distal tibial, proximal femur, and any other areas in bones set forth in Table 11 below.

Illustrative bones Si in which apparatus and methods in accordance with the principles of the invention may be used are included in Table 11 below. Table 11 may include a partial list of bones Si.

TABLE 11
Bones Sᵢ.

| Bone | Reference numeral in FIG. 106 |
|---|---|
| Distal Radius | S₀ |
| Humerus | S₁ |
| Proximal Radius and Ulna (Elbow) | S₂ |
| Metacarpals | S₃ |
| Clavicle | S₄ |
| Ribs | S₅ |
| Vertebrae | S₆ |
| Ulna | S₇ |
| Hip | S₈ |
| Femur | S₉ |
| Tibia | S₁₀ |
| Fibula | S₁₁ |
| Metatarsals | S₁₂ |

Apparatus and methods described herein may be utilized in any bone Si included in Table 11 above, in any other bone in the human body, and in any suitable bone in an animal.

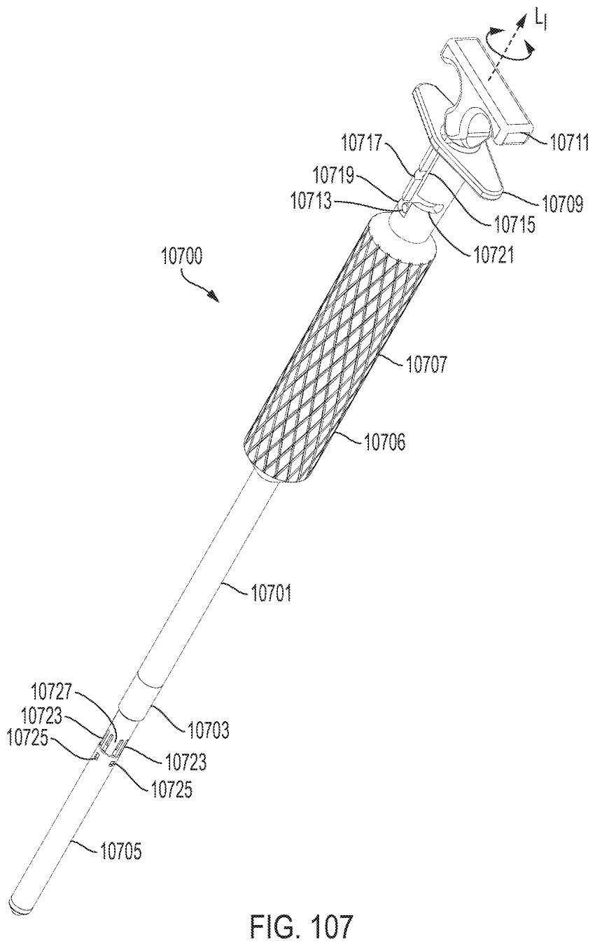

FIG. 107 shows illustrative inserter 10700. Inserter 10700 includes guide tube 10701. Inserter 10700 includes collar 10703. Collar 10703 is affixed to guide tube 10701. Implant 10705 is shown mounted on a distal end of inserter 10700.

As used herein, a “distal” portion of an apparatus generally means the end of the apparatus that is inserted or is to be inserted to into a patient, bone or other body. A “distal” portion of an apparatus may also refer to an end of the apparatus that is typically positioned most distant from a practitioner handling the apparatus. As used herein, a “proximal” portion of an apparatus generally means an end of an apparatus that is typically positioned closest to a practitioner handling the device. A “proximal” portion of an apparatus may also refer to an end of the apparatus that is typically positioned most distant from a patient, bone or other body being acted upon by the practitioner. For example, a direction extending from handle 10711 to implant 10705 will be referred to as a “distal direction.” The opposite direction, from implant 10705 to handle 10711, will be referred to as a “proximal direction.”

Collar 10703 includes retention fingers 10723 that are engaged with windows 10725 of implant 10705. Collar 10703 includes guide segment 10727.

Guide tube 10701 includes barrel 10706. Barrel 10706 includes grip pattern 10707. A practitioner may utilize grip pattern 10707 to hold barrel 10706. A practitioner may hold barrel 10706 while actuating handle 10711. Handle 10711 may be moved axially along longitudinal axis L_{I}. L_{I} may be a central longitudinal axis of inserter 10700. Handle 10711 is affixed to a shaft (not shown). The shaft is positioned within guide tube 10701. Movement of handle 10711 along L_{I} may shift a position of the shaft.

Dowel 10713 may protrude from the shaft. Moving handle 10711 along L_{I} moves dowel 10713 within guide channel 10715. Handle 10711 may be rotated about axis L_{I}. Handle 10711 may be rotated to shift a position of dowel 10713 into one of locking channels 10717, 10719 or 10721. Each of locking channels 10717, 10719 or 10721 may be utilized for a different size implant mounted on a distal end of inserter 10700.

For example, when dowel 10713 is positioned in locking channel 10719, the shaft positioned within guide tube 10701 may protrude a greater distance from a distal end of collar 10703 than when dowel 10713 is positioned in locking channel 10721. Locking channel 10719 may be utilized for a longer implant than an implant utilized in connection with locking channel 10721.

When dowel 10713 is positioned in locking channel 10721, the shaft affixed to handle 10711 (and positioned within guide tube 10701) may protrude a greater distance from a distal end of collar 10703 than when dowel 10713 is positioned in locking channel 10717. Locking channel 10721 may be used in connection with an implant that is longer than an implant used in connection with locking channel 10717.

Collar 10703 includes guide segment 10727. Guide segment 10727 may support a segment of the shaft that protrudes from a distal end of collar 10703.

Rotating dowel 10713 into a locking channel may fix a position of handle 10711 along L_{I}. Rotating dowel 10713 into a locking channel may fix a position of the shaft within guide tube 10701. Rotating dowel 10713 into a locking channel may secure implant 10705 to a distal end of inserter 10700.

Handle 10711 may also be actuated by a thumb or other part of a practitioner's hand. Flange 10709 may be held by fingers or one or more other parts of the same hand. Handle 10711 and flange 10709 may be positioned such that a practitioner may hold flange 10709 and actuate handle 10711 using “one handed” operation.

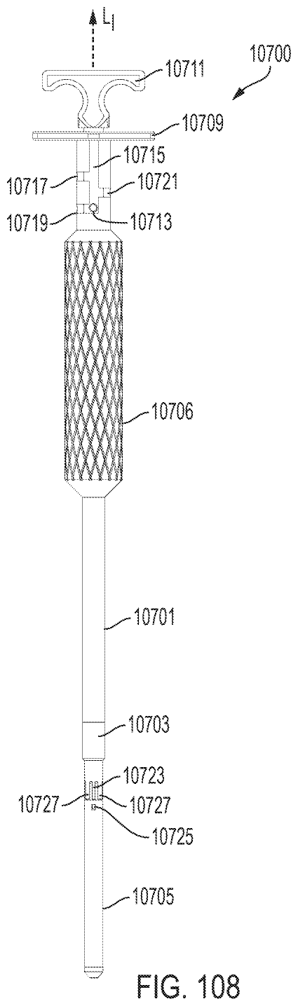

FIG. 108 shows another view of inserter 10700. FIG. 108 shows that locking channels 10719, 10717 and 10721 are at different positions along axis L_{I}. Handle 10711 may be rotated about axis L_{I} to position dowel 10713 in a desired locking channel.

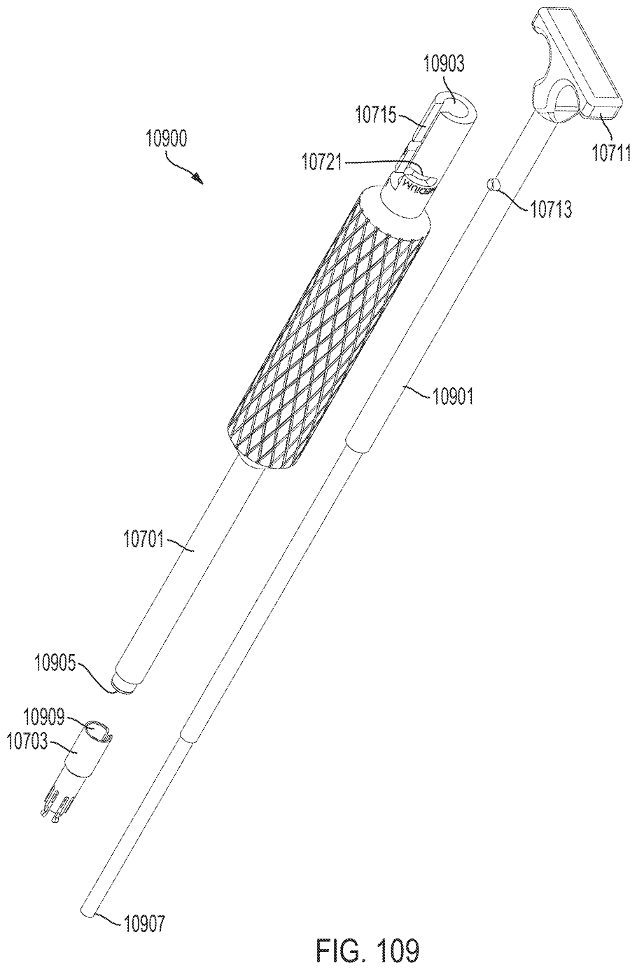

FIG. 109 shows an exploded view 10900 of components of inserter 10700. View 10900 shows guide tube 10701 and collar 10703. Guide tube 10701 includes recessed extension 10905. Recessed extension 10905 may be press-fit into collar 10703. Friction may hold collar 10703 to recessed extension 10905. Collar 10703 includes slot 10909. Slot 10909 may be configured to mate with a key (not shown) protruding from recessed extension 10905. Engagement of slot 10909 and the key may prevent collar 10703 from rotating, about axis L_{I}, with respect to guide tube 10701.

In some embodiments, collar 10703 and recessed extension 10905 may each be tapped. Recessed extension 10905 may include male threads. Collar 10703 may include female threads. Collar 10703 and recessed extension 10905 may be fixed to each other by threading recessed extension 10905 into collar 10703.

View 10900 shows shaft 10901. Shaft 10901 is affixed to handle 10711. Shaft 10901 may be fixed to handle 10711 using a press-fit. Shaft 10901 may be positioned inside guide tube 10701. Distal end 10907 may be inserted into cannula 10903 defined by guide tube 10701. Shaft 10901 may be seated in guide tube 10701 such that dowel 10713 is positioned in guide channel 10715. Shaft 10901 may have a length such that distal end 10907 protrudes distally beyond collar 10703 when dowel 10713 is positioned in any one of locking channels 10717, 10719 or 10721.

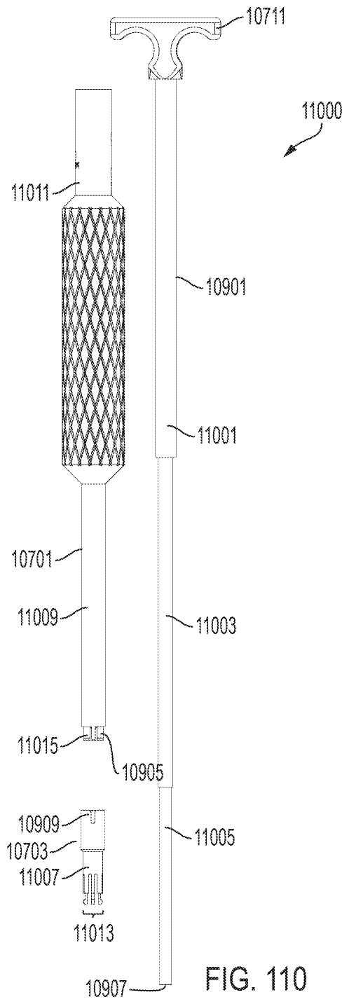

FIG. 110 shows illustrative view 11000 of components of inserter 10700. View 11000 shows that shaft 10901 includes segments 11001, 11003 and 11005. Shaft 10901 may be cylindrical. Segment 11001 may have a larger diameter than segment 11003. Segment 11003 may have a larger diameter than segment 11005. Segment 11001 may be configured to be seated within segment 11011 of guide tube 10701. Segment 11003 may be configured to be seated within segment 11009 of guide tube 10701. Segment 11005 may be configured to be seated within segment 11007 of collar 10703. In some embodiments, shaft 10901 may have a uniform diameter.

View 11000 shows that a distal end of retention fingers 10723 includes protrusions 11013. Protrusions 11013 may be configured to fit into windows 10725 of implant 10705. When dowel 10713 is positioned in a locking channel, distal end 10907 of shaft 10901 may protrude beyond protrusions 11013 and into an interior of implant 10705.

View 11000 shows key 11015. Key 11015 protrudes from a surface of recessed extension 10905. Key 11015 is configured to be seated in slot 10909 of collar 10703. When key 11015 is seated in slot 10909, collar 10703 and guide tube 10701 may be rotationally fixed, about axis L_{I}, with respect to each other.

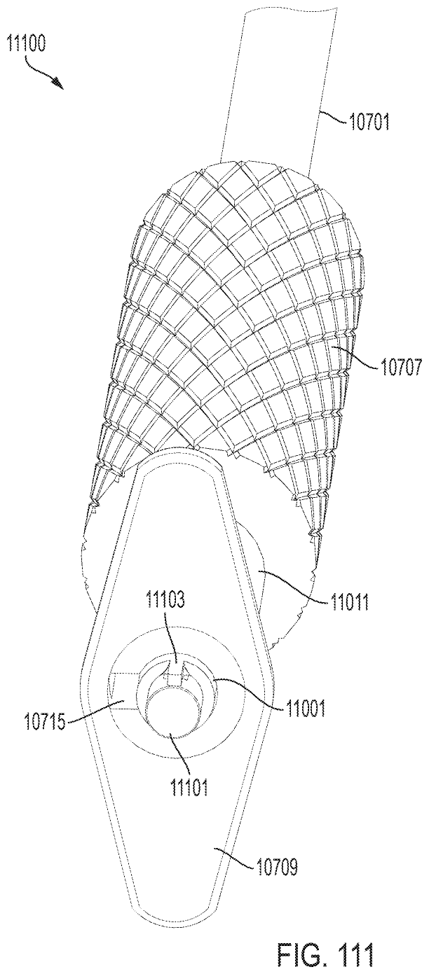

FIG. 111 shows illustrative view 11100 of components of inserter 10700. View 11100 shows shaft 11001 positioned within guide tube 10701. View 11100 shows shaft segment 10901 positioned within guide tube segment 11001. Shaft segment 11003 may be positioned within guide tube segment 11009. View 11100 shows a texture of grip pattern 10707. Grip pattern 10707 is illustrative and an exterior of guide tube 10701 may include any suitable grip pattern or no grip pattern.

View 11100 shows a proximal end of shaft 10901 protruding proximally beyond flange 10709. Proximal end 10901 includes recessed extension 11101. A diameter of recessed extension 11101 may be less than a diameter of shaft segment 11001. Recessed extension 11101 may be configured to fit within a cannula defined by handle 10711. Proximal end 10901 includes key 11103. Key 11103 may be configured to be seated in a slot defined by handle 10711.

View 11100 also shows guide channel 10715. Dowel 10713 may be configured to slide within guide channel 10715.

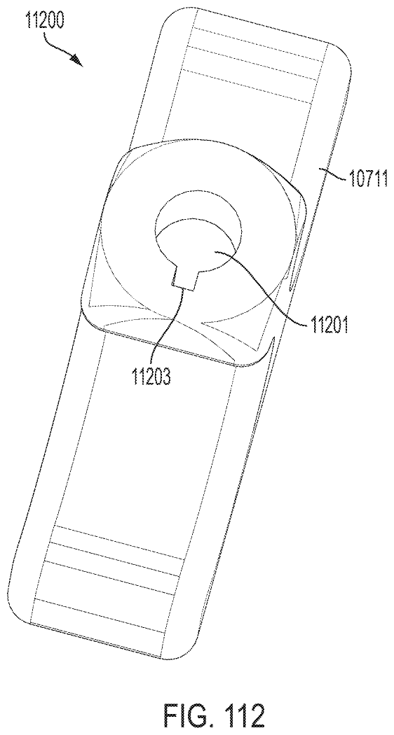

FIG. 112 shows illustrative view 11200 of handle 10711. View 11200 shows that handle 10701 includes cannula 11201. Cannula 11201 may be configured to receive recessed extension 11101 of shaft 10901. Recessed extension 11101 may be press-fit into cannula 11201. Friction may hold handle 10701 to recessed extension 11101.

View 11200 shows that handle 10711 includes slot 11203. Slot 11203 may be configured to receive key 11103. When key 11103 is seated in slot 11203, shaft 10901 and handle 10711 may be rotationally fixed, about axis L_{I}, with respect to each other. Rotation of handle 10711 may rotate shaft 10901. Rotation of handle 10711 may shift dowel 10713 in to a locking channel (e.g., 10717, 10719 or 10721).

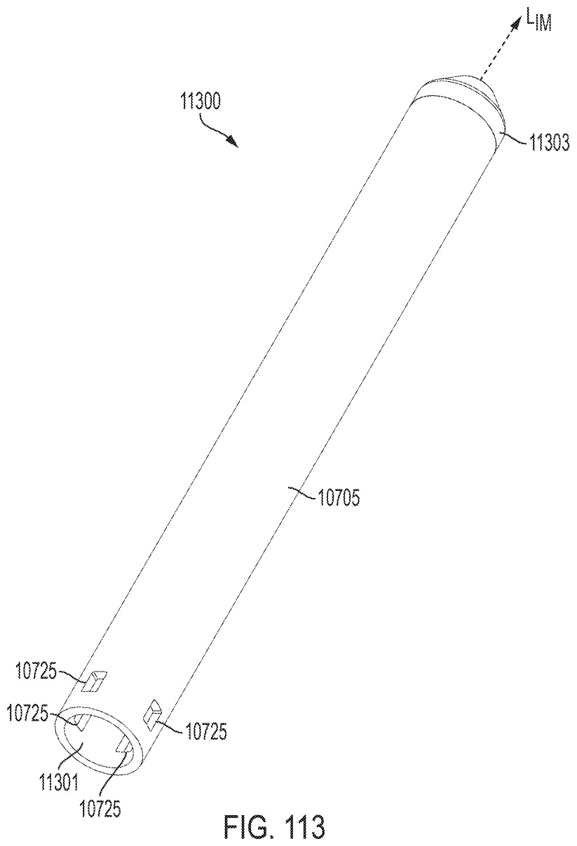

FIG. 113 shows illustrative view 11300 of implant 10705. Implant 10705 may be the implant. For example, implant 10705 may include the mesh, the hub, and the base.

View 11300 shows implant 10705 in a collapsed state. Implant 10705 may be configured to expand from a collapsed state into an expanded state. Implant 10705 may be configured to self-expand from a collapsed state into an expanded state. In such embodiments, a default state of implant 10705 may be an expanded state. It may be advantageous to insert implant 10705 into a bone in a collapsed state. For example, a smaller access hole in the bone is needed to insert implant 10705 into the bone in a collapsed state than in an expanded state.

View 11300 shows that implant 10705 includes windows 10725 in proximal base 11403. Windows 10725 may be configured to be engaged by protrusions 11013 on a distal end of retention fingers 10723. Retention fingers 10723 may fit into cannula 11301 defined by implant 10705. Cannula 11301 may have a smaller diameter than a diameter defined by protrusions 11013. Retention fingers 10723 may be configured to be compressed about axis L_{I} (shown in FIG. 107) as protrusions 11013 are inserted into cannula 11301. Retention fingers 10723 may be biased to push protrusions 11013 into windows 10725. When protrusions 11013 are seated in windows 10725, implant 10705 may be fixed to collar 10703 of inserter 10700.

When implant 10705 is fixed to inserter 10700, handle 10711 may be used to move shaft 10901 into cannula 11301. Shaft 10901 may be moved through an interior of implant 10705 such that distal end 10907 contacts or is positioned adjacent to hub 11303. When implant 10705 is in an expanded state and fixed to collar 10703 of inserter 10700, pushing distal end 10907 against hub 11303 may collapse implant 10705. Handle 10711 may be twisted to position dowel 10713 in a locking channel to lock implant 10705 in a collapsed state.

When mounted on inserter 10700, implant 10705 may be inserted into a bone in a collapsed state. After positioning implant 10705 inside the bone, handle 10711 may be twisted to move dowel 10713 out of a locking channel and into guide channel 10715. Handle 10711 may then be pulled axially along axis L_{I} to retract shaft segment 11001 out of guide tube segment 11011. Retracting shaft 10901 out of guide tube segment 11011 may allow hub 11303 to move distal end 10907. Retracting shaft segment 11001 out of guide tube segment 11011 may allow implant 10705 to expand.

Distal end 10907 may be retracted into collar 10703. Distal end 10907 may be retracted into collar 10703 such that retaining fingers 10723 may be compressed about axis L_{I} and protrusions 11013 released from windows 10725.

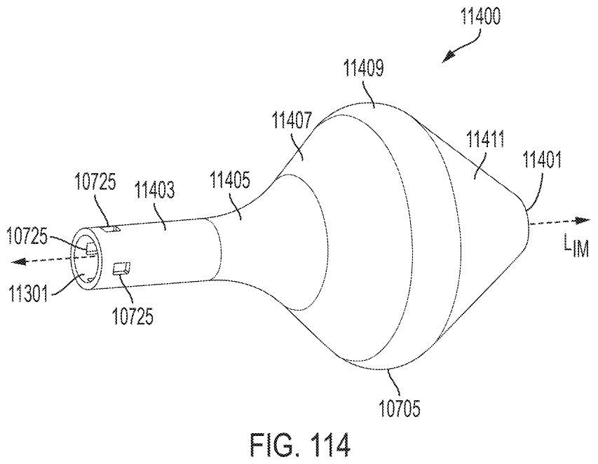

FIG. 114 shows implant 10705 in expanded state 11400. FIG. 114 also shows that implant 10705 includes an expandable implant head that includes expansion bands 11405, 11407, 11409 and 11411. Implant 10705 may include hub 11303 (not shown in FIG. 114). Embodiments of an implant head may include any suitable number of expansion bands. Each expansion band may be configured to expand to a maximum distance from axis L_{IM}. Each expansion band may be associated with a different maximum expansion distance. The maximum expansion distances may provide an implant head with a specific shape, when the implant head is expanded. The specific shape may be configured to provide a targeted clinical benefit. Each expansion band may correspond to a different laser-cut pattern in a tube, such as a nitinol tube. Two or more expansion bands may have the same laser-cut pattern.

When a practitioner desires to remove implant 10705 from the bone, implant 10705 may be in an expanded state, as shown in view 11400. It may be desirable to collapse implant 10705 before removal. Collapsing implant 10705 before removal may allow implant 10705 to be removed using a smaller access hole in the bone.

To collapse implant 10705, retaining fingers 10723 may be inserted into cannula 11301 and protrusions 11013 seated within windows 10725. Handle 10711 may be used to push distal end 10907 of shaft 10901 into cannula 11301 and against distal end 11401 of implant 10705. Because proximal base 11403 is fixed to collar 10703 (via retaining fingers 10723), pushing distal end 10907 against distal end 11401 and along axis L_{IM} collapses implant 10705. After implant 10705 is collapsed, dowel 10713 may be positioned in a locking channel to lock implant 10705 in the collapsed state. After locking implant 10705 in the collapsed state, a practitioner may grasp grip pattern 10707 to remove implant 10705 from the bone.

After implant 10705 is removed from the bone, shaft 10901 may be retracted into collar 10703 and retaining fingers 10723 released from windows 10725.

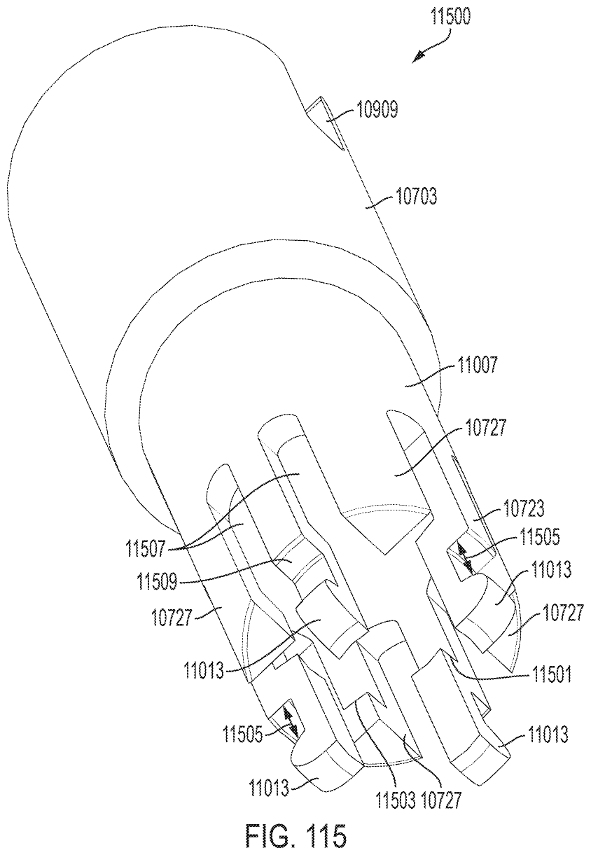

FIG. 115 shows illustrative view 11500 of collar 10703. View 11500 shows guide segments 10727. View 11500 shows retaining fingers 10723. View 11500 shows protrusions 11013 on a distal end of retaining fingers 10723.

View 11500 shows that retaining fingers 10723 include cut-out 11505. When protrusions 11013 are seated in windows 10725, a proximal portion of base 11403 may be seated in cut-out 11505. View 11500 shows that retaining fingers 10723 define outer edge 11509. Outer edge 11509 may prevent base 11403 from moving in a proximal direction along axis L_{I} when base 11403 is affixed to collar 10703.

View 11500 also shows that retaining fingers 10723 define inner edge 11501. View 11500 shows that guide segments 10727 define inner edge 11503. Inner edges 11501 and 11503 may be sized to allow shaft segment 11005 to pass through collar 10703 and distal end 10907 to extend distally beyond protrusions 11013. Inner edges 11501 and 11503 may be sized to restrain shaft segment 11003.

View 11500 shows that collar 10703 includes cut-outs 11507 between guide segments 10727 and retaining fingers 10723. Cut-outs 11507 may provide a “flex-zone” that allows retaining fingers 10723 to be compressed about axis L_{I}. Retaining fingers 10723 may be compressed about axis L_{I} to position protrusions 11013 in windows 10725.

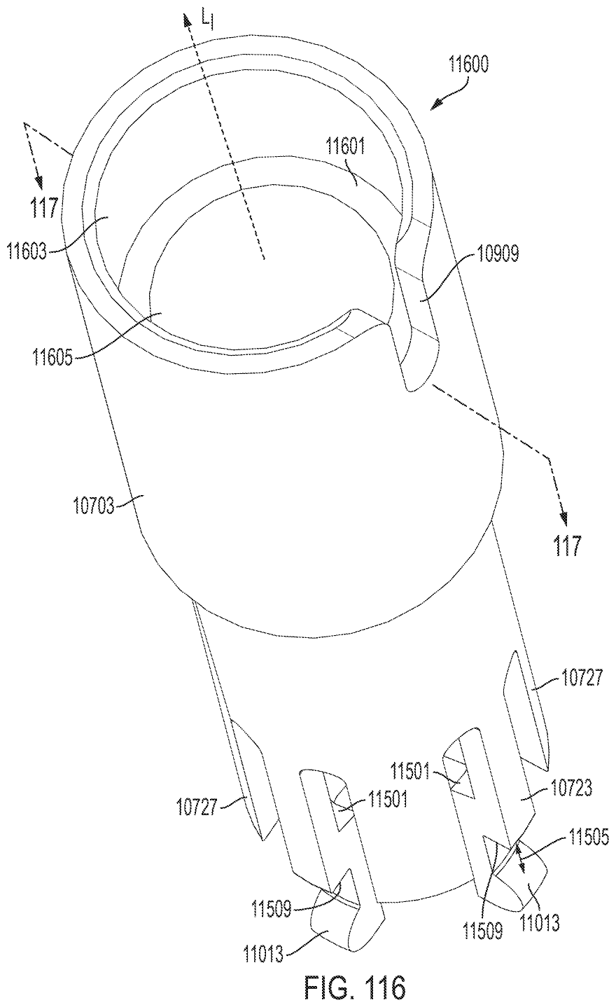

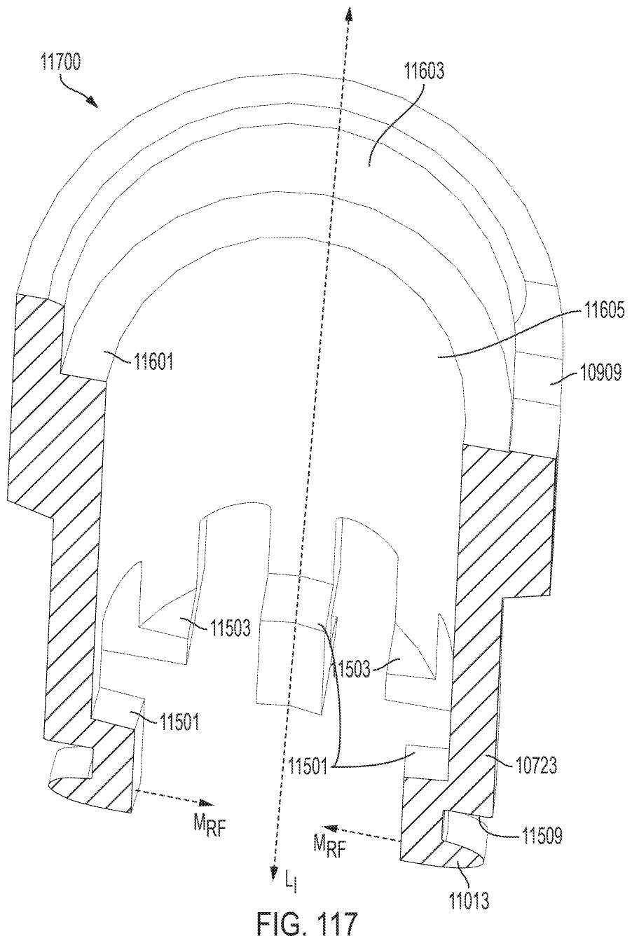

FIG. 116 shows illustrative view 11600 of collar 10703. View 11600 shows that collar 10703 includes collar segment 11603. Collar segment 11603 may be configured to receive recessed extension 10905 of guide tube 10701. Collar segment 11603 may be configured to form a press-fit with recessed extension 10905. Recessed extension 10905 may include key 11015 configured to be seated in slot 10909.

View 11600 shows that collar 10703 includes collar segment 11605. Collar segment 11605 may have a smaller diameter than segment 11603. Inner edge 11601 may be positioned along axis L_{I} between segment 11603 and segment 11605. A distal edge of recessed extension 10905 may be configured to be seated on inner edge 11601.

FIG. 117 shows illustrative cross-sectional view 11700. View 11700 is taken along lines 117-117 shown in FIG. 116. View 11700 shows retaining fingers 10723 is default position about axis L_{I}. View 11700 shows retaining fingers 10723 may be compressed about axis L_{I} in direction M_{RF}. Retaining fingers 10723 may be biased to return to the default position. Compressing retaining fingers 10723 about axis L_{I} may allow protrusions 11013 to be inserted into cannula 11301 (defined by proximal base 11403) and be seated in windows 10725.

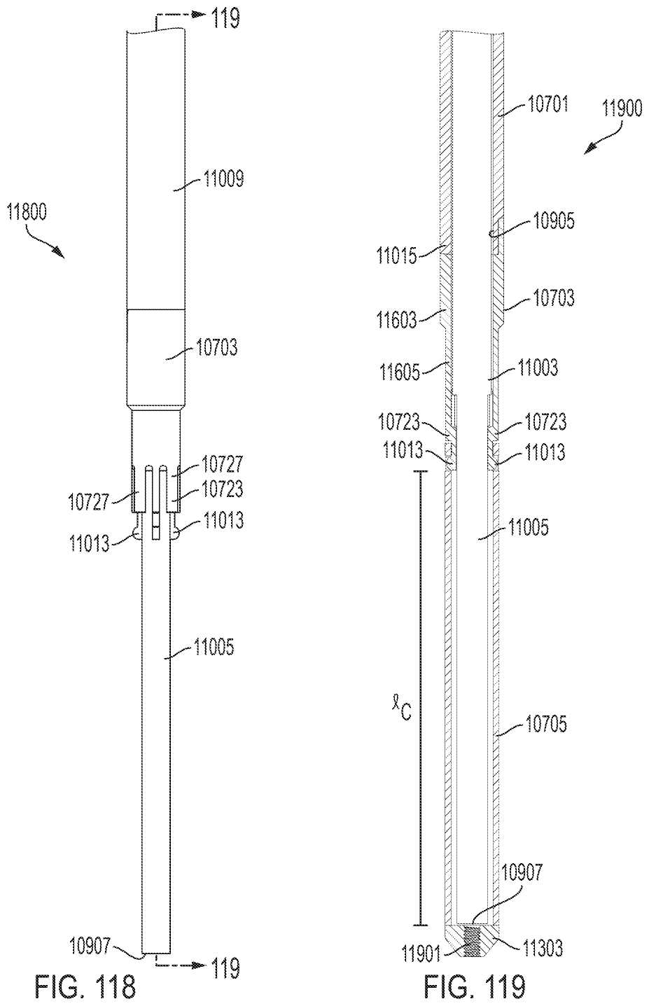

FIG. 118 shows illustrative view 11800. View 11800 shows shaft segment 11005 extending distally beyond protrusions 11013. View 11800 also shows that when shaft segment 11005 extends distally beyond protrusions 11013, shaft segment 11005 may prevent retaining fingers 10723 from compressing about axis L_{I}. When protrusions 11013 are seated in windows 10725 and shaft segment 11005 extends distally beyond protrusions 11013, implant 10705 may be locked to collar 10703.

FIG. 119 shows illustrative cross-sectional view 11900. View 11900 is taken along lines 119-119 shown in FIG. 118. Although not shown in FIG. 118, view 11900 includes a cross-section of implant 10705 when implant 10705 is affixed to collar 10705, as shown in FIG. 107.

View 11900 shows protrusions 11013 seated in windows 10725 of implant 10705. View 11900 shows shaft segment 11005 passing beyond protrusions 11013. View 11900 shows that shaft segment 11005 prevents retaining fingers 10723 from being compressed about axis L_{I} and disengaging protrusions 11013 from windows 10725. View 11900 shows that distal end 10907 is positioned such that distal hub 11303 of implant 10705 cannot move in a proximal direction. The position of distal end 10907 shown in view 11900 may be locked by positioning dowel 10713 in a locking channel. Locking a position of distal end 10907 shown in view 11900 may lock implant 10705 in a collapsed state. Implant 10705 may be inserted into a bone when locked in the collapsed state.

View 11900 shows guide tube 10701 connected to collar 10703. View 11900 shows recess extension 10905 positioned within collar segment 11603. View 11900 also shows key 11015 seated in slot 10909. View 11900 shows that when implant 10705 is collapsed, protrusions 11013 are spaced distance l_{C} apart from hub 11303. View 11900 shows that shaft segment 11005 extends along distance l_{C}. Distal end 10907 may keep hub 11303 spaced apart from protrusions 11013. Implant 10705 may be biased to self-expand and move hub 11303 closer to protrusions 11013.

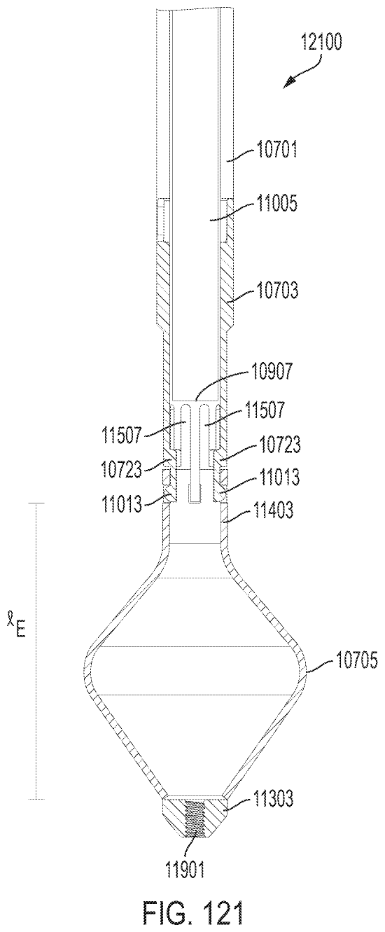

Shaft 10901 may be locked (e.g., by positioning dowel 10713 is a locking channel) to maintain a position of shaft segment 1105 shown in FIG. 119 and prevent hub 11303 from moving closer to protrusions 11013. Distance l_{C} is longer than distance l_{E} (shown in FIG. 121) spacing hub 11303 apart from protrusions 11013 when implant 10705 in an expanded state.

View 11900 shows that distal hub 11303 includes threads 11901. After implant 10705 is positioned in a bone and expanded, shaft segment 11005 may be unlocked and retracted into collar 10703. Shaft segment 11005 may be retracted into collar 10703 such that distal end 10907 is positioned proximal to cut-outs 11507. After shaft segment 11005 is retracted, retaining fingers 10723 may be compressed toward axis L_{I} and inserter 10700 pulled proximally to release protrusions 11013 from windows 10725.

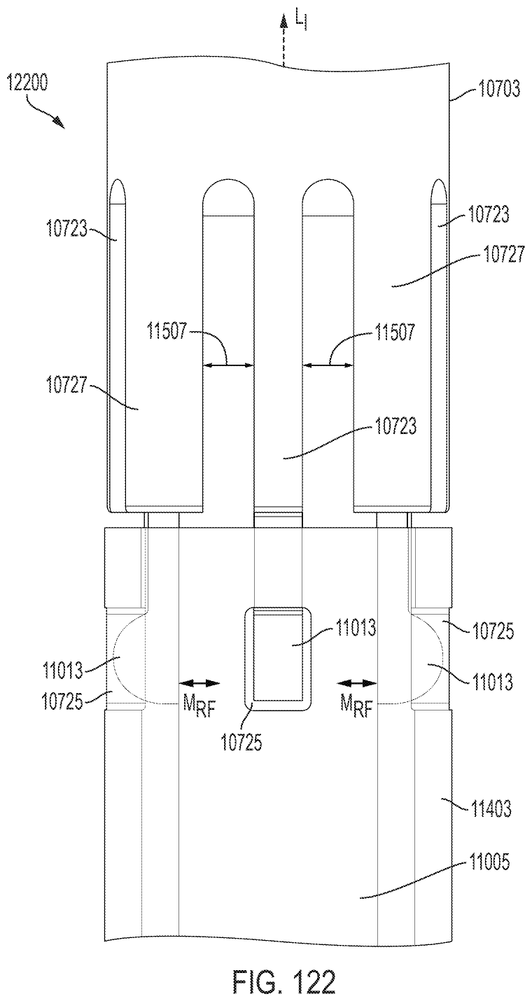

Protrusions 11013 include rounded edges (see FIG. 122) that allow protrusions 11013 to move out of windows 10725 and retaining fingers 10723 to be collapsed toward axis L_{I} when shaft segment 11005 has been retracted and inserter 10700 is pulled apart from implant 10705. After inserter 10700 is removed from implant 10705, locking components may be inserted into implant 10705. The locking components may be threaded into distal hub 11303. The locking components may lock implant 10705 in an expanded state inside the bone.

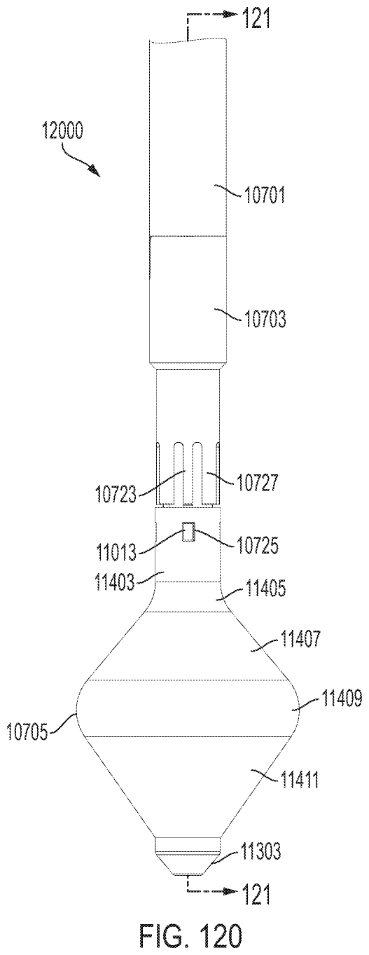

FIG. 120 shows illustrative view 12000. View 12000 shows implant 10705 in an expanded state. View 12000 shows implant 10705 affixed to collar 10703.

FIG. 121 shows cross-sectional view 12100 taken along lines 121-121 shown in FIG. 120. View 12100 shows that shaft 10901 has been retracted into collar 10703. View 12100 shows that shaft 10901 has been retracted into collar 10703 such that distal end 10907 is positioned proximal to cut-outs 11507. Cut-outs 11507 may provide a “flex-zone” that allows retaining fingers 10723 to be compressed about axis L_{I}.

When distal end 10907 is positioned proximal to cut-outs 11507, implant 10705 may expand. In some embodiments, movement of distal end 10907 may be used to control expansion of implant 10705. Distal end 10907 may be moved proximally at a rate that does not allow implant 10705 to fully expand. Implant 10705 may fully expand when distal end 10907 is positioned in proximal base 11403. However, implant 10705 may not be removed from collar 10703 unless distal end 10907 is positioned proximal to cut-outs 11507.

When distal end 10907 is positioned proximal to cut-outs 11507, then retaining fingers 10723 may be compressed toward axis L_{I} to move protrusions 11013 out of windows 10725. After protrusions are moved out of windows 10725, inserter 17000 may be removed from implant 10705.

View 12100 shows that when implant 10705 is expanded, protrusions 11013 are spaced distance l_{E} apart from hub 11303. View 12100 shows that distal end 10907 has been retracted proximally into collar 10703. Implant 10705 may be biased to self-expand and move hub 11303 closer to protrusions 11013. Because distal end 10907 is no longer preventing hub 11303 from moving toward protrusions 11013, implant 10705 expands. Distance l_{E} is shorter than distance l_{C} (shown in FIG. 119).

Shaft 10901 may be locked (e.g., by positioning dowel 10713 is a locking channel) to maintain a position of shaft segment 1105 shown in FIG. 119 and prevent hub 11303 from moving closer to protrusions 11013.

FIG. 122 shows illustrative view 12200. View 12200 shows proximal base 11403 of implant 10705 affixed to collar 10703. View 12200 shows proximal base 11403 in transparency so that retaining fingers 10723 and protrusions 11013 are visible through proximal base 11403. View 12200 shows that retaining fingers 10723 may be compressed about axis L_{I}. Compression about axis L_{I} may move protrusions 11013 out of windows 10725, allowing implant 10705 to be separated from collar 10703.

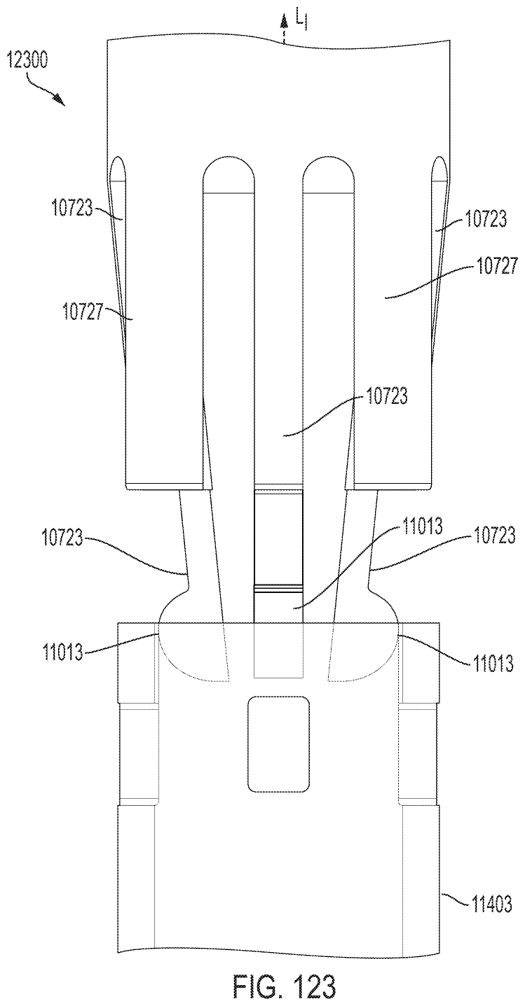

FIG. 123 shows illustrative view 12300. View 12300 shows retaining fingers 10723 being compressed about axis L_{I}. View 12300 shows that protrusions 11013 have been moved out of windows 10725. Retaining fingers 10723 may be compressed by moving implant 10705 distally, along axis L_{I} and relative to collar 10703. Retaining fingers 10723 may be compressed by moving guide tube 10703 proximally along axis L_{I} and relative to implant 10705.

FIGS. 122 and 123 shows that protrusions 11013 include rounded edges that allow protrusions to move out of windows 10725 and retaining fingers 10723 to be compressed toward axis L_{I} when shaft segment 11005 has been retracted and inserter 10700 is pulled apart from collar 10703. Protrusions 11013 include rounded edges that allow protrusions to move out of windows 10725 and retaining fingers 10723 to be compressed toward axis L_{I} when shaft segment 11005 has been retracted and implant 10705 is pulled apart from collar 10703. After implant 10705 is removed from collar 10705, retaining fingers 10723 may return to a default position. The default position may be a position of retaining fingers 10723 shown in FIGS. 116 and 122.

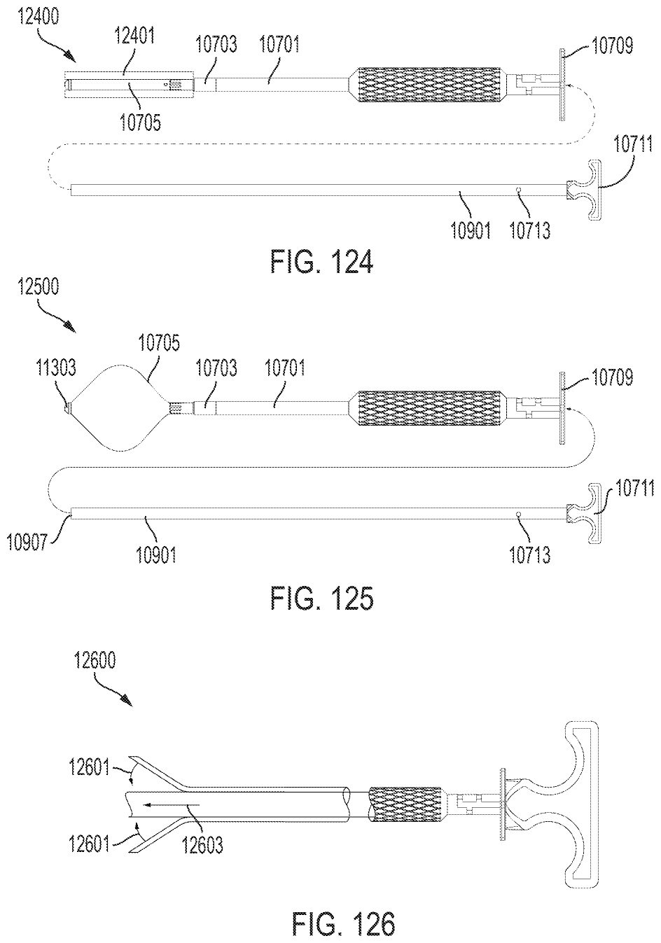

FIG. 124 shows steps in illustrative process 12400 for inserting implant 10705 into a bone. Process 12400 shows implant 10705 in a collapsed state. Process 12400 shows implant 10705 in containment tube 12401. Containment tube 12400 is shown in cross-section so that implant 10705 is visible inside containing tube 12400. Process 12400 shows that collar 10703 may be coupled to implant 10705 while implant 10705 is within containment tube 12401.

Collar 10703 may be coupled to implant 10705 by pushing retaining fingers 10723 into proximal base 11403. Pushing retaining fingers 10723 into proximal base 11403 may compress retaining fingers about axis L_{I}. Rounded edges of protrusions 11013 (shown, for example, in FIG. 122) may compress retaining fingers 10723 toward axis L_{I} as protrusions 11313 are inserted into proximal base 11403. Retaining fingers 10723 may be biased so that protrusions 11013 “snap” into windows 10725 when protrusions 11013 are provided relief by windows 10725.

Rounded edges of protrusions 11013 may ease removal of implant 10705 from retaining fingers 10723. Rounded edges of protrusions 11013 may allow protrusions 11013 to move out of windows 10725 and compress retaining fingers 10723 toward axis L_{I} as collar 10703 is pulled apart from implant 10705.

After implant 10705 is coupled to collar 10703, shaft 10901 may be inserted into guide tube 10701. Shaft 10901 may be inserted into guide tube 10701 such that dowel 10713 is positioned in guide channel 10715. Shaft 10901 may be moved distally until dowel 10713 is aligned with one of locking channels 10717, 10719 or 10721. Handle 10711 may be twisted to position dowel 10713 in a locking channel. After dowel is positioned in a locking channel, implant 10705 may be removed from containment tube 12401 and remain: (1) coupled to inserter 10700 and (2) in a collapsed state. Implant 10705 may then be inserted (in a collapsed state) into a bone.

FIG. 125 shows steps of illustrative process 12500 for removing implant 10705 from a bone. Process 12500 shows implant 10705 in an expanded state. Implant 10705 may be positioned in a bone. Collar 10703 may be coupled to implant 10705. Prior to coupling collar 10705 to implant 10705, implant 10705 may be unlocked by removing locking components from implant 10705. Illustrative locking components may include the support. Illustrative locking components may include the support and the fixation member. Illustrative locking components may include the support, the extension member and the fixation member.

Collar 10703 may be coupled to implant 10705 by pushing retaining fingers 10723 into proximal base 11403. Pushing retaining fingers 10723 into proximal base 11403 may compress retaining fingers about axis L_{I}. Rounded edges of protrusions 11013 (shown, for example, in FIG. 122) may compress retaining fingers 10723 toward axis L_{I} as protrusions 11313 are moved into proximal base 11403. Retaining fingers 10723 may be biased so that protrusions 11013 “snap” into windows 10725 when protrusions 11013 are provided relief by windows 10725.

After implant 10705 is coupled to collar 10703, shaft 10901 may be inserted into guide tube 10701. Shaft 10901 may be inserted into guide tube 10701 such that dowel 10713 is positioned in guide channel 10715. Shaft 10901 may be moved distally until dowel 10713 is aligned with one of locking channels 10717, 10719 or 10721. Moving shaft 10901 distally such that dowel 10713 is aligned with a locking channel may collapse implant 10705. To align dowel 10713 with a locking channel, distal end 10907 may push hub 11303 apart from collar 10703. Because a proximal end of implant 10705 is coupled to collar 10703, implant 10705 collapses as hub 11303 is pushed further apart from collar 10703.

Handle 10711 may be twisted to position dowel 10713 in a locking channel. After dowel is positioned in a locking channel, implant 10705 may be removed from the bone in a collapsed state.

FIG. 126 shows distal end 10907 of shaft 10901 pushing hub 11303. Arrows 12601 show that as shaft 10901 moves distally in direction 12603, distal end 10907 pushes hub 11303 in distal direction 12603 and implant 10705 collapses. A practitioner may continue to push distal end 10907 and hub 11303 distally until dowel 10713 is aligned with one of locking channels 10717, 10719 or 10721. The practitioner may twist handle 10711 to position dowel 10713 in a locking channel and lock implant 10705 in a collapsed state. Implant 10705 may be removed from a bone in the collapsed state.

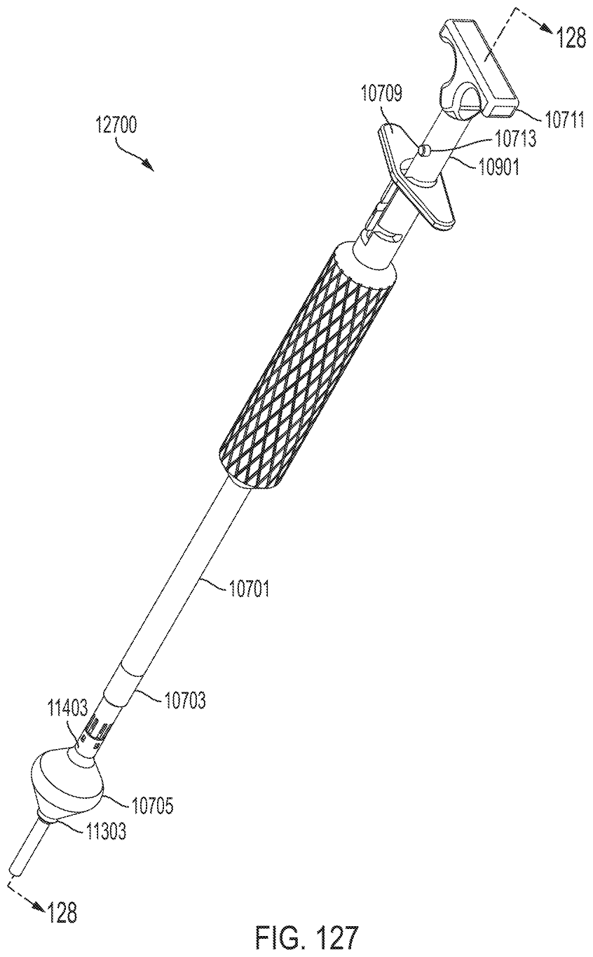

FIG. 127 shows illustrative view 12701. View 12701 shows shaft 10901 retracted out of guide tube 10701. View 12701 shows that implant 10705 is coupled to collar 10703. View 12700 shows implant 10705 in an expanded state. When shaft 10901 is retracted out of guide tube 10701, distal end 10907 may move proximally allowing distal hub 11303 and proximal base 11403 to move towards each other. Implant 10705 may self-expand when distal hub 11303 and proximal base 11403 move towards each other.

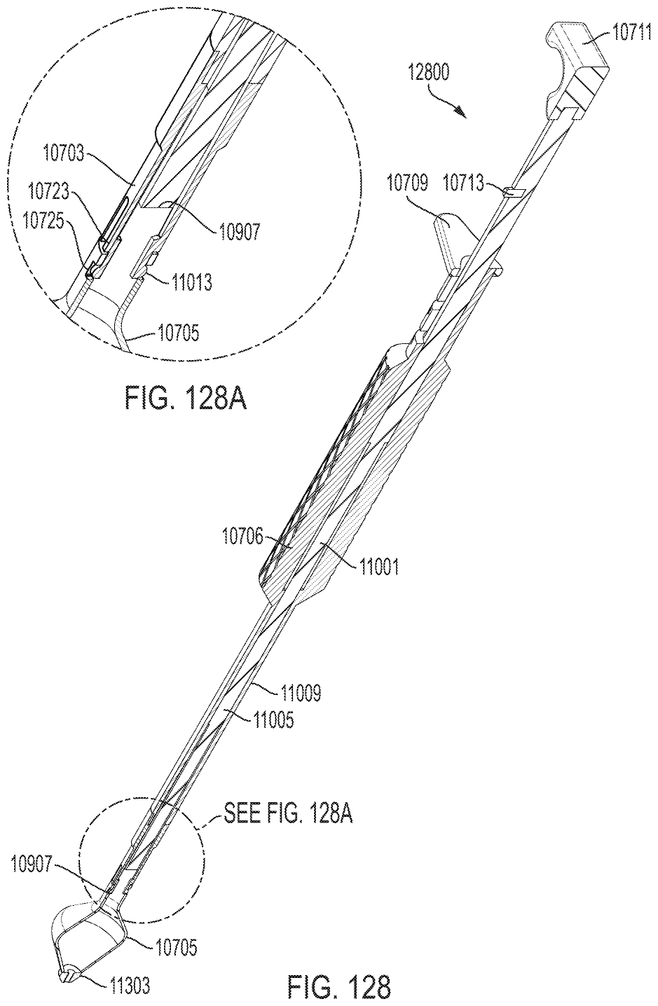

FIG. 128 shows illustrative cross-sectional view 12800. View 12800 is taken along lines 128-128 shown in FIG. 127.

View 12800 shows distal end 10907 of shaft 10901 in a retracted position (shifted proximally) inside collar 10703. View 12800 shows that implant 10705 may be in an expanded state when distal end 10907 is retracted into collar 10703. View 12800 also shows that dowel 10713 has been moved out of a locking channel to allow distal end 10907 to move proximally into collar 10703.

A practitioner may push distal end 10907 distally and contact hub 11303 to collapse implant 10705. A practitioner may manipulate distal end 10907 with one hand using flange 10709 and handle 107011. The practitioner may partially expand and/or partially collapse implant 10705 to position implant 10705 in a bone. Implant 10705 may be partially collapsed by pushing distal end 10907 against hub 11303 so that hub 11303 and protrusions 11313 are separated by a distance that is greater than l_{E} (shown in FIG. 121) and less than l_{C} (shown in FIG. 119).

FIG. 128A shows a close-up view of detail depicted in FIG. 128. FIG. 128A shows that distal end 10907 has been retracted into collar 10703 proximally past cut-outs 11507 (shown in FIG. 115) between guide segments 10727 and retaining fingers 10723. Cut-outs 11507 may provide a “flex-zone” that allows retaining fingers 10723 to be compressed about axis L_{I}. When distal end 10907 has been retracted into collar 10703 proximally past cut-outs 11507, retaining fingers 10723 may be compressed toward axis L_{I}, protrusions 11013 moved out of windows 10725 and implant 10705 removed from collar 10703.

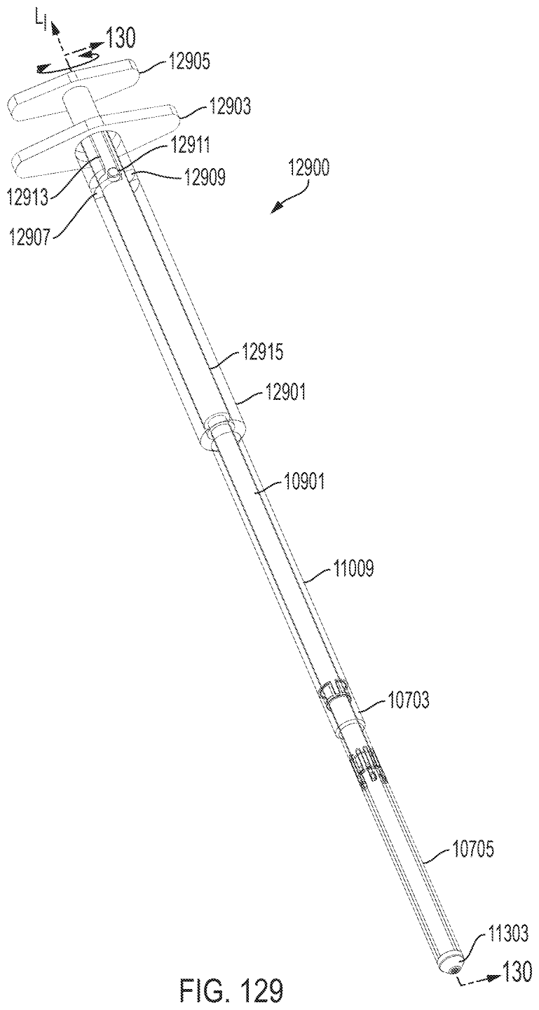

FIG. 129 shows illustrative inserter 12900. FIG. 129 shows inserter 12900 in transparency so that internal components of inserter 12900 are visible. Inserter 12900 may include one or more features of inserter 10700 (shown in FIG. 107). Inserter 12900 is shown coupled to implant 10705 via collar 10703. Implant 10705 is shown in a collapsed state.

Inserter 12900 includes guide tube segment 11009. Inserter 12900 includes guide tube segment 12901. Guide tube segment 12901 includes guide channel 12913. Inserter 12900 includes dowel 12911. A first end of dowel 12911 is shown protruding from a surface of shaft 12915. Dowel 12911 may penetrate shaft 12915 and a second end of dowel 12911 may protrude from an opposing surface (not shown) of shaft 12915. Guide tube channel 12913 may direct movement of the first end of dowel 12911 along axis L_{I}.

Guide tube segment 12901 may include another guide channel (not shown) on an opposing side (not shown) of guide tube segment 12901. The other guide channel may direct movement of the second end of dowel 12911 along axis L_{I}. Handle 12905 may be used to move shaft 12915 and dowel 12911 distally or proximally. For example, a practitioner may grip flange 12903 and actuate handle 12905 to move shaft 12915 and dowel 12911. A practitioner may grip flange 12903 and actuate handle 12905 with one hand.

Inserter 12900 includes locking channels 12907 and 12909. Twisting handle 12905 about axis L_{I} may move first end of dowel 12911 into locking channel 12907. Twisting handle 12905 about axis L_{I} may move second end of dowel 12911 into locking channel 12909. Twisting handle 12905 about axis L_{I} may simultaneously move first end of dowel 12911 into locking channel 12907 and second end of dowel 12911 into locking channel 12909.

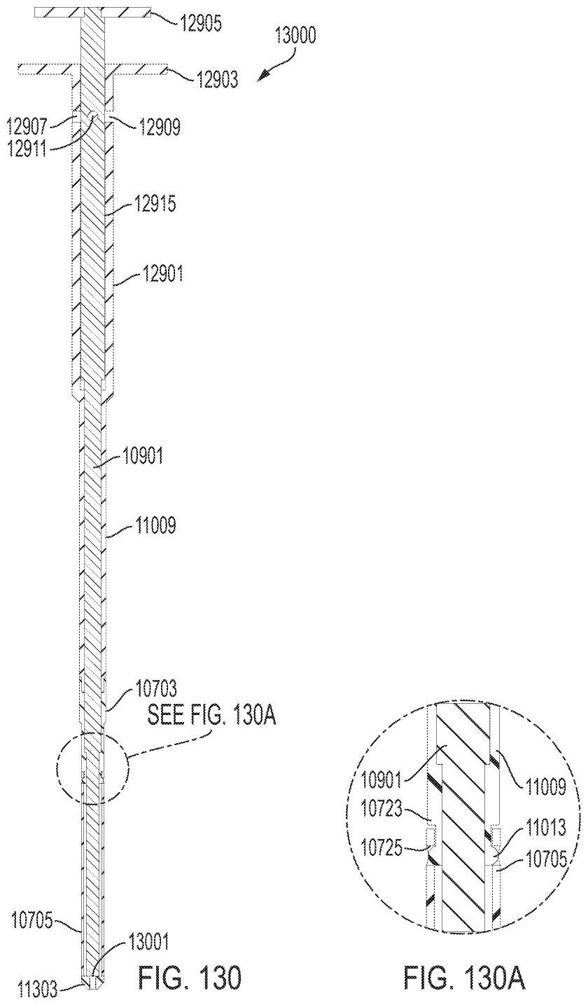

FIG. 130 shows illustrative cross-sectional view 13000 of inserter 12900. View 13000 is taken along lines 130-130 shown in FIG. 129. View 13000 shows that distal end 13001 of shaft 12915 extends distally beyond collar 10703 into implant 10705. View 13000 shows that distal end 13001 is in contact with hub 11303. Distal end 13001 may space hub 11303 apart from windows 10725 such that implant 10705 is in a collapsed state. Distal end 13001 may space hub 11303 apart from protrusions 11013 by distance l_{C} (shown in FIG. 119).

When implant 10705 is in a collapsed state, handle 12905 may be twisted to position first end of dowel 12911 in locking channel 12907 and second end of dowel 12911 in locking channel 12909. Positioning the ends of dowel 12911 in the locking channels may lock implant 10705 in a collapsed state. Implant 10705 may then be removed from a bone or inserted into a bone.

Shaft 10901 may be moved through an interior of implant 10705 such that distal end 10907 contacts or is positioned adjacent to hub 11303. When implant 10705 is in an expanded state and fixed to collar 10703 of inserter 10700, pushing distal end 10907 against hub 11303 may collapse implant 10705. Handle 10711 may be twisted to position dowel 10713 in a locking channel to lock implant 10705 in a collapsed state.

When mounted on inserter 10700, implant 10705 may be inserted into a bone in a collapsed state. After positioning implant 10705 inside the bone, handle 10711 may be twisted to move dowel 10713 out of a locking channel and into guide channel 10715. Handle 10711 may then be pulled axially along axis L_{I} to retract shaft segment 11001 out of guide tube segment 11011. Retracting shaft 10901 out of guide tube segment 11011 may allow hub 11303 to move distal end 10907. Retracting shaft segment 11001 out of guide tube segment 11011 may allow implant 10705 to expand.

Distal end 10907 may be retracted into collar 10703. Distal end 10907 may be retracted into collar 10703 such that retaining fingers 10723 may be compressed about axis L_{I} and protrusions 11013 released from windows 10725.

FIG. 130A shows a close-up view of detail depicted in FIG. 130. FIG. 130A shows that shaft 10901 extends distally past cut-outs 11507 (shown in FIG. 115) between guide segments 10727 and retaining fingers 10723. Cut-outs 11507 may provide a “flex-zone” that allows retaining fingers 10723 to be compressed about axis L_{I}. When shaft 10901 extends distally proximally past cut-outs 11507, retaining fingers 10723 may be prevented from compressed toward axis L_{I}, locking protrusions 11013 in windows 10725 and securing implant 10705 to collar 10703.

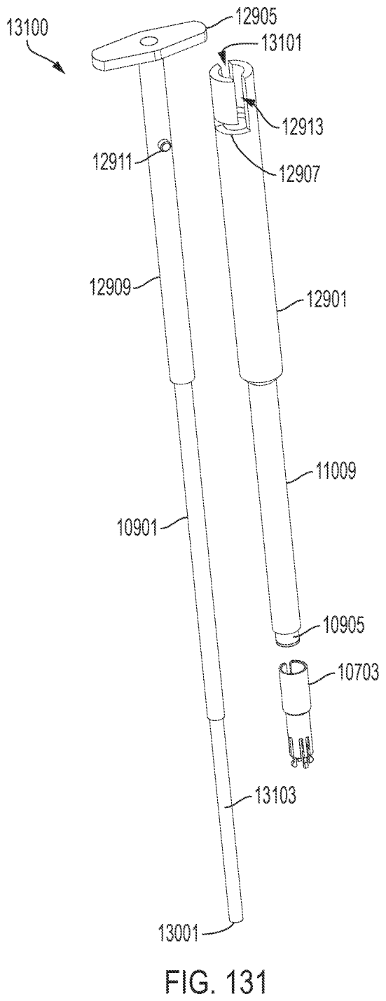

FIG. 131 shows exploded view 13100 of components of inserter 12900 (shown in FIG. 129). View 13100 shows collar 10703. Collar 10703 may be configured to mate with recessed extension 10905 extending from guide tube segment 11009. View 13000 shows that guide tube segment 12901 defines guide channels 12913 and 13101. Guide channel 12913 may be configured to guide a first end of dowel 12911 along axis L_{I}. Guide channel 13101 may be configured to guide a second end of dowel (not shown) along axis L_{I}.

Shaft 12915 may be configured to be separable from guide tube segments 12901 and 11009. Such embodiments may allow components of inserter 12900 to be easily cleaned and sterilized after or before use. Ends of dowel 12911 may be removed from a proximal end of guide channels 12913 and 13101.

In some embodiments, guide tube segment 12901 may include a detent (not shown) that catches an end of dowel 12911 and prevents shaft 12909 from being separated from guide tube segment 12901. Flange 12903 may be configured to provide such a detent. Such embodiments may be beneficial where inserter 12900 is disposable.

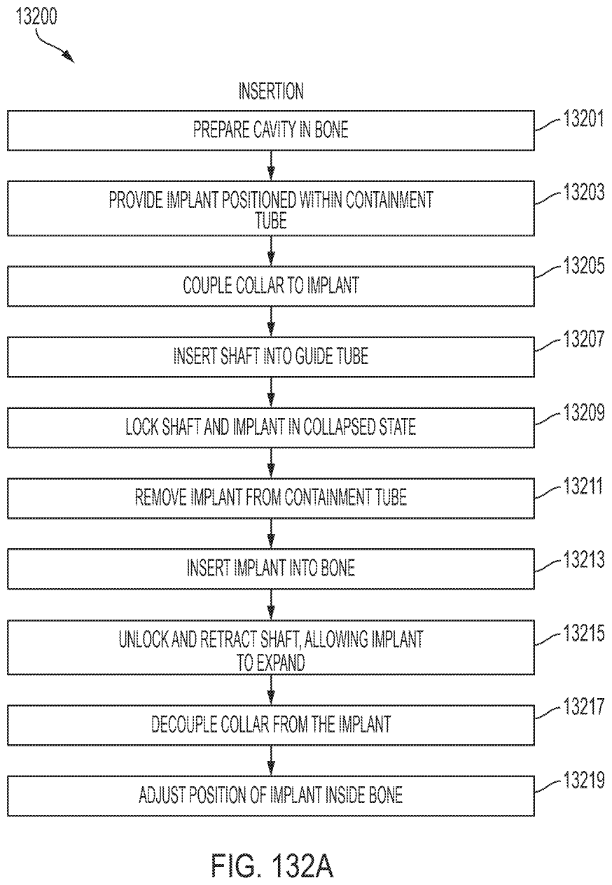

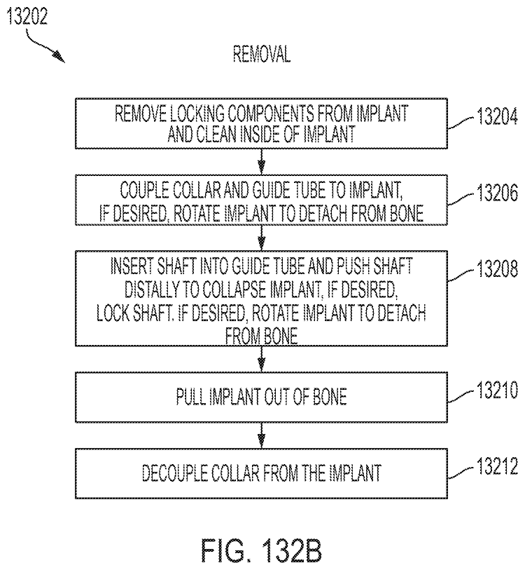

FIG. 132A shows illustrative process 13200 for inserting an implant into a bone. Process 13200 begins at step 13201. At step 13201, a practitioner prepares a cavity in a bone for receiving the implant. At step 13203, the practitioner is provided an implant within a containment tube. The containment tube may hold the implant in a collapsed state.

At step 13205, the practitioner couples an inserter device to the implant. The practitioner may couple a collar of the inserter to a proximal base of an implant. The collar may be coupled to the implant by pushing the collar into a base of the implant. Pushing the collar into the base compresses retaining fingers of the collar and positions protrusions in windows of the base.

The implant may include a self-expanding implant head. The implant may be provided in a containment tube that holds the implant in a collapsed state. In some embodiments, the inserter may be provided to the practitioner pre-coupled to a base of the implant.

At step 13207, the practitioner inserts a shaft into a guide tube of the inserter. The practitioner may insert the shaft into the guide tube such that a dowel protruding from a surface of the shaft is positioned in a guide channel defined by the guide tube. The practitioner may insert the shaft into the guide tube until the dowel is aligned with a locking channel defined by the guide tube.

At step 13209, the practitioner may twist the shaft to position the dowel in a locking channel. By positioning the dowel in the locking channel, the practitioner locks a position of the shaft relative to the collar. By positioning the dowel in the locking channel, the practitioner locks a position of the shaft such that a distal hub of the implant is locked at a fixed distance from a proximal base of the implant. By locking the distance between the distal hub and proximal base, the implant is locked in the collapsed state.

At step 13211, the practitioner removes the implant from the containment tube. Because the shaft is locked, when the implant is removed from the containment tube, the implant remains in the collapsed state.

At step 13213, the practitioner inserts the implant, in the collapsed state, into the bone. At step 13213, the practitioner may also adjust an orientation of the implant inside the bone by manipulating the inserter, which may still be coupled to the implant.

At step 13215, the practitioner unlocks the shaft by moving the dowel out of the locking channel. After the shaft is unlocked, the practitioner may retract the shaft into the collar. The practitioner may control a speed at which the shaft is retracted into the collar. Controlling the speed at which the shaft is retracted may control a rate at which the implant expands inside the bone. The practitioner may allow self-expansion of the implant to control a rate at which the shaft is retracted into the collar.

At step 13217, after the implant is expanded inside the bone, the practitioner decouples the collar from the implant. The collar may be decoupled from the implant by pulling the collar away from the implant. Pulling the collar away from the implant compresses retaining fingers of the collar and releases protrusions from windows in a base of the implant.

After decoupling the collar from the implant, at step 13219, the practitioner may adjust a position of the implant inside the bone using locking components of the implant. For example, the practitioner may use the support, or the extension member to further expand the implant as described herein. Further expanding the implant may draw the hub of the implant towards the base.

FIG. 132B shows illustrative method steps 13202 for removing an implant from a bone. Process 13202 begins at step 13204. At step 13204, a practitioner removes locking components from implant positioned inside the bone. After removing the locking components, the practitioner may clean out, or break up any matter, such as bone ingrowth, that may have accumulated inside the implant using the broaching tool. The broaching tool may be used to broach, clean out, or break up, the matter.

At step 13206, the practitioner couples an inserter to the implant. The practitioner may couple a collar of the inserter to a proximal base of the implant. The collar may be coupled to the implant by pushing the collar into the base of the implant. Pushing the collar into the base compresses retaining fingers of the collar and positions protrusions in windows of the implant.

If desired, after the inserter has been coupled to the implant, the practitioner may rotate the implant, while in the expanded state, inside the bone. Rotating the expanded implant may detach the implant head, or other implant component, from the bone.

At step 13208, the practitioner inserts a shaft into a guide tube of the inserter. The practitioner may insert the shaft into the guide tube such that a dowel protruding from a surface of the shaft is positioned in a guide channel defined by the guide tube. The practitioner may push the shaft further into the guide tube until a distal end of the shaft contacts a distal hub of the implant. The practitioner may continue to push the shaft against the hub, moving the distal hub further apart from the proximal base and the collar. The practitioner may squeeze the shaft by holding a flange and handle of the inserter with one hand.

The practitioner may push the shaft distally, moving the distal hub further apart from the proximal base, until the implant is in a collapsed state. If desired, after the implant has been collapsed, the practitioner may lock a position of the shaft such that the implant is locked in the collapsed state. The practitioner may continue to squeeze the flange and handle of the inserter to maintain the implant in the collapsed state without locking the shaft.

If desired, after the implant has been collapsed, the practitioner may rotate the implant inside the bone. The implant may be rotated by twisting the inserter. Rotating the implant may detach the distal hub, or other implant component, from the bone.

At step 13210, the practitioner pulls the inserter in a proximal direction to remove the implant from the bone. At step 13212, if the implant or inserter is to be reused, the practitioner may decouple the implant from the inserter. The implant may be decoupled from the inserter by retracting the shaft and pulling the implant apart from the collar. Pulling the implant apart from the collar compresses retaining fingers of the collar and moves protrusions out of windows of the implant.

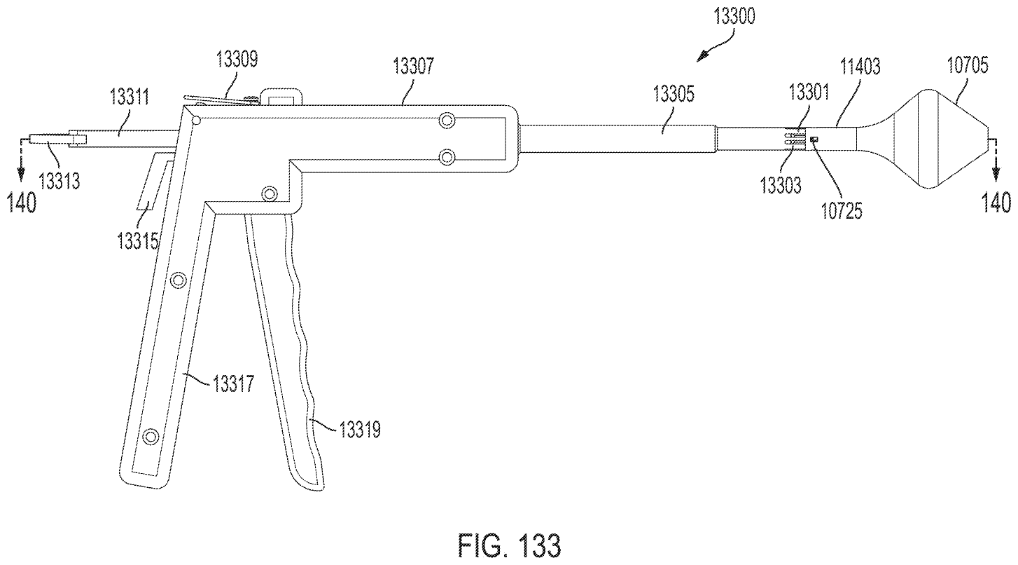

FIG. 133 shows an illustrative “pistol grip” inserter 13300. Inserter 13300 may be used to insert implant 10705 into a bone. A base 11403 of implant 10705 may be held in position by retaining fingers 13303 of inserter 13300. Squeezing trigger 13319 of inserter 13300 may move inserter shaft 13311, and support pusher 13511 mounted at an end of inserter shaft 13311, distally out of a guide tube 13305.

Implant 10705 is shown in FIG. 133 in an expanded state. Implant 10705 may be provided to a practitioner in a collapsed state. For example, implant 10705 may be self-expanding and may be provided to the practitioner in containment tube 12401 (shown in FIG. 124) that maintains the implant 10705 in the collapsed state. When implant 10705 is in the collapsed state a distal end of the implant 10705 (e.g., hub 11303 shown in FIG. 113) may be spaced a first distance apart from base 11403. Moving inserter shaft 13311 out of guide tube 13305 may position support pusher 13511 against a support (e.g., 208, 2203 or 15203) of implant 10705 and maintain the first distance as implant 10705 is withdrawn from containment tube 12401.

A flat spring 13309 may push pawl 13402 into locking grooves in shaft 13311. Positioning pawl 13402 in a groove may lock a position of the shaft 13311 relative to the barrel 13307 and guide tube 13305. Teeth that define each groove in shaft 13311 may allow the shaft 13311 to move past pawl 13402 when the trigger 13319 is squeezed. The teeth and flat spring 13309 may not allow pawl 13402 to move out of a groove when the trigger 13319 is released.

Pawl 13402 may be moved out of a groove by actuating the shaft lock release 13315. After pawl 13402 is disengaged from a groove, a practitioner may grasp handle 13313 and retract shaft 13311 into guide tube 13305. Retraction of the shaft 13311 may allow base 11403 of the implant to move closer to a distal end of implant 10705, and allow the implant 10705 to self-expand.

Continued retraction of the shaft 13311 into guide tube 13305 may position cut-out 13801 of the pusher support 13511 against retaining fingers 13303. Cut-outs 13801 of the pusher support 13511 are configured to compress retaining fingers 13303 toward central longitudinal axis L_{I} of inserter shaft 13311 and release retaining fingers 13303 from windows 10725 in base 11403 of implant 10705. After retaining fingers 13303 are released from base 11403, inserter 13300 may be withdrawn from implant 10705. After inserter 13300 is withdrawn, implant 10705 may be in an expanded state (inside or outside a bone).

The implant as illustrated and described herein may be coupled to the “pistol grip” inserter 13300.

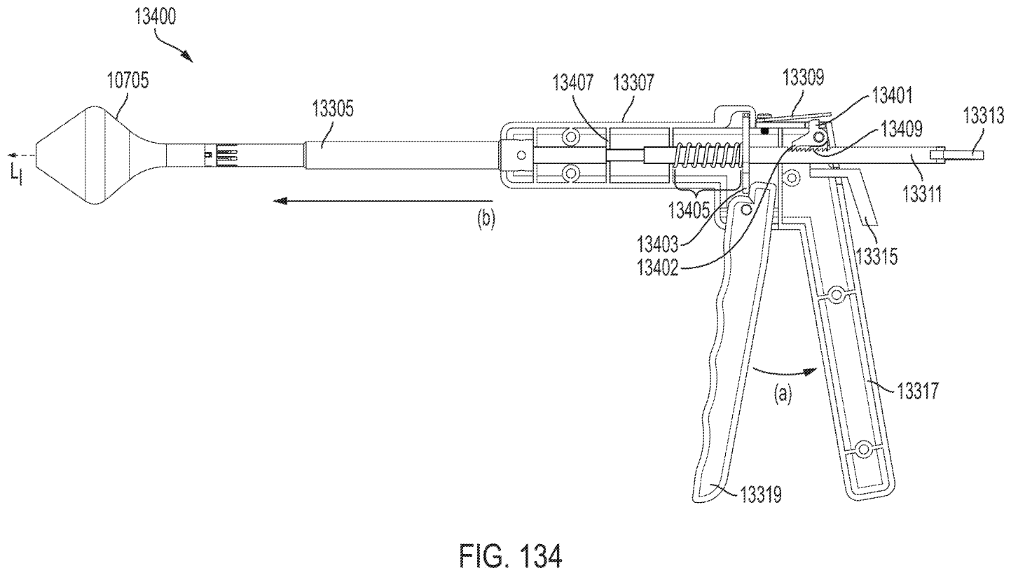

FIG. 134 shows an inside of barrel 13307. Spring 13405 is positioned to push the grip plate 13403 against trigger 13319 and space trigger 13319 apart from handle 13317. Grip plate 13403 includes a pass-through hole (not shown) for shaft 13311 to pass through grip plate 13403.

Squeezing the trigger 13319 in direction (a) shifts a position of grip plate 13403. The shifting may include rotating grip plate 13403. Grip plate 13403 may be rotated about the pass-through hole. Squeezing trigger 13319 may position grip plate 13403 at an oblique angle to central longitudinal axis L_{I} of inserter shaft 13311. When a position of the grip plate 13403 shifts, the edges of the pass-through hole in the grip plate 13403 may form a friction fit with an outer surface of shaft 13311. The friction fit may move the inserter shaft 13311 in direction (b) out of guide tube 13305 and into an interior of implant 10705 when the trigger 13319 is squeezed.

FIG. 134 also shows pawl 13402 of the shaft lock 13401 engaged with a groove between teeth 13409 of shaft 13311. Teeth 13409 of shaft 13311 may be configured such that as inserter shaft 13311 moves out of guide tube 13305, flat spring 13309 pushes pawl 13402 into an adjacent groove. Teeth 13409 may be configured such that pawl 13402 and shaft 13311 are locked in a position that prevents proximal retraction of the shaft 13311 into guide tube 13305 when trigger 13319 is released. Actuating shaft lock 13401 may move pawl 13402 out of teeth 13409 and allow shaft 13311 to be proximally retracted back into guide tube 13305.

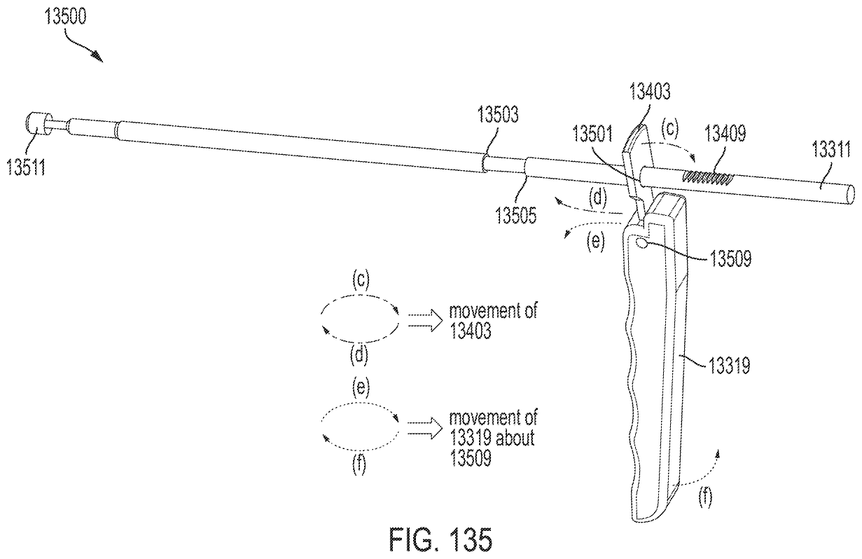

FIG. 135 shows view 13500 of components of inserter 13300. View 13500 shows shaft 13311. Pusher 13511 is affixed to a distal end of shaft 13311. Shaft 13311 is shown passing through grip plate 13403. View 13500 shows that when trigger 13319 is rotated about pivot point 13509 (movements (e) and (f)), grip plate 13403 is rotated (movements (c) and (d)). When grip plate 13403 is rotated, edge 13501 (a perimeter of pass-through hole in grip plate 13403) may form a friction fit with an outer surface of shaft 13311. The friction fit may move inserter shaft 13311 axially and move pusher 13511 into an interior of an implant.

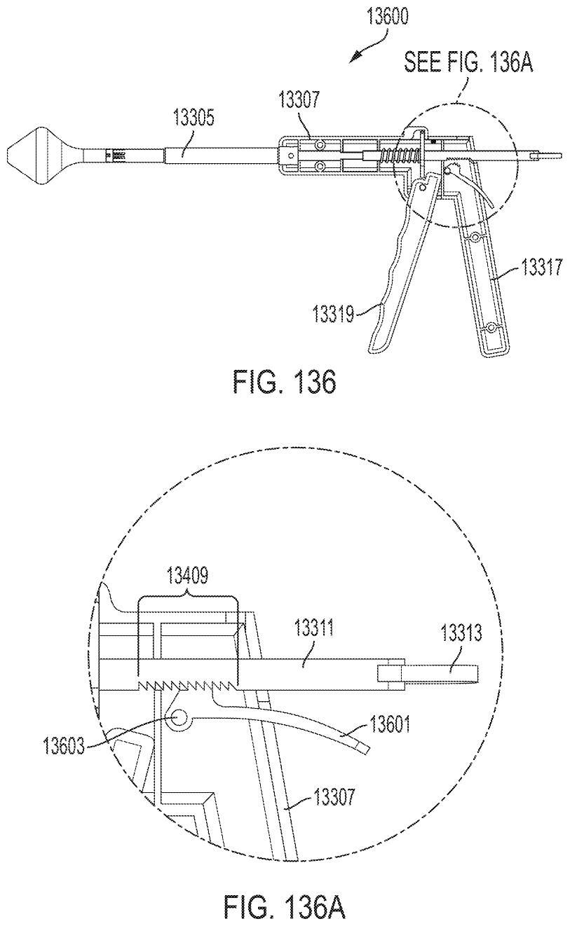

FIG. 136 shows illustrative inserter 13600. Inserter 13600 includes components shown in connection with inserter 13300 (shown in FIG. 133). Inserter 13600 shows an alternative configuration for locking components 136A.

FIG. 136A shows an illustrative configuration for locking components that is different from a configuration shown in FIG. 133. FIG. 136A shows shaft lock 13601 engaged with grooves 13409 in shaft 13311. FIG. 136 shows shaft lock 13601 disengaged from grooves 13409. When shaft lock 13601 is disengaged, shaft 13311 may be moved proximally relative to guide tube 13305 by pulling on handle 13313. When shaft lock 13601 is disengaged, shaft 13311 may be moved distally, relative to guide tube 13305, by squeezing trigger 13319. Shaft lock 13601 may be actuated from an engaged position (as shown in FIG. 136A) to a disengaged position (as shown in FIG. 136) by rotation about pivot point 13603. Shaft lock 13601 may be biased (e.g., by a spring) to engage grooves 13409.

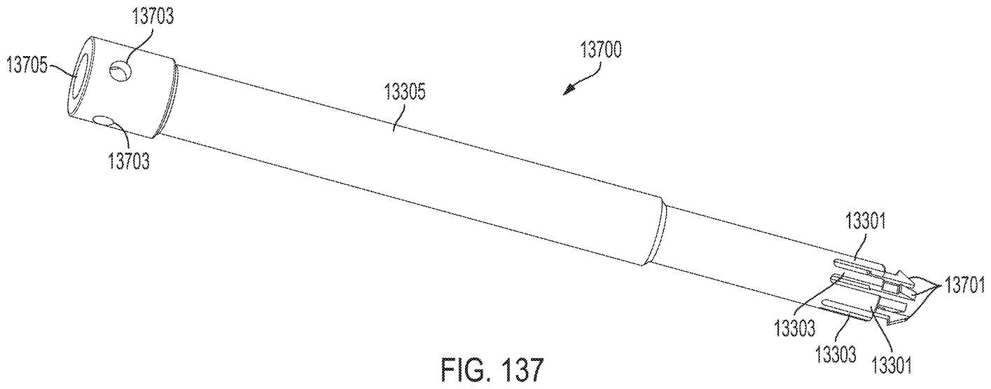

FIG. 137 shows an illustrative guide tube 13305. Guide tube 13305 includes retaining fingers 13303. Each of retaining fingers includes a protrusion 13701. Protrusions 13701 may be configured to engage windows 10725 in implant base 11403. When protrusion 13701 are engaged with windows 10725, retaining fingers 13303 may hold base 11403 in a fixed position relative to guide tube 13305 as shaft 13311 is moved relative to guide tube 13305. Guide tube 13305 may include guide segments 13301. Guide segments 13301 may guide shaft 13311 as it moves relative to guide tube 13305.

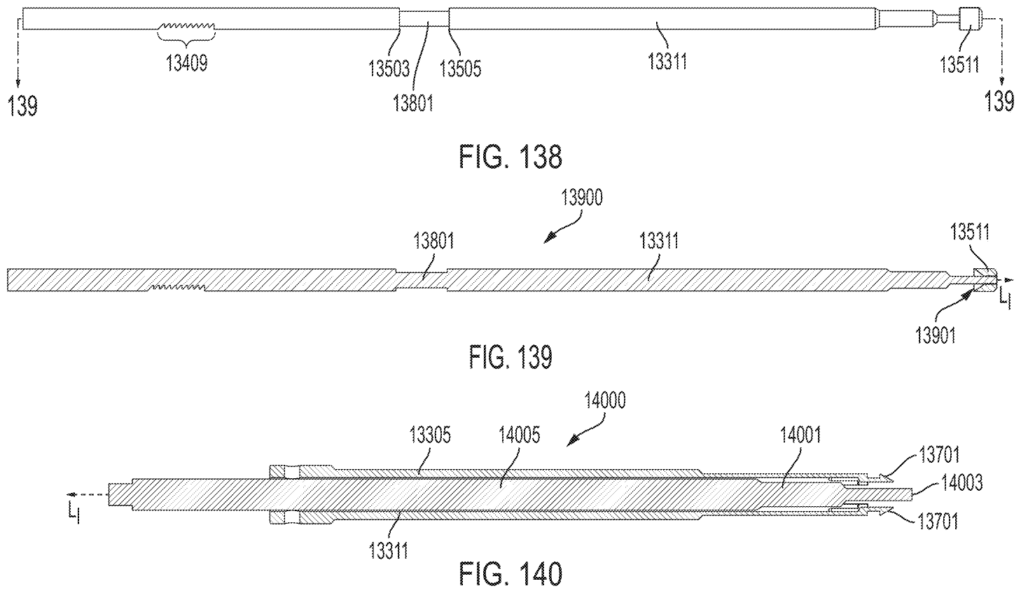

FIG. 138 shows illustrative shaft 13311. Inserter shaft 13311 may include cut-out 13801. Cut-out 13801 defines shoulders 13503 and 13505. Barrel wall 13407 may act as detent, engaging shoulders 13503 or 13505 to limit a maximum travel distance shaft 13311 may move relative to guide tube 13305. For example, shoulder 13505 may not pass through internal barrel wall 13407.

Internal barrel wall 13407 may limit the distance the inserter shaft 13311 extends distally out of guide tube 13305 and thereby prevents a practitioner from applying excessive tension (e.g., “over-collapsing”) between the base 11403 and a distal end of implant 10705.

Shoulder 13503 may not pass through the internal barrel wall 13407. Interaction of internal barrel wall 13407 and shoulder 13503 may prevent shaft 13311 from being removed from a proximal end of barrel 13307 when a practitioner releases the shaft lock 13401 and pulls on handle 11313.

FIG. 139 shows cross-sectional view 13900 taken along lines 139-139 shown in FIG. 138. View 13900 shows that pusher 13511 includes cut-out 13901. Shaft 13311 may be retracted proximally into guide tube 13700 such that cut-out 13901 bend distal tips of retaining fingers 13303 toward axis L_{I}, compressing protrusions 13701 toward axis L_{I}. When protrusions 13701 are compressed toward axis L_{I}, protrusions 13701 are disengaged from windows 10725 of implant 10705, allowing implant 10705 to be decoupled from guide tube 13305.

FIG. 140 shows an illustrative cross-sectional view 14000 of shaft 13311 positioned in guide tube 13305. View 14000 shows that shaft 13311 includes shaft segments 14005, 14001 and 14003. Shaft segment 14005 may stabilize shaft 13311 within guide tube 13305. Shaft segment 14001 may be configured to be shifted distally and prevent retaining fingers 13701 from bending toward axis L_{I}. Shaft segment 14003 may be affixed to a pusher, such as pusher 13511. As shaft 13311 is shifted distally beyond retaining fingers 13701, pusher 13511 or a distal end of shaft segment 14003 may push a hub of an implant further apart from a base of the implant coupled to retaining fingers 13701. Increasing a distance between a base and hub of an implant may collapse the implant.

After the implant is collapsed, it may be inserted into a bone. Retracting shaft 13311 distally into guide tube 13305 may allow the base and hub to move closer to each other, allowing the implant to self-expand inside the bone. In embodiments that include pusher 13511, retraction of shaft segment 14003 may release retaining fingers 13701 from windows 10725.

To remove an implant from a bone, retaining fingers 13701 may be coupled to the base of the implant. Shaft 13311 may be moved distally to collapse the implant inside the bone. After the implant is in a collapsed state, the implant may be removed from the bone.

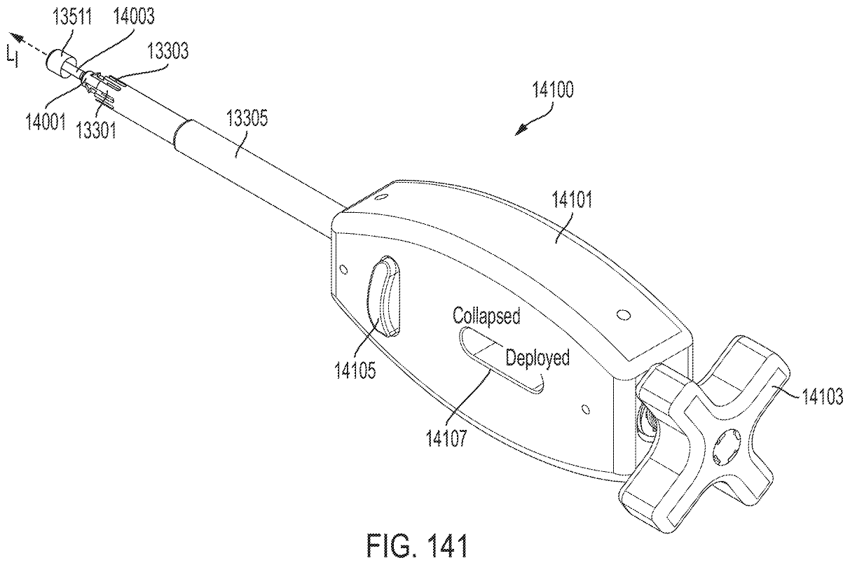

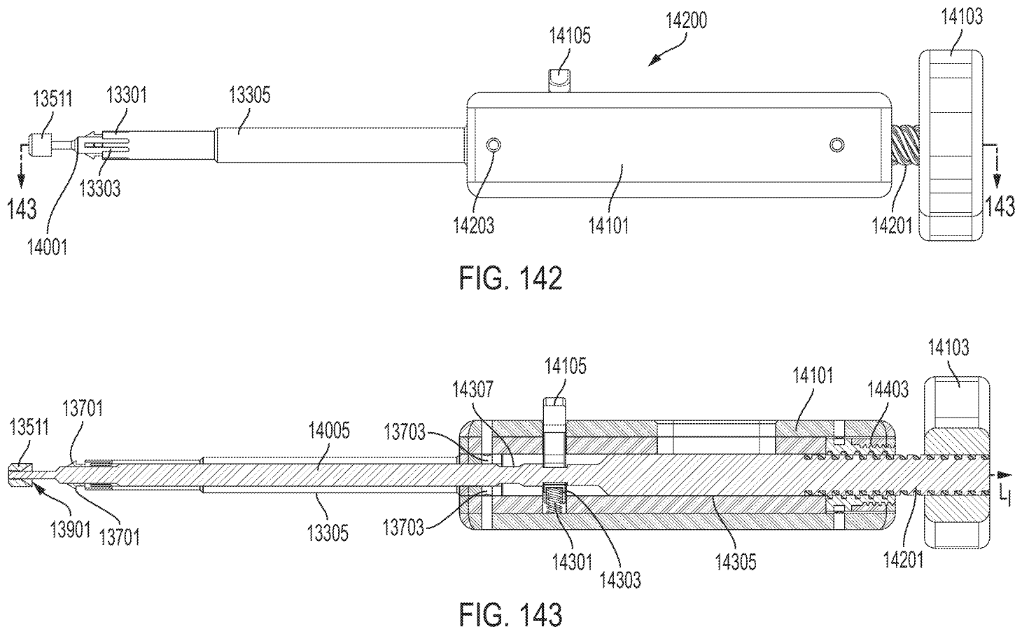

FIG. 141 shows “knob type” inserter 14100. Twisting actuator 14103 may move shaft segment 14003 relative to an inserter housing 14101 and guide tube 13305. Twisting actuator 14103 may move shaft segment 14003 distally out of guide tube 13305. Twisting actuator 14103 may retract shaft segment 13311 proximally into guide tube 13305. Shaft segment 14003 may include drive threads 14201 (shown in FIG. 142) configured to engage complementary threads in housing 14101. Twisting actuator 14103 may move the shaft segment 14003 in a manner defined by the drive threads 14201 and complementary threads 14403 (shown in FIG. 142) of housing 14101.

Inserter 14100 may include shaft lock 14301 (shown in FIG. 143) that locks an axial position of shaft 14305 (also shown in FIG. 143) with respect to housing 14101. FIG. 141 shows button 14105 protruding from housing 14101. Depressing button 14105 may release the shaft lock and allow shaft 14305 to move.

FIG. 141 shows that housing 14101 includes “collapsed” and “deployed” indicators. Movement of shaft 14305 through housing 14101 may be visible to a practitioner. Shaft 14305 may include markings, that when aligned with “collapsed” and “deployed” indicators, indicate when shaft 14305 is in a position that would maintain an implant coupled to guide tube 13305.

When markings on shaft 14305 are aligned with the “deployed” indicator, pusher 13511 may be compressing retaining fingers 13303 and protrusions 13701 toward axis L_{I}, allowing an implant to be decoupled from guide tube 13305. When markings on shaft 14305 are aligned with “deployed,” an implant coupled to guide tube 13305 may be in an expanded state. When markings on shaft 14305 are aligned with “collapsed,” an implant coupled to guide tube 13305 is in collapsed state.

Implants illustrated and described herein may be coupled to “knob type” inserter 14200.

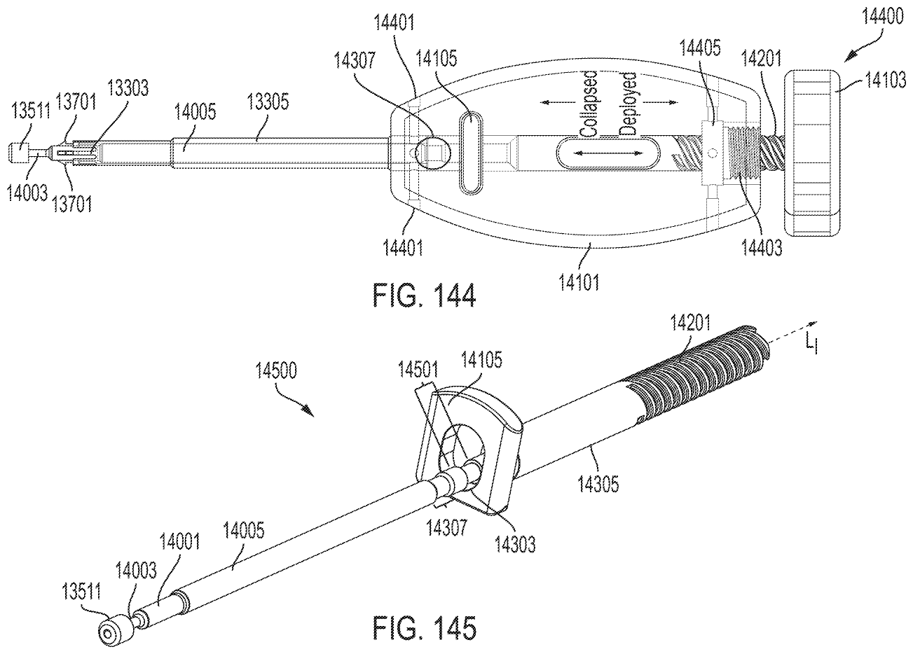

FIG. 142 shows another view 14200 of inserter 14100. View 14200 shows that shaft segment 14003 includes drive threads 14201 (shown in FIG. 142) configured to engage complementary threads in housing 14101. Twisting actuator 14103 may move the shaft segment 14003 in a manner defined by the drive threads 14201 and complementary threads 14403 (shown in FIG. 144) in housing 14101.

View 14200 also shows connector 14203. Connector 14203 may be a dowel. Connector 14203 may connect housing 14101 to apertures 13703 (shown in FIG. 137) of guide tube 13305. Apertures 13703 may receive connectors 14203, a screw, bolt or any other fastener to secure guide tube 13305 to housing 14101 or barrel 13307.

View 14200 also shows shaft segment 14001 positioned to prevent retaining finger 13303 from being compressed or bent toward axis L_{I} (shown in FIGS. 139-140).

FIG. 143 shows cross-sectional view 14300 of inserter 14100 taken along lines 143-143 shown in FIG. 142. View 14300 shows that inserter 14100 includes shaft 14305. Shaft 14305 may include one or more features of shaft 13311 (shown in FIG. 138) or any other shaft described herein. For example, shaft 14305 includes shaft segments 14005 and pusher 13511. Shaft 13311 or any other shaft described herein may include one or more features of shaft 14305.

Shaft 14305 includes threaded segment 14201. Threaded segment 14201 is configured to engage threads 14403 fixed to housing 14101. Twisting knob 14103 moves shaft 14305 proximally or distally (along longitudinal inserter axis L_{I}) with respect to guide tube 13305 and housing 14101. Whether shaft 14305 moves proximally or distally along longitudinal inserter axis L_{I} will depend on which direction, clockwise or counter-clockwise, a practitioner twists knob 14103.

Inserter 14101 includes shaft lock 14303. Shaft lock 14303 is loaded by spring 14301 to move into cut-out 14501 (shown in FIG. 145) in shaft 14305. When shaft lock 14303 is positioned in cut-out 14501, shaft 14301 is locked and may not be moved axially along axis L_{I} by twisting knob 14103. Depressing button 14105 compresses spring 14301 and moves shaft lock 14303 out of cut-out 14501, allowing shaft 14305 to move axially along axis L_{I}.

Shaft 14305 may include two or more cut-outs that may be engaged by shaft lock 14301. Each cut-out may be configured to lock shaft 14305 at a different position along axis L_{I} with respect to guide tube 13305 and housing 14101. A cut-out may be configured to provide different levels of resistance to movement when shaft lock 14301 is positioned in the cut-out. For example, a cut-out, such as cut-out 14501, may include shoulders that extend perpendicular to longitudinal inserter axis L_{I}. When shaft lock 14301 is positioned in cut-out 14501, a practitioner will feel a relatively high resistance when attempting to twist knob 14103 and move of shaft 14305.

A cut-out, such as cut-out 14307, may include shoulders that extend at an oblique angle to longitudinal inserter axis L_{I}. When shaft lock 14301 is positioned in cut-out 14307, a practitioner will feel a relatively less resistance (compared to resistance provided by cut-out 14501) when attempting to twist knob 14103 and move of shaft 14305. If a practitioner applies additional force to knob 14103, shaft lock 14303 may slide over the shoulders of cut-out 14307 and onto a smooth surface of shaft 14305.

View 14300 shows apertures 13703 in guide tube 13305. Apertures 13703 may receive connectors 14203, a screw, bolt or any other fastener to secure guide tube 13305 to housing 14101 or barrel 13307.

FIG. 144 shows illustrative view 14400 of inserter 14100, with housing 14101 and guide tube 13305 shown in transparency. View 1440 shows complementary threads 14403 embedded in housing 14101. Complementary threads 14403 may engage threads 14201 and move shaft 14305 relative to guide tube 13305 (or housing 14101) when knob 14103 is twisted. Complementary threads 14403 are formed in bushing 14405, which is affixed to housing 14101. In some embodiments, complementary threads 14403 may be formed directly in housing 14101.

View 14400 shows cannulas 14401 in housing 14101. Cannulas 14401 are configured to be aligned apertures 13703 in guide tube 13305. A connector (such as connector 14203), dowel, screw, bolt or any other fastener may be inserted into one of cannulas 14401 and apertures 13703 to secure guide tube 13305 to housing 14101.

FIG. 145 shows view 14500 of illustrative inserter components. View 14500 shows shaft lock 14303 positioned relative to cut-out 14501. Cut-out 14501 includes shoulders that extend perpendicular to longitudinal inserter axis L_{I}. When shaft lock 14301 is positioned in cut-out 14501, a practitioner will feel a relatively high resistance when attempting to twist knob 14103 and move of shaft 14305.

View 14500 also shows cut-out 14307. Cut-out 14307 includes shoulders that extend at an oblique angle to longitudinal inserter axis L_{I}. When shaft lock 14301 is positioned in cut-out 14307, a practitioner will feel a relatively less resistance (compared to resistance provided by cut-out 14501) when attempting to twist knob 14103 and move of shaft 14305. If a practitioner applies additional force to knob 14103, shaft lock 14303 may slide over the shoulders of cut-out 14307 and onto a smooth surface of shaft 14305.

View 14500 also shows that depressing button 14105 may move shaft lock 14303 out of a cut-out in shaft 14305.

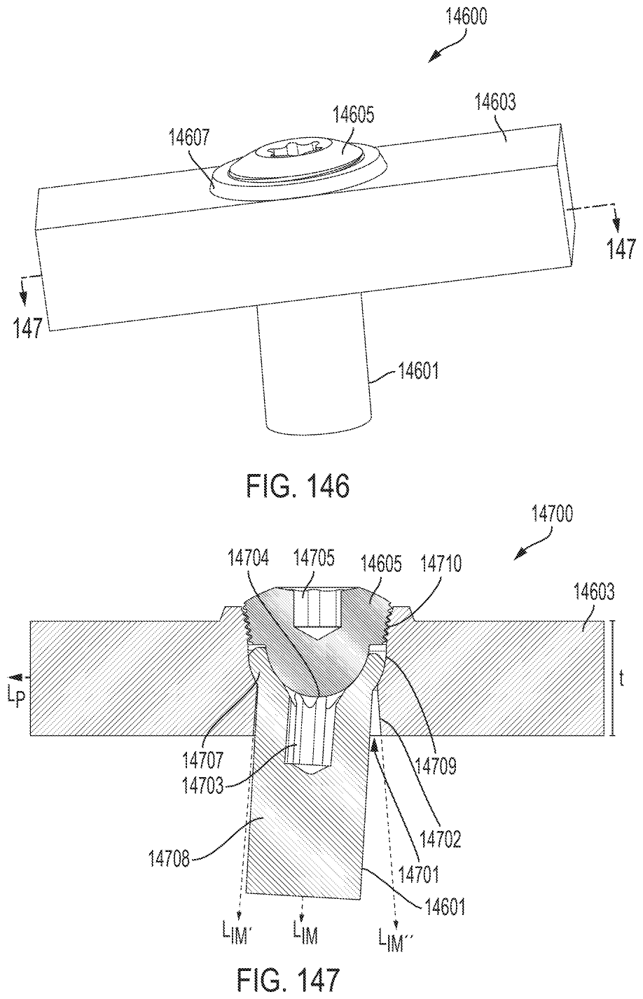

FIG. 146 shows illustrative apparatus 14600 for locking an implant to plate 14603. The implant may be the implant described herein. Illustrative implants may include the implant shown in FIG. 1, 8, or any other figure showing an implant. Plate 14603 may be any illustrative plate disclosed herein. Apparatus 14600 may be configured to lock a longitudinal implant axis (e.g., L_{IM}) with respect to a longitudinal plate axis (e.g., L_{P}). Apparatus 14600 may allow a position of an implant to be adjusted in the bone relative to a position of the plate. The position of the implant with respect to the plate may be described as an angle between the longitudinal implant axis and the longitudinal plate axis. Apparatus 14600 may lock the angular position such that the longitudinal implant axis is fixed with respect to the longitudinal plate axis.

Apparatus 14600 may allow an implant to be positioned in the bone such that the longitudinal implant axis forms an angle with respect to the longitudinal plate axis. The angle may be 0, 5, 10, 15, 30 or 45 degrees. The angle may be any value between 0 and 90 degrees. The angle may be any value between 0 and 45 degrees. The angle may be any value between 0 and 30 degrees. The angle may be any value between 0 and 15 degrees. The angle may be any value between 0 and 10 degrees.

Apparatus 14600 may allow the longitudinal implant axis to be positioned at any angle, with respect to the longitudinal plate axis, within an angular range. An illustrative angular range may be 15 degrees between the implant and plate longitudinal axes. Apparatus 14600 may lock the implant to the plate such that the longitudinal implant axis is fixed with respect to the longitudinal plate axis.

Apparatus 14600 includes elongated member 14601. Elongated member 14601 may be the support (as shown, for example, in FIGS. 2 and 9). Elongated member 14601 may be the extension member (as shown, for example, in FIG. 9). Elongated member 14601 may be coupled to the implant. Elongated member 14601 may define a longitudinal implant axis. Apparatus 14600 includes locking cap 14605. Locking cap 14605 may be threadedly engaged with plate 14603. Plate 14603 includes rim 14607. Rim 14607 may extend a threaded surface within a thickness of plate 14603. Rim 14607 may provide a tactile marker for locating locking 14605 after a bone repair is completed.

FIG. 147 shows cross-sectional view 14700 taken along lines 147-147 shown in FIG. 146. View 14700 shows that elongated member 14601 includes cylindrical body 14708. Cylindrical body 14708 may be fixed to an implant by engaging drive 14703 with a tool (not shown) configured to mate with a shape of drive 14703. An illustrative tool may include a cruciform or hexagonal shaped screwdriver or bit. Using the tool, a practitioner may rotate cylindrical body 14708. Twisting cylindrical body 14703 may threadedly engage a distal end (not shown) of cylindrical body 14708 with the implant hub (e.g., hub 203 shown in FIG. 2), support (e.g., support 208 shown in FIG. 2) or other component of the implant.

Elongated member 14601 includes flared end 14707. Flared end 14707 is configured to be seated on supporting articular surface 14709. Elongated member 14601 may be inserted into an opening in plate 14603 and seated on supporting surface 14709 after plate 14603 is secured to a bone.

Plate 14603 includes relief surface 14702. Relief surface may allow cylindrical body 14708, and a longitudinal axis of the elongated member (e.g., L_{IM}), to be adjusted relative to plate 14603 and longitudinal plate axis L_{P}. Relief surface may allow axis L_{IM} to be positioned at a range of angles relative to longitudinal plate axis L_{P}.

Flared end 14707 and supporting articular surface 14709 may be curved. A curvature of flared end 14707 and supporting articular surface 14709 may allow flared end 14707 to articulate against supporting articular surface 14709. A curvature of flared end 14707 and supporting articular surface 14709 may allow flared end 14707 to be supported by supporting articular surface 14709 at any angle within an angular range. The angular range may be determined by a difference between an outer surface of cylindrical body 14708 and relief surface 14702.

A position of axis L_{IM} with respect to axis L_{P} may be fixed by locking cap 14605. Locking cap 14605 may be threaded into threaded surface 14710 of plate 14603. Locking cap 14605 may be threaded into plate 14603 by engaging drive 14705 with a tool (not shown) configured mate with a shape of drive 14705. An illustrative tool may include a cruciform or hexagonal shaped screwdriver or bit. The tool may rotate locking cap 14605 and drive locking cap 14605 through a thickness of plate 14603.

Locking surface 14704 of locking cap 14605 is configured to engage an inner articular surface of flared end 14707. Threaded engagement of locking cap 14605 and threaded surface 14710 may press locking surface 14708 against an inner articular surface of flared end 14707. Threaded engagement of locking cap 14605 and threaded surface 14710 may press an outer articular surface of flared end 14707 against supporting articular surface 14702.

Plate 14603 has thickness t. A first segment of the thickness, a second segment of the thickness, a third segment of the thickness together may define an opening that penetrates thickness t. The first segment may include threaded surface 14710. The second segment may include supporting articular surface 14709. The third segment may include relief surface 14702. Elongated member 14601 may be inserted into the opening such that flared end 14707 is supported by supporting articular surface 14709.

Threaded engagement of locking cap 14605 into thickness t may apply, and sustain, pressure that fixes a position of elongated member 14601 with respect to plate 14603. the pressure may fix an angle of L_{IM} with respect to L_{P}. Threaded engagement of locking cap 14605 and threaded surface 14710 may drive locking cap 14605 from outside plate 14603 into thickness t of plate 14603. Driving locking cap 14605 into thickness t may press locking surface 14704 against the inner articular surface of flared end 14707. The pressure may increase slip-resistance between the inner articular surface of flared end 14707 and locking surface 14704.

Driving locking cap 14605 into thickness t may press outer articular surface of flared end 14707 against supporting articular surface 14702. The pressure may increase slip-resistance between the outer articular surface of flared end 14707 and supporting articular surface 14702.

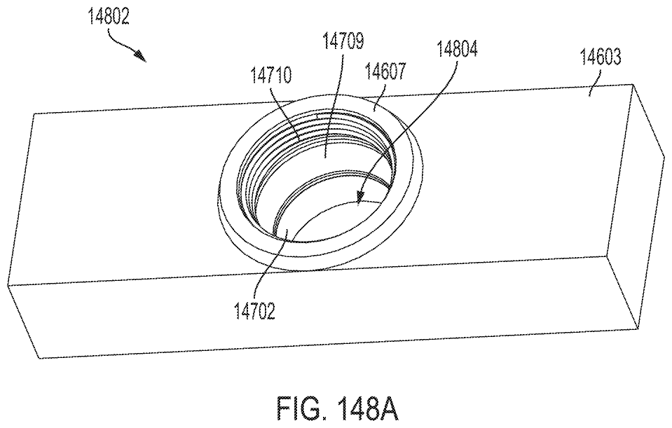

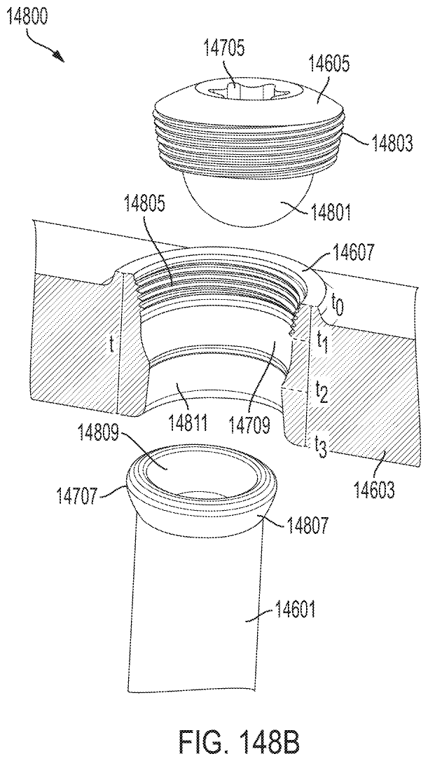

FIG. 148A shows illustrative view 14802 of plate 14603. View 14802 shows opening 14804. Opening 14804 penetrates through thickness t of plate 14603. A first segment of thickness t, a second segment of thickness t and a third segment of thickness t together may define an opening that penetrates thickness t. View 14802 shows that the first segment may include threaded surface 14710. View 14802 shows that the second segment may include supporting articular surface 14709. View 14802 shows that the third segment may include relief surface 14702. Elongated member 14601 may be inserted into opening 14804 such that flared end 14707 is supported by supporting articular surface 14709.

FIG. 148B shows exploded view 14800 of apparatus shown in FIG. 146. Exploded view 14800 shows plate 14603 in cross-section.

View 14800 shows that locking cap 14605 includes threads 14803. Threads 14803 are configured to engage threaded surface 14710 of plate 14603.

View 14800 shows that thickness t includes three segments. Segment t₀₋₁ includes threaded surface 14710. Threads 14803 may engage threaded surface 14710 as locking cap is driven from t₀ to t₁. Engagement of threads 14803 and threaded surface 14805 may hold locking cap 14605 within opening 14894 as locking surface 14801 presses against inner articular surface 14809.

Segment t₀₋₁ includes a thickness of rim 14607. In some embodiments, thickness t may not include a thickness of rim 14607. In some embodiments, plate 14603 may not include rim 14607.

View 14800 shows segment t₁₋₂ includes supporting articular surface 14709. View 14800 shows that supporting articular surface 14709 defines a circumference (or diameter) that varies along thickness t between t₁ and t₂. View 14800 shows that a circumference (or diameter) defined by supporting articular surface 14709 decreases along thickness t from t₁ to t₂.

View 14800 shows segment t₂₋₃ includes relief surface 14811. View 14800 shows that relief surface 14811 defines a circumference (or diameter) that varies along thickness t between t₂ and t₃. View 14800 shows that a circumference (or diameter) defined by relief surface 14811 increases along thickness t from t₂ to t₃. In three-dimensional space, relief surface 14811 may define a conically shaped volume. Outer articular surface 14807 and supporting articular surface 14709 may allow axis L_{IM} to be oriented in different positions within the conically shaped volume.

View 14800 shows that flared end 14707 includes outer articular surface 14807. Outer articular surface 14807 may be seated on supporting articular surface 14709. Curvatures of outer articular surface 14807 and supporting articular surface 14709 may allow axis L_{IM} (shown in FIG. 147) to be positioned at any angle within a range of angles to axis L_{P} (also shown in FIG. 147). The angular range may be defined by relief surface 14811. Supporting articular surface 14709 may support elongated member 14601 at any angle within the range.

Curvatures of outer articular surface 14807 and supporting articular surface 14709 may allow axis L_{IM} (shown in FIG. 147) to be oriented in different positions within a conically shaped volume defined by relief surface 14811. Supporting articular surface 14709 may support elongated member 14601 at any orientation within the conically shaped volume defined by relief surface 14811.

View 14800 shows that flared end 14707 includes inner articular surface 14809. Locking surface 14801 may articulate against inner articular surface 14809 as locking cap 14605 is driven into opening 14804 from t₀ towards t₁. Driving locking cap 14605 from t₀ towards t₁ may press locking surface 14801 against inner articular surface 14809. Driving locking cap 14605 from to towards t₁ may press outer articular surface 14807 against supporting articular surface 14709.

Locking surface 14704 and inner articular surface 14809 may each have a lower coefficient of friction than supporting articular surface 14709. Locking surface 14704 and inner articular surface 14809 may each have a lower coefficient of friction than outer articular surface 14807. For example, inner articular surface 14809 and locking surface 14704 may be smoother surfaces than outer articular surface 14807 or supporting articular surface 14702.

Locking surface 14704 and inner articular surface 14809 may each have a lower static coefficient of friction than outer articular surface 14807 or supporting articular surface 14709. Locking surface 14704 and inner articular surface 14809 may each have a lower kinetic coefficient of friction than outer articular surface 14807 or supporting articular surface 14709.

Inner articular surface 14809 and locking surface 14704 may each be considered “low friction” surfaces relative to supporting articular surface 14709 or outer articular surface 14807. Inner articular surface 14809 and locking surface 14704 may each have a coefficient of friction that allows locking surface 14704 to articulate against inner articular surface 14809 as locking cap 14605 is driven into opening 14804 from t₀ to t₁.

Outer articular surface 14807 and supporting articular surface 14702 may each be “high friction” surfaces relative to locking surface 14801 or inner articular surface 14809. A “high friction” surface may have a higher coefficient of friction compared to a “low friction” surface. For example, outer articular surface 14807 and supporting articular surface 14702 may be rougher surfaces than inner articular surface 14809 or locking surface 14704.

Outer articular surface 14809 and supporting articular surface 14709 may each have a coefficient of friction high enough to allow locking surface 14704 to articulate against inner articular surface 14709 without substantially disturbing an orientation of elongated member 14601 relative to plate 14603. Outer articular surface 14809 and supporting articular surface 14709 may each have a coefficient of friction that allows locking surface 14704 to articulate against inner articular surface 14709 without substantially disturbing an orientation of L_{IM} relative to L_{P}.

Driving locking cap 14605 into opening 14804 from t₀ to t₁ increases slip-resistance between outer articular surface 14807 and supporting articular surface 14709. Driving locking cap 14605 from t₀ to t₁ may press locking surface 14801 against inner articular surface 14809, which in turn presses outer articular surface 14807 against supporting articular surface 14709. Driving locking cap 14605 from t₀ to t₁ may increase a normal force applied to supporting articular surface 14709, thereby increasing a frictional force needed to move elongated member 14601.

The following equation shows an illustrative mathematical model that may be used to construct apparatus 14600: F_{f}≤μFₙ

where:

F_{f} is a force of friction exerted by each surface on the other;

μ is a coefficient of friction, which is an empirical property of the contacting surfaces; and

Fₙ is a normal force exerted by each surface on the other, directed perpendicular (normal) to the surface.

Increased values of F_{f} may provide greater slip-resistance. F_{f} may be increased by adjusting constructing surfaces having a larger μ magnitude or by constructing surfaces such that as locking cap 14605 is driven from t₀ to t₁, a greater Fₙ is applied to a particular surface.

In some embodiments, as locking cap 14605 is driven from t₀ to t₁, Fₙ applied to surfaces 14801, 14809, 14709 or 14807 may not sufficiently differ to substantially change F_{f} for each surface. Thus, maintaining a position of elongated member 14601 as locking cap 14605 is driven from t₀ to t₁ may predominately be a function of μ for each surface. Generally, a static value of μ (μₛ) is larger for surfaces at rest relative to each other than a kinetic value of μ (μₖ) for surfaces in motion relative to each other.

As locking cap 14605 is driven from t₀ to t₁, locking surface 14801 and inner articular surface 14809 may be in relative motion. To maintain a position of elongated member 14601, outer articular surface 14807 and supporting articular surface 14709 should preferably remain at rest relative to each other as locking cap 14605 is driven from t₀ to t₁. Surfaces 14709 and 14807 may be constructed of material that has a μₛ value that is greater than a μₖ value of surfaces 14801 and 14809.

In some embodiments, surfaces 14801, 14809, 14709 or 14807 may be contoured such that, as locking cap 14605 is driven from t₀ to t₁, Fₙ applied to each surface is different. For example, surfaces 14801 and 14809 may be designed to have a curvature such that Fₙ applied to those surfaces is less than the Fₙ applied to surfaces 14709 or 14807. Thus, due to differences in Fₙ and, despite differences in μₛ or μₖ values, as locking cap 14605 is driven from t₀ to t₁, F_{f} may be greater for surfaces 14709 and 14807 than F_{f} for surfaces 14801 and 14809. Because F_{f} is greater for surfaces 14709 and 14807 than F_{f} for surfaces 14801 and 14809, outer articular surface 14807 and supporting articular surface 14709 may remain at rest relative to each other as locking cap 14605 is driven from t₀ to t₁.

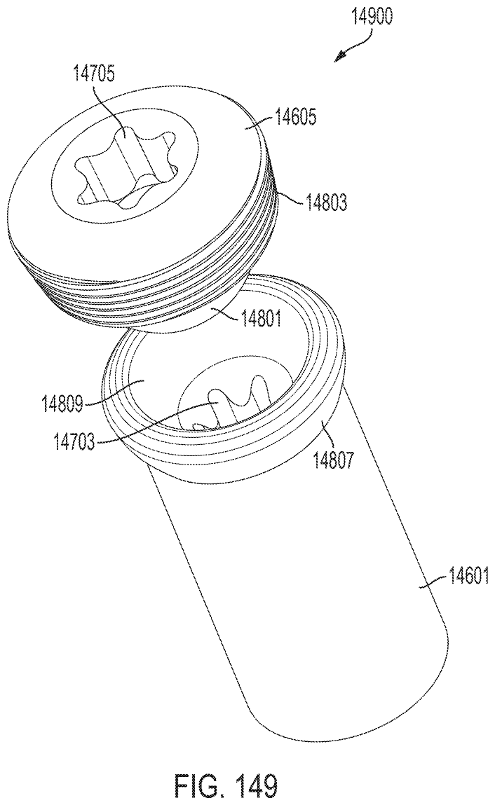

FIG. 149 shows illustrative view 14900 of elongated member 14601 and locking cap 14605. View 14900 shows that locking cap 14605 includes hexagonal shaped drive 14705. Drive 14705 may be engaged by a tool, such as a hexagonal shaped screwdriver, that drives locking cap 14605 into opening 14804 in plate 14603.

View 14900 shows that elongated member includes drive 14703. Drive 14703 may be engaged by a tool, such as an appropriately shaped screwdriver, that rotates elongated member 14601. Rotation of elongated member 14601 may drive a threaded end of elongated member 14601 into a hub of an implant. Rotation of elongated member 14601 may drive a threaded end of elongated member 14601 into a support of an implant.

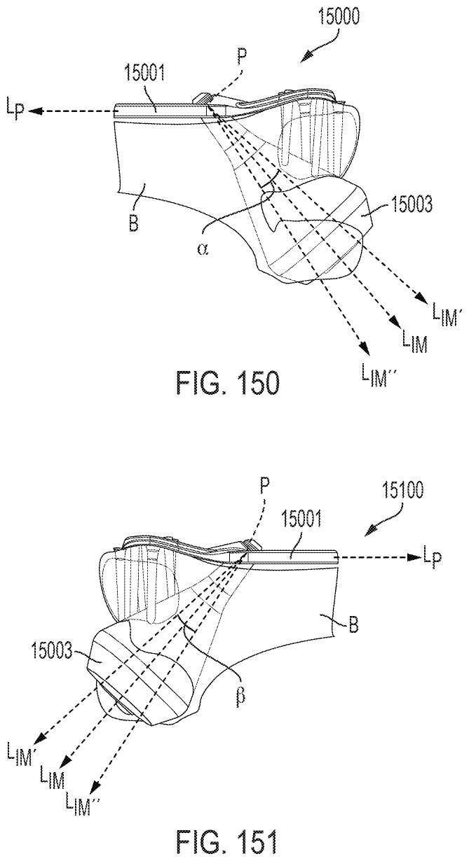

FIG. 150 shows illustrative view 15000 of implant 15003 secured to plate 15001 and positioned inside bone B. Implant 15003 may be any suitable implant describe herein. Plate 15001 may be any suitable plate described herein. In view 15000, implant 15003 is shown in transparency, showing a longitudinal axis, L_{IM} that passes through a center of implant 15003.

A position of implant 15003 may be defined based on an angle between longitudinal implant axis L_{IM} and longitudinal plate axis L_{P}. Axes L_{IM′} and L_{IM″} represent a range of alternative positions for a longitudinal axis L_{IM}. Apparatus disclosed herein may lock axis L_{IM} to plate 15001 at positions between axes L_{IM′} and L_{IM″}. View 15000 shows that axes L_{IM′} and L_{IM″} represent an angular range for axis L_{IM} of α.

View 15000 is a posterior-anterior view of an illustrative angular displacement range for L_{IM}. In some embodiments, after being inserted into bone B, implant 15003 may be rotated about pivot point P and L_{IM} locked any suitable position between L_{IM′} and L_{IM″}.

Apparatus disclosed herein may allow axis L_{IM} to be locked in position, with respect to axis L_{P}, in various orientations in three-dimensional space. For example, in three-dimensional space, L_{IM′} and L_{IM″} may extend along an outer face of a conical volume. Apparatus may fix the L_{IM} in any suitable position within the conical volume.

The apparatus may provide L_{IM} with a range of movement and lock L_{IM} at any position within the range. The range of movement for L_{IM} may be defined by an angular displacement between L_{IM′} and L_{IM″}. An illustrative angular displacement may be 15°. For example, in FIG. 158, the range of movement of L_{IM} may be a shift toward L_{IM′} (e.g., by 7.5°) or a shift toward L_{IM″} (e.g., by 7.5°). An illustrative angular displacement may be 5°. An illustrative angular displacement may be 10°. An illustrative angular displacement may be 30°. Apparatus disclosed herein may be configured to provide any suitable angular displacement for a given clinical application.

FIG. 151 shows illustrative view 15100 of implant 15003 secured to plate 15001 and positioned inside bone B. In view 15100, implant 15003 is shown in transparency, showing longitudinal axis L_{IM} passing through a center of implant 15003. View 15100 is an anterior-posterior view of an angular displacement range for L_{IM}.

Pivot point P shown in FIGS. 150 and 151 is shown as an illustrative frame of reference for describing an angular displacement range for L_{IM}. Apparatus may allow L_{IM} to be moveable axially along L_{P} until locking an orientation of L_{IM}. Until L_{IM} is locked, an orientation of L_{IM} may be adjusted relative to L_{P} and relative to L_{IM′}/L_{IM″}. View 15100 shows that axes L_{IM′} and L_{IM″} represent an angular range for axis L_{IM} of β. In some embodiments, angular range β shown in anterior-posterior view 15100 may be different than angular range α shown in posterior-anterior view 15000. In some embodiments, angular range β shown in anterior-posterior view 15100 may be the same as angular range α shown in posterior-anterior view 15000.

Driving locking cap 14605 into threaded surface 14710 may lock an orientation of L_{IM} relative to L_{P}. Driving the locking cap 14605 into threaded surface 14710 may lock an orientation of L_{I} relative to L_{IM′}/L_{IM″}. Driving the locking cap 14605 into the locking socket 15211 may lock an orientation of L_{I} relative to L_{P} and relative to L_{IM′}/L_{IM″}.

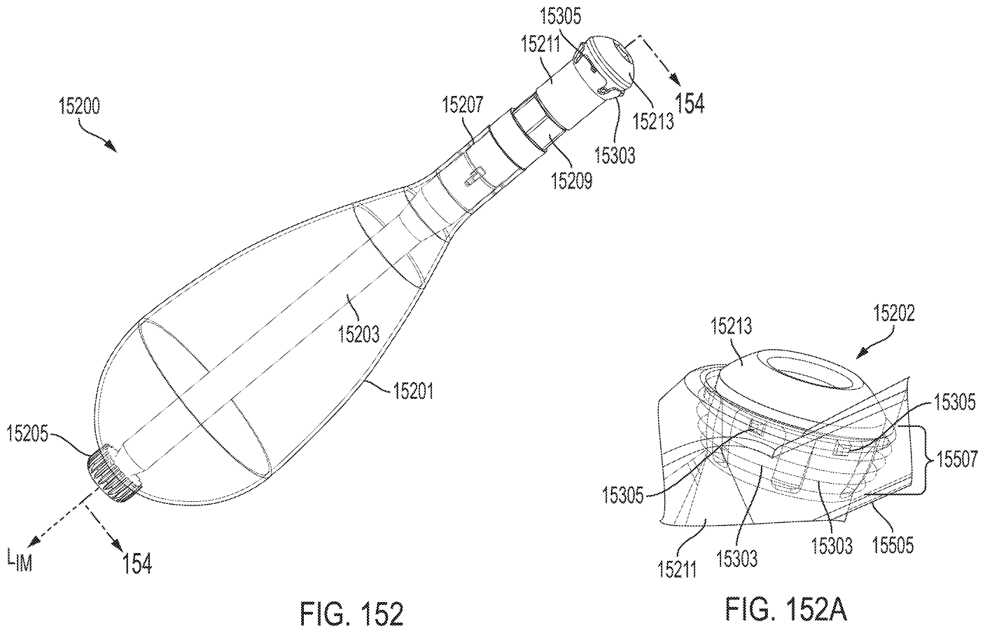

FIGS. 152 and 152A show illustrative apparatus 15200 and 15202 for locking implant 15201 to plate 15505. Implant 15201 may be any suitable implant described herein. Apparatus 15200 may be used to lock implant 15201 to a plate at a position between L_{IM′} and L_{IM″} (shown in FIGS. 147, 150 and 151).

Apparatus 15200 includes locking socket 15211. Locking socket 15211 may include fingers 15303 and, in some embodiments, a locking tooth 15305 on each finger 15303. In some embodiments, each finger 15303 may include multiple locking teeth 15305. In some embodiments, a subset of fingers 15303 may include a locking tooth 15305.

Apparatus 15200 includes locking cap 15213. Locking cap 15213 is configured to be threaded into locking socket 15211. A head of locking cap 15213 may be shaped such that driving locking cap 15213 into the locking socket 15211 may push fingers 15303 away from their default position relative to axis L_{IM}. When fingers 15303 are pushed away from axis L_{IM}, teeth 15303 mounted on fingers 15303 may be positioned in grooves 15507 in plate 15505. Plate 15505 is shown in transparency so that fingers 15303, teeth 15305 and grooves 15507 inside plate 15505 are visible in FIG. 152A.

Positioning teeth 15303 in grooves 15507 fixes a position of locking socket 15211 with respect to plate 15505. Locking socket 15211 may be fixed to extension member 15209. Extension member 15209 may be fixed to support 15203. Support 15203 may be fixed to hub 15205. Thus, fixing a position of locking socket 15211 with respect to plate 15505 may fix a position of implant 15201 with respect to plate 15505.

Each tooth 15303 may be positioned in a different one of grooves 15507. It may not be necessary to position all of teeth 15305 in grooves 15507 to lock a position of implant 15201 (or axis L_{IM}) relative to plate 15505 (or a longitudinal axis L_{P} defined by plate 15505).

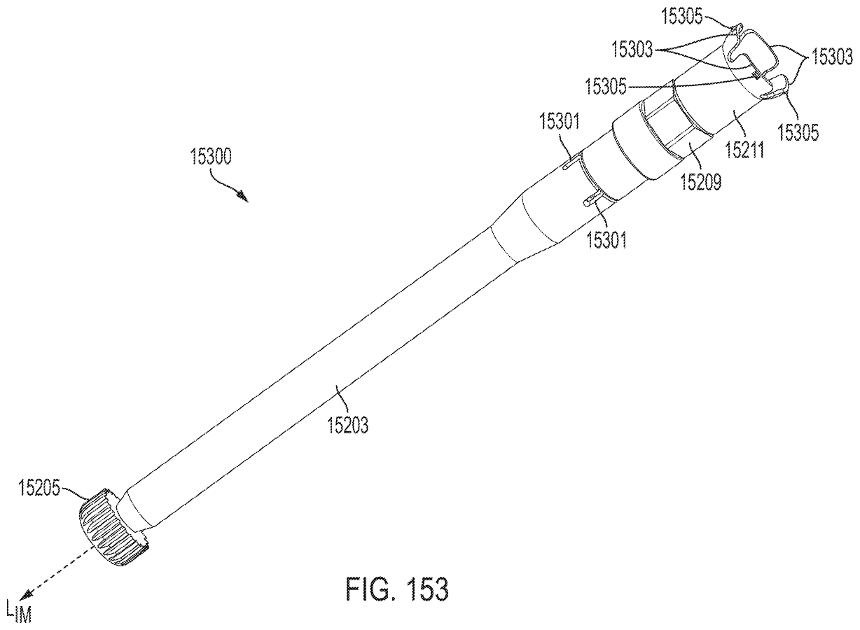

FIG. 153 shows illustrative components 15300 that may be used to in connection with fixing a position of implant 15201 in a bone. Components 15300 includes extension member 15209. Different length extension members 15209 may space implant 15201 inside a bone at different distances from plate 15505.

Support 15203 may include slots or kerf cuts 15301. Kerf cuts 15301 may be engaged during a procedure for removing implant 15201 from a bone. During the removal procedure, kerf cuts 15301 may be used to turn the support 15203 and disengage it from hub 15205. Support 15203 may be removed and a broaching tool inserted into a head of implant 15201. The broaching tool may broach bone ingrowth that penetrated a head of implant 15201. An inserter device, such as inserters shown in FIG. 107 129, 133 or 141, may be affixed to implant 15201.

The inserter device may collapse implant 15201 while implant 15201 is inside the bone. After collapsing implant 15201, the inserter may be used to withdraw implant 15201 from the bone. The methods may include, after the mounting, twisting the implant to separate a head of the implant from the bone.

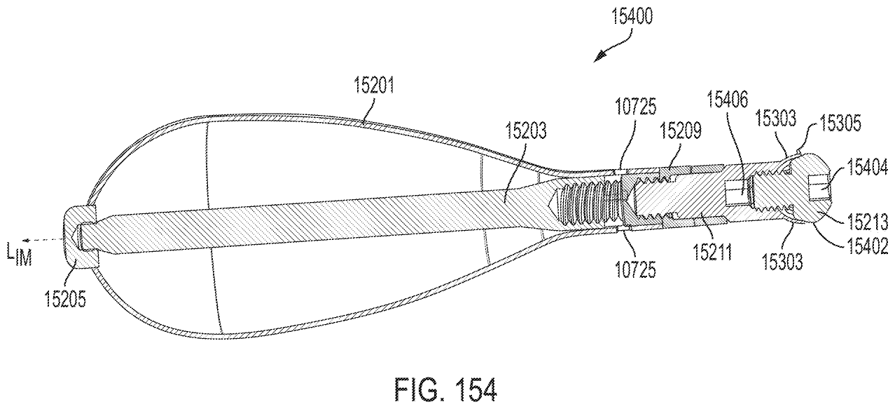

FIG. 154 shows a cross-sectional view 15400 of apparatus 15200 taken along line 154-154. View 15400 shows that locking socket 15211 may threadedly engage to extension member 15209. Drive 15404 may be engaged to threadedly couple locking socket 15211 and extension member 14601.

Extension member 15209 may threadedly engage support 15203. Drive 15406 may be engaged to threadedly engage extension member 14601 and support 15203. Support 15203 may threadedly engage hub 15205. During a removal procedure, kerf cuts 15301 may be used to rotate support 15203 and threadedly disengage it from hub 15205.

View 15400 shows locking cap 15213 engaged with the locking socket 15211.

Locking cap 15213 includes threads configured to engage complementary threads inside locking socket 15211. A head of locking cap 15213 includes surface 15402 that is configured to push fingers 15303 of the locking socket 15211 away from axis L_{IM}. As the locking cap 14605 is threaded into the locking socket 15211, surface 15402 is shaped to push fingers 15303 away from L_{IM}. The deeper the locking cap 15213 is threaded into locking socket 15211, the further surface 15402 pushes fingers 15303 away from L_{IM}. By pushing fingers 15303 away from L_{IM}, surface 15402 positions locking teeth 15305 in groove 15507 on plate 15505 and fixes a position of locking socket 15211 relative to plate 15505.

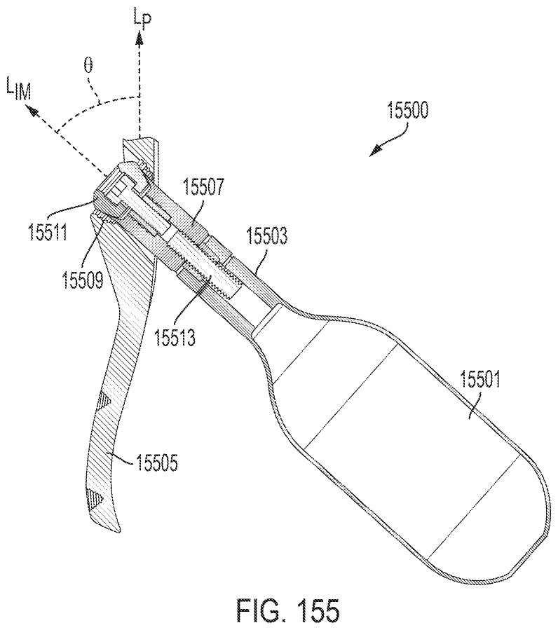

FIG. 155 shows an illustrative cross-sectional view 15500 of implant 15501 affixed to plate 15505. Axis L_{IM} of implant 15501 is locked relative to axis L_{P} of plate 15505 by teeth of locking socket 15507 being secured by locking cap 15511 in grooves 15509. Locking socket 15507 is secured to proximal base 15503 of implant 15501 by threaded member 15513. View 15500 shows that locking cap 15511 has locked implant 15501 such that L_{IM} and L_{P} are oriented at angle θ with respect to each other.

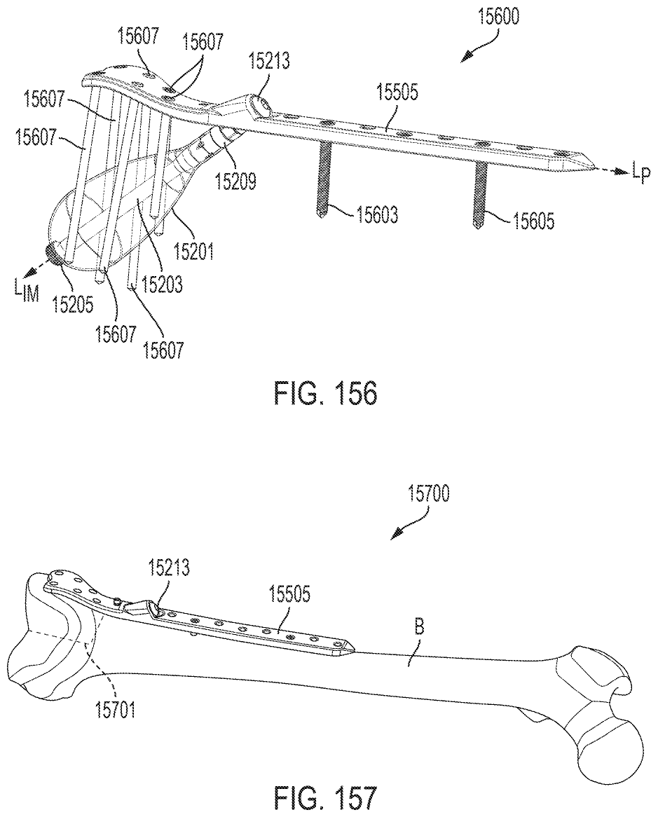

FIG. 156 shows illustrative therapeutic scenario 15600. Scenario shows that implant 15201 is fixed with respect to plate 15505. Scenario 15600 shows that plate 15505 is affixed to a bone using screws 15603 and 15606. Scenario also shows that a fracture in a bone may be repaired by anchoring screws 15607 to implant 15201. Implant 15201 may provide a secure substrate for screws 15607. Implant 15201 may be secured to plate 15505 by driving locking cap 15213 into locking socket 15211. Driving locking cap 15213 into locking socket 15211 may fix axis L_{IM} relative to axis L_{P}.

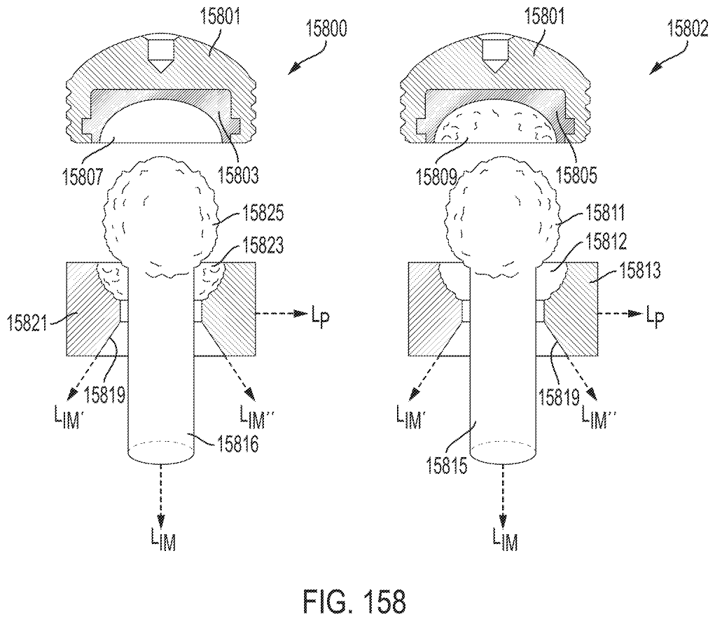

FIG. 157 shows therapeutic scenario 15600 in connection with bone B. FIG. 157 shows that therapeutic scenario 15600 has been used to repair fracture 15701 in bone B.

FIG. 158 shows illustrative apparatus 15800 and 15802 for locking axis L_{IM} of elongated members 15816 and 15815 relative to axis L_{P} of plates 15821 and 15813 at a range of positions between L_{IM′} and L_{IM″}. Elongated members 15816 and 15815 may be coupled to a support of an implant. Elongated members 15816 and 15815 may be coupled to a hub of an implant. The position of the implant relative to plate 15821 or 15813 may be defined based on an angle between axis L_{IM} axis L_{P}.

Apparatus 15800 and 15802 may fix a position of L_{IM} at positions between L_{IM′} and L_{IM″}. Apparatus 15800 and 15802 may allow elongated members 15816 and 15815 to be locked in position, with respect to plates 15821 and 15813, in various orientations in three-dimensional space. In three dimensions, axes L_{IM′} and L_{IM″} may extend along an outer face of a conical volume. Apparatus 15800 and 15802 may fix elongated members 15816 and 15815 in any position within the conical volume.

The apparatus may provide L_{IM} with a range of movement and lock L_{IM} at any position within the range. The range of movement for L_{IM} may be defined as an angular displacement. An illustrative angular displacement may be 15°. For example, in FIG. 158, the range of movement may allow L_{IM} to be shifted toward L_{IM′} (e.g., by 7.5°) or shifted toward L_{IM″} (e.g., by 7.5°). An illustrative angular displacement may be 5°. An illustrative angular displacement may be 10°. An illustrative angular displacement may be 30°. Apparatus may be configured to provide any suitable angular displacement for a given clinical application.

Curvature of dimpled head 15825 and curvature of dimpled surface 15809 may allow longitudinal axis L_{IM} to be positioned at any position allowed by relief surface 15819. Locking cap 15801 may be threaded into female threads (not shown) in plates 15821 or 15813. Threading locking cap 15801 into the female threads of plate 15821, presses dimpled head 15825 against dimpled surface 15823. Pressing dimpled head 15825 against dimpled surface 15823 locks elongated member 15816 in a position relative to plate 15821.

Threading locking cap 15801 into the female threads of plate 15813, presses dimpled head 15811 against dimpled surface 15809. Pressing dimpled head 15811 against dimpled surface 15809 locks elongated member 15815 in a position relative to plate 15813.

FIG. 158 shows that locking cap 15801 may be compatible with insert 15803 and insert 15805. Insert 15803 may be configured to rotate about dimpled head 15825 while dimpled head 15825 is held in position by dimpled surface 15823. Insert 15805 may be configured to rotate dimpled head 15811 relative to smooth surface 15812.

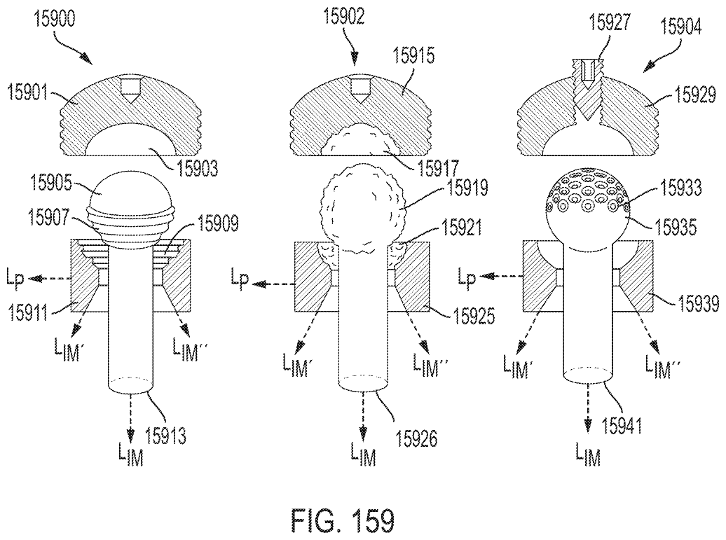

FIG. 159 shows illustrative apparatus 15900, 15902 and 15904 for locking an elongated member to a plate. Implant 15201 may be any suitable implant described herein.

Apparatus 15900 shows that plate 15911 includes features 15909 and a head of elongated member 15913 includes features 15907. Before tightening locking cap 15901, elongated member 15913 may be moveable between L_{IM′} and L_{IM″}. Threading locking cap 15901 into plate 15911 may press features 15907 and 15909 against each other and thereby lock a position of L_{IM} relative to L_{P}. Smooth surfaces 15905 on the head of elongated member 15913 and smooth surface and 15903 of locking cap 15901 allows locking cap 15901 to rotate relative to the head of elongated member 15913.

In some embodiments, a head of the elongated member 15913 may include a surface that is deformable by features 15909 of plate 15911 in response to a tightening of locking cap 14605. The deformable surface may be configured to mold to features 15909, thereby forming features in the head and locking a position of the elongated member 15913 relative to plate 15911.

Apparatus 15902 shows dimpled surface 15917 inside locking cap 15915. Apparatus 15902 also shows dimpled surface 15921 inside plate 15925. A head of elongated member 15926 includes dimpled surface 15919. A dimpled surface may include protrusions and dips. An illustrative dimpled surface may be created by machining, additive fabrication or other finish processes such as shot-peening or etching. Tightening locking cap 15915 may press the protrusions of one dimpled surface into dips of the other dimpled surface and thereby lock a position of L_{IM} (between L_{IM} and L_{IM″}) relative to L_{P} of plate 15925.

Apparatus 15904 shows an illustrative locking screw 15927 that includes a tip configured to fit into a pit, such as pit 15933 in pitted head 15935 of elongated member 15941. Locking cap 15929 includes a threaded bore configured to receive locking screw 15927. Tightening locking cap 15929 into plate 15939 and threading locking screw 15927 into the bore may press the tip of locking screw 15927 into one of the pits of pitted head 15935, thereby locking a position of L_{IM} (between L_{IM′} and L_{IM″}) relative to L_{P} of plate 15939.

In some embodiments, locking screw 15927 and locking cap 15929 may be used together with an elongated member having a head with a dimpled surface such as elongated member 15926. Locking screw 15927 may be driven through locking cap 15929. A tip of the locking screw may fit into a dimple in the dimpled surface to lock a position of the elongated member relative to a plate.

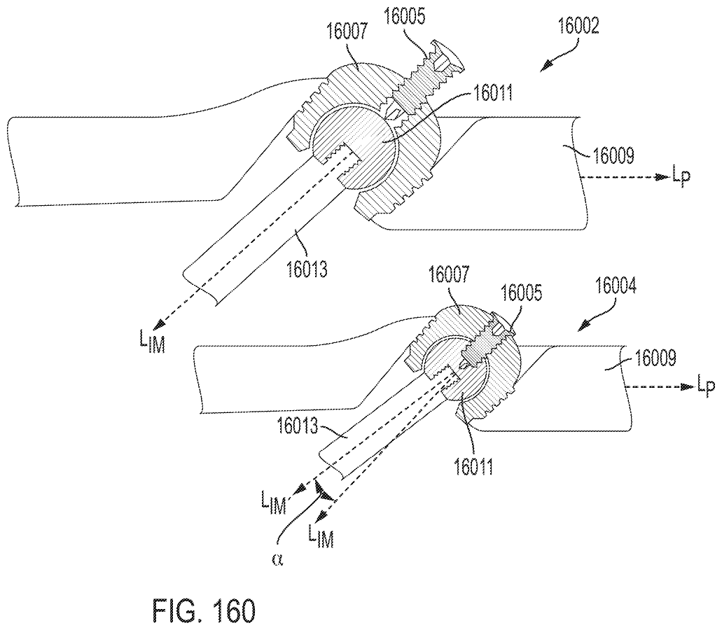

FIG. 160 shows illustrative scenarios 16002 and 16004 for locking an implant to a plate. Scenario 16002 includes locking cap 16007 having a threaded bore for receiving self-threading locking screw 16005. Elongated member 16013 includes “soft” head 16011. Soft head 16011 may be constructed from plastic material or any other suitable material. When elongated member 16013 is in a desired angular position with respect to plate 16009, self-threading locking screw 16005 may be driven into “soft” head 16011 to lock axis L_{IM} of elongated member 16013 in a position relative to axis L_{P} of plate 16009. In scenario 16002 elongated member 16013 has not been locked and is aligned with axis L_{IM}.

Scenario 16004 shows that after locking screw 16005 is driven into soft head 16007, a longitudinal axis of elongated member 16013 has been locked along axis L_{IM′}. Elongated member 16013 has been shifted by angle α from being aligned with axis L_{IM} in connection with scenario 16002.

Thus, apparatus and methods for bone fracture repair have been provided.

Persons skilled in the art will appreciate that the present invention can be practiced by other than the described examples, which are presented for purposes of illustration rather than of limitation.

1-382. (canceled)

383. Apparatus for bone repair, the apparatus defining a longitudinal axis and comprising:
a mesh anchoring substrate;
a base;
a hub;
a support; and
a flange;
wherein:
the mesh anchoring substrate:
is configured to expand away from a central axis of the mesh; and
extends longitudinally between the base and the hub;
the base includes:
a base first end adjacent the mesh; and
a base second end opposite the base first end;
the base defines a cannula that extends between the base first end and the base second end;
the support includes:
an elongated body, a maximum diameter of the elongated body being not greater than a diameter of the cannula; and
a support end that is configured to be removably coupled to the hub;
the flange is configured to be:
seated against the base second end; and,
longitudinally fixed to the hub;
when the mesh is in an expanded state, coupling the end of the support to the hub and seating the flange against the base sets an upper limit of separation along the longitudinal axis between the hub and the base.

384. The apparatus of claim 383 wherein, when the mesh is in an expanded state, coupling the support end to the hub and seating the flange against the base does not prevent sliding movement of the base, over the elongated body, toward the hub.

385. The apparatus of claim 383 wherein:
the cannula is cylindrical and extends along a central axis of the base;
the elongated body is cylindrical;
the cannula does not define a protrusion for being seated in an indentation in the elongated body; and
the elongated body does not define a protrusion for being seated in an indentation extending through an inner face of the base, the inner face defining the cannula.

386. The apparatus of claim 383 wherein the elongated body has a uniform circumference.

387. The apparatus of claim 386 wherein the circumference is sized such that, when the elongated body is advanced through, and positioned in, the cannula, the elongated body abuts the cannula.

388. The apparatus of claim 383 wherein the flange has a circumference greater than a circumference of the cannula.

389. The apparatus of claim 383 wherein:
the flange is cylindrical and has a first circumference;
the base is cylindrical and has a second circumference; and
the first circumference is substantially equal to the second circumference.

390. The apparatus of claim 383 wherein:
the hub has a thickness and defines a threaded bore extending through the thickness, the threaded bore having a first length;
the hub includes a coupling mechanism, the coupling mechanism including the threaded bore;
the support end includes a threaded surface and a tip, the threaded surface being threaded to mate with the threaded bore, the threaded surface and tip, together, having a second length greater than the first length; and
in operation, the mesh is expanded, the threaded surface is in threaded engagement with the threaded bore and the tip is positioned outside the hub.

391. The apparatus of claim 383 wherein:
the hub includes:
a hub first end;
opposite the hub first end, a hub second end disposed more proximate the base than is the hub first end; and
a thickness extending between the hub first end and the hub second end;
a recess extends through the hub second end and through some, but not all of, the thickness;
the recess including a threaded bore for threadedly engaging the support end; and
the support end is threaded to mate with the threaded bore.

392. The apparatus of claim 383 wherein the hub includes:
a hub first end;
a hub second end;
a cylindrical section defining a constant circumference and extending away from the hub second end; and
a tapered section extending away from the cylindrical section toward the hub first end, a diameter of the tapered section decreasing along a central axis of the hub toward the hub first end;
wherein:
the hub second end is:
opposite the hub first end; and
more proximate the base than is the hub first end;
a thickness extends between the hub first end and the hub second end; and
the hub includes a coupling mechanism extending through the hub second end and into the thickness.

393. The apparatus of claim 383 wherein:
the support end is a support first end;
the flange is positioned at a support second end; and
the elongated body extends away from the flange to the support first end.

394. The apparatus of claim 383 wherein the support end is configured to be removably coupled to a coupling mechanism of the hub

395. The apparatus of claim 393 wherein the support second end comprises a bore.

396. The apparatus of claim 395 wherein:
the flange includes a flange first face configured to be seated against the base second end and a flange second face, opposite, along a central axis of the support, the flange first face;
the flange second face is the support second end; and
the bore traverses the flange second face and extends through the flange.

397. The apparatus of claim 396 wherein the bore is threaded.

398. The apparatus of claim 397 further comprising a fixation member including:
a head;
a threaded surface configured for threaded engagement with the bore; and
a body disposed between the head and the threaded surface.

399. The apparatus of claim 398 wherein, in operation, when the support is coupled to the hub and seated against the base:
the head of the fixation member is seated in an opening defined in a plate;
the threaded surface of the fixation member is engaged with the bore; and
the flange is positioned, along the longitudinal axis, between the head of the fixation member and the base second end.

400. The apparatus of claim 383 wherein fixing the upper limit of separation prevents the mesh from collapsing.

401. The apparatus of claim 383 wherein the elongated body defines at least two circumferences along a length of the elongated body.

402. The apparatus of claim 383 wherein the support defines a transverse opening that in operation is disposed between the base and the hub and is sized for clearance of a screw, the screw being configured to penetrate and engage the mesh.

403. The apparatus of claim 383 further comprising a fixation member;
wherein:
the cannula has a cannula circumference;
the hub includes a first coupling mechanism;
the support end is a support first end and is configured to be removably coupled to the first coupling mechanism;
the support includes a support second end, opposite the support first end, the support second end including a second coupling mechanism;
a maximum circumference of the support is not greater than the cannula circumference;
the fixation member includes:
a body extending between a head and an elongated member;
the elongated member is configured to be coupled to the second coupling mechanism; and
the head includes the flange, and is configured to be seated against the base second end; and,
when the mesh is in an expanded state, coupling the support first end to the hub, and coupling the fixation member to the support such that the head of the fixation member is seated against the base, sets the upper limit of separation along the longitudinal axis between the hub and the base.

404. The apparatus of claim 403 wherein:
a distance between the hub and the base second end defines, when the mesh is expanded, a first length;
the support has a second length;
the second length is smaller than the first length; and,
when the mesh is in an expanded state, coupling the support first end to the hub positions the support second end in the cannula.

405. The apparatus of claim 403 wherein:
the head of the fixation member is configured to be seated against the base second end; and
the body of the fixation member is configured to be seated in, and in abutment with, the cannula.

406. The apparatus of claim 403 wherein:
the first coupling mechanism includes a threaded bore; and
the support first end includes a threaded surface for threadedly engaging the threaded bore.

407. The apparatus of claim 406 wherein:
the threaded bore is a first threaded bore;
the second coupling mechanism is a second threaded bore; and
the elongated member includes threads configured to threadedly engage the second threaded bore.

408. The apparatus of claim 403 further comprising a plate, wherein:
a circumference of the head of the fixation member is configured to be seated in an opening defined in the plate; and,
in operation, the circumference is seated in the opening and a bottom face of the head is seated against the base second end.

409. The apparatus of claim 383 further comprising an extension member including the flange, wherein:
the support end is a support first end configured to be removably coupled to the hub;
the support includes a support second end, the extension member configured to be removably coupled to the second end;
wherein:
coupling the support first end to the hub, the extension member to the support second end and seating the flange on the base second end sets the upper limit of separation along the longitudinal axis between the hub and the base.

410. The apparatus of claim 409 wherein the support second end terminates inside the base when the support first end is coupled to the hub.

411. The apparatus of claim 409 further comprising a fixation member configured to be removably coupled to the extension member, wherein a head of the fixation member is configured to mate with a plate positioned on an outer surface of the bone.

412. The apparatus of claim 409 wherein:
the support first end includes a first support engagement feature configured to engage with the hub;
the support second end includes a second support engagement feature;
a first end of the extension member includes a first extension member engagement feature configured to engage the second support engagement feature;
a second end of the extension member includes a second extension member engagement feature configured to engage the fixation member;
wherein:
the flange is disposed between the first extension member engagement feature and the second extension member engagement feature.
